(19) United States
(12) Patent Application Publication
(10) Pub. No.: US 2024/0043382 A1
(43) Pub. Date: Feb. 8, 2024

(54) MODIFIED INDOLE ALKALOIDS FOR THERAPEUTIC USES

(71) Applicant: COMPASS PATHFINDER LIMITED, Altrincham, Cheshire (GB)

(72) Inventor: Ryan PROTZKO, Berkeley, CA (US)

(21) Appl. No.: 18/267,827

(22) PCT Filed: Dec. 17, 2021
(86) PCT No.: PCT/US2021/064209

§ 371 Date: Jun. 16, 2023

Related U.S. Application Data
(60) Provisional application No. 63/163,590, filed on Mar. 19, 2021.
(60) Provisional application No. 63/127,852, filed on Dec. 18, 2020.

(51) Int. Cl. C07D 209/16; C07D 471/14; C07D 471/18; C12P 19/60; C12P 17/18; C12P 17/10; C12P 11/00; C12P 9/00; C07F 9/572; C07H 17/02; C12N 9/12; C12N 9/10; C12N 9/88

(52) U.S. Cl. CPC C07D 209/16; C07D 471/14; C07D 471/18; C12P 19/60; C12P 17/182; C12P 17/10; C12P 11/00; C12P 9/00; C07F 9/5728; C07H 17/02; C12N 9/1205; C12N 9/1007; C12N 9/13; C12N 9/1033; C12N 9/1051; C12N 9/88; C12Y 201/01049; C12Y 201/01004; C12Y 203/01084; C12Y 204/01017; C12Y 402/0102

(57) ABSTRACT

Disclosed herein are enzymatic and modified host cell methods for prepared modified indole alkaloids. Also disclosed herein are modified indole alkaloids and their therapeutic use for treating diseases and disorders.

### CROSS-REFERENCES

This application claims the benefit of U.S. Provisional Application No. 63/127,852, filed Dec. 18, 2020, and U.S. Provisional Application No. 63/163,590, filed Mar. 19, 2021, each of which is hereby incorporated by reference in its entirety herein.

### SEQUENCE LISTINGS

The instant application contains a Sequence Listing which has been submitted electronically in ASCII format and is hereby incorporated by reference in its entirety. Said ASCII copy, created on Dec. 16, 2021, is named 54033-703_601_SL.txt and is 161,974 bytes in size.

### BACKGROUND

Indole alkaloids are a class of alkaloids containing a structural moiety of indole: many indole alkaloids also include isoprene groups and are thus called terpene indole or secologanin tryptamine alkaloids. Containing more than 4100 known different compounds, it is one of the largest classes of alkaloids. Many of them possess significant physiological activity and some of them are used in medicine. The amino acid tryptophan is the biochemical precursor of indole alkaloids.

The simple and widespread indole derivatives are the biogenic amines, tryptamine and 5-hydroxytryptamine (serotonin). The tryptamine skeleton is part of the vast majority of indole alkaloids. For example, N,N-dimethyltryptamine (DMT), psilocin and its phosphorylated psilocybin are simple derivatives of tryptamine. Another class includes β-carboline alkaloids which are accessed from tryptamine. One route includes the intramolecular Mannich reaction. Simple (non-isoprenoid) β-carboline derivatives include harmine, harmaline, harmane and a slightly more complex structure of canthinone. Harmaline was first isolated in 1838 by Göbel and harmine in 1848 by Fritzche.

A more complex group of indole alkaloids include ergot alkaloids. Ergot alkaloids are a class of hemiterpenoid indole alkaloids related to lysergic acid, which, in turn, is formed in multistage biosynthetic reactions involving tryptophan and DMAPP. Many ergot alkaloids are amides of lysergic acid. The simplest such amide is ergine. More complex groups including water-soluble amino alcohol derivatives, such as ergometrine and its isomer ergometrinine. Complex water-insoluble groups include the ergotamine group (including ergotamine, ergosine and their isomers), the ergoxine groups (including ergostine, ergoptine, ergonine and their isomers) and the ergotoxine group (including ergocristine, α-ergocryptine, β-ergocryptine, ergocomine and their isomers).

Mitragyna alkaloids are another indole-based alkaloid class and are abundant active alkaloids in the Southeast Asian plant Mitragyna speciosa, commonly known as kratom. The total alkaloid concentration in dried leaves ranges from 0.5 to 1.5%. In Thai varieties, mitragynine is the most abundant component (up to 66% of total alkaloids) while 7-hydroxymitragynine is a minor constituent (up to 2% of total alkaloid content).

Modifying indole alkaloids can significantly change their chemical and biological properties. For example, indole and indole alkaloids alone are scarcely soluble in water, whereas addition of a charged chemical functional group, such as a phosphate or carbohydrate, can increase water solubility. For biological systems, addition of such modifying functional groups can significantly alter the resulting biological activity or tissue targeting. For the end use of these compounds, modifications have major impacts on downstream formulations, preparations, pharmacokinetics, pharmacodynamics, and ultimate end uses. The modified indole alkaloids discussed herein have therapeutic uses including, but not limited to, treatment of major depression, treatment resistant depression, addiction, anxiety, post-traumatic stress disorder, mania, psychosis, insomnia, hypersomnia, pain, Alzheimer's disease, Parkinson's disease, cluster headaches, binge eating, migraine headaches, irritable bowel syndrome, and other neurological disorders. The modified indole alkaloids discussed herein may, in some cases, induce dendritic spine growth in neurons. In some cases, the therapeutic target of modified indole alkaloids is aminergic G-couple protein receptors (GPCRs). In some cases, the modified indole alkaloids are metabolized by the body and the resulting metabolite targets a protein or receptor implicated in disease. In some cases, the receptor is a GPCR. GPCRs implicated in disease that are therapeutic targets for modified indole alkaloids or respective metabolites include, but are not limited to, 5-hydroxytryptamine receptors. Modified indole alkaloids and resulting metabolites can be used for therapeutic targeting of HTR2A. Modified indole alkaloids and resulting metabolites can be used for therapeutic targeting of serotonin receptors. Modified indole alkaloids and resulting metabolites can be used for therapeutic targeting of melatonin receptors, including, but not limited to, MT1, MT2, and MT3. Modified indole alkaloids and resulting metabolites can be used for therapeutic targeting of opioid receptors, including, but not limited to, delta, kappa, mu, zeta, and nociceptin receptors.

In addition, solutions of known unmodified indole alkaloids have been reported to be unstable due to oxidation upon exposure to air and light. Novel modified indole alkaloids can overcome this lack of stability. Enzymatic modification of indole alkaloids is a strategy to alter the physicochemical properties of indole alkaloids, improving their stability and aqueous solubility (see, e.g., Gotvaldova et al., Drug Test Anal., 2021, 13, 439-446; Anastos et al., Science & Justice, 2006, 46(2), 91-96)

The synthesis of modified indole alkaloids, particularly esterification of indole alkaloids, is difficult by traditional organic synthesis methods. For example, the organic synthesis of psilocybin, the phosphorylated ester of psilocin, involves a 7-step synthesis protocol involving highly reactive substrates with protecting and de-protecting steps. However, the conversion of psilocin to psilocybin by enzymatic or bioconversion methods can be accessed in one step using aqueous and neutral reaction conditions. This is one example, among many, of the advantages afforded by enzymatic and biological production of esterified indole alkaloids. With the growing need for novel therapeutic indole alkaloids with diverse properties, there is a need in the art for expanded modification of indole alkaloids by biological conversion and to obtain modified indole alkaloids with improved physicochemical properties (e.g., stability to oxidation).

### SUMMARY

In one aspect, provided herein is a compound of Formula (Ia):

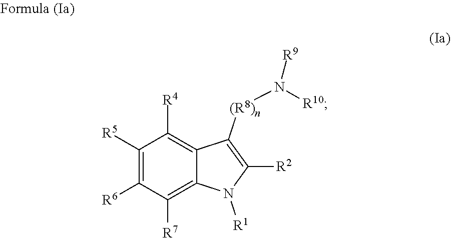

or a pharmaceutically acceptable salt thereof, wherein,
R¹ and R¹⁰ are independently selected from hydrogen, C₁-C₆ alkyl, C₂-C₆ alkenyl, and C₂-C₆ alkynyl, wherein alkyl, alkenyl, and alkynyl are optionally substituted with one or more substituents independently selected from halo, —OMe, —CN, —NH₂, and —NO₂;
R⁸ is —CR′₂—, wherein each R′ is independently selected from hydrogen, halo, haloalkyl, alkoxy, haloalkoxy, and amine;
n is selected from 2, 3, and 4;
R⁹ is selected from C₂-C₆ alkyl, C₂-C₆ alkenyl, and C₂-C₆ alkynyl, wherein alkyl, alkenyl, and alkynyl are optionally substituted with one or more substituents independently selected from halo, —OMe, —CN, —NH₂, —NO₂, and 3- to 8-membered heterocycle, and wherein 3- to 8-membered heterocycle is optionally substituted with one or more substituents independently selected from halo, —OMe, —CN, —NH₂, and —NO₂;
R² is selected from hydrogen, halogen, C₁-C₆ alkyl, and C₁-C₆ haloalkyl;
R⁴, R₅, R⁶, and R⁷ are each independently selected from hydrogen, C₁-C₆ alkyl, A, J, Q, and X;
A is

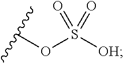 [SMILES: *OS(=O)(=O)O]

J is

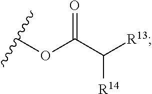

Q is

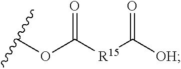

X is selected from glucose, xylose, galactose, rhamnose, rutinose, and disaccharide;
R¹³ is selected from hydrogen, and C₁-C₆ alkyl optionally substituted with one or more substituents independently selected from C₁-C₆ alkyl, oxo, halo, —OMe, —CN, —NH₂, and —NO₂;
R¹⁴ is selected from C₁-C₆ alkyl and C₂-C₆ alkenyl, wherein C₁-C₆ alkyl and C₂-C₆ alkenyl are optionally substituted with one or more substituents independently selected from halo, —OMe, —CN, —NH₂, and —NO₂;
or R¹³ and R¹⁴ taken together with the atom to which they are attached to form a substituted or unsubstituted C₃-C₈cycloalkyl or substituted or unsubstituted 3- to 8-membered heterocycloalkyl having 1 or 2 heteroatoms each independently selected from N, O, and S;
R¹⁵ is selected from C₁-C₆ alkylene and C₂-C₆ alkenylene, wherein C₁-C₆ alkylene and C₂-C₆ alkenylene are optionally substituted with one or more substituents independently selected from C₁-C₆ alkyl, halo, —OMe, —CN, —NH₂, and —NO₂; and
   wherein at least one of R⁴, R⁵, R⁶, and R₇ is A, J, Q, or X.

In certain embodiments, provided herein is a compound of Formula (I):

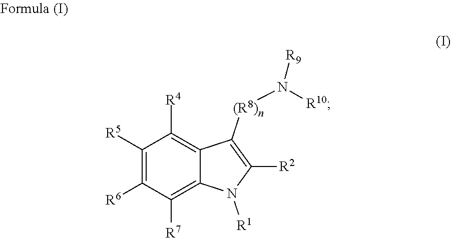

or a pharmaceutically acceptable salt thereof, wherein,
R¹ and R¹⁰ are independently selected from hydrogen, C₁-C₆ alkyl, C₂-C₆ alkenyl, and C₂-C₆ alkynyl, wherein alkyl, alkenyl, and alkynyl are optionally substituted with one or more substituents independently selected from halo, —OMe, —CN, —NH₂, and —NO₂;
R⁸ is —CR′₂—, wherein each R′ is independently selected from hydrogen, halo, haloalkyl, alkoxy, haloalkoxy, and amine;
n is selected from 2, 3, and 4;
R⁹ is selected from C₂-C₆ alkyl, C₂-C₆ alkenyl, and C₂-C₆ alkynyl, wherein alkyl, alkenyl, and alkynyl are optionally substituted with one or more substituents independently selected from halo, —OMe, —CN, —NH₂, —NO₂, and 3- to 8-membered heterocycle, and wherein 3- to 8-membered heterocycle is optionally substituted with one or more substituents independently selected from halo, —OMe, —CN, —NH₂, and —NO₂;
R² is selected from hydrogen, halogen, C₁-C₆ alkyl, and C₁-C₆ haloalkyl;
R⁴, R⁵, R⁶, and R⁷ are each independently selected from hydrogen, C₁-C₆ alkyl, A, J, Q, and X;
A is

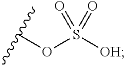 [SMILES: *OS(=O)(=O)O]

J is

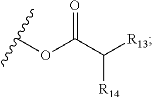

Q is

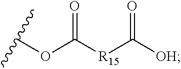

X is selected from glucose, xylose, galactose, rhamnose, and rutinose;
R¹³ is selected from hydrogen, and C₁-C₆ alkyl optionally substituted with one or more substituents independently selected from halo, —OMe, —CN, —NH₂, and —NO₂;
R¹⁴ is selected from C₁-C₆ alkyl and C₂-C₆ alkenyl, wherein C₁-C₆ alkyl and C₂-C₆ alkenyl are optionally substituted with one or more substituents independently selected from halo, —OMe, —CN, —NH₂, and —NO₂;
R¹⁵ is selected from C₁-C₆ alkylene and C₂-C₆ alkenylene, wherein C₁-C₆ alkylene and C₂-C₆ alkenylene are optionally substituted with one or more substituents independently selected from halo, —OMe, —CN, —NH₂, and —NO₂; and
   wherein at least one of R⁴, R⁵, R⁶, and R⁷ is A or Q, or at least one of R⁵, R⁶, and R⁷ is J or X.

In some embodiments, R¹ is selected from hydrogen and C₁-C₃ alkyl. In some embodiments, R¹ is hydrogen.

In some embodiments, R¹⁰ is independently selected from hydrogen, C₁-C₃ alkyl, and C₂-C₃ alkenyl, wherein alkyl and alkenyl are optionally substituted with one or more substituents independently selected from halo, —OMe, and —CN.

In some embodiments, each R′ is independently selected from hydrogen, halo, and haloalkyl. In some embodiments, each R′ is hydrogen.

In some embodiments, n is selected from 2 and 3. In some embodiments, n is 2.

In some embodiments, R^{y} is selected from C₂-C₃ alkyl and C₂-C₃ alkenyl, wherein alkyl and alkenyl are optionally substituted with one or more substituents independently selected from halo, —OMe, and —CN. In some embodiments, R⁹ is C₂-C₃ alkyl.

In some embodiments, R² is selected from hydrogen, halogen, and C₁-C₆ alkyl. In some embodiments, R² is hydrogen.

In some embodiments. R⁴, R⁵, R⁶, and R⁷ are each independently selected from hydrogen, A, J, Q, and X. In some embodiments, R⁴, R⁵, R⁶, and R⁷ are each independently selected from hydrogen, J, and Q. In some embodiments, at least one of R⁴, R⁵, R⁶, and R⁷ is A or Q. In some embodiments, at least one of R⁵, R⁶, and R⁷ is J or X.

In some embodiments, R¹³ is selected from hydrogen and C₁-C₃ alkyl optionally substituted with one or more substituents independently selected from halo, —OMe, and —CN. In some embodiments, R¹³ is hydrogen. In some embodiments, R¹³ is C₁-C₃ alkyl.

In some embodiments, R¹⁴ is C₁-C₃ alkyl optionally substituted with one or more substituents independently selected from halo, —OMe, and —CN.

In some embodiments, R¹⁵ is C₁-C₃ alkylene optionally substituted with one or more substituents independently selected from halo, —OMe, and —CN.

In some embodiments, X is selected from glucose, galactose, and rhamnose.

In another aspect, provided herein is a method of treating a disease or disorder in a subject in need thereof comprising administering a compound of Formula (Ia):

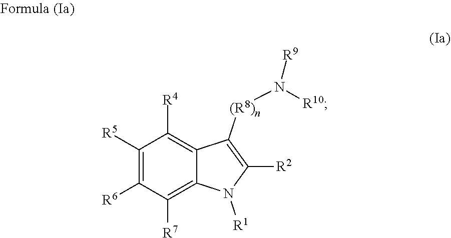

or a pharmaceutically acceptable salt thereof, wherein,
R¹ and R¹⁰ are independently selected from hydrogen, C₁-C₆ alkyl, C₂-C₆ alkenyl, and C₂-C₆ alkynyl, wherein alkyl, alkenyl, and alkynyl are optionally substituted with one or more substituents independently selected from halo, —OMe, —CN, —NH₂, and —NO₂;
R⁸ is —CR′₂—, wherein each R′ is independently selected from hydrogen, halo, haloalkyl, alkoxy, haloalkoxy, and amine;
n is selected from 2, 3, and 4;
R⁹ is selected from C₂-C₆ alkyl, C₂-C₆ alkenyl, and C₂-C₆ alkynyl, wherein alkyl, alkenyl, and alkynyl are optionally substituted with one or more substituents independently selected from halo, —OMe, —CN, —NH₂, —NO₂, and 3- to 8-membered heterocycle, and wherein 3- to 8-membered heterocycle is optionally substituted with one or more substituents independently selected from halo, —OMe, —CN, —NH₂, and —NO₂;
R² is selected from hydrogen, halogen, C₁-C₆ alkyl, and C₁-C₆ haloalkyl;
R⁴, R⁵, R⁶, and R⁷ are each independently selected from hydrogen, C₁-C₆ alkyl, A, J, Q, and X;
A is

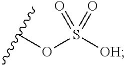 [SMILES: *OS(=O)(=O)O]

J is

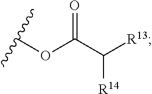

Q is

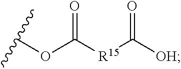

X is selected from glucose, xylose, galactose, rhamnose, rutinose, and disaccharide;
R¹³ is selected from hydrogen, and C₁-C₆ alkyl optionally substituted with one or more substituents independently selected from C₁-C₆ alkyl, oxo, halo, —OMe, —CN, —NH₂, and —NO₂;
R¹⁴ is selected from C₁-C₆ alkyl and C₂-C₆ alkenyl, wherein C₁-C₆ alkyl and C₂-C₆ alkenyl are optionally substituted with one or more substituents independently selected from halo, —OMe, —CN, —NH₂, and —NO₂;
or R¹³ and R^{1′} taken together with the atom to which they are attached to form a substituted or unsubstituted C₃-C₈cycloalkyl or substituted or unsubstituted 3- to 8-membered heterocycloalkyl having 1 or 2 heteroatoms each independently selected from N, O, and S;
R¹⁵ is selected from C₁-C₆ alkylene and C₂-C₆ alkenylene, wherein C₁-C₆ alkylene and C₂-C₆ alkenylene are optionally substituted with one or more substituents independently selected from C₁-C₆ alkyl, halo, —OMe, —CN, —NH₂, and —NO₂; and
wherein at least one of R⁴, R⁵, R⁶, and R is A, J, Q, or X.

In certain embodiments, provided herein is a method of treating a disease or disorder in a subject in need thereof comprising administering a compound of Formula (I):

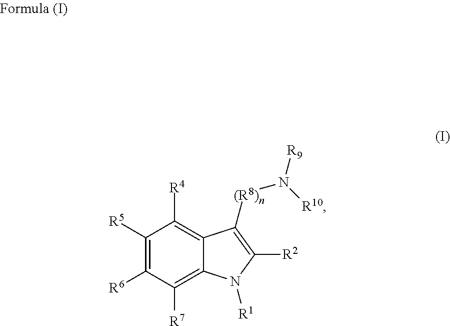

or a pharmaceutically acceptable salt thereof, wherein,
R¹ and R¹⁰ are independently selected from hydrogen, C₁-C₆ alkyl, C₂-C₆ alkenyl, and C₂-C₆ alkynyl, wherein alkyl, alkenyl, and alkynyl are optionally substituted with one or more substituents independently selected from halo, —OMe, —CN, —NH₂, and —NO₂;
R⁸ is —CR′₂—, wherein each R′ is independently selected from hydrogen, halo, haloalkyl, alkoxy, haloalkoxy, and amine;
n is selected from 2, 3, and 4;
R⁹ is selected from C₂-C₆ alkyl, C₂-C₆ alkenyl, and C₂-C₆ alkynyl, wherein alkyl, alkenyl, and alkynyl are optionally substituted with one or more substituents independently selected from halo, —OMe, —CN, —NH₂, —NO₂, and 3- to 8-membered heterocycle, and wherein 3- to 8-membered heterocycle is optionally substituted with one or more substituents independently selected from halo, —OMe, —CN, —NH₂, and —NO₂;
R² is selected from hydrogen, halogen, C₁-C₆ alkyl, and C₁-C₆ haloalkyl;
R⁴, R⁵, R⁶, and R⁷ are each independently selected from hydrogen, C₁-C₆ alkyl, A, J, Q, and X;
A is

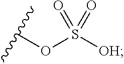 [SMILES: *OS(=O)(=O)O]

J is

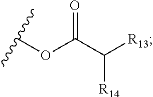

Q is

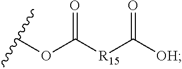

X is selected from glucose, xylose, galactose, rhamnose, and rutinose;
R¹³ is selected from hydrogen, and C₁-C₆ alkyl optionally substituted with one or more substituents independently selected from halo, —OMe, —CN, —NH₂, and —NO₂;
R¹⁴ is selected from C₁-C₆ alkyl and C₂-C₆ alkenyl, wherein C₁-C₆ alkyl and C₂-C₆ alkenyl are optionally substituted with one or more substituents independently selected from halo, —OMe, —CN, —NH₂, and —NO₂;
R¹⁵ is selected from C₁-C₆ alkylene and C₂-C₆ alkenylene, wherein C₁-C₆ alkylene and C₂-C₆ alkenylene are optionally substituted with one or more substituents independently selected from halo, —OMe, —CN, —NH₂, and —NO₂; and
   wherein at least one of R⁴, R⁵, R⁶, and R⁷ is A or Q, or at least one of R⁵, R⁶, and R⁷ is J or X.

In some embodiments, R¹ is selected from hydrogen and C₁-C₃ alkyl. In some embodiments, R¹ is hydrogen.

In some embodiments, R¹⁰ is independently selected from hydrogen, C₁-C₃ alkyl, and C₂-C₃ alkenyl, wherein alkyl and alkenyl are optionally substituted with one or more substituents independently selected from halo, —OMe, and —CN.

In some embodiments, each R′ is independently selected from hydrogen, halo, and haloalkyl. In some embodiments, each R′ is hydrogen.

In some embodiments, n is selected from 2 and 3. In some embodiments, n is 2.

In some embodiments, R⁹ is selected from C₂-C₃ alkyl and C₂-C₃ alkenyl, wherein alkyl and alkenyl are optionally substituted with one or more substituents independently selected from halo, —OMe, and —CN. In some embodiments, R⁹ is C₂-C₃ alkyl.

In some embodiments, R² is selected from hydrogen, halogen, and C₁-C₆ alkyl. In some embodiments, R² is hydrogen.

In some embodiments, R⁴, R⁵, R⁶, and R⁷ are each independently selected from hydrogen, A, J, Q, and X. In some embodiments, R⁴, R⁵, R⁶, and R⁷ are independently selected from hydrogen, J, and Q. In some embodiments, at least one of R⁴, R⁵, R⁶, and R⁷ is A or Q. In some embodiments, at least one of R⁵, R⁶, and R⁷ is J or X.

In some embodiments, R¹³ is selected from hydrogen and C₁-C₃ alkyl optionally substituted with one or more substituents independently selected from halo, —OMe, and —CN. In some embodiments, R¹³ is hydrogen. In some embodiments, R¹³ is C₁-C₃ alkyl.

In some embodiments, R¹⁴ is C₁-C₃ alkyl optionally substituted with one or more substituents independently selected from halo, —OMe, and —CN.

In some embodiments, R¹⁵ is C₁-C₃ alkylene optionally substituted with one or more substituents independently selected from halo, —OMe, and —CN.

In some embodiments, X is selected from glucose, galactose, and rhamnose.

In some embodiments, the disease or disorder is major depression, treatment resistant depression, addiction, anxiety, post-traumatic stress disorder, prolonged grief disorder, complicated grief disorder, mania, psychosis, insomnia, hypersomnia, pain, Alzheimer's disease, Parkinson's disease, burnout, cluster headaches, binge eating, migraine headaches, or irritable bowel syndrome. In some embodiments, the disease or disorder is major depression, treatment resistant depression, addiction, anxiety, post-traumatic stress disorder, prolonged grief disorder, complicated grief disorder, or binge eating.

In another aspect, provided herein is a method of treating a disease or disorder in a subject in need thereof comprising administering a modified indole alkaloid.

In some embodiments, the modified indole alkaloid is a modified tryptamine alkaloid, a modified ibogamine alkaloid, a modified ergoline alkaloid, a modified beta-carboline alkaloid, or a modified mitragynine alkaloid.

In some embodiments, the modified indole alkaloid is an acetylated indole alkaloid, an acylated indole alkaloid, a methylated indole alkaloid, a phosphorylated indole alkaloid, a sulfonated indole alkaloid, or a glycosylated indole alkaloid.

In yet another aspect, provided herein are a method of enzymatically preparing an indole alkaloid, comprising:
contacting a compound of Formula (Ia′) with an enzyme and a co-substrate;
wherein the compound of Formula (Ia′) has a structure of:

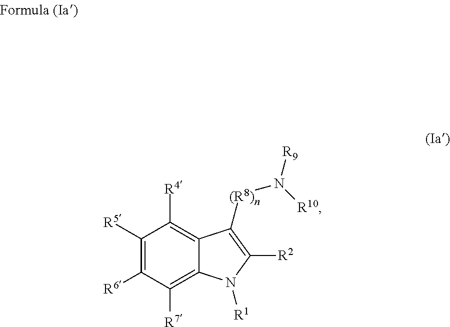

or a pharmaceutically acceptable salt thereof, wherein,
   R¹ and R¹⁰ are independently selected from hydrogen, C₁-C₆ alkyl, C₂-C₆ alkenyl, and C₂-C₆ alkynyl, wherein alkyl, alkenyl, and alkynyl are optionally substituted with one or more substituents independently selected from halo, —OMe, —CN, —NH₂, and —NO₂;
   R⁸ is —CR′₂—, wherein each R′ is independently selected from hydrogen, halo, haloalkyl, alkoxy, haloalkoxy, and amine;
   n is selected from 2, 3, and 4;
   R⁹ is selected from C₂-C₆ alkyl, C₂-C₆ alkenyl, and C₂-C₆ alkynyl, wherein alkyl, alkenyl, and alkynyl are optionally substituted with one or more substituents independently selected from halo, —OMe, —CN, —NH₂, —NO₂, and 3- to 8-membered heterocycle, and wherein 3- to 8-membered heterocycle is optionally substituted with one or more substituents independently selected from halo, —OMe, —CN, —NHL, and —NO₂;
   R² is selected from hydrogen, halogen, C₁-C₆ alkyl, and C₁-C₆ haloalkyl;
   R⁴, R⁵, R⁶, and R⁷ are each independently selected from hydrogen, —OH, and C₁-C₆ alkyl; and wherein at least one of R⁴, R⁵, R⁶, and R⁷ is —OH.

In some embodiments, R⁴ is —OH.

In some embodiments, R³ is —OH.

In some embodiments, R⁶ is —OH.

In some embodiments, R⁷ is —OH.

In some embodiments, the enzyme is a 4-hydroxytryptamine kinase.

In some embodiments, the enzyme is an acetylserotonin O-methyltransferase.

In some embodiments, the enzyme is a tryptamine n-methyltransferase.

In some embodiments, the enzyme is a sulfotransferase 1 A1.

In some embodiments, the enzyme is a sulfotransferase 1A3.

In some embodiments, the enzyme is an alcohol O-acetyltransferase 1.

In some embodiments, the enzyme is a chloramphenicol acetyltransferase.

In some embodiments, the enzyme is an UDP-glucuronosyltransferase. In some embodiments, the UDP-glucuronosyltransferase is an UDP-glucuronosyltransferase 1-6. In some embodiments, the UDP-glucuronosyltransferase is an UDP-glucuronosyltransferase 1-9.

In some embodiments, the UDP-glucuronosyltransferase is an UDP-glucuronosyltransferase 1-10.

In some embodiments, the enzyme is an oleandomycin glycosyltransferase.

In some embodiments, the enzyme is a glycosyltransferase.

In some embodiments, the enzyme is a 4-dimethylallyl tryptophan synthase.

In some embodiments, the enzyme is a 7-dimethylallyltryptophan synthase.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows SDS-PAGE analysis of purified transferase proteins compared to standard sized markers (L). Purified bands of protein product from their respective expression plasmids are indicated by arrows in each SDS-PAGE gel.

FIGS. 2A-2C show analysis of enzymatically produced 3-((3-(2-(diisopropylamino)ethyl)-1H-indol-4-yl)oxy)-3-oxopropanoic acid. FIG. 2A: LC-MS traces of the negative controls with uncharged CoA (reaction 1), heat-inactivated enzyme (reaction 2), and of product (reaction 3); FIG. 2B: MS spectrum of product in Reaction 3: FIG. 2C: UV-vis absorption spectrum of product in Reaction 3.

FIG. 3 illustrates relative ion counts of 2-((3-(2-(diisopropylamino)ethyl)-1H-indol-4-yl)oxy)-6-(hydroxymethyl)tetrahydro-2H-pyran-3,4,5-triol product in Reactions 4-8, in which various glucosyltransferase enzymes were applied to enzymatically glycosylate 4-hydroxy-N,N-diisopropyltryptamine.

FIG. 4 illustrates relative ion counts of 3-(2-(diisopropylamino)ethyl)-1H-indol-4-yl hydrogen sulfate product in Reactions 9-11, in which various sulfotransferase enzymes were applied to enzymatically sulfonate 4-hydroxy-N,N-diisopropyltryptamine.

FIG. 5 shows functional agonism of 5HT2A receptor, measured by peak calcium flux response, by 4-3-(2-(dipropylamino)ethyl)-1H-indol-4-yl dihydrogen phosphate (triangle) and 4-3-(2-(diispropylamino)ethyl)-1H-indol-4-yl dihydrogen phosphate (square) when treated with calf alkaline phosphatase, as compared to 5-hydroxytryptamine (circle).

FIGS. 6A-6B show LC-MS traces of 2-((3-(2-(diisopropylamino)ethyl)-1H-indol-4-yl)oxy)-6-(hydroxymethyl)tetrahydro-2H-pyran-3,4,5-triol (Compound C) sample alone (Traces A and C) and the sample treated with human saliva samples (Traces B and D). FIG. 6A: traces A and B, a m/z of 423.24, which corresponds to the glycosylated product from Reaction 47, was extracted. FIG. 6B: traces C and D, a m/z of 261.19, which corresponds to the deglycosylated product 4-hydroxy-N,N-diisopropyltryptamine, was extracted.

FIGS. 7A-7B show LC-MS traces of 3-((3-(2-(diisopropylamino)ethyl)-1H-indol-4-yl)oxy)-3-oxopropanoic acid (Compound D) sample alone (Traces A and C) and the sample treated with human saliva samples (Traces B and D). FIG. 7A: traces A and B, a m/z of 347.19, which corresponds to the malonylated product from Reaction 3, was extracted. FIG. 7B: traces C and D, a m/z of 261.19, which corresponds to the hydrolyzed product 4-hydroxy-N,N-diisopropyltryptamine, was extracted.

FIGS. 8A-8B show LC-MS traces of 3-(2-(diisopropylamino)ethyl)-1H-indol-4-yl 3-oxobutanoate (Compound E) sample alone (Traces A and C) and the sample treated with human saliva samples (Traces B and D). FIG. 8A: traces A and B, a m/z of 345.21, which corresponds to the acetylated product from Reaction 3, was extracted. FIG. 8B: traces C and D, a m/z of 261.19, which corresponds to the hydrolyzed product 4-hydroxy-N,N-diisopropyltryptamine, was extracted.

### DETAILED DESCRIPTION

Provided herein are novel methods for production of modified indole alkaloids with therapeutic properties. The modification of indole alkaloids with these methods can lead to enhanced therapeutic features such as increased solubility, increased bioavailability, concentrating drug to therapeutic targets within the body, and therapeutic pharmacokinetic profiles. The methods described herein can modify indole alkaloids in chemical classes including, but not limited to, tryptamine, ergoline, mitragyna alkaloid, β-carboline, and ibogamine compound classes.

Definitions

Unless defined otherwise, all technical and scientific terms used herein have the same meaning as is commonly understood by one of skill in the art to which this disclosure belongs.

As used herein, the singular form “a”, “an” and “the” includes plural references unless the context clearly dictates otherwise.

The term “C_{x-y}” when used in conjunction with a chemical moiety, such as alkyl, alkenyl, or alkynyl is meant to include groups that contain from x to y carbons in the chain. For example, the term “C₁₋₆alkyl” refers to substituted or unsubstituted saturated hydrocarbon groups, including straight-chain alkyl and branched-chain alkyl groups that contain from 1 to 6 carbons.

The term −C_{x-y}alkylene—refers to a substituted or unsubstituted alkylene chain with from x to y carbons in the alkylene chain. For example —C₁₋₆alkylene—may be selected from methylene, ethylene, propylene, butylene, pentylene, and hexylene, any one of which is optionally substituted.

“Alkyl” refers to substituted or unsubstituted saturated hydrocarbon groups, including straight-chain alkyl and branched-chain alkyl groups. An alkyl group may contain from one to twelve carbon atoms (e.g., C₁₋₁₂ alkyl), such as one to eight carbon atoms (C₁₋₈ alkyl) or one to six carbon atoms (C₁₋₆ alkyl). Exemplary alkyl groups include methyl, ethyl, n-propyl, isopropyl, n-butyl, isobutyl, sec-butyl, tert-butyl, pentyl, isopentyl, neopentyl, hexyl, septyl, octyl, nonyl, and decyl. An alkyl group is attached to the rest of the molecule by a single bond. Unless stated otherwise specifically in the specification, an alkyl group is optionally substituted by one or more substituents such as those substituents described herein.

“Alkylene” refers to a straight or branched divalent hydrocarbon chain. Unless stated otherwise specifically in the specification, an alkylene group may be optionally substituted by one or more substituents such as those substituents described herein.

“Haloalkyl” refers to an alkyl group that is substituted by one or more halogens. Exemplary haloalkyl groups include trifluoromethyl, difluoromethyl, trichloromethyl, 2,2,2-trifluoroethyl, 1,2-difluoroethyl, 3-bromo-2-fluoropropyl, and 1,2-dibromoethyl.

“Alkenyl” refers to substituted or unsubstituted hydrocarbon groups, including straight-chain or branched-chain alkenyl groups containing at least one double bond. An alkenyl group may contain from two to twelve carbon atoms (e.g., C₂₋₁₂ alkenyl). Exemplary alkenyl groups include ethenyl (i.e., vinyl), prop-1-enyl, but-1-enyl, pent-1-enyl, penta-1,4-dienyl, and the like. Unless stated otherwise specifically in the specification, an alkenyl group is optionally substituted by one or more substituents such as those substituents described herein.

“Alkenylene” refers to a straight or branched divalent hydrocarbon chain containing at least one double bond. Unless stated otherwise specifically in the specification, an alkenylene group may be optionally substituted by one or more substituents such as those substituents described herein.

“Alkynyl” refers to substituted or unsubstituted hydrocarbon groups, including straight-chain or branched-chain alkynyl groups containing at least one triple bond. An alkynyl group may contain from two to twelve carbon atoms (e.g., C₂₋₁₂ alkynyl). Exemplary alkynyl groups include ethynyl, propynyl, butynyl, pentynyl, hexynyl, and the like. Unless stated otherwise specifically in the specification, an alkynyl group is optionally substituted by one or more substituents such as those substituents described herein.

“Alkynylene” refers to a straight or branched divalent hydrocarbon chain containing at least one triple bond. Unless stated otherwise specifically in the specification, an alkynylene group may be optionally substituted by one or more substituents such as those substituents described herein.

“Aryl” refers to an aromatic ring wherein each of the atoms forming the ring is a carbon atom. Aryl groups can be optionally substituted. Examples of aryl groups include, but are not limited to, phenyl and naphthyl. In some embodiments, the aryl is phenyl. Depending on the structure, an aryl group can be a monoradical or a diradical (i.e., an arylene group). Unless stated otherwise specifically in the specification, the term “aryl” or the prefix “ar-” (such as in “aralkyl”) is meant to include aryl radicals that are optionally substituted.

“Heteroaryl” refers to a 3- to 12-membered aromatic ring that comprises at least one heteroatom wherein each heteroatom may be independently selected from N, O, and S. As used herein, the heteroaryl ring may be selected from monocyclic or bicyclic and fused or bridged ring systems wherein at least one of the rings in the ring system is aromatic, i.e., it contains a cyclic, delocalized (4n+2)π-electron system in accordance with the Hickel theory. The heteroatom(s) in the heteroaryl may be optionally oxidized. One or more nitrogen atoms, if present, are optionally quaternized. The heteroaryl may be attached to the rest of the molecule through any atom of the heteroaryl, valence permitting, such as a carbon or nitrogen atom of the heteroaryl. Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pyridinyl, and thiophenyl (i.e. thienyl). Unless stated otherwise specifically in the specification, a heteroaryl is optionally substituted by one or more substituents such as those substituents described herein.

The term “cycloalkyl” refers to a monocyclic or polycyclic non-aromatic radical, wherein each of the atoms forming the ring (i.e. skeletal atoms) is a carbon atom. In some embodiments, cycloalkyls are saturated or partially unsaturated. In some embodiments, cycloalkyls are spirocyclic or bridged compounds. In some embodiments, cycloalkyls are fused with an aromatic ring (in which case the cycloalkyl is bonded through a non-aromatic ring carbon atom). Cycloalkyl groups include groups having from 3 to 10 ring atoms. Representative cycloalkyls include, but are not limited to, cycloalkyls having from three to ten carbon atoms, from three to eight carbon atoms, from three to six carbon atoms, or from three to five carbon atoms. Monocyclic cycloalkyl radicals include, for example, cyclopropyl, cyclobutyl, cyclopentyl, cyclohexyl, cycloheptyl, and cyclooctyl. Polycyclic radicals include, for example, adamantyl, 1,2-dihydronaphthalenyl, 1,4-dihydronaphthalenyl, tetrainyl, decalinyl, 3,4-dihydronaphthalenyl-1(2H)-one, spiro[2.2]pentyl, norbornyl and bicycle[1.1.1]pentyl. Unless otherwise stated specifically in the specification, a cycloalkyl group may be optionally substituted.

The term “heterocycloalkyl” refers to a cycloalkyl group that includes at least one heteroatom selected from nitrogen, oxygen, and sulfur. Unless stated otherwise specifically in the specification, the heterocycloalkyl radical may be a monocyclic, or bicyclic ring system, which may include fused (when fused with an aryl or a heteroaryl ring, the heterocycloalkyl is bonded through a non-aromatic ring atom) or bridged ring systems. The nitrogen, carbon or sulfur atoms in the heterocyclyl radical may be optionally oxidized. The nitrogen atom may be optionally quaternized. The heterocycloalkyl radical may be partially or fully saturated. Examples of heterocycloalkyl radicals include, but are not limited to, dioxolanyl, thienyl[1,3]dithianyl, tetrahydroquinolyl, tetrahydroisoquinolyl, decahydroquinolyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl. The term heterocycloalkyl also includes all ring forms of carbohydrates, including but not limited to monosaccharides, disaccharides and oligosaccharides. Unless otherwise noted, heterocycloalkyls have from 2 to 12 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Unless stated otherwise specifically in the specification, a heterocycloalkyl group may be optionally substituted.

An “alkoxy” refers to a “—O-alkyl” group, where alkyl is as defined herein.

The term “halo” or, alternatively, “halogen” means fluoro, chloro, bromo and iodo.

The term “substituted” refers to moieties having substituents replacing a hydrogen on one or more carbons or heteroatoms of the structure. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, a carbocycle, a heterocycle, a cycloalkyl, a heterocycloalkyl, an aromatic and heteroaromatic moiety.

It will be understood by those skilled in the art that substituents can themselves be substituted, if appropriate. Unless specifically stated as “unsubstituted,” references to chemical moieties herein are understood to include substituted variants. For example, reference to a “heteroaryl” group or moiety implicitly includes both substituted and unsubstituted variants.

Where substituent groups are specified by their conventional chemical formulae, written from left to right, they equally encompass the chemically identical substituents that would result from writing the structure from right to left, e.g., —CH₂O— is equivalent to —OCH₂—.

“Optional” or “optionally” means that the subsequently described event of circumstances may or may not occur, and that the description includes instances where the event or circumstance occurs and instances in which it does not. For example, “optionally substituted aryl” means that the aryl group may or may not be substituted and that the description includes both substituted aryl groups and aryl groups having no substitution.

Compounds of the present disclosure also include crystalline and amorphous forms of those compounds, pharmaceutically acceptable salts, and active metabolites of these compounds having the same type of activity, including, for example, polymorphs, pseudopolymorphs, solvates, hydrates, unsolvated polymorphs (including anhydrates), conformational polymorphs, and amorphous forms of the compounds, as well as mixtures thereof.

The compounds described herein may exhibit their natural isotopic abundance, or one or more of the atoms may be artificially enriched in a particular isotope having the same atomic number, but an atomic mass or mass number different from the atomic mass or mass number predominantly found in nature. All isotopic variations of the compounds of the present disclosure, whether radioactive or not, are encompassed within the scope of the present disclosure. For example, hydrogen has three naturally occurring isotopes, denoted ¹H (protium), ²H (deuterium), and ³H (tritium). Protium is the most abundant isotope of hydrogen in nature.

Enriching for deuterium may afford certain therapeutic advantages, such as increased in vivo half-life and/or exposure, or may provide a compound useful for investigating in vivo routes of drug elimination and metabolism. Isotopically-enriched compounds may be prepared by conventional techniques well known to those skilled in the art.

“Isomers” are different compounds that have the same molecular formula. “Stereoisomers” are isomers that differ only in the way the atoms are arranged in space. “Enantiomers” are a pair of stereoisomers that are non-superimposable mirror images of each other. A 1:1 mixture of a pair of enantiomers is a “racemic” mixture. The term “(+)” is used to designate a racemic mixture where appropriate. “Diastereoisomers” or “diastereomers” are stereoisomers that have at least two asymmetric atoms but are not mirror images of each other. The absolute stereochemistry is specified according to the Cahn-Ingold-Prelog R-S system. When a compound is a pure enantiomer, the stereochemistry at each chiral carbon can be specified by either R or S. Resolved compounds whose absolute configuration is unknown can be designated (+) or (−) depending on the direction (dextro- or levorotatory) in which they rotate plane polarized light at the wavelength of the sodium D line. Certain compounds described herein contain one or more asymmetric centers and can thus give rise to enantiomers, diastereomers, and other stereoisomeric forms, the asymmetric centers of which can be defined, in terms of absolute stereochemistry, as (R)- or (S)-. The present chemical entities, pharmaceutical compositions and methods are meant to include all such possible stereoisomers, including racemic mixtures, optically pure forms, mixtures of diastereomers and intermediate mixtures. Optically active (R)- and (S)-isomers can be prepared using chiral synthons or chiral reagents, or resolved using conventional techniques. The optical activity of a compound can be analyzed via any suitable method, including but not limited to chiral chromatography and polarimetry, and the degree of predominance of one stereoisomer over the other isomer can be determined.

Chemical entities having carbon-carbon double bonds or carbon-nitrogen double bonds may exist in Z- or E- form (or cis- or trans-form). Furthermore, some chemical entities may exist in various tautomeric forms. Unless otherwise specified, chemical entities described herein are intended to include all Z-, E- and tautomeric forms as well.

Isolation and purification of the chemical entities and intermediates described herein can be effected, if desired, by any suitable separation or purification procedure such as, for example, filtration, extraction, crystallization, column chromatography, thin-layer chromatography or thick-layer chromatography, or a combination of these procedures. Specific illustrations of suitable separation and isolation procedures can be had by reference to the examples herein below. However, other equivalent separation or isolation procedures can also be used.

When stereochemistry is not specified, certain small molecules described herein include, but are not limited to, when possible, their isomers, such as enantiomers and diastereomers, mixtures of enantiomers, including racemates, mixtures of diastereomers, and other mixtures thereof, to the extent they can be made by one of ordinary skill in the art by routine experimentation. In those situations, the single enantiomers or diastereomers, i.e., optically active forms, can be obtained by asymmetric synthesis or by resolution of the racemates or mixtures of diastereomers. Resolution of the racemates or mixtures of diastereomers, if possible, can be accomplished, for example, by conventional methods such as crystallization in the presence of a resolving agent, or chromatography, using, for example, a chiral high-pressure liquid chromatography (HPLC) column. Furthermore, a mixture of two enantiomers enriched in one of the two can be purified to provide further optically enriched form of the major enantiomer by recrystallization and/or trituration. In addition, such certain small molecules include Z- and E-forms (or cis- and trans-forms) of certain small molecules with carbon-carbon double bonds or carbon-nitrogen double bonds. Where certain small molecules described herein exist in various tautomeric forms, the term “certain small molecule” is intended to include all tautomeric forms of the certain small molecule.

The term “salt” or “pharmaceutically acceptable salt” refers to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine.

In some embodiments, the pharmaceutically acceptable base addition salt is chosen from ammonium, potassium, sodium, calcium, and magnesium salts.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “effective amount” or “therapeutically effective amount” refers to that amount of a compound described herein that is sufficient to affect the intended application, including but not limited to disease treatment, as defined below. The therapeutically effective amount may vary depending upon the intended treatment application (in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of platelet adhesion and/or cell migration. The specific dose will vary depending on the particular compounds chosen, the dosing regimen to be followed, whether it is administered in combination with other compounds, timing of administration, the tissue to which it is administered, and the physical delivery system in which it is carried.

As used herein, “treatment” or “treating” refers to an approach for obtaining beneficial or desired results with respect to a disease, disorder, or medical condition including but not limited to a therapeutic benefit and/or a prophylactic benefit. A therapeutic benefit can include, for example, the eradication or amelioration of the underlying disorder being treated. Also, a therapeutic benefit can include, for example, the eradication or amelioration of one or more of the physiological symptoms associated with the underlying disorder such that an improvement is observed in the subject, notwithstanding that the subject may still be afflicted with the underlying disorder. In certain embodiments, for prophylactic benefit, the compositions are administered to a subject at risk of developing a particular disease, or to a subject reporting one or more of the physiological symptoms of a disease, even though a diagnosis of this disease may not have been made.

### Modified Indole Alkaloids

Functional groups can be transferred from donor molecules to acceptor indole alkaloids using enzymes and biological systems containing enzymes to form modified indole alkaloids. The indole alkaloid has the basic structure of indole. In some cases, the indole alkaloid includes substituted indoles containing acceptor functional groups for transferred donor molecules, forming modified indole alkaloids. In some cases, the indole alkaloid includes substituted tryptamines containing acceptor functional groups for transferred donor molecules, forming modified tryptamines. In some cases, the indole alkaloid includes substituted beta-carbolines containing acceptor functional groups for transferred donor molecules, forming modified beta-carbolines. In some cases, the indole alkaloid includes substituted ergolines containing acceptor functional groups for transferred donor molecules, forming modified ergolines. In some cases, the indole alkaloid includes myragyna alkaloids containing acceptor functional groups for transferred donor molecules, forming modified myragyna alkaloids. In some cases, the indole alkaloid includes ibogamine alkaloids containing acceptor functional groups for transferred donor molecules, forming modified ibogamine alkaloids.

The action of transferring functional groups can include glycosylation, in which a carbohydrate, i.e. a glycosyl donor, is attached to a hydroxyl or other functional group of another molecule (a glycosyl acceptor). This forms a glucoside form of an indole alkaloid. This action of transferring functional groups can include phosphorylation, in which a kinase or phosphotransferase enzyme transfers a phosphoryl group to a hydroxyl or other functional group (a phosphoryl acceptor). This forms a phosphorylated form of the indole alkaloid. This action of transferring functional groups can include sulfonation, in which a sulfate group, i.e. sulfate donor, is attached to a hydroxyl or other functional group of another molecule (a sulfate acceptor). This forms a sulfate form of an indole alkaloid. This action of transferring functional groups can include methylation, in which a methyltransferase enzyme transfers a methyl group, i.e. methyl donor, to a hydroxyl or other functional group (a methyl acceptor). This forms a methylated form of the indole alkaloid. This action of transferring functional groups can include acylation, in which an acyl group, i.e, acyl donor, is attached to a hydroxyl or other functional group of another molecule (an acyl acceptor). This forms an acyl form of an indole alkaloid.

In one aspect, provided herein is a compound of Formula (Ia):

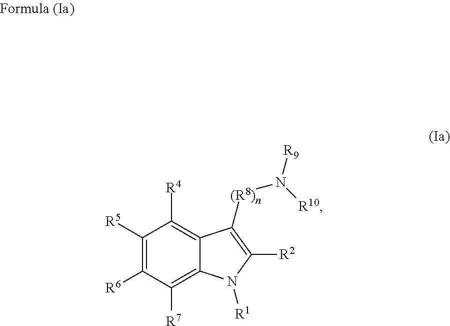

or a pharmaceutically acceptable salt thereof, wherein,
R¹ and R¹⁰ are independently selected from hydrogen, C₁-C₆ alkyl, C₂-C₆ alkenyl, and C₂-C₆ alkynyl, wherein alkyl, alkenyl, and alkynyl are optionally substituted with one or more substituents independently selected from halo, —OMe, —CN, —NH₂, and —NO₂;
R⁸ is —CR′₂—, wherein each R′ is independently selected from hydrogen, halo, haloalkyl, alkoxy, haloalkoxy, and amine;
n is selected from 2, 3, and 4;
R⁹ is selected from C₂-C₆ alkyl, C₂-C₆ alkenyl, and C₂-C₆ alkynyl, wherein alkyl, alkenyl, and alkynyl are optionally substituted with one or more substituents independently selected from halo, —OMe, —CN, —NH₂, —NO₂, and 3- to 8-membered heterocycle, and wherein 3- to 8-membered heterocycle is optionally substituted with one or more substituents independently selected from halo, —OMe, —CN, —NH₂, and —NO₂;
R² is selected from hydrogen, halogen, C₁-C₆ alkyl, and C₁-C₆ haloalkyl;
R⁴, R⁵, R⁶, and R⁷ are each independently selected from hydrogen, C₁-C₆ alkyl, A, J, Q, and X;
A is

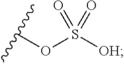 [SMILES: *OS(=O)(=O)O]

J is

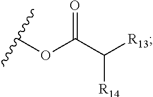

Q is

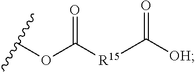

X is selected from glucose, xylose, galactose, rhamnose, rutinose, and disaccharide;
R¹³ is selected from hydrogen, and C₁-C₆ alkyl optionally substituted with one or more substituents independently selected from C₁-C₆ alkyl, oxo, halo, —OMe, —CN, —NH₂, and —NO₂;
R¹⁴ is selected from C₁-C₆ alkyl and C₂-C₆ alkenyl, wherein C₁-C₆ alkyl and C₂-C₆ alkenyl are optionally substituted with one or more substituents independently selected from halo, —OMe, —CN, —NH₂, and —NO₂;
or R¹³ and R¹⁴ taken together with the atom to which they are attached to form a substituted or unsubstituted C₃-C₈cycloalkyl or substituted or unsubstituted 3- to 8-membered heterocycloalkyl having 1 or 2 heteroatoms each independently selected from N, O, and S;
R¹⁵ is selected from C₁-C₆ alkylene and C₂-C₆ alkenylene, wherein C₁-C₆ alkylene and C₂-C₆ alkenylene are optionally substituted with one or more substituents independently selected from C₁-C₆ alkyl, halo, —OMe, —CN, —NH₂, and —NO₂; and
   wherein at least one of R⁴, R⁵, R⁶, and R⁷ is A, J, Q, or X.

In certain embodiments, provided herein is a compound of Formula (I):

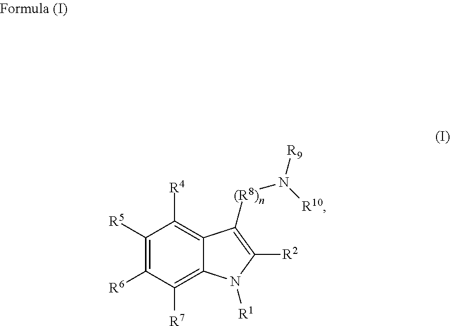

or a pharmaceutically acceptable salt thereof, wherein,
R¹ and R¹⁰ are independently selected from hydrogen, C₁-C₆ alkyl, C₂-C₆ alkenyl, and C₂-C₆ alkynyl, wherein alkyl, alkenyl, and alkynyl are optionally substituted with one or more substituents independently selected from halo, —OMe, —CN, —NH₂, and —NO₂;
R⁸ is —CR′₂—, wherein each R′ is independently selected from hydrogen, halo, haloalkyl, alkoxy, haloalkoxy, and amine;
n is selected from 2, 3, and 4;
R⁹ is selected from C₂-C₆ alkyl, C₂-C₆ alkenyl, and C₂-C₆ alkynyl, wherein alkyl, alkenyl, and alkynyl are optionally substituted with one or more substituents independently selected from halo, —OMe, —CN, —NH₂, —NO₂, and 3- to 8-membered heterocycle, and wherein 3- to 8-membered heterocycle is optionally substituted with one or more substituents independently selected from halo, —OMe, —CN, —NH₂, and —NO₂;
R² is selected from hydrogen, halogen, C₁-C₆ alkyl, and C₁-C₆ haloalkyl;
R⁴, R⁵, R⁶, and R⁷ are each independently selected from hydrogen, C₁-C₆ alkyl, A, J, Q, and X;
A is

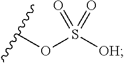 [SMILES: *OS(=O)(=O)O]

J is

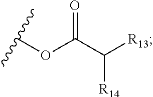

Q is

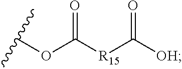

X is selected from glucose, xylose, galactose, rhamnose, and rutinose;
R¹³ is selected from hydrogen, and C₁-C₆ alkyl optionally substituted with one or more substituents independently selected from halo, —OMe, —CN, —NH₂, and —NO₂;
R¹⁴ is selected from C₁-C₆ alkyl and C₂-C₆ alkenyl, wherein C₁-C₆ alkyl and C₂-C₆ alkenyl are optionally substituted with one or more substituents independently selected from halo, —OMe, —CN, —NH₂, and —NO₂;
R¹⁵ is selected from C₁-C₆ alkylene and C₂-C₆ alkenylene, wherein C₁-C₆ alkylene and C₂-C₆ alkenylene are optionally substituted with one or more substituents independently selected from halo, —OMe, —CN, —NH₂, and —NO₂; and
   wherein at least one of R⁴, R⁵, R⁶, and R⁷ is A or Q, or at least one of R⁵, R⁶, and R⁷ is J or X.

In some embodiments, R¹ is selected from hydrogen, C₁-C₆ alkyl, C₂-C₆ alkenyl, and C₂-C₆ alkynyl, wherein alkyl, alkenyl, and alkynyl are optionally substituted with one or more substituents independently selected from halo, —OMe, —CN, —NH₂, and —NO₂. In some embodiments, R¹ is selected from hydrogen, C₁-C₆ alkyl, and C₂-C₆ alkenyl, wherein alkyl and alkenyl are optionally substituted with one or more substituents independently selected from halo, —OMe, —CN, —NH₂, and —NO₂. In some embodiments, R¹ is selected from hydrogen and C₁-C₆ alkyl, wherein alkyl is optionally substituted with one or more substituents independently selected from halo. —OMe, and —CN. In some embodiments, R¹ is selected from hydrogen and C₁-C₃ alkyl. In some embodiments, R¹ is hydrogen. In some embodiments, R¹ is C₁-C₃ alkyl.

In some embodiments, R¹ is selected from hydrogen. C₁-C₆ alkyl, C₂-C₆ alkenyl, and C₂-C₆ alkynyl, wherein alkyl, alkenyl, and alkynyl are optionally substituted with one or more substituents independently selected from halo, —OMe, —CN, —NH₂, and —NO₂. In some embodiments, R¹⁰ is selected from hydrogen, C₁-C₆ alkyl, and C₂-C₆ alkenyl, wherein alkyl and alkenyl are optionally substituted with one or more substituents independently selected from halo, —OMe, —CN, —NH₂, and —NO₂. In some embodiments, R¹⁰ is selected from hydrogen, C₁-C₃ alkyl, and C₂-C₃ alkenyl, wherein alkyl and alkenyl are optionally substituted with one or more substituents independently selected from halo, —OMe, and —CN. In some embodiments, R¹⁰ is independently selected from hydrogen, C₁-C₃ alkyl, and C₂-C₃ alkenyl. In some embodiments, R¹⁰ is hydrogen. In some embodiments, R¹⁰ is C₁-C₃ alkyl. In some embodiments, R¹⁰ is C₂-C₃ alkenyl.

In some embodiments, each R′ is independently selected from hydrogen, halo, haloalkyl, alkoxy, haloalkoxy, and amine. In some embodiments, each R′ is independently selected from hydrogen, halo, and haloalkyl. In some embodiments, each R′ is hydrogen. In some embodiments, each R′ is halo. In some embodiments, each R′ is haloalkyl. In some embodiments, each R′ is alkoxy. In some embodiments, each R′ is haloalkoxy. In some embodiments, each R′ is amine.

In some embodiments, n is selected from 2, 3, and 4. In some embodiments, n is selected from 2 and 3. In some embodiments, n is 2. In some embodiments, n is 3. In some embodiments, n is 4.

In some embodiments, R⁹ is selected from C₂-C₆ alkyl, C₂-C₆ alkenyl, and C₂-C₆ alkynyl, wherein alkyl, alkenyl, and alkynyl are optionally substituted with one or more substituents independently selected from halo, —OMe, —CN, —NH₂, —NO₂, and 3- to 8-membered heterocycle, and wherein 3- to 8-membered heterocycle is optionally substituted with one or more substituents independently selected from halo, —OMe, —CN, —NH₂, and —NO₂. In some embodiments, R⁹ is selected from C₂-C₆ alkyl, C₂-C₆ alkenyl, and C₂-C₆ alkynyl, wherein alkyl, alkenyl, and alkynyl are optionally substituted with one or more substituents independently selected from halo, —OMe, —CN, —NH₂, —NO₂, and 3- to 8-membered heterocycle, and wherein 3-to 8-membered heterocycle is optionally substituted with one or more substituents independently selected from halo, —OMe, and —CN. In some embodiments, R⁹ is selected from C₂-C₆ alkyl and C₂-C₆ alkenyl, wherein alkyl and alkenyl are optionally substituted with one or more substituents independently selected from halo, —OMe, —CN, and —NH₂. In some embodiments, R⁹ is selected from C₂-C₃ alkyl and C₂-C₃ alkenyl, wherein alkyl and alkenyl are optionally substituted with one or more substituents independently selected from halo, —OMe, and —CN. In some embodiments, R⁹ is C₂-C₃ alkyl. In some embodiments, R⁹ is C₂-C₃ alkenyl.

In some embodiments, R² is selected from hydrogen, halogen, C₁-C₆ alkyl, and C₁-C₆ haloalkyl. In some embodiments, R² is selected from hydrogen, halogen, and C₁-C₆ alkyl. In some embodiments, R² is hydrogen. In some embodiments, R² is halogen. In some embodiments, R² is C₁-C₆ alkyl. In some embodiments, R² is C₁-C₆ haloalkyl.

In some embodiments. R⁴, R⁵, R⁶, and R⁷ are each independently selected from hydrogen, A, J, Q, and X. In some embodiments, R⁴, R⁵, R⁶, and R⁷ are each independently selected from hydrogen, J, and Q. In some embodiments, at least one of R⁴, R⁵, R⁶, and R⁷ is A or Q. In some embodiments, at least one of R⁵, R⁶, and R⁷ is J or X. In some embodiments, at least one of R⁴, R⁵, R⁶, and R⁷ is A. In some embodiments, R⁴ is A. In some embodiments, R⁵ is A. In some embodiments, R⁶ is A. In some embodiments, R⁷ is A. In some embodiments, at least one of R⁴, R⁵, R⁶, and R⁷ is Q. In some embodiments, R⁴ is Q. In some embodiments, R⁵ is Q. In some embodiments, R⁶ is Q. In some embodiments, R⁷ is Q. In some embodiments, at least one of R⁵, R⁶, and R⁷ is J. In some embodiments, R⁵ is J. In some embodiments, R⁶ is J. In some embodiments, R⁷ is J. In some embodiments, at least one of R⁵, R⁶, and R⁷ is X. In some embodiments, R⁵ is X. In some embodiments, R⁶ is X. In some embodiments, R⁷ is X.

In some embodiments, at least one of R⁴, R⁵, R⁶, and R⁷ is A, J, Q, or X. In some embodiments, at least one of R⁴, R⁵, and R⁶ is A, J, Q, or X. In some embodiments, at least one of R⁴, R⁵, and R⁷ is A, J, Q, or X. In some embodiments, at least one of R⁵, R⁶, and R⁷ is A, J, Q, or X. In some embodiments, at least one of R⁴ and R₇ is A, J, Q, or X. In some embodiments, at least one of R⁴ and R⁵ is A, J, Q, or X. In some embodiments, at least one of R⁴ and R⁶ is A, J, Q, or X.

In some embodiments, R⁴ is A, J, Q, or X, and R⁵, R⁶, and R⁷ are hydrogen or C₁-C₆ alkyl. In some embodiments, R⁴ is A, J, Q, or X, and R⁵, R⁶, and R⁷ are hydrogen. In some embodiments, R⁵ is A, J, Q, or X, and R⁵, R⁶, and R⁷ are hydrogen or C₁-C₆ alkyl. In some embodiments, R⁵ is A, J, Q, or X, and R⁴, R⁶, and R⁷ are hydrogen. In some embodiments, R⁶ is A, J, Q, or X, and R⁴, R⁵, and R⁷ are hydrogen or C₁-C₆ alkyl. In some embodiments, R⁶ is A, J, Q, or X, and R⁴, R⁵, and R⁷ are hydrogen. In some embodiments, R⁷ is A, J, Q, or X, and R⁴, R⁵, and R⁶ are hydrogen or C₁-C₆ alkyl. In some embodiments, R⁷ is A, J, Q, or X, and R⁴, R⁵, and R⁶ are hydrogen.

In some embodiments, R¹³ is selected from hydrogen, and C₁-C₆ alkyl optionally substituted with one or more substituents independently selected from C₁-C₆ alkyl, oxo, halo, —OMe, —CN, —NH₂, and —NO₂. In some embodiments, R¹³ is C₁-C₆ alkyl optionally substituted with one or more substituents independently selected from C₁-C₆ alkyl, oxo, halo, —OMe, —CN, —NH₂, and —NO₂. In some embodiments, R¹³ is C₁-C₆ alkyl optionally substituted with one or more C₁-C₆ alkyl or oxo. In some embodiments, R¹³ is C₁-C₆ alkyl optionally substituted with one or more C₁-C₆ alkyl. In some embodiments, R³ is C₁-C₆ alkyl optionally substituted with one or more oxo.

In some embodiments, R¹³ is selected from hydrogen, and C₁-C₆ alkyl optionally substituted with one or more substituents independently selected from halo, —OMe, —CN, —NH₂, and —NO₂. In some embodiments, R¹³ is selected from hydrogen, and C₁-C₆ alkyl optionally substituted with one or more substituents independently selected from halo, —OMe, —CN, and —NH₂. In some embodiments, R¹³ is selected from hydrogen and C₁-C₃ alkyl optionally substituted with one or more substituents independently selected from halo, —OMe, and —CN. In some embodiments, R¹³ is hydrogen. In some embodiments, R¹³ is C₁-C₃ alkyl.

In some embodiments, R⁴ is selected from C₁-C₆ alkyl and C₂-C₆ alkenyl, wherein C₁-C₆ alkyl and C₂-C₆ alkenyl are optionally substituted with one or more substituents independently selected from halo, —OMe, —CN, —NH₂, and —NO₂. In some embodiments, R¹⁴ is selected from C₁-C₆ alkyl and C₂-C₆ alkenyl, wherein C₁-C₆ alkyl and C₂-C₆ alkenyl are optionally substituted with one or more substituents independently selected from halo, —OMe, and —CN. In some embodiments, R¹⁴ is C₁-C₃ alkyl optionally substituted with one or more substituents independently selected from halo, —OMe, and —CN. In some embodiments, R¹⁴ is C₁-C₃ alkyl. In some embodiments, R¹⁴ is C₂-C₃ alkenyl.

In some embodiments, R¹³ and R¹⁴ taken together with the atom to which they are attached to form a substituted or unsubstituted C₃-C₈cycloalkyl or substituted or unsubstituted 3-to 8-membered heterocycloalkyl having 1 or 2 heteroatoms each independently selected from N, O, and S. In some embodiments, R¹³ and R¹⁴ taken together with the atom to which they are attached to form a substituted or unsubstituted C₃-C₈cycloalkyl. In some embodiments, R¹³ and R¹⁴ taken together with the atom to which they are attached to form a substituted or unsubstituted 3- to 8-membered heterocycloalkyl having 1 or 2 heteroatoms each independently selected from N, O, and S.

In some embodiments, R¹³ and R¹⁴ taken together with the atom to which they are attached to form cyclopropyl, cyclobutyl, cyclopentyl, cyclopentenyl, cyclohexyl, cyclohexenyl, cycloheptyl, cyclooctyl, spiro[2.2]pentyl, norbornyl or bicycle[1.1.1]pentyl. In some embodiments, R¹³ and R¹⁴ taken together with the atom to which they are attached to form cyclopropyl. In some embodiments, R¹¹ and R¹⁴ taken together with the atom to which they are attached to form cyclobutyl. In some embodiments, R¹³ and R¹⁴ taken together with the atom to which they are attached to form cyclopentyl. In some embodiments, R¹³ and R¹⁴ taken together with the atom to which they are attached to form cyclohexyl.

In some embodiments, R¹³ and R¹⁴ taken together with the atom to which they are attached to form dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl.

In some embodiments, R¹⁵ is selected from hydrogen, and C₁-C₆ alkyl optionally substituted with one or more substituents independently selected from C₁-C₆ alkyl, oxo, halo, —OMe, —CN, —NH₂, and —NO₂. In some embodiments, R¹⁵ is C₁-C₆ alkyl optionally substituted with one or more substituents independently selected from C₁-C₆ alkyl, oxo, halo, —OMe, —CN, —NH₂, and —NO₂. In some embodiments, R¹⁵ is C₁-C₆ alkyl optionally substituted with one or more C₁-C₆ alkyl or oxo. In some embodiments, R¹⁵ is C₁-C₆ alkyl optionally substituted with one or more C₁-C₆ alkyl. In some embodiments, R¹⁵ is C₁-C₆ alkyl optionally substituted with one or more oxo.

In some embodiments, R¹⁵ is selected from C₁-C₆ alkylene and C₁-C₆ alkenylene, wherein C₁-C₆ alkylene and C₂-C₆ alkenylene are optionally substituted with one or more substituents independently selected from halo, —OMe, —CN, —NH₂, and —NO₂. In some embodiments, R¹⁵ is selected from C₁-C₆ alkylene and C₂-C₆ alkenylene, wherein C₁-C₆ alkylene and C₂-C₃ alkenylene are optionally substituted with one or more substituents independently selected from halo, —OMe, and —CN. In some embodiments, R¹⁵ is selected from C₁-C₃ alkylene and C₂-C₃ alkenylene, wherein C₁-C₃ alkylene and C₂-C₃ alkenylene are optionally substituted with one or more substituents independently selected from halo, —OMe, and —CN. In some embodiments, R¹⁵ is C₁-C₃ alkylene optionally substituted with one or more substituents independently selected from halo, —OMe, and —CN. In some embodiments, R¹⁵ is C₁-C₃ alkylene.

In some embodiments, R¹⁵ is C₂-C₃ alkenylene.

In some embodiments, X is selected from glucose, xylose, galactose, rhamnose, rutinose, and disaccharide. In some embodiments, X is selected from glucose, galactose, rhamnose, rutinose, and disaccharide. In some embodiments, X is selected from glucose, xylose, rhamnose, rutinose, and disaccharide. In some embodiments, X is selected from glucose, xylose, galactose, rutinose, and disaccharide. In some embodiments, X is selected from glucose, xylose, galactose, rhamnose, and disaccharide.

In some embodiments, X is disaccharide.

In some embodiments, X is disaccharide selected from the group consisting of Sucrose, Lactose, Maltose, Trehalose, Cellobiose, Chitobiose, Kojibiose, Nigerose, Isomaltose, β,β-Trehalose, α,β-Trehalose, Sophorose, Laminaribiose, Gentiobiose, Trehalulose, Turanose, Maltulose, Leucrose, Isomaltulose, Gentiobiulose, Mannobiose, Melibiose, Melibiulose, Rutinose, Rutinulose, and Xylobiose.

In some embodiments, X is disaccharide selected from the group consisting of Sucrose, Lactose, Maltose, Trehalose, Cellobiose, and Chitobiose.

In some embodiments, X is selected from glucose, xylose, galactose, rhamnose, and rutinose. In some embodiments, X is selected from glucose, galactose, and rhamnose. In some embodiments, X is glucose. In some embodiments, X is xylose. In some embodiments, X is galactose. In some embodiments, X is rhamnose. In some embodiments, X is rutinose.

In certain embodiments, the compound of Formula (I) is a compound of Formula (Ib):

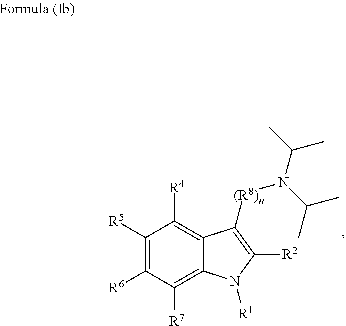

wherein R¹, R², R³, R⁴, R⁵, R⁶, R⁷, and R⁸ are defined herein above.

In certain embodiments, the compound of Formula (I) is a compound of Formula (Ib-1):

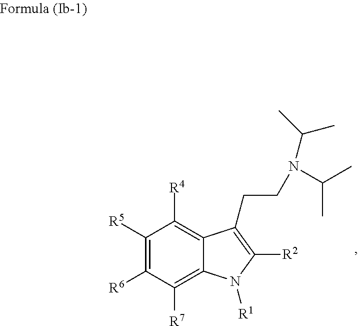

wherein R¹, R², R³, R⁴, R⁵, R⁶, and R⁷ are defined herein above.

In certain embodiments, the compound of Formula (I) is a compound of Formula (Ib-2):

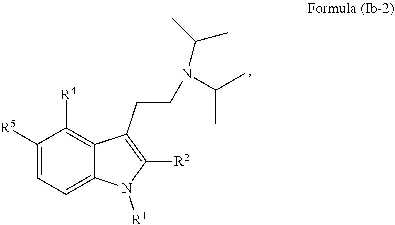

wherein R¹, R², R⁴, and R⁵ are defined herein above.

In certain embodiments, the compound of Formula (I) is a compound of Formula (Ic):

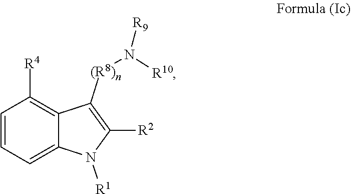

R¹ and R¹⁰ are independently selected from hydrogen, C₁-C₆ alkyl, C₂-C₆ alkenyl, and C₂-C₆ alkynyl, wherein alkyl, alkenyl, and alkynyl are optionally substituted with one or more substituents independently selected from halo, —OMe, —CN, —NH₂, and —NO₂;
R⁸ is —CR′₂—, wherein each R′ is independently selected from hydrogen, halo, haloalkyl, alkoxy, haloalkoxy, and amine;
n is selected from 2, 3, and 4;
R₉ is selected from C₂-C₆ alkyl, C₂-C₆ alkenyl, and C₂-C_{6F} alkynyl, wherein alkyl, alkenyl, and alkynyl are optionally substituted with one or more substituents independently selected from halo, —OMe, —CN, —NH₂, —NO₂, and 3- to 8-membered heterocycle, and wherein 3- to 8-membered heterocycle is optionally substituted with one or more substituents independently selected from halo, —OMe, —CN, —NH₂, and —NO₂;
R² is selected from hydrogen, halogen, C₁-C₆ alkyl, and C₁-C₆, haloalkyl;
R⁴ is selected from A, J, Q, and X;
A is

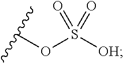 [SMILES: *OS(=O)(=O)O]

J is

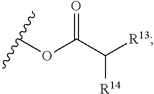

Q is

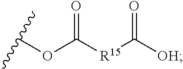

X is selected from glucose, xylose, galactose, rhamnose, rutinose, and disaccharide;
R¹³ is selected from hydrogen, and C₁-C₆ alkyl optionally substituted with one or more substituents independently selected from C₁-C₆ alkyl, oxo, halo, —OMe, —CN, —NH₂, and —NO₂;
R¹⁴ is selected from C₁-C₆ alkyl and C₂₋₆ alkenyl, wherein C₁-C₆ alkyl and C₂-C₆ alkenyl are optionally substituted with one or more substituents independently selected from halo, —OMe, —CN, —NH₂, and —NO₂;
R¹⁵ is selected from C₁-C₆ alkylene and C₂-C₆ alkenylene, wherein C₁-C₆ alkylene and C₂-C₆ alkenylene are optionally substituted with one or more substituents independently selected from C₁-C₆ alkyl, halo, —OMe, —CN, —NH₂, and —NO₂.

In certain embodiments, the compound of Formula (Ic) is a compound of Formula (Ic-1):

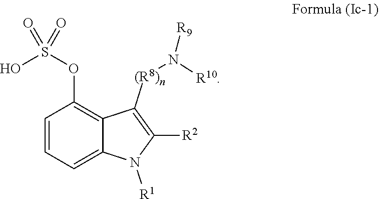

In certain embodiments, the compound of Formula (Ic) is a compound of Formula (Ic-2):

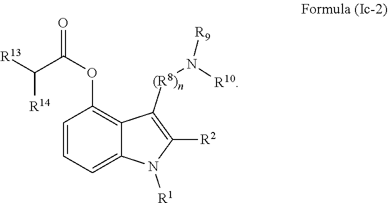

In certain embodiments, the compound of Formula (Ic) is a compound of Formula (Ic-3):

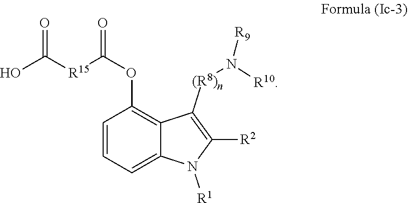

In certain embodiments, the compound of Formula (Ic) is a compound of Formula (Ic-4):

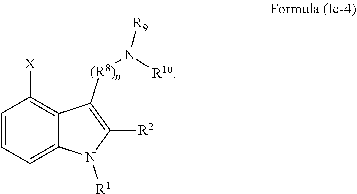

In certain embodiments, the compound of Formula (Ic) is a compound of Formula (Ic-4a):

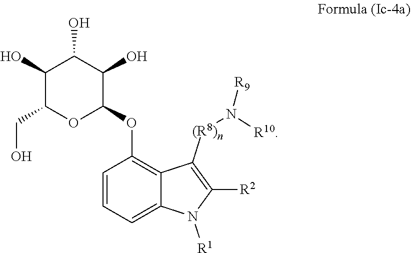

In certain embodiments, the compound of Formula (I) is a compound of Formula (Id):

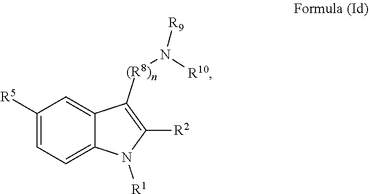

R¹ and R¹⁰ are independently selected from hydrogen, C₁-C₆ alkyl, C₂-C₆ alkenyl, and C₂-C₆ alkynyl, wherein alkyl, alkenyl, and alkynyl are optionally substituted with one or more substituents independently selected from halo, —OMe, —CN, —NH₂, and —NO₂;
R⁸ is —CR′₂—, wherein each R′ is independently selected from hydrogen, halo, haloalkyl, alkoxy, haloalkoxy, and amine;
n is selected from 2, 3, and 4;
R⁹ is selected from C₂-C₆ alkyl, C₂-C₆ alkenyl, and C₂-C₆ alkynyl, wherein alkyl, alkenyl, and alkynyl are optionally substituted with one or more substituents independently selected from halo, —OMe, —CN, —NH₂, —NO₂, and 3- to 8-membered heterocycle, and wherein 3- to 8-membered heterocycle is optionally substituted with one or more substituents independently selected from halo, —OMe, —CN, —NH₂, and —NO₂;
R² is selected from hydrogen, halogen, C₁-C₆ alkyl, and C₁-C₆ haloalkyl;
R⁴ is selected from A, J, Q, and X:
A is

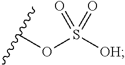 [SMILES: *OS(=O)(=O)O]

J is

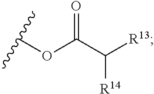

Q is

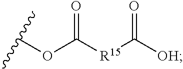

X is selected from glucose, xylose, galactose, rhamnose, rutinose, and disaccharide;
R¹³ is selected from hydrogen, and C₁-C₆ alkyl optionally substituted with one or more substituents independently selected from C₁-C₆ alkyl, oxo, halo, —OMe, —CN, —NH₂, and —NO₂;
R¹⁴ is selected from C₁-C₆ alkyl and C₂-C₆ alkenyl, wherein C₁-C₆ alkyl and C₂-C₆ alkenyl are optionally substituted with one or more substituents independently selected from halo, —OMe, —CN, —NH₂, and —NO₂;
R¹⁵ is selected from C₁-C₆ alkylene and C₂-C₆ alkenylene, wherein C₁-C₆ alkylene and C₂-C₆ alkenylene are optionally substituted with one or more substituents independently selected from C₁-C₆ alkyl, halo, —OMe, —CN, —NH₂, and —NO₂.

Also provided herein, in another aspect, is an indole alkaloid. In some embodiments, an indole alkaloid provided herein is a compound of Formula (II):

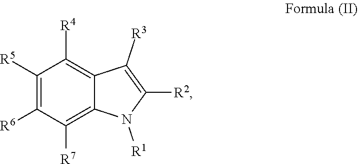

or a pharmaceutically acceptable salt thereof, wherein R¹, R², R³, R⁴, R¹, R⁶, and R are each independently selected from halo, —OH, C₁₋₅ alkyl, C₁₋₅ alkoxy, C₂₋₅ alkenyl, —C(O)(C₁₋₈ alkyl), optionally substituted C₆₋₁₀ aryl, 5- to 10-membered heteroaryl, C₃₋₁₀ cycloalkyl, 3- to 10-membered heterocycloalkyl, NO₂, NH₂, COOH, CN, —SH, SO₃, SO₄, and PO₄. In some embodiments, the substituent on the indole alkaloid acts as an acceptor functional group for enzymes to transfer donor functional groups.

Provided herein, in another aspect, is tryptamine alkaloid. In some embodiments, a tryptamine alkaloid provided herein is a compound of Formula (III):

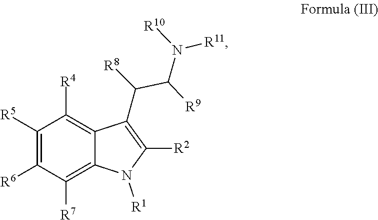

or a pharmaceutically acceptable salt thereof, wherein R¹, R², R³, R⁴, R⁵, R⁶, R⁷, R⁸, R⁹, R¹⁰, and R¹¹ are each independently selected from halo, —OH, C₁₋₅ alkyl, C₁₋₅ alkoxy, C₂₋₅ alkenyl, —C(O(C₁₋₈ alkyl), optionally substituted C₆₋₁₀ aryl, 5- to 10-membered heteroaryl, C₃₋₁₀ cycloalkyl, 3- to 10-membered heterocycloalkyl, NO₂, NH₂, COOH, CN, —SH, SO₃, SO₄, and PO₄. In some embodiments, the substituent on the tryptamine alkaloid acts as an acceptor functional group for enzymes to transfer donor functional groups.

Provided herein, in another aspect, is an ergoline alkaloid. In some embodiments, an ergoline alkaloid provided herein is a compound of Formula (IV):

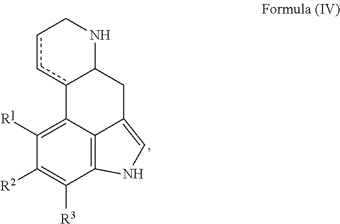

or a pharmaceutically acceptable salt thereof, wherein [symbol] is a single bond or a double bond and R¹, R², and R³ are each independently selected from halo, —OH, C₁₋₅ alkyl, C₁₋₅ alkoxy, C₂₋₅ alkenyl, —C(O)(C₁₋₈ alkyl), optionally substituted C₆₋₁₀ aryl, 5- to 10-membered heteroaryl, C₃₋₁₀ cycloalkyl, 3- to 10-membered heterocycloalkyl, NO₂, NH₂, COOH, CN, —SH, SO₃, SO₄, and PO₄. In some embodiments, the substituent on the ergoline alkaloid acts as an acceptor functional group for enzymes to transfer donor functional groups.

Provided herein, in another aspect, is a beta-carboline alkaloid. In some embodiments, a beta-carboline alkaloid provided herein is a compound of Formula (V):

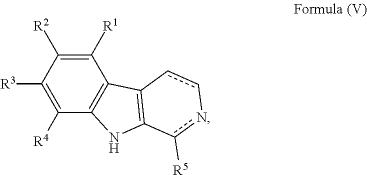

or a pharmaceutically acceptable salt thereof, wherein [symbol] is a single bond or a double bond and R¹, R², R³, and R⁴ are each independently selected from halo, —OH, C₁₋₅ alkyl, C₁₋₄ alkoxy, C₂₋₅ alkenyl, —C(O)(C₁₋₈ alkyl), optionally substituted C₆₋₁₀ aryl, 5- to 10-membered heteroaryl, C₃₋₁₀ cycloalkyl, 3- to 10-membered heterocycloalkyl, NO₂, NH₂, COOH, CN, —SH, SO₃, SO₄, and PO₄ and R₅ is H or methyl. In some embodiments, the substituent on the beta-carboline alkaloid acts as an acceptor functional group for enzymes to transfer donor functional groups.

Provided herein, in another aspect, is an ibogamine alkaloid. In some embodiments, an ibogamine alkaloid provided herein is a compound of Formula (VI):

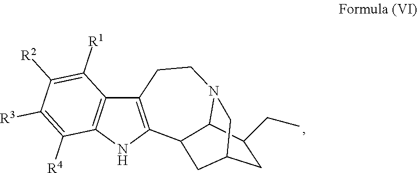

or a pharmaceutically acceptable salt thereof, wherein R¹, R², R³, and R⁴ are each independently selected from halo, —OH, C₁₋₅ alkyl, C₁₋₅ alkoxy, C₂₋₅ alkenyl, —C(O)(C₁₋₈ alkyl), optionally substituted C₆₋₁₀ aryl, 5- to 10-membered heteroaryl, C₃₋₁₀ cycloalkyl, 3- to 10-membered heterocycloalkyl, NO₂, NH₂, COOH, CN, —SH, SO₃, SO₄, and PO₄. In some embodiments, the substituent on the ibogamine alkaloid acts as an acceptor functional group for enzymes to transfer donor functional groups.

Provided herein, in another aspect, is a mitragynine alkaloid. In some embodiments, a mitragynine alkaloid provided herein is a compound of Formula (VII):

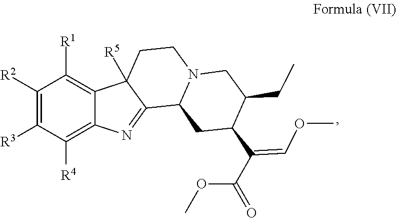

or a pharmaceutically acceptable salt thereof, wherein R¹, R², R³, R⁴, and R⁵ are each independently selected from halo, —OH, C₁₋₅ alkyl, C₁₋₅ alkoxy, C₂₋₅ alkenyl, —C(O)(C₁₋₈ alkyl), optionally substituted C₆₋₁₀ aryl, 5- to 10-membered heteroaryl, C₃₋₁₀ cycloalkyl, 3- to 10-membered heterocycloalkyl, NO₂, NH₂, COOH, CN, —SH, SO₃, SO₄, and PO₄. In some embodiments, the substituent on the mitragynine alkaloid acts as an acceptor functional group for enzymes to transfer donor functional groups.

In some embodiments, a compound provided herein is selected from 2-(4-methoxy-1H-indol-3-yl)-N,N-dimethylethan-1-amine, 3-(2-(dimethylamino)ethyl)-1H-indol-4-yl dihydrogen phosphate, 3-(2-(dimethylamino)ethyl)-1H-indol-4-yl acetate, 3-(2-(dimethylamino)ethyl)-1H-indol-4-yl propionate, 3-(2-(dimethylamino)ethyl)-1H-indol-4-yl butyrate, 3-(2-(dimethylamino)ethyl)-1H-indol-4-yl isobutyrate, 3-((3-(2-(dimethylamino)ethyl)-1H-indol-4-yl)oxy)-3-oxopropanoic acid, 4-((3-(2-(dimethylamino)ethyl)-1H-indol-4-yl)oxy)-4-oxobutanoic acid, 5-((3-(2-(dimethylamino)ethyl)-1H-indol-4-yl)oxy)-5-oxopentanoic acid, 6-((3-(2-(dimethylamino)ethyl)-1H-indol-4-yl)oxy)-6-oxohexanoic acid, 2-((3-(2-(dimethylamino)ethyl)-1H-indol-4-yl)oxy)-6-(hydroxymethyl)tetrahydro-2H-pyran-3,4,5-triol, 2-((3-(2-(dimethylamino)ethyl)-1H-indol-4-yl)oxy)-6-methyltetrahydro-2H-pyran-3,4,5-triol, N-(2-(4-methoxy-1H-indol-3-yl)ethyl)-N-propylpropan-1-amine, 3-(2-(dipropylamino)ethyl)-1H-indol-4-yl dihydrogen phosphate, 3-(2-(dipropylamino)ethyl)-1H-indol-4-yl acetate, 3-(2-(dipropylamino)ethyl)-1H-indol-4-yl propionate, 3-(2-(dipropylamino)ethyl)-1H-indol-4-yl butyrate, 3-(2-(dipropylamino)ethyl)-1H-indol-4-yl isobutyrate, 3-((3-(2-(dipropylamino)ethyl)-1H-indol-4-yl)oxy)-3-oxopropanoic acid, 4-((3-(2-(dipropylamino)ethyl)-1-indol-4-yl)oxy)-4-oxobutanoic acid, 5-((3-(2-(dipropylamino)ethyl)-1H-indol-4-yl)oxy)-5-oxopentanoic acid, 6-((3-(2-dipropylamino)ethyl)-1H-indol-4-yl)oxy)-6-oxohexanoic acid, 2-((3-(2-(dipropylamino)ethyl)-1H-indol-4-yl)oxy)-6-(hydroxymethyl)tetrahydro-2H-pyran-3,4,5-triol, 2-((3-(2-(dipropylamino)ethyl)-1H-indol-4-yl)oxy)-6-methyltetrahydro-2H-pyran-3,4,5-triol, N-allyl-N-(2-(4-methoxy-1H-indol-3-yl)ethyl)prop-2-en-1-amine, 3-(2-(diallylamino)ethyl)-1H-indol-4-yl dihydrogen phosphate, 3-(2-(diallylamino)ethyl)-1H-indol-4-yl acetate, 3-(2-(diallylamino)ethyl)-1H-indol-4-yl propionate, 3-(2-(diallylamino)ethyl)-1H-indol-4-yl butyrate, 3-(2-(diallylamino)ethyl)-1H-indol-4-yl isobutyrate, 3-((3-(2-(diallylamino)ethyl)-1H-indol-4-yl)oxy)-3-oxopropanoic acid, 4-4(3-(2-(diallylamino)ethyl)-1H-indol-4-yl)oxy)-4-oxobutanoic acid, 5-((3-(2-(diallylamino)ethyl)-1H-indol-4-yl)oxy)-5-oxopentanoic acid, 6-((3-(2-(diallylamino)ethyl)-1H-indol-4-yl)oxy)-6-oxohexanoic acid, 2-((3-(2-(diallylamino)ethyl)-1H-indol-4-yl)oxy)-6-(hydroxymethyl)tetrahydro-2H-pyran-3,4,5-triol, 2-((3-(2-(diallylamino)ethyl)-1H-indol-4-yl)oxy)-6-methyltetrahydro-2H-pyran-3,4,5-triol, N-(2-(4-methoxy-1H-indol-3-yl)ethyl)-N-methylpropan-2-amine, 3-(2-(isopropyl(methyl)amino)ethyl)-1H-indol-4-yl dihydrogen phosphate, 3-(2-(isopropyl(methyl)amino)ethyl)-1H-indol-4-yl acetate, 3-(2-(isopropyl(methyl)amino)ethyl)-1H-indol-4-yl propionate, 3-(2-(isopropyl(methyl)amino)ethyl)-1H-indol-4-yl butyrate, 3-(2-(isopropyl(methyl)amino)ethyl)-1H-indol-4-yl isobutyrate, 3-4(3-(2-(isopropyl(methyl)amino)ethyl)-1H-indol-4-yl)oxy)-3-oxopropanoic acid, 4-((3-(2-(isopropyl(methyl)amino)ethyl)-1H-indol-4-yl)oxy)-4-oxobutanoic acid, 5-((3-(2-(isopropyl(methyl)amino)ethyl)-1H-indol-4-yl)oxy)-5-oxopentanoic acid, 6-((3-(2-(isopropyl(methyl)amino)ethyl)-1H-indol-4-yl)oxy)-6-oxohexanoic acid, 2-((3-(2-(isopropyl(methyl)amino)ethyl)-1H-indol-4-yl)oxy)-6-(hydroxymethyl)tetrahydro-2H-pyran-3,4,5-triol, 2-((3-(2-(isopropyl(methyl)amino)ethyl)-1H1-indol-4-yl)oxy)-6-methyltetrahydro-2H-pyran-3,4,5-triol, N-ethyl-N-(2-(4-methoxy-1H-indol-3-yl)ethyl)propan-2-amine, 3-(2-(ethyl(isopropyl)amino)ethyl)-1H-indol-4-yl dihydrogen phosphate, 3-(2-(ethyl(isopropyl)amino)ethyl)-1H-indol-4-yl acetate, 3-(2-(ethyl(isopropyl)amino)ethyl)-1H-indol-4-yl propionate, 3-(2-(ethyl(isopropyl)amino)ethyl)-1H-indol-4-yl butyrate, 3-(2-(ethyl(isopropyl)amino)ethyl)-1H-indol-4-yl isobutyrate, 3-((3-(2-(ethyl(isopropyl)amino)ethyl)-1H-indol-4-yl)oxy)-3-oxopropanoic acid, 4-((3-(2-(ethyl(isopropyl)amino)ethyl)-1H-indol-4-yl)oxy)-4-oxobutanoic acid, 4-((3-(2-(ethyl(isopropyl)amino)ethyl)-1H1-indol-4-yl)oxy)-5-oxopentanoic acid, 4-((3-(2-(ethyl(isopropyl)amino)ethyl)-1H-indol-4-yl)oxy)-6-oxohexanoic acid, 2-((3-(2-(ethyl(isopropyl)amino)ethyl)-1H-indol-4-yl)oxy)-6-(hydroxymethyl)tetrahydro-2H-pyran-3,4,5-triol, 2-((3-(2-(ethyl(isopropyl)amino)ethyl)-1H-indol-4-yl)oxy)-6-methyltetrahydro-2H-pyran-3,4,5-triol, N-isopropyl-N-(2-(4-methoxy-1H-indol-3-yl)ethyl)propan-2-amine, 3-(2-(diisopropylamino)ethyl)-1H-indol-4-yl dihydrogen phosphate, 3-(2-(diisopropylamino)ethyl)-1H-indol-4-yl acetate, 3-(2-(diisopropylamino)ethyl)-1H-indol-4-yl propionate, 3-(2-(diisopropylamino)ethyl)-1H-indol-4-yl butyrate, 3-(2-(diisopropylamino)ethyl)-1H-indol-4-yl isobutyrate, 3-((3-(2-(diisopropylamino)ethyl)-1H-indol-4-yl)oxy)-3-oxopropanoic acid, 4-((3-(2-(diisopropylamino)ethyl)-1H-indol-4-yl)oxy)-4-oxobutanoic acid, 5-((3-(2-(diisopropylamino)ethyl)-1H-indol-4-yl)oxy)-5-oxopentanoic acid, 6-((3-(2-(diisopropylamino)ethyl)-1H-indol-4-yl)oxy)-6-oxohexanoic acid, 2-((3-(2-(diisopropylamino)ethyl)-1-indol-4-yl)oxy)-6-(hydroxymethyl)tetrahydro-2H-pyran-3,4,5-triol, 2-((3-(2-(diisopropylamino)ethyl)-1H-indol-4-yl)oxy)-6-methyltetrahydro-2H-pyran-3,4,5-triol, N,N-diethyl-2-(4-methoxy-1H-indol-3-yl)ethan-1-amine, 3-(2-(diethylamino)ethyl)-1H-indol-4-yl dihydrogen phosphate, 3-(2-(diethylamino)ethyl)-1H-indol-4-yl acetate, 3-(2-(diethylamino)ethyl)-1H-indol-4-yl propionate, 3-(2-(diethylamino)ethyl)-1H-indol-4-yl butyrate, 3-(2-(diethylamino)ethyl)-1H-indol-4-yl isobutyrate, 3-((3-(2-(diethylamino)ethyl)-1H-indol-4-yl)oxy)-3-oxopropanoic acid, 4-((3-(2-(diethylamino)ethyl)-1H-indol-4-yl)oxy)-4-oxobutanoic acid, 5-((3-(2-(diethylamino)ethyl)-1H-indol-4-yl)oxy)-5-oxopentanoic acid, 6-((3-(2-(diethylamino)ethyl)-1l-indol-4-yl)oxy)-6-oxohexanoic acid, 2-((3-(2-(diethylamino)ethyl)-1H-indol-4-yl)oxy)-6-(hydroxymethyl)tetrahydro-2H-pyran-3,4,5-triol, 2-((3-(2-(diethylamino)ethyl)-1H-indol-4-yl)oxy)-6-methyltetrahydro-2H-pyran-3,4,5-triol, N-ethyl-N-(2-(4-methoxy-1H-indol-3-yl)ethyl)propan-1-amine, 3-(2-(ethyl(propyl)amino)ethyl)-1H-indol-4-yl dihydrogen phosphate, 3-(2-(ethyl(propyl)amino)ethyl)-1H-indol-4-yl acetate, 3-(2-(ethyl(propyl)amino)ethyl)-1H-indol-4-yl propionate, 3-(2-(ethyl(propyl)amino)ethyl)-1H-indol-4-yl butyrate, 3-(2-(ethyl(propyl)amino)ethyl)-1H-indol-4-yl isobutyrate, 3-((3-(2-(ethyl(propyl)amino)ethyl)-1H-indol-4-yl)oxy)-3-oxopropanoic acid, 4-((3-(2-(ethyl(propyl)amino)ethyl)-1H-indol-4-yl)oxy)-4-oxobutanoic acid, 5-((3-(2-(ethyl(propyl)amino)ethyl)-1H-indol-4-yl)oxy)-5-oxopentanoic acid, 6-((3-(2-(ethyl(propyl)amino)ethyl)-1H-indol-4-yl)oxy)-6-oxohexanoic acid, 2-((3-(2-(ethyl(propyl)amino)ethyl)-1H-indol-4-yl)oxy)-6-(hydroxymethyl)tetrahydro-2H-pyran-3,4,5-triol, 2-((3-(2-(ethyl(propyl)amino)ethyl)-1H-indol-4-yl)oxy)-6-methyltetrahydro-2H-pyran-3,4,5-triol, N-(2-(4-methoxy-1H-indol-3-yl)ethyl)-N-methylcyclopropanamine, 3-(2-(cyclopropyl(methyl)amino)ethyl)-1H-indol-4-yl dihydrogen phosphate, 3-(2-(cyclopropyl(methyl)amino)ethyl)-1H-indol-4-yl acetate, 3-(2-(cyclopropyl(methyl)amino)ethyl)-1H-indol-4-yl propionate, 3-(2-(cyclopropyl(methyl)amino)ethyl)-1H-indol-4-yl butyrate, 3-(2-(cyclopropyl(methyl)amino)ethyl)-1H-indol-4-yl isobutyrate, 3-((3-(2-(cyclopropyl(methyl)amino)ethyl)-1H-indol-4-yl)oxy)-3-oxopropanoic acid, 4-((3-(2-(cyclopropyl(methyl)amino)ethyl)-1H-indol-4-yl)oxy)-4-oxobutanoic acid, 5-((3-(2-(cyclopropyl(methyl)amino)ethyl)-1H-indol-4-yl)oxy)-5-oxopentanoic acid, 6-((3-(2-(cyclopropyl(methyl)amino)ethyl)-1H-indol-4-yl)oxy)-6-oxohexanoic acid, 2-((3-(2-(cyclopropyl(methyl)amino)ethyl)-1H-indol-4-yl)oxy)-6-(hydroxymethyl)tetrahydro-2H-pyran-3,4,5-triol, 2-((3-(2-(cyclopropyl(methyl)amino)ethyl)-1H-indol-4-yl)oxy)-6-methyltetrahydro-2H-pyran-3,4,5-triol, N-ethyl-2-(4-methoxy-1H-indol-3-yl)-N-methylethan-1-amine, 3-(2-(ethyl(methyl)amino)ethyl)-1H-indol-4-yl dihydrogen phosphate, 3-(2-(ethyl(methyl)amino)ethyl)-1H-indol-4-yl acetate, 3-(2-(ethyl(methyl)amino)ethyl)-1H-indol-4-yl propionate, 3-(2-(ethyl(methyl)amino)ethyl)-1H-indol-4-yl butyrate, 3-(2-(ethyl(methyl)amino)ethyl)-1H-indol-4-yl isobutyrate, 3-((3-(2-(ethyl(methyl)amino)ethyl)-1H-indol-4-yl)oxy)-3-oxopropanoic acid, 4-((3-(2-(ethyl(methyl)amino)ethyl)-1H-indol-4-yl)oxy)-4-oxobutanoic acid, 4-((3-(2-(ethyl(methyl)amino)ethyl)-1H-indol-4-yl)oxy)-4-oxobutanoic acid, 6-((3-(2-(ethyl(methyl)amino)ethyl)-1H-indol-4-yl)oxy)-6-oxohexanoic acid, 2-((3-(2-(ethyl(methyl)amino)ethyl)-1H1-indol-4-yl)oxy)-6-(hydroxymethyl)tetrahydro-2H-pyran-3,4,5-triol, 2-((3-(2-(ethyl(methyl)amino)ethyl)-1H-indol-4-yl)oxy)-6-methyltetrahydro-2H-pyran-3,4,5-triol, N-(2-(4-methoxy-1 f-indol-3-yl)ethyl)-N-methylprop-2-en-1-amine, 3-(2-(allyl(methyl)amino)ethyl)-1-indol-4-yl dihydrogen phosphate, 3-(2-(allyl(methyl)amino)ethyl)-1H-indol-4-yl acetate, 3-(2-(allyl(methyl)amino)ethyl)-1H-indol-4-yl propionate, 3-(2-(allyl(methyl)amino)ethyl)-1H-indol-4-yl butyrate, 3-(2-(allyl(methyl)amino)ethyl)-1H-indol-4-yl isobutyrate, 3-((3-(2-(allyl(methyl)amino)ethyl)-1H-indol-4-yl)oxy)-3-oxopropanoic acid, 4-((3-(2-(allyl(methyl)amino)ethyl)-1H-indol-4-yl)oxy)-4-oxobutanoic acid, 5-((3-(2-(allyl(methyl)amino)ethyl)-1H-indol-4-yl)oxy)-5-oxopentanoic acid, 6-((3-(2-(allyl(methyl)amino)ethyl)-1H-indol-4-yl)oxy)-6-oxohexanoic acid, 2-((3-(2-(allyl(methyl)amino)ethyl)-1H-indol-4-yl)oxy)-6-(hydroxymethyl)tetrahydro-2H-pyran-3,4,5-triol, 2-((3-(2-(allyl(methyl)amino)ethyl)-1H-indol-4-yl)oxy)-6-methyltetrahydro-2H-pyran-3,4,5-triol, N-ethyl-N-(2-(4-methoxy-1H-indol-3-yl)ethyl)prop-2-en-1-amine, 3-(2-(allyl(ethyl)amino)ethyl)-1H-indol-4-yl dihydrogen phosphate, 3-(2-(allyl(ethyl)amino)ethyl)-1H-indol-4-yl acetate, 3-(2-(allyl(ethyl)amino)ethyl)-1H-indol-4-yl propionate, 3-(2-(allyl(ethyl)amino)ethyl)-1H-indol-4-yl butyrate, 3-(2-(allyl(ethyl)amino)ethyl)-1H1-indol-4-yl isobutyrate, 3-((3-(2-(allyl(ethyl)amino)ethyl)-1H-indol-4-yl)oxy)-3-oxopropanoic acid, 4-((3-(2-(allyl(ethyl)amino)ethyl)-1H-indol-4-yl)oxy)-4-oxobutanoic acid, 5-((3-(2-(allyl(ethyl)amino)ethyl)-1H1-indol-4-yl)oxy)-5-oxopentanoic acid, 6-((3-(2-(allyl(ethyl)amino)ethyl)-1H-indol-4-yl)oxy)-6-oxohexanoic acid, 2-((3-(2-(allyl(ethyl)amino)ethyl)-1H-indol-4-yl)oxy)-6-(hydroxymethyl)tetrahydro-2H-pyran-3,4,5-triol, 2-((3-(2-(allyl(ethyl)amino)ethyl)-1H-indol-4-yl)oxy)-6-methyltetrahydro-2H-pyran-3,4,5-triol, 4-methoxy-3-(2-(pyrrolidin-1-yl)ethyl)-1H-indole, 3-(2-(pyrrolidin-1-yl)ethyl)-1H-indol-4-yl dihydrogen phosphate, 3-(2-(pyrrolidin-1-yl)ethyl)-1H-indol-4-yl acetate, 3-(2-(pyrrolidin-1-yl)ethyl)-1H-indol-4-yl propionate, 3-(2-(pyrrolidin-1-yl)ethyl)-1H-indol-4-yl butyrate, 3-(2-(pyrrolidin-1-yl)ethyl)-1H-indol-4-yl isobutyrate, 3-oxo-3-((3-(2-(pyrrolidin-1-yl)ethyl)-1H-indol-4-yl)oxy)propanoic acid, 4-oxo-4-((3-(2-(pyrrolidin-1-yl)ethyl)-1H-indol-4-yl)oxy)butanoic acid, 5-oxo-5-((3-(2-(pyrrolidin-1-yl)ethyl)-1H-indol-4-yl)oxy)pentanoic acid, 6-oxo-6-((3-(2-(pyrrolidin-1-yl)ethyl)-1H-indol-4-yl)oxy)hexanoic acid, 2-(hydroxymethyl)-6-((3-(2-(pyrrolidin-1-yl)ethyl)-1H1-indol-4-yl)oxy)tetrahydro-2H-pyran-3,4,5-triol, 2-methyl-6-((3-(2-(pyrrolidin-1-yl)ethyl)-1H-indol-4-yl)oxy)tetrahydro-2H-pyran-3,4,5-triol, N-(2-(4-methoxy-1H-indol-3-yl)ethyl)acetamide, 3-(2-acetamidoethyl)-1H-indol-4-yl dihydrogen phosphate, 3-(2-acetamidoethyl)-1N-indol-4-yl acetate, 3-(2-acetamidoethyl)-1H-indol-4-yl propionate, 3-(2-acetamidoethyl)-1H-indol-4-yl butyrate, 3-(2-acetamidoethyl)-1H-indol-4-yl isobutyrate, 3-((3-(2-acetamidoethyl)-1H-indol-4-yl)oxy)-3-oxopropanoic acid, 4-((3-(2-acetamidoethyl)-1H-indol-4-yl)oxy)-4-oxobutanoic acid, 5-((3-(2-acetamidoethyl)-1H1-indol-4-yl)oxy)-5-oxopentanoic acid, 6-((3-(2-acetamidoethyl)-1H-indol-4-yl)oxy)-6-oxohexanoic acid, N-(2-(4-((3,4,5-trihydroxy-6-methyltetrahydro-2H-pyran-2-yl)oxy)-1H-indol-3-yl)ethyl)acetamide, N-(2-(4-((3,4,5-trihydroxy-6-(hydroxymethyl)tetrahydro-2H-pyran-2-yl)oxy)-1H-indol-3-yl)ethyl)acetamide, 2-(5-methoxy-1H-indol-3-yl)-N,N-dimethylethan-1-amine, 3-(2-(dimethylamino)ethyl)-1H-indol-5-yl dihydrogen phosphate, 3-(2-(dimethylamino)ethyl)-1H-indol-5-yl acetate, 3-(2-(dimethylamino)ethyl)-1H-indol-5-yl propionate, 3-(2-(dimethylamino)ethyl)-1H-indol-5-yl butyrate, 3-(2-(dimethylamino)ethyl)-1H-indol-5-yl isobutyrate, 3-((3-(2-(dimethylamino)ethyl)-1H-indol-5-yl)oxy)-3-oxopropanoic acid, 4-((3-(2-(dimethylamino)ethyl)-1H-indol-5-yl)oxy)-4-oxobutanoic acid, 5-((3-(2-(dimethylamino)ethyl)-1H-indol-5-yl)oxy)-5-oxopentanoic acid, 6-((3-(2-(dimethylamino)ethyl)-1H-indol-5-yl)oxy)-6-oxohexanoic acid, 2-((3-(2-(dimethylamino)ethyl)-1H-indol-5-yl)oxy)-6-(hydroxymethyl)tetrahydro-2H-pyran-3,4,5-triol, 2-((3-(2-(dimethylamino)ethyl)-1H-indol-5-yl)oxy)-6-methyltetrahydro-2H1-pyran-3,4,5-triol, N-(2-(5-methoxy-1H-indol-3-yl)ethyl)-N-propylpropan-1-amine, 3-(2-(dipropylamino)ethyl)-1H-indol-5-yl dihydrogen phosphate, 3-(2-(dipropylamino)ethyl)-1H-indol-5-yl acetate, 3-(2-(dipropylamino)ethyl)-1H-indol-5-yl propionate, 3-(2-(dipropylamino)ethyl)-1H-indol-5-yl butyrate, 3-(2-(dipropylamino)ethyl)-1H-indol-5-yl isobutyrate, 3-((3-(2-(dipropylamino)ethyl)-1H-indol-5-yl)oxy)-3-oxopropanoic acid, 4-((3-(2-(dipropylamino)ethyl)-1H-indol-5-yl)oxy)-4-oxobutanoic acid, 5-((3-(2-(dipropylamino)ethyl)-1H-indol-5-yl)oxy)-5-oxopentanoic acid, 6-((3-(2-(dipropylamino)ethyl)-1H-indol-5-yl)oxy)-6-oxohexanoic acid, 2-((3-(2-(dipropylamino)ethyl)-1H-indol-5-yl)oxy)-6-(hydroxymethyl)tetrahydro-2H-pyran-3,4,5-triol, 2-((3-(2-(dipropylamino)ethyl)-1H-indol-5-yl)oxy)-6-methyltetrahydro-2H1-pyran-3,4,5-triol, N-allyl-N-(2-(5-methoxy-1H-indol-3-yl)ethyl)prop-2-en-1-amine, 3-(2-(diallylamino)ethyl)-1H-indol-5-yl dihydrogen phosphate, 3-(2-(diallylamino)ethyl)-1H-indol-5-yl acetate, 3-(2-(diallylamino)ethyl)-1H-indol-5-yl propionate, 3-(2-(diallylamino)ethyl)-1H-indol-5-yl butyrate, 3-(2-(diallylamino)ethyl)-1H-indol-5-yl isobutyrate, 3-((3-(2-(diallylamino)ethyl)-1H-indol-5-yl)oxy)-3-oxopropanoic acid, 4-((3-(2-(diallylamino)ethyl)-1H-indol-5-yl)oxy)-4-oxobutanoic acid, 5-((3-(2-(diallylamino)ethyl)-1H-indol-5-yl)oxy)-5-oxopentanoic acid, 6-((3-(2-(diallylamino)ethyl)-1H-indol-5-yl)oxy)-6-oxohexanoic acid, 2-((3-(2-(diallylamino)ethyl)-1H-indol-5-yl)oxy)-6-(hydroxymethyl)tetrahydro-2H-pyran-3,4,5-triol, 2-((3-(2-(diallylamino)ethyl)-1H-indol-5-yl)oxy)-6-methyltetrahydro-2H-pyran-3,4,5-triol, N-(2-(5-methoxy-1H-indol-3-yl)ethyl)-N-methylpropan-2-amine, 3-(2-(isopropyl(methyl)amino)ethyl)-1H-indol-5-yl dihydrogen phosphate, 3-(2-(isopropyl(methyl)amino)ethyl)-1H-indol-5-yl acetate, 3-(2-(isopropyl(methyl)amino)ethyl)-1H-indol-5-yl propionate, 3-(2-(isopropyl(methyl)amino)ethyl)-1H-indol-5-yl butyrate, 3-(2-(isopropyl(methyl)amino)ethyl)-1H-indol-5-yl isobutyrate, 3-((3-(2-(isopropyl(methyl)amino)ethyl)-1H-indol-5-yl)oxy)-3-oxopropanoic acid, 4-((3-(2-(isopropyl(methyl)amino)ethyl)-1H-indol-5-yl)oxy)-4-oxobutanoic acid, 5-((3-(2-(isopropyl(methyl)amino)ethyl)-1H-indol-5-yl)oxy)-5-oxopentanoic acid, 6-((3-(2-(isopropyl(methyl)amino)ethyl)-1H-indol-5-yl)oxy)-6-oxohexanoic acid, 2-((3-(2-(isopropyl(methyl)amino)ethyl)-1H-indol-5-yl)oxy)-6-(hydroxymethyl)tetrahydro-2H-pyran-3,4,5-triol, 2-((3-(2-(isopropyl(methyl)amino)ethyl)-1H-indol-5-yl)oxy)-6-methyltetrahydro-2H-pyran-3,4,5-triol, N-ethyl-N-(2-(5-methoxy-1H-indol-3-yl)ethyl)propan-2-amine, 3-(2-(ethyl(isopropyl)amino)ethyl)-1H-indol-5-yl dihydrogen phosphate, 3-(2-(ethyl(isopropyl)amino)ethyl)-1H-indol-5-yl acetate, 3-(2-(ethyl(isopropyl)amino)ethyl)-1H-indol-5-yl propionate, 3-(2-(ethyl(isopropyl)amino)ethyl)-1H-indol-5-yl butyrate, 3-(2-(ethyl(isopropyl)amino)ethyl)-1H-indol-5-yl isobutyrate, 3-((3-(2-(ethyl(isopropyl)amino)ethyl)-1H-indol-5-yl)oxy)-3-oxopropanoic acid, 4-(3-(2-(ethyl(isopropyl)amino)ethyl)-1H-indol-5-yl)oxy)-4-oxobutanoic acid, 4-((3-(2-(ethyl(isopropyl)amino)ethyl)-1H-indol-5-yl)oxy)-5-oxopentanoic acid, 4-((3-(2-(ethyl(isopropyl)amino)ethyl)-1H-indol-5-yl)oxy)-6-oxohexanoic acid, 2-((3-(2-(ethyl(isopropyl)amino)ethyl)-1H-indol-5-yl)oxy)-6-(hydroxymethyl)tetrahydro-2H-pyran-3,4,5-triol, 2-((3-(2-(ethyl(isopropyl)amino)ethyl)-1H-indol-5-yl)oxy)-6-methyltetrahydro-2H-pyran-3,4,5-triol, N-isopropyl-N-(2-(5-methoxy-1H-indol-3-yl)ethyl)propan-2-amine, 3-(2-(diisopropylamino)ethyl)-1H-indol-5-yl dihydrogen phosphate, 3-(2-(diisopropylamino)ethyl)-1H-indol-5-yl acetate, 3-(2-(diisopropylamino)ethyl)-1H-indol-5-yl propionate, 3-(2-(diisopropylamino)ethyl)-1H-indol-5-yl butyrate, 3-(2-(diisopropylamino)ethyl)-1H-indol-5-yl isobutyrate, 3-((3-(2-(diisopropylamino)ethyl)-1H-indol-5-yl)oxy)-3-oxopropanoic acid, 4-((3-(2-(diisopropylamino)ethyl)-1H-indol-5-yl)oxy)-4-oxobutanoic acid, 5-((3-(2-(diisopropylamino)ethyl)-1H-indol-5-yl)oxy)-5-oxopentanoic acid, 6-((3-(2-(diisopropylamino)ethyl)-1H-indol-5-yl)oxy)-6-oxohexanoic acid, 2-((3-(2-(diisopropylamino)ethyl)-1H-indol-5-yl)oxy)-6-(hydroxymethyl)tetrahydro-2H-pyran-3,4,5-triol, 2-((3-(2-(diisopropylamino)ethyl)-1H-indol-5-yl)oxy)-6-methyltetrahydro-2H-pyran-3,4,5-triol, N,N-diethyl-2-(5-methoxy-1H-indol-3-yl)ethan-1-amine, 3-(2-(diethylamino)ethyl)-1H-indol-5-yl dihydrogen phosphate, 3-(2-(diethylamino)ethyl)-1H-indol-5-yl acetate, 3-(2-(diethylamino)ethyl)-1H-indol-5-yl propionate, 3-(2-(diethylamino)ethyl)-1H-indol-5-yl butyrate, 3-(2-(diethylamino)ethyl)-1H-indol-5-yl isobutyrate, 3-((3-(2-(diethylamino)ethyl)-1H-indol-5-yl)oxy)-3-oxopropanoic acid, 4-((3-(2-(diethylamino)ethyl)-1H-indol-5-yl)oxy)-4-oxobutanoic acid, 5-((3-(2-(diethylamino)ethyl)-1H-indol-5-yl)oxy)-5-oxopentanoic acid, 6-((3-(2-(diethylamino)ethyl)-1H-indol-5-yl)oxy)-6-oxohexanoic acid, 2-((3-(2-(diethylamino)ethyl)-1H-indol-5-yl)oxy)-6-(hydroxymethyl)tetrahydro-2H-pyran-3,4,5-triol, 2-((3-(2-(diethylamino)ethyl)-1H-indol-5-yl)oxy)-6-methyltetrahydro-2H-pyran-3,4,5-triol, N-ethyl-N-(2-(5-methoxy-1H-indol-3-yl)ethyl)propan-1-amine, 3-(2-(ethyl(propyl)amino)ethyl)-1H-indol-5-yl dihydrogen phosphate, 3-(2-(ethyl(propyl)amino)ethyl)-1H-indol-5-yl acetate, 3-(2-(ethyl(propyl)amino)ethyl)-1H-indol-5-yl propionate, 3-(2-(ethyl(propyl)amino)ethyl)-1H-indol-5-yl butyrate, 3-(2-(ethyl(propyl)amino)ethyl)-1H-indol-5-yl isobutyrate, 3-((3-(2-(ethyl(propyl)amino)ethyl)-1H-indol-5-yl)oxy)-3-oxopropanoic acid, 4-((3-(2-(ethyl(propyl)amino)ethyl)-1H-indol-5-yl)oxy)-4-oxobutanoic acid, 5-((3-(2-(ethyl(propyl)amino)ethyl)-1H-indol-5-yl)oxy)-5-oxopentanoic acid, 6-((3-(2-(ethyl(propyl)amino)ethyl)-1H-indol-5-yl)oxy)-6-oxohexanoic acid, 2-((3-(2-(ethyl(propyl)amino)ethyl)-1H-indol-5-yl)oxy)-6-(hydroxymethyl)tetrahydro-2H-pyran-3,4,5-triol, 2-((3-(2-(ethyl(propyl)amino)ethyl)-1H-indol-5-yl)oxy)-6-methyltetrahydro-2H-pyran-3,4,5-triol, N-(2-(5-methoxy-1H-indol-3-yl)ethyl)-N-methylcyclopropanamine, 3-(2-(cyclopropyl(methyl)amino)ethyl)-1H-indol-5-yl dihydrogen phosphate, 3-(2-(cyclopropyl(methyl)amino)ethyl)-1H-indol-5-yl acetate, 3-(2-(cyclopropyl(methyl)amino)ethyl)-1H-indol-5-yl propionate, 3-(2-(cyclopropyl(methyl)amino)ethyl)-1H-indol-5-yl butyrate, 3-(2-(cyclopropyl(methyl)amino)ethyl)-1H-indol-5-yl isobutyrate, 3-((3-(2-(cyclopropyl(methyl)amino)ethyl)-1H-indol-5-yl)oxy)-3-oxopropanoic acid, 4-((3-(2-(cyclopropyl(methyl)amino)ethyl)-1H-indol-5-yl)oxy)-4-oxobutanoic acid, 5-((3-(2-(cyclopropyl(methyl)amino)ethyl)-1H-indol-5-yl)oxy)-5-oxopentanoic acid, 6-((3-(2-(cyclopropyl(methyl)amino)ethyl)-1H-indol-5-yl)oxy)-6-oxohexanoic acid, 2-((3-(2-(cyclopropyl(methyl)amino)ethyl)-1H-indol-5-yl)oxy)-6-(hydroxymethyl)tetrahydro-2H-pyran-3,4,5-triol, 2-((3-(2-(cyclopropyl(methyl)amino)ethyl)-1H-indol-5-yl)oxy)-6-methyltetrahydro-2H-pyran-3,4,5-triol, N-ethyl-2-(5-methoxy-1H-indol-3-yl)-N-methylethan-1-amine, 3-(2-(ethyl(methyl)amino)ethyl)-1H-indol-5-yl dihydrogen phosphate, 3-(2-(ethyl(methyl)amino)ethyl)-1H-indol-5-yl acetate, 3-(2-(ethyl(methyl)amino)ethyl)-1H-indol-5-yl propionate, 3-(2-(ethyl(methyl)amino)ethyl)-1H-indol-5-yl butyrate, 3-(2-(ethyl(methyl)amino)ethyl)-1H-indol-5-yl isobutyrate, 3-((3-(2-(ethyl(methyl)amino)ethyl)-1H-indol-5-yl)oxy)-3-oxopropanoic acid, 4-((3-(2-(ethyl(methyl)amino)ethyl)-1H-indol-5-yl)oxy)-4-oxobutanoic acid, 4-((3-(2-(ethyl(methyl)amino)ethyl)-1H-indol-5-yl)oxy)-4-oxobutanoic acid, 6-((3-(2-(ethyl(methyl)amino)ethyl)-1H-indol-5-yl)oxy)-6-oxohexanoic acid, 2-((3-(2-(ethyl(methyl)amino)ethyl)-1H-indol-5-yl)oxy)-6-(hydroxymethyl)tetrahydro-2H-pyran-3,4,5-triol, 2-((3-(2-(ethyl(methyl)amino)ethyl)-1H-indol-5-yl)oxy)-6-methyltetrahydro-2H-pyran-3,4,5-triol, N-(2-(5-methoxy-1H-indol-3-yl)ethyl)-N-methylprop-2-en-1-amine, 3-(2-(allyl(methyl)amino)ethyl)-1H-indol-5-yl dihydrogen phosphate, 3-(2-(allyl(methyl)amino)ethyl)-1H-indol-5-yl acetate, 3-(2-(allyl(methyl)amino)ethyl)-1H-indol-5-yl propionate, 3-(2-(allyl(methyl)amino)ethyl)-1H-indol-5-yl butyrate, 3-(2-(allyl(methyl)amino)ethyl)-1H-indol-5-yl isobutyrate, 3-((3-(2-(allyl(methyl)amino)ethyl)-1H-indol-5-yl)oxy)-3-oxopropanoic acid, 4-((3-(2-(allyl(methyl)amino)ethyl)-1H-indol-5-yl)oxy)-4-oxobutanoic acid, 5-((3-(2-(allyl(methyl)amino)ethyl)-1H-indol-5-yl)oxy)-5-oxopentanoic acid, 6-((3-(2-(allyl(methyl)amino)ethyl)-1H-indol-5-yl)oxy)-6-oxohexanoic acid, 2-((3-(2-(allyl(methyl)amino)ethyl)-1H-indol-5-yl)oxy)-6-(hydroxymethyl)tetrahydro-2H-pyran-3,4,5-triol, 2-((3-(2-(allyl(methyl)amino)ethyl)-1H-indol-5-yl)oxy)-6-methyltetrahydro-2H-pyran-3,4,5-triol, N-ethyl-N-(2-(5-methoxy-1H-indol-3-yl)ethyl)prop-2-en-1-amine, 3-(2-(allyl(ethyl)amino)ethyl)-1H-indol-5-yl dihydrogen phosphate, 3-(2-(allyl(ethyl)amino)ethyl)-1H-indol-5-yl acetate, 3-(2-(allyl(ethyl)amino)ethyl)-1H-indol-5-yl propionate, 3-(2-(allyl(ethyl)amino)ethyl)-1H-indol-5-yl butyrate, 3-(2-(allyl(ethyl)amino)ethyl)-1H-indol-5-yl isobutyrate, 3-((3-(2-(allyl(ethyl)amino)ethyl)-1H-indol-5-yl)oxy)-3-oxopropanoic acid, 4-((3-(2-(allyl(ethyl)amino)ethyl)-1H-indol-5-yl)oxy)-4-oxobutanoic acid, 5-((3-(2-(allyl(ethyl)amino)ethyl)-1H-indol-5-yl)oxy)-5-oxopentanoic acid, 6-((3-(2-(allyl(ethyl)amino)ethyl)-1H-indol-5-yl)oxy)-6-oxohexanoic acid, 2-((3-(2-(allyl(ethyl)amino)ethyl)-1H-indol-5-yl)oxy)-6-(hydroxymethyl)tetrahydro-2H-pyran-3,4,5-triol, 2-((3-(2-(allyl(ethyl)amino)ethyl)-1H-indol-5-yl)oxy)-6-methyltetrahydro-2H-pyran-3,4,5-triol, 4-methoxy-3-(2-(pyrrolidin-1-yl)ethyl)-1H-indole, 3-(2-(pyrrolidin-1-yl)ethyl)-1H-indol-5-yl dihydrogen phosphate, 3-(2-(pyrrolidin-1-yl)ethyl)-1H-indol-5-yl acetate, 3-(2(pyrrolidin-1-yl)ethyl)-1H-indol-5-yl propionate, 3-(2-(pyrrolidin-1-yl)ethyl)-1H-indol-5-yl butyrate, 3-(2-(pyrrolidin-1-yl)ethyl)-1H-indol-5-yl isobutyrate, 3-oxo-3-((3-(2-(pyrrolidin-1-yl)ethyl)-1H-indol-5-yl)oxy)propanoic acid, 4-oxo-4-((3-(2-(pyrrolidin-1-yl)ethyl)-1H-indol-5-yl)oxy)butanoic acid, 5-oxo-5-((3-(2-(pyrrolidin-1-yl)ethyl)-1H-indol-5-yl)oxy)pentanoic acid, 6-oxo-6-((3-(2-(pyrrolidin-1-yl)ethyl)-1H-indol-5-yl)oxy)hexanoic acid, 2-(hydroxymethyl)-6-((3-(2-(pyrrolidin-1-yl)ethyl)-1H-indol-5-yl)oxy)tetrahydro-2H-pyran-3,4,5-triol, 2-methyl-6-((3-(2-(pyrrolidin-1-yl)ethyl)-1H-indol-5-yl)oxy)tetrahydro-2H-pyran-3,4,5-triol, N-(2-(5-methoxy-1H-indol-3-yl)ethyl)acetamide, 3-(2-acetamidoethyl)-1H-indol-5-yl dihydrogen phosphate, 3-(2-acetamidoethyl)-1H-indol-5-yl acetate, 3-(2-acetamidoethyl)-1H-indol-5-yl propionate, 3-(2-acetamidoethyl)-1H-indol-5-yl butyrate, 3-(2-acetamidoethyl)-1H-indol-5-yl isobutyrate, 3-((3-(2-acetamidoethyl)-1H-indol-5-yl)oxy)-3-oxopropanoic acid, 4-((3-(2-acetamidoethyl)-1H-indol-5-yl)oxy)-4-oxobutanoic acid, 5-((3-(2-acetamidoethyl)-1H-indol-5-yl)oxy)-5-oxopentanoic acid, 6-((3-(2-acetamidoethyl)-1H-indol-5-yl)oxy)-6-oxohexanoic acid, N-(2-(5-((3,4,5-trihydroxy-6-methyltetrahydro-2H-pyran-2-yl)oxy)-1H-indol-3-yl)ethyl)acetamide, N-(2-(5-((3,4,5-trihydroxy-6-(hydroxymethyl)tetrahydro-2H-pyran-2-yl)oxy)-1H-indol-3-yl)ethyl)acetamide, 2-(6-methoxy-1H-indol-3-yl)-N,N-dimethylethan-1-amine, 3-(2-(dimethylamino)ethyl)-1H-indol-6-yl dihydrogen phosphate, 3-(2-(dimethylamino)ethyl)-1H-indol-6-yl acetate, 3-(2-(dimethylamino)ethyl)-1H-indol-6-yl propionate, 3-(2-(dimethylamino)ethyl)-1H-indol-6-yl butyrate, 3-(2-(dimethylamino)ethyl)-1H-indol-6-yl isobutyrate, 3-((3-(2-(dimethylamino)ethyl)-1H-indol-6-yl)oxy)-3-oxopropanoic acid, 4-((3-(2-(dimethylamino)ethyl)-1H-indol-6-yl)oxy)-4-oxobutanoic acid, 5-((3-(2-(dimethylamino)ethyl)-1H-indol-6-yl)oxy)-5-oxopentanoic acid, 6-((3-(2-(dimethylamino)ethyl)-1H-indol-6-yl)oxy)-6-oxohexanoic acid, 2-((3-(2-(dimethylamino)ethyl)-1H-indol-6-yl)oxy)-6-(hydroxymethyl)tetrahydro-2H-pyran-3,4,5-triol, 2-((3-(2-(dimethylamino)ethyl)-1H-indol-6-yl)oxy)-6-methyltetrahydro-2H1-pyran-3,4,5-triol, N-(2-(6-methoxy-1H-indol-3-yl)ethyl)-N-propylpropan-1-amine, 3-(2-(dipropylamino)ethyl)-1H-indol-6-yl dihydrogen phosphate, 3-(2-(dipropylamino)ethyl)-1H-indol-6-yl acetate, 3-(2-(dipropylamino)ethyl)-1H-indol-6-yl propionate, 3-(2-(dipropylamino)ethyl)-1H-indol-6-yl butyrate, 3-(2-(dipropylamino)ethyl)-1H-indol-6-yl isobutyrate, 3-((3-(2-(dipropylamino)ethyl)-1H-indol-6-yl)oxy)-3-oxopropanoic acid, 4-((3-(2-(dipropylamino)ethyl)-1H-indol-6-yl)oxy)-4-oxobutanoic acid, 5-((3-(2-(dipropylamino)ethyl)-1H-indol-6-yl)oxy)-5-oxopentanoic acid, 6-((3-(2-(dipropylamino)ethyl)-1H-indol-6-yl)oxy)-6-oxohexanoic acid, 2-((3-(2-(dipropylamino)ethyl)-1H-indol-6-yl)oxy)-6-(hydroxymethyl)tetrahydro-2H-pyran-3,4,5-triol, 2-((3-(2-(dipropylamino)ethyl)-1H-indol-6-yl)oxy)-6-methyltetrahydro-2H1-pyran-3,4,5-triol, N-allyl-N-(2-(6-methoxy-1H-indol-3-yl)ethyl)prop-2-en-1-amine, 3-(2-(diallylamino)ethyl)-1H-indol-6-yl dihydrogen phosphate, 3-(2-(diallylamino)ethyl)-1H-indol-6-yl acetate, 3-(2-(diallylamino)ethyl)-1H-indol-6-yl propionate, 3-(2-(diallylamino)ethyl)-1H-indol-6-yl butyrate, 3-(2-(diallylamino)ethyl)-1H-indol-6-yl isobutyrate, 3-((3-(2-(diallylamino)ethyl)-1H-indol-6-yl)oxy)-3-oxopropanoic acid, 4-((3-(2-(diallylamino)ethyl)-1H-indol-6-yl)oxy)-4-oxobutanoic acid, 5-((3-(2-(diallylamino)ethyl)-1H-indol-6-yl)oxy)-5-oxopentanoic acid, 6-((3-(2-(diallylamino)ethyl)-1H-indol-6-yl)oxy)-6-oxohexanoic acid, 2-((3-(2-(diallylamino)ethyl)-1H-indol-6-yl)oxy)-6-(hydroxymethyl)tetrahydro-2H-pyran-3,4,5-triol, 2-((3-(2-(diallylamino)ethyl)-1H-indol-6-yl)oxy)-6-methyltetrahydro-2H-pyran-3,4,5-triol, N-(2-(6-methoxy-1H-indol-3-yl)ethyl)-N-methylpropan-2-amine, 3-(2-(isopropyl(methyl)amino)ethyl)-1H-indol-6-yl dihydrogen phosphate, 3-(2-(isopropyl(methyl)amino)ethyl)-1H-indol-6-yl acetate, 3-(2-(isopropyl(methyl)amino)ethyl)-1H-indol-6-yl propionate, 3-(2-(isopropyl(methyl)amino)ethyl)-1H-indol-6-yl butyrate, 3-(2-(isopropyl(methyl)amino)ethyl)-1H-indol-6-yl isobutyrate, 3-((3-(2-(isopropyl(methyl)amino)ethyl)-1H-indol-6-yl)oxy)-3-oxopropanoic acid, 4-((3-(2-(isopropyl(methyl)amino)ethyl)-1H-indol-6-yl)oxy)-4-oxobutanoic acid, 5-((3-(2-(isopropyl(methyl)amino)ethyl)-1H-indol-6-yl)oxy)-5-oxopentanoic acid, 6-((3-(2-(isopropyl(methyl)amino)ethyl)-1H-indol-6-yl)oxy)-6-oxohexanoic acid, 2-((3-(2-(isopropyl(methyl)amino)ethyl)-1H-indol-6-yl)oxy)-6-(hydroxymethyl)tetrahydro-2H-pyran-3,4,5-triol, 2-((3-(2-(isopropyl(methyl)amino)ethyl)-1H-indol-6-yl)oxy)-6-methyltetrahydro-2H-pyran-3,4,5-triol, N-ethyl-N-(2-(6-methoxy-1H-indol-3-yl)ethyl)propan-2-amine, 3-(2-(ethyl(isopropyl)amino)ethyl)-1H-indol-6-yl dihydrogen phosphate, 3-(2-(ethyl(isopropyl)amino)ethyl)-1H-indol-6-yl acetate, 3-(2-(ethyl(isopropyl)amino)ethyl)-1H-indol-6-yl propionate, 3-(2-(ethyl(isopropyl)amino)ethyl)-1H-indol-6-yl butyrate, 3-(2-(ethyl(isopropyl)amino)ethyl)-1H-indol-6-yl isobutyrate, 3-((3-(2-(ethyl(isopropyl)amino)ethyl)-1H-indol-6-yl)oxy)-3-oxopropanoic acid, 4-((3-(2-(ethyl(isopropyl)amino)ethyl)-1H-indol-6-yl)oxy)-4-oxobutanoic acid, 4-((3-(2-(ethyl(isopropyl)amino)ethyl)-1H-indol-6-yl)oxy)-5-oxopentanoic acid, 4-((3-(2-(ethyl(isopropyl)amino)ethyl)-1H-indol-6-yl)oxy)-6-oxohexanoic acid, 2-((3-(2-(ethyl(isopropyl)amino)ethyl)-1H-indol-6-yl)oxy)-6-(hydroxymethyl)tetrahydro-2H-pyran-3,4,5-triol, 2-((3-(2-(ethyl(isopropyl)amino)ethyl)-1H-indol-6-yl)oxy)-6-methyltetrahydro-2H-pyran-3,4,5-triol, N-isopropyl-N-(2-(6-methoxy-1H-indol-3-yl)ethyl)propan-2-amine, 3-(2-(diisopropylamino)ethyl)-1H-indol-6-yl dihydrogen phosphate, 3-(2-(diisopropylamino)ethyl)-1H-indol-6-yl acetate, 3-(2-(diisopropylamino)ethyl)-1H-indol-6-yl propionate, 3-(2-(diisopropylamino)ethyl)-1H-indol-6-yl butyrate, 3-(2-(diisopropylamino)ethyl)-1H-indol-6-yl isobutyrate, 3-((3-(2-(diisopropylamino)ethyl)-1H-indol-6-yl)oxy)-3-oxopropanoic acid, 4-((3-(2-(diisopropylamino)ethyl)-1H-indol-6-yl)oxy)-4-oxobutanoic acid, 5-((3-(2-(diisopropylamino)ethyl)-1H-indol-6-yl)oxy)-5-oxopentanoic acid, 6-((3-(2-(diisopropylamino)ethyl)-1H-indol-6-yl)oxy)-6-oxohexanoic acid, 2-((3-(2-(diisopropylamino)ethyl)-1H-indol-6-yl)oxy)-6-(hydroxymethyl)tetrahydro-2H-pyran-3,4,5-triol, 2-((3-(2-(diisopropylamino)ethyl)-1H-indol-6-yl)oxy)-6-methyltetrahydro-2H-pyran-3,4,5-triol, N,N-diethyl-2-(6-methoxy-1H-indol-3-yl)ethan-1-amine, 3-(2-(diethylamino)ethyl)-1H-indol-6-yl dihydrogen phosphate, 3-(2-(diethylamino)ethyl)-1H-indol-6-yl acetate, 3-(2-(diethylamino)ethyl)-1H-indol-6-yl propionate, 3-(2-(diethylamino)ethyl)-1H-indol-6-yl butyrate, 3-(2-(diethylamino)ethyl)-1H-indol-6-yl isobutyrate, 3-((3-(2-(diethylamino)ethyl)-1H-indol-6-yl)oxy)-3-oxopropanoic acid, 4-((3-(2-(diethylamino)ethyl)-1H-indol-6-yl)oxy)-4-oxobutanoic acid, 5-((3-(2-(diethylamino)ethyl)-1H-indol-6-yl)oxy)-5-oxopentanoic acid, 6-((3-(2-(diethylamino)ethyl)-1H-indol-6-yl)oxy)-6-oxohexanoic acid, 2-((3-(2-(diethylamino)ethyl)-1H-indol-6-yl)oxy)-6-(hydroxymethyl)tetrahydro-2H-pyran-3,4,5-triol, 2-((3-(2-(diethylamino)ethyl)-1H-indol-6-yl)oxy)-6-methyltetrahydro-2H-pyran-3,4,5-triol, N-ethyl-N-(2-(6-methoxy-1H-indol-3-yl)ethyl)propan-1-amine, 3-(2-(ethyl(propyl)amino)ethyl)-1H-indol-6-yl dihydrogen phosphate, 3-(2-(ethyl(propyl)amino)ethyl)-1H-indol-6-yl acetate, 3-(2-(ethyl(propyl)amino)ethyl)-1H-indol-6-yl propionate, 3-(2-(ethyl(propyl)amino)ethyl)-1H-indol-6-yl butyrate, 3-((2-(ethyl(propyl)amino)ethyl)-1H-indol-6-yl isobutyrate, 3-((3-(2-(ethyl(propyl)amino)ethyl)-1H-indol-6-yl)oxy)-3-oxopropanoic acid, 4-((3-(2-(ethyl(propyl)amino)ethyl)-1H-indol-6-yl)oxy)-4-oxobutanoic acid, 5-((3-(2-(ethyl(propyl)amino)ethyl)-1H-indol-6-yl)oxy)-5-oxopentanoic acid, 6-((3-(2-(ethyl(propyl)amino)ethyl)-1H-indol-6-yl)oxy)-6-oxohexanoic acid, 2-((3-(2-(ethyl(propyl)amino)ethyl)-1H-indol-6-yl)oxy)-6-(hydroxymethyl)tetrahydro-2H-pyran-3,4,5-triol, 2-((3-(2-(ethyl(propyl)amino)ethyl)-1H-indol-6-yl)oxy)-6-methyltetrahydro-2H-pyran-3,4,5-triol, N-(2-(6-methoxy-1H-indol-3-yl)ethyl)-N-methylcyclopropanamine, 3-(2-(cyclopropyl(methyl)amino)ethyl)-1H-indol-6-yl dihydrogen phosphate, 3-(2-(cyclopropyl(methyl)amino)ethyl)-1H-indol-6-yl acetate, 3-(2-(cyclopropyl(methyl)amino)ethyl)-1H-indol-6-yl propionate, 3-(2-(cyclopropyl(methyl)amino)ethyl)-1H-indol-6-yl butyrate, 3-(2-(cyclopropyl(methyl)amino)ethyl)-1H-indol-6-yl isobutyrate, 3-((3-(2-(cyclopropyl(methyl)amino)ethyl)-1H-indol-6-yl)oxy)-3-oxopropanoic acid, 4-((3-(2-(cyclopropyl(methyl)amino)ethyl)-1H-indol-6-yl)oxy)-4-oxobutanoic acid, 5-((3-(2-(cyclopropyl(methyl)amino)ethyl)-1H-indol-6-yl)oxy)-5-oxopentanoic acid, 6-((3-(2-(cyclopropyl(methyl)amino)ethyl)-1H-indol-6-yl)oxy)-6-oxohexanoic acid, 2-((3-(2-(cyclopropyl(methyl)amino)ethyl)-1H-indol-6-yl)oxy)-6-(hydroxymethyl)tetrahydro-2H-pyran-3,4,5-triol, 2-((3-(2-(cyclopropyl(methyl)amino)ethyl)-1H-indol-6-yl)oxy)-6-methyltetrahydro-2H-pyran-3,4,5-triol, N-ethyl-2-(6-methoxy-1H-indol-3-yl)-N-methylethan-1-amine, 3-(2-(ethyl(methyl)amino)ethyl)-1H-indol-6-yl dihydrogen phosphate, 3-(2-(ethyl(methyl)amino)ethyl)-1H-indol-6-yl acetate, 3-(2-(ethyl(methyl)amino)ethyl)-1H-indol-6-yl propionate, 3-(2-(ethyl(methyl)amino)ethyl)-1H-indol-6-yl butyrate, 3-(2-(ethyl(methyl)amino)ethyl)-1H-indol-6-yl isobutyrate, 3-((3-(2-(ethyl(methyl)amino)ethyl)-1H-indol-6-yl)oxy)-3-oxopropanoic acid, 4-((3-(2-(ethyl(methyl)amino)ethyl)-1H-indol-6-yl)oxy)-4-oxobutanoic acid, 4-((3-(2-(ethyl(methyl)amino)ethyl)-1H-indol-6-yl)oxy)-4-oxobutanoic acid, 6-((3-(2-(ethyl(methyl)amino)ethyl)-1H-indol-6-yl)oxy)-6-oxohexanoic acid, 2-((3-(2-(ethyl(methyl)amino)ethyl)-1H-indol-6-yl)oxy)-6-(hydroxymethyl)tetrahydro-2H-pyran-3,4,5-triol, 2-((3-(2-(ethyl(methyl)amino)ethyl)-1H-indol-6-yl)oxy)-6-methyltetrahydro-2H-pyran-3,4,5-triol, N-(2-(6-methoxy-1H-indol-3-yl)ethyl)-N-methylprop-2-en-1-amine, 3-(2-(allyl(methyl)amino)ethyl)-1H-indol-6-yl dihydrogen phosphate, 3-(2-(allyl(methyl)amino)ethyl)-1H-indol-6-yl acetate, 3-(2-allyl(methyl)amino)ethyl)-1H-indol-6-yl propionate, 3-(2-(allyl(methyl)amino)ethyl)-1H-indol-6-yl butyrate, 3-(2-(allyl(methyl)amino)ethyl)-1H-indol-6-yl isobutyrate, 3-((3-(2-(allyl(methyl)amino)ethyl)-1H-indol-6-yl)oxy)-3-oxopropanoic acid, 4-((3-(2-(allyl(methyl)amino)ethyl)-1H-indol-6-yl)oxy)-4-oxobutanoic acid, 5-((3-(2-(allyl(methyl)amino)ethyl)-1H-indol-6-yl)oxy)-5-oxopentanoic acid, 6-((3-(2-(allyl(methyl)amino)ethyl)-1H-indol-6-yl)oxy)-6-oxohexanoic acid, 2-((3-(2-(allyl(methyl)amino)ethyl)-1H-indol-6-yl)oxy)-6-(hydroxymethyl)tetrahydro-2H-pyran-3,4,5-triol, 2-((3-(2-(allyl(methyl)amino)ethyl)-1H-indol-6-yl)oxy)-6-methyltetrahydro-2H-pyran-3,4,5-triol, N-ethyl-N-(2-(6-methoxy-1H-indol-3-yl)ethyl)prop-2-en-1-amine, 3-(2-(allyl(ethyl)amino)ethyl)-1H-indol-6-yl dihydrogen phosphate, 3-(2-(allyl(ethyl)amino)ethyl)-1H-indol-6-yl acetate, 3-(2-(allyl(ethyl)amino)ethyl)-1H-indol-6-yl propionate, 3-(2-(allyl(ethyl)amino)ethyl)-1H-indol-6-yl butyrate, 3-(2-(allyl(ethyl)amino)ethyl)-1H-indol-6-yl isobutyrate, 3-((3-(2-(allyl(ethyl)amino)ethyl)-1H-indol-6-yl)oxy)-3-oxopropanoic acid, 4-((3-(2-(allyl(ethyl)amino)ethyl)-1H-indol-6-yl)oxy)-4-oxobutanoic acid, 5-((3-(2-(allyl(ethyl)amino)ethyl)-1H-indol-6-yl)oxy)-5-oxopentanoic acid, 6-((3-(2-(allyl(ethyl)amino)ethyl)-1H-indol-6-yl)oxy)-6-oxohexanoic acid, 2-((3-(2-(allyl(ethyl)amino)ethyl)-1H-indol-6-yl)oxy)-6-(hydroxymethyl)tetrahydro-2H-pyran-3,4,5-triol, 2-((3-(2-(allyl(ethyl)amino)ethyl)-1H-indol-6-yl)oxy)-6-methyltetrahydro-2H-pyran-3,4,5-triol, 4-methoxy-3-(2-(pyrrolidin-1-yl)ethyl)-1H-indole, 3-(2-(pyrrolidin-1-yl)ethyl)-1H-indol-6-yl dihydrogen phosphate, 3-(2-(pyrrolidin-1-yl)ethyl)-1H-indol-6-yl acetate, 3-(2-(pyrrolidin-1-yl)ethyl)-1H-indol-6-yl propionate, 3-(2-(pyrrolidin-1-yl)ethyl)-1H-indol-6-yl butyrate, 3-(2-(pyrrolidin-1-yl)ethyl)-1H-indol-6-yl isobutyrate, 3-oxo-3-((3-(2-(pyrrolidin-1-yl)ethyl)-1H-indol-6-yl)oxy)propanoic acid, 4-oxo-4-((3-(2-(pyrrolidin-1-yl)ethyl)-1H-indol-6-yl)oxy)butanoic acid, 5-oxo-5-((3-(2-(pyrrolidin-1-yl)ethyl)-1H-indol-6-yl)oxy)pentanoic acid, 6-oxo-6-((3-(2-(pyrrolidin-1-yl)ethyl)-1H-indol-6-yl)oxy)hexanoic acid, 2-(hydroxymethyl)-6-((3-(2-(pyrrolidin-1-yl)ethyl)-1H-indol-6-yl)oxy)tetrahydro-2H-pyran-3,4,5-triol, 2-methyl-6-((3-(2-(pyrrolidin-1-yl)ethyl)-1H-indol-6-yl)oxy)tetrahydro-2H-pyran-3,4,5-triol, N-(2-(6-methoxy-1H-indol-3-yl)ethyl)acetamide, 3-(2-acetamidoethyl)-1H-indol-6-yl dihydrogen phosphate, 3-(2-acetamidoethyl)-1H-indol-6-yl acetate, 3-(2-acetamidoethyl)-1H-indol-6-yl propionate, 3-(2-acetamidoethyl)-1H-indol-6-yl butyrate, 3-(2-acetamidoethyl)-1H-indol-6-yl isobutyrate, 3-((3-(2-acetamidoethyl)-1H-indol-6-yl)oxy)-3-oxopropanoic acid, 4-((3-(2-acetamidoethyl)-1H-indol-6-yl)oxy)-4-oxobutanoic acid, 5-((3-(2-acetamidoethyl)-1H-indol-6-yl)oxy)-5-oxopentanoic acid, 6-((3-(2-acetamidoethyl)-1H-indol-6-yl)oxy)-6-oxohexanoic acid, N-(2-(6-((3,4,5-trihydroxy-6-methyltetrahydro-2H-pyran-2-yl)oxy)-1H-indol-3-yl)ethyl)acetamide, N-(2-(6-((3,4,5-trihydroxy-6-(hydroxymethyl)tetrahydro-2H-pyran-2-yl)oxy)-1H-indol-3-yl)ethyl)acetamide, 2-(7-methoxy-1H-indol-3-yl)-N,N-dimethylethan-1-amine, 3-(2-(dimethylamino)ethyl)-1H-indol-7-yl dihydrogen phosphate, 3-(2-(dimethylamino)ethyl)-1H-indol-7-yl acetate, 3-(2-(dimethylamino)ethyl)-1H-indol-7-yl propionate, 3-(2-(dimethylamino)ethyl)-1H-indol-7-yl butyrate, 3-(2-(dimethylamino)ethyl)-1H-indol-7-yl isobutyrate, 3-((3-(2-(dimethylamino)ethyl)-1H-indol-7-yl)oxy)-3-oxopropanoic acid, 4-((3-(2-(dimethylamino)ethyl)-1H-indol-7-yl)oxy)-4-oxobutanoic acid, 5-((3-(2-(dimethylamino)ethyl)-1H-indol-7-yl)oxy)-5-oxopentanoic acid, 6-((3-(2-(dimethylamino)ethyl)-1H-indol-7-yl)oxy)-6-oxohexanoic acid, 2-((3-(2-(dimethylamino)ethyl)-1H-indol-7-yl)oxy)-6-(hydroxymethyl)tetrahydro-2H-pyran-3,4,5-triol, 2-((3-(2-(dimethylamino)ethyl)-1H-indol-7-yl)oxy)-6-methyltetrahydro-2H1-pyran-3,4,5-triol, N-(2-(7-methoxy-1H-indol-3-yl)ethyl)-N-propylpropan-1-amine, 3-(2-(dipropylamino)ethyl)-1H-indol-7-yl dihydrogen phosphate, 3-(2-(dipropylamino)ethyl)-1H-indol-7-yl acetate, 3-(2-(dipropylamino)ethyl)-1H-indol-7-yl propionate, 3-(2-(dipropylamino)ethyl)-1H-indol-7-yl butyrate, 3-(2-(dipropylamino)ethyl)-1H-indol-7-yl isobutyrate, 3-((3-(2-(dipropylamino)ethyl)-1H-indol-7-yl)oxy)-3-oxopropanoic acid, 4-((3-(2-(dipropylamino)ethyl)-1H-indol-7-yl)oxy)-4-oxobutanoic acid, 5-((3-(2-(dipropylamino)ethyl)-1H-indol-7-yl)oxy)-5-oxopentanoic acid, 6-((3-(2-(dipropylamino)ethyl)-1H-indol-7-yl)oxy)-6-oxohexanoic acid, 2-((3-(2-(dipropylamino)ethyl)-1H-indol-7-yl)oxy)-6-(hydroxymethyl)tetrahydro-2H-pyran-3,4,5-triol, 2-((3-(2-(dipropylamino)ethyl)-1H-indol-7-yl)oxy)-6-methyltetrahydro-2H1-pyran-3,4,5-triol, N-allyl-N-(2-(7-methoxy-1H-indol-3-yl)ethyl)prop-2-en-1-amine, 3-(2-(diallylamino)ethyl)-1H-indol-7-yl dihydrogen phosphate, 3-(2-(diallylamino)ethyl)-1H-indol-7-yl acetate, 3-(2-(diallylamino)ethyl)-1H-indol-7-yl propionate, 3-(2-(diallylamino)ethyl)-1H-indol-7-yl butyrate, 3-(2-(diallylamino)ethyl)-1H-indol-7-yl isobutyrate, 3-((3-(2-(diallylamino)ethyl)-1H-indol-7-yl)oxy)-3-oxopropanoic acid, 4-((3-(2-(diallylamino)ethyl)-1H-indol-7-yl)oxy)-4-oxobutanoic acid, 5-((3-(2-(diallylamino)ethyl)-1H-indol-7-yl)oxy)-5-oxopentanoic acid, 6-((3-(2-(diallylamino)ethyl)-1H-indol-7-yl)oxy)-6-oxohexanoic acid, 2-((3-(2-(diallylamino)ethyl)-1H-indol-7-yl)oxy)-6-(hydroxymethyl)tetrahydro-2H-pyran-3,4,5-triol, 2-((3-(2-(diallylamino)ethyl)-1H-indol-7-yl)oxy)-6-methyltetrahydro-2H-pyran-3,4,5-triol, N-(2-(7-methoxy-1H-indol-3-yl)ethyl)-N-methylpropan-2-amine, 3-(2-(isopropyl(methyl)amino)ethyl)-1H-indol-7-yl dihydrogen phosphate, 3-(2-(isopropyl(methyl)amino)ethyl)-1H-indol-7-yl acetate, 3-(2-(isopropyl(methyl)amino)ethyl)-1H-indol-7-yl propionate, 3-(2-(isopropyl(methyl)amino)ethyl)-1H-indol-7-yl butyrate, 3-(2-(isopropyl(methyl)amino)ethyl)-1H-indol-7-yl isobutyrate, 3-((3-(2-(isopropyl(methyl)amino)ethyl)-1H-indol-7-yl)oxy)-3-oxopropanoic acid, 4-((3-(2-(isopropyl(methyl)amino)ethyl)-1H-indol-7-yl)oxy)-4-oxobutanoic acid, 5-((3-(2-(isopropyl(methyl)amino)ethyl)-1H-indol-7-yl)oxy)-5-oxopentanoic acid, 6-((3-(2-(isopropyl(methyl)amino)ethyl)-1H-indol-7-yl)oxy)-6-oxohexanoic acid, 2-((3-(2-(isopropyl(methyl)amino)ethyl)-1H-indol-7-yl)oxy)-6-(hydroxymethyl)tetrahydro-2H-pyran-3,4,5-triol, 2-((3-(2-(isopropyl(methyl)amino)ethyl)-1H-indol-7-yl)oxy)-6-methyltetrahydro-2H-pyran-3,4,5-triol, N-ethyl-N-(2-(7-methoxy-1H-indol-3-yl)ethyl)propan-2-amine, 3-(2-(ethyl(isopropyl)amino)ethyl)-1H-indol-7-yl dihydrogen phosphate, 3-(2-(ethyl(isopropyl)amino)ethyl)-1H-indol-7-yl acetate, 3-(2-(ethyl(isopropyl)amino)ethyl)-1H-indol-7-yl propionate, 3-(2-(ethyl(isopropyl)amino)ethyl)-1H-indol-7-yl butyrate, 3-(2-(ethyl(isopropyl)amino)ethyl)-1H-indol-7-yl isobutyrate, 3-((3-(2-(ethyl(isopropyl)amino)ethyl)-1H-indol-7-yl)oxy)-3-oxopropanoic acid, 4-(3-(2-(ethyl(isopropyl)amino)ethyl)-1H-indol-7-yl)oxy)-4-oxobutanoic acid, 4-((3-(2-(ethyl(isopropyl)amino)ethyl)-1H-indol-7-yl)oxy)-5-oxopentanoic acid, 4-((3-(2-(ethyl(isopropyl)amino)ethyl)-1H-indol-7-yl)oxy)-6-oxohexanoic acid, 2-((3-(2-(ethyl(isopropyl)amino)ethyl)-1H-indol-7-yl)oxy)-6-(hydroxymethyl)tetrahydro-2H-pyran-3,4,5-triol, 2-((3-(2-(ethyl(isopropyl)amino)ethyl)-1H-indol-7-yl)oxy)-6-methyltetrahydro-2H-pyran-3,4,5-triol, N-isopropyl-N-(2-(7-methoxy-1H-indol-3-yl)ethyl)propan-2-amine, 3-(2-(diisopropylamino)ethyl)-1H-indol-7-yl dihydrogen phosphate, 3-(2-(diisopropylamino)ethyl)-1H-indol-7-yl acetate, 3-(2-(diisopropylamino)ethyl)-1H-indol-7-yl propionate, 3-(2-(diisopropylamino)ethyl)-1H-indol-7-yl butyrate, 3-(2-(diisopropylamino)ethyl)-1H-indol-7-yl isobutyrate, 3-((3-(2-(diisopropylamino)ethyl)-1H-indol-7-yl)oxy)-3-oxopropanoic acid, 4-((3-(2-(diisopropylamino)ethyl)-1H-indol-7-yl)oxy)-4-oxobutanoic acid, 5-((3-(2-(diisopropylamino)ethyl)-1H-indol-7-yl)oxy)-5-oxopentanoic acid, 6-((3-(2-(diisopropylamino)ethyl)-1H-indol-7-yl)oxy)-6-oxohexanoic acid, 2-((3-(2-(diisopropylamino)ethyl)-1H-indol-7-yl)oxy)-6-(hydroxymethyl)tetrahydro-2H-pyran-3,4,5-triol, 2-((3-(2-(diisopropylamino)ethyl)-1H-indol-7-yl)oxy)-6-methyltetrahydro-2H-pyran-3,4,5-triol, N,N-diethyl-2-(7-methoxy-1H-indol-3-yl)ethan-1-amine, 3-(2-(diethylamino)ethyl)-1H-indol-7-yl dihydrogen phosphate, 3-(2-(diethylamino)ethyl)-1H-indol-7-yl acetate, 3-(2-(diethylamino)ethyl)-1H-indol-7-yl propionate, 3-(2-(diethylamino)ethyl)-1H-indol-7-yl butyrate, 3-(2-(diethylamino)ethyl)-1H-indol-7-yl isobutyrate, 3-((3-(2-(diethylamino)ethyl)-1H-indol-7-yl)oxy)-3-oxopropanoic acid, 4-((3-(2-(diethylamino)ethyl)-1H-indol-7-yl)oxy)-4-oxobutanoic acid, 5-((3-(2-(diethylamino)ethyl)-1H-indol-7-yl)oxy)-5-oxopentanoic acid, 6-((3-(2-(diethylamino)ethyl)-1H-indol-7-yl)oxy)-6-oxohexanoic acid, 2-((3-(2-(diethylamino)ethyl)-1H-indol-7-yl)oxy)-6-(hydroxymethyl)tetrahydro-2H-pyran-3,4,5-triol, 2-((3-(2-(diethylamino)ethyl)-1H-indol-7-yl)oxy)-6-methyltetrahydro-2H-pyran-3,4,5-triol, N-ethyl-N-(2-(7-methoxy-1H-indol-3-yl)ethyl)propan-1-amine, 3-(2-(ethyl(propyl)amino)ethyl)-1H-indol-7-yl dihydrogen phosphate, 3-(2-(ethyl(propyl)amino)ethyl)-1H-indol-7-yl acetate, 3-(2-(ethyl(propyl)amino)ethyl)-1H-indol-7-yl propionate, 3-(2-(ethyl(propyl)amino)ethyl)-1H-indol-7-yl butyrate, 3-(2-(ethyl(propyl)amino)ethyl)-1H-indol-7-yl isobutyrate, 3-((3-(2-(ethyl(propyl)amino)ethyl)-1H-indol-7-yl)oxy)-3-oxopropanoic acid, 4-((3-(2-(ethyl(propyl)amino)ethyl)-1H-indol-7-yl)oxy)-4-oxobutanoic acid, 5-((3-(2-(ethyl(propyl)amino)ethyl)-1H-indol-7-yl)oxy)-5-oxopentanoic acid, 6-((3-(2-(ethyl(propyl)amino)ethyl)-1H-indol-7-yl)oxy)-6-oxohexanoic acid, 2-((3-(2-(ethyl(propyl)amino)ethyl)-1H-indol-7-yl)oxy)-6-(hydroxymethyl)tetrahydro-2H-pyran-3,4,5-triol, 2-((3-(2-(ethyl(propyl)amino)ethyl)-1H-indol-7-yl)oxy)-6-methyltetrahydro-2H-pyran-3,4,5-triol, N-(2-(7-methoxy-1H-indol-3-yl)ethyl)-N-methylcyclopropanamine, 3-(2-(cyclopropyl(methyl)amino)ethyl)-1H-indol-7-yl dihydrogen phosphate, 3-(2-(cyclopropyl(methyl)amino)ethyl)-1H-indol-7-yl acetate, 3-(2-(cyclopropyl(methyl)amino)ethyl)-1H-indol-7-yl propionate, 3-(2-(cyclopropyl(methyl)amino)ethyl)-1H-indol-7-yl butyrate, 3-(2-(cyclopropyl(methyl)amino)ethyl)-1H-indol-7-yl isobutyrate, 3-((3-(2-(cyclopropyl(methyl)amino)ethyl)-1H-indol-7-yl)oxy)-3-oxopropanoic acid, 4-((3-(2-(cyclopropyl(methyl)amino)ethyl)-1H-indol-7-yl)oxy)-4-oxobutanoic acid, 5-((3-(2-(cyclopropyl(methyl)amino)ethyl)-1H-indol-7-yl)oxy)-5-oxopentanoic acid, 6-((3-(2-(cyclopropyl(methyl)amino)ethyl)-1H-indol-7-yl)oxy)-6-oxohexanoic acid, 2-((3-(2-(cyclopropyl(methyl)amino)ethyl)-1H-indol-7-yl)oxy)-6-(hydroxymethyl)tetrahydro-2H-pyran-3,4,5-triol, 2-((3-(2-(cyclopropyl(methyl)amino)ethyl)-1H-indol-7-yl)oxy)-6-methyltetrahydro-2H-pyran-3,4,5-triol, N-ethyl-2-(7-methoxy-1H-indol-3-yl)-N-methylethan-1-amine, 3-(2-(ethyl(methyl)amino)ethyl)-1H-indol-7-yl dihydrogen phosphate, 3-(2-(ethyl(methyl)amino)ethyl)-1H-indol-7-yl acetate, 3-(2-(ethyl(methyl)amino)ethyl)-1H-indol-7-yl propionate, 3-(2-(ethyl(methyl)amino)ethyl)-1H-indol-7-yl butyrate, 3-(2-(ethyl(methyl)amino)ethyl)-1H-indol-7-yl isobutyrate, 3-((3-(2-(ethyl(methyl)amino)ethyl)-1H-indol-7-yl)oxy)-3-oxopropanoic acid, 4-((3-(2-(ethyl(methyl)amino)ethyl)-1H-indol-7-yl)oxy)-4-oxobutanoic acid, 4-((3-(2-(ethyl(methyl)amino)ethyl)-1H-indol-7-yl)oxy)-4-oxobutanoic acid, 6-((3-(2-(ethyl(methyl)amino)ethyl)-1H-indol-7-yl)oxy)-6-oxohexanoic acid, 2-((3-(2-(ethyl(methyl)amino)ethyl)-1H-indol-7-yl)oxy)-6-(hydroxymethyl)tetrahydro-2H-pyran-3,4,5-triol, 2-((3-(2-(ethyl(methyl)amino)ethyl)-1H-indol-7-yl)oxy)-6-methyltetrahydro-2H-pyran-3,4,5-triol, N-(2-(7-methoxy-1H-indol-3-yl)ethyl)-N-methylprop-2-en-1-amine, 3-(2-(allyl(methyl)amino)ethyl)-1H-indol-7-yl dihydrogen phosphate, 3-(2-(allyl(methyl)amino)ethyl)-1H-indol-7-yl acetate, 3-(2-allyl(methyl)amino)ethyl)-1H-indol-7-yl propionate, 3-(2-(allyl(methyl)amino)ethyl)-1H-indol-7-yl butyrate, 3-(2-(allyl(methyl)amino)ethyl)-1H-indol-7-yl isobutyrate, 3-((3-(2-(allyl(methyl)amino)ethyl)-1H-indol-7-yl)oxy)-3-oxopropanoic acid, 4-((3-(2-(allyl(methyl)amino)ethyl)-1H-indol-7-yl)oxy)-4-oxobutanoic acid, 5-((3-(2-(allyl(methyl)amino)ethyl)-1H-indol-7-yl)oxy)-5-oxopentanoic acid, 6-((3-(2-(allyl(methyl)amino)ethyl)-1H-indol-7-yl)oxy)-6-oxohexanoic acid, 2-((3-(2-(allyl(methyl)amino)ethyl)-1H-indol-7-yl)oxy)-6-(hydroxymethyl)tetrahydro-2H-pyran-3,4,5-triol, 2-((3-(2-(allyl(methyl)amino)ethyl)-1H-indol-7-yl)oxy)-6-methyltetrahydro-2H-pyran-3,4,5-triol, N-ethyl-N-(2-(7-methoxy-1H-indol-3-yl)ethyl)prop-2-en-1-amine, 3-(2-(allyl(ethyl)amino)ethyl)-1H-indol-7-yl dihydrogen phosphate, 3-(2-(allyl(ethyl)amino)ethyl)-1H-indol-7-yl acetate, 3-(2-(allyl(ethyl)amino)ethyl)-1H-indol-7-yl propionate, 3-(2-(allyl(ethyl)amino)ethyl)-1H-indol-7-yl butyrate, 3-(2-(allyl(ethyl)amino)ethyl)-1H-indol-7-yl isobutyrate, 3-((3-(2-(allyl(ethyl)amino)ethyl)-1H-indol-7-yl)oxy)-3-oxopropanoic acid, 4-((3-(2-(allyl(ethyl)amino)ethyl)-1H-indol-7-yl)oxy)-4-oxobutanoic acid, 5-((3-(2-(allyl(ethyl)amino)ethyl)-1H-indol-7-yl)oxy)-5-oxopentanoic acid, 6-((3-(2-(allyl(ethyl)amino)ethyl)-1H-indol-7-yl)oxy)-6-oxohexanoic acid, 2-((3-(2-(allyl(ethyl)amino)ethyl)-1H-indol-7-yl)oxy)-6-(hydroxymethyl)tetrahydro-2H-pyran-3,4,5-triol, 2-((3-(2-(allyl(ethyl)amino)ethyl)-1H-indol-7-yl)oxy)-6-methyltetrahydro-2H-pyran-3,4,5-triol, 4-methoxy-3-(2-(pyrrolidin-1-yl)ethyl)-1H-indole, 3-(2-(pyrrolidin-1-yl)ethyl)-1H-indol-7-yl dihydrogen phosphate, 3-(2-(pyrrolidin-1-yl)ethyl)-1H-indol-7-yl acetate, 3-(2-(pyrrolidin-1-yl)ethyl)-1H-indol-7-yl propionate, 3-(2-(pyrrolidin-1-yl)ethyl)-1H-indol-7-yl butyrate, 3-(2-(pyrrolidin-1-yl)ethyl)-1H-indol-7-yl isobutyrate, 3-oxo-3-((3-(2-(pyrrolidin-1-yl)ethyl)-1H-indol-7-yl)oxy)propanoic acid, 4-oxo-4-((3-(2-(pyrrolidin-1-yl)ethyl)-1H-indol-7-yl)oxy)butanoic acid, 5-oxo-5-((3-(2-(pyrrolidin-1-yl)ethyl)-1H-indol-7-yl)oxy)pentanoic acid, 6-oxo-6-((3-(2-(pyrrolidin-1-yl)ethyl)-1H-indol-7-yl)oxy)hexanoic acid, 2-(hydroxymethyl)-6-((3-(2-(pyrrolidin-1-yl)ethyl)-1H-indol-7-yl)oxy)tetrahydro-2H-pyran-3,4,5-triol, 2-methyl-6-((3-(2-(pyrrolidin-1-yl)ethyl)-1H-indol-7-yl)oxy)tetrahydro-2H-pyran-3,4,5-triol, N-(2-(7-methoxy-1H-indol-3-yl)ethyl)acetamide, 3-(2-acetamidoethyl)-1H-indol-7-yl dihydrogen phosphate, 3-(2-acetamidoethyl)-1H-indol-7-yl acetate, 3-(2-acetamidoethyl)-1H-indol-7-yl propionate, 3-(2-acetamidoethyl)-1H-indol-7-yl butyrate, 3-(2-acetamidoethyl)-1H-indol-7-yl isobutyrate, 3-((3-(2-acetamidoethyl)-1H-indol-7-yl)oxy)-3-oxopropanoic acid, 4-((3-(2-acetamidoethyl)-1H-indol-7-yl)oxy)-4-oxobutanoic acid, 5-((3-(2-acetamidoethyl)-1H-indol-7-yl)oxy)-5-oxopentanoic acid, 6-((3-(2-acetamidoethyl)-1H-indol-7-yl)oxy)-6-oxohexanoic acid, N-(2-(7-((3,4,5-trihydroxy-6-methyltetrahydro-2H-pyran-2-yl)oxy)-1H-indol-3-yl)ethyl)acetamide, and N-(2-(7-((3,4,5-trihydroxy-6-(hydroxymethyl)tetrahydro-2H-pyran-2-yl)oxy)-1H-indol-3-yl)ethyl)acetamide, or a pharmaceutically acceptable salt thereof.

In some embodiments, a compound provided herein is a modified ibogamine alkaloid. Examples of modified ibogamine alkaloids include, but are not limited to, 3-(((6R,6aS,7S9R,11S)-7-ethyl-6,6a,7,8,9,10,12,13-octahydro-5H-6,9-methanopyrido[1′,2′:1,2]azepino[4,5-b]indol-2-yl)oxy)-3-oxopropanoic acid, 2-(((6R,6aS,7S,9R,11S)-7-ethyl-6,6a,7,8,9,10,12,13-octahydro-5H-6,9-methanopyrido[1′,2′:1,2]azepino[4,5-b]indol-2-yl)oxy)-6-(hydroxymethyl)tetrahydro-2H-pyran-3,4,5-triol, 4-(((6R,6aS,7S,9R,11S)-7-ethyl-6,6a,7,8,9,10,12,13-octahydro-5H-6,9-methanopyrido[1′,2′:1,2]azepino[4,5-b]indol-2-yl)oxy)-4-oxobutanoic acid, 2-(((6R,6aS,7S,9R,11S)-7-ethyl-6,6a,7,8,9,10,12,13-octahydro-5H-6,9-methanopyrido[1′,2′:1,2]azepino[4,5-b]indol-2-yl)oxy)-6-methyltetrahydro-2H-pyran-3,4,5-triol, and 5-(((6R,6aS,7S,9R,11S)-7-ethyl-6,6a,7,8,9,10,12,13-octahydro-5H-6,9-methanopyrido[1′,2′:1,2]azepino[4,5-b]indol-2-yl)oxy)-5-oxopentanoic acid.

In some embodiments, a compound provided herein is a modified mitragynine alkaloid. Examples of modified mitragynine alkaloids include, but are not limited to, methyl (E)-2-((2S,3S,7aS,2bS)-3-ethyl-8-methoxy-7a-(sulfooxy)-1,2,3,4,6,7,7a,12b-octahydroindolo[2,3-a]quinolizin-2-yl)-3-methoxyacrylate, methyl (E)-2-((2S,3S,7aS,12bS)-3-ethyl-8-methoxy-7a-(propionyloxy)-1,2,3,4,6,7,7a,12b-octahydroindolo[2,3-a]quinolizin-2-yl)-3-methoxyacrylate, 4-(((2S,3S,7aS,12bS)-2-((E)-1,3-dimethoxy-3-oxoprop-1-en-2-yl)-3-ethyl-8-methoxy-1,3,4,6,7,12b-hexahydroindolo[2,3-a]quinolizin-7a(2H)-yl)oxy)-4-oxobutanoic acid, methyl (E)-2-((2S,3S,7aS,12bS)-7a-acetoxy-3-ethyl-8-methoxy-1,2,3,4,6,7,7a,12b-octahydroindolo[2,3-a]quinolizin-2-yl)-3-methoxyacrylate, methyl (E)-2-((2S,3S,7aS,12bS)-3-ethyl-7a-(isobutyryloxy)-8-methoxy-1,2,3,4,6,7,7a,12b-octahydroindolo[2,3-a]quinolizin-2-yl)-3-methoxyacrylate, 5-(((2S,3S,7aS,12bS)-2-((E)-1,3-dimethoxy-3-oxoprop-1-en-2-yl)-3-ethyl-8-methoxy-1,3,4,6,7,12b-hexahydroindolo[2,3-a]quinolizin-7a(2H)-yl)oxy)-5-oxopentanoic acid, 3-(((2S,3S,7aS,12bS)-2-((E)-1,3-dimethoxy-3-oxoprop-1-en-2-yl)-3-ethyl-8-methoxy-1,3,4,6,7,12b-hexahydroindolo[2,3-a]quinolizin-7a(2H)-yl)oxy)-3-oxopropanoic acid, methyl (E)-2-((2S,3S,7aS,12bS)-3-ethyl-8-methoxy-7a-((3,4,5-trihydroxy-6-(hydroxymethyl)tetrahydro-2H-pyran-2-yl)oxy)-1,2,3,4,6,7,7a,12b-octahydroindolo[2,3-a]quinolizin-2-yl)-3-methoxyacrylate and methyl (E)-2-((2S,3S,7aS,12bS)-3-ethyl-8-methoxy-7a-((3,4,5-trihydroxy-6-methyltetrahydro-2H-pyran-2-yl)oxy)-1,2,3,4,6,7,7a,12b-octahydroindolo[2,3-a]quinolizin-2-yl)-3-methoxyacrylate.

In certain embodiments, the compound provided herein is selected from the group consisting of:

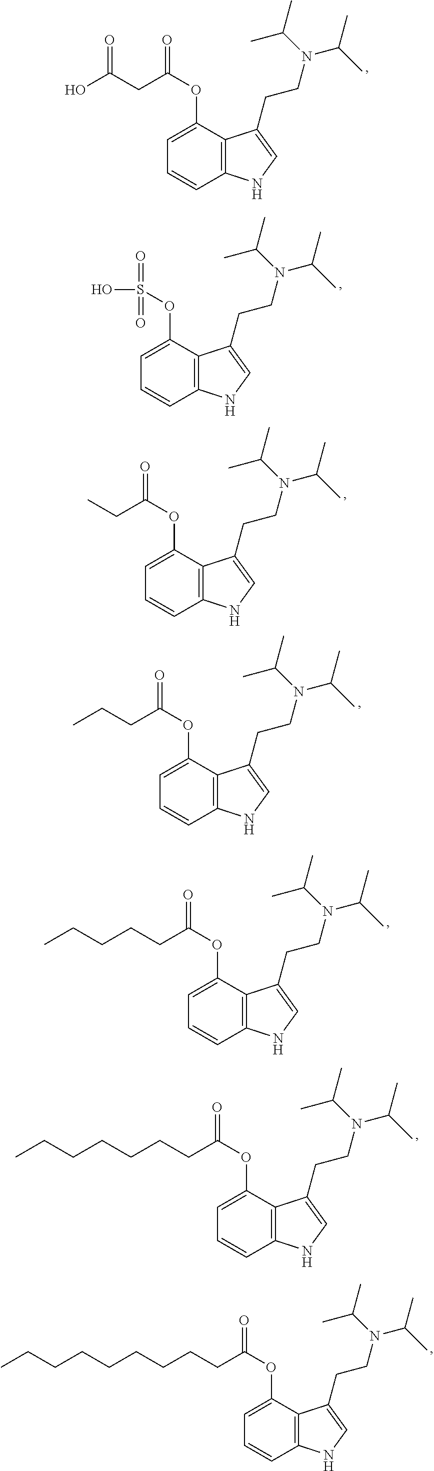

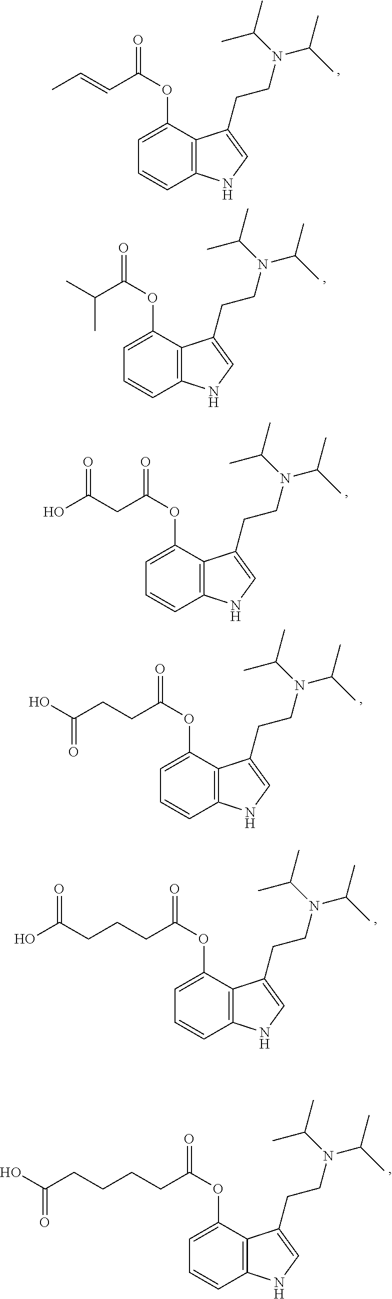

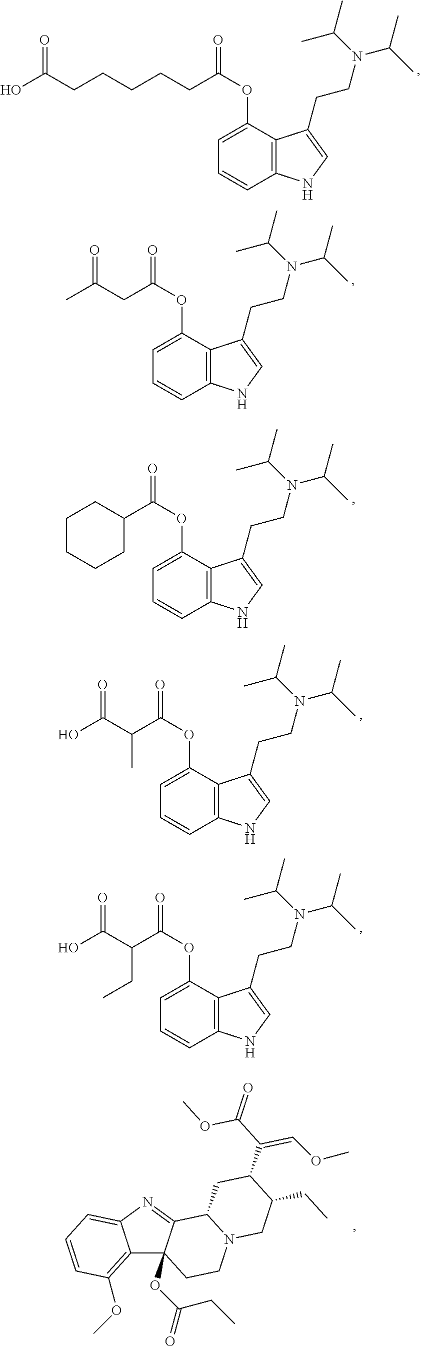

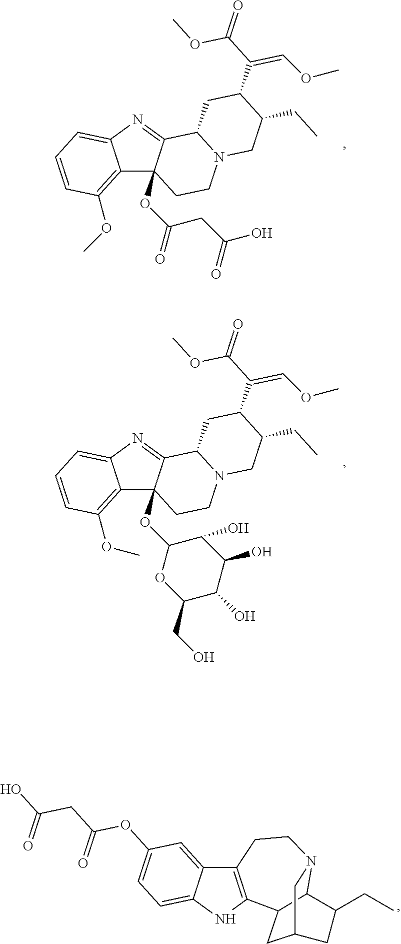 [SMILES: CC[C@@H]1CN2CC[C@@]3(OC(=O)CC(=O)O)C(=Nc4cccc(OC)c43)[C@@H]2C[C@@H]1/C(=C\OC)C(=O)OC ; CC[C@@H]1CN2CC[C@@]3(OC4O[C@H](CO)[C@@H](O)[C@H](O)[C@H]4O)C(=Nc4cccc(OC)c43)[C@@H]2C[C@@H]1/C(=C\OC)C(=O)OC ; CCC1CC2CC3c4[nH]c5ccc(OC(=O)CC(=O)O)cc5c4CCN(C2)C13]

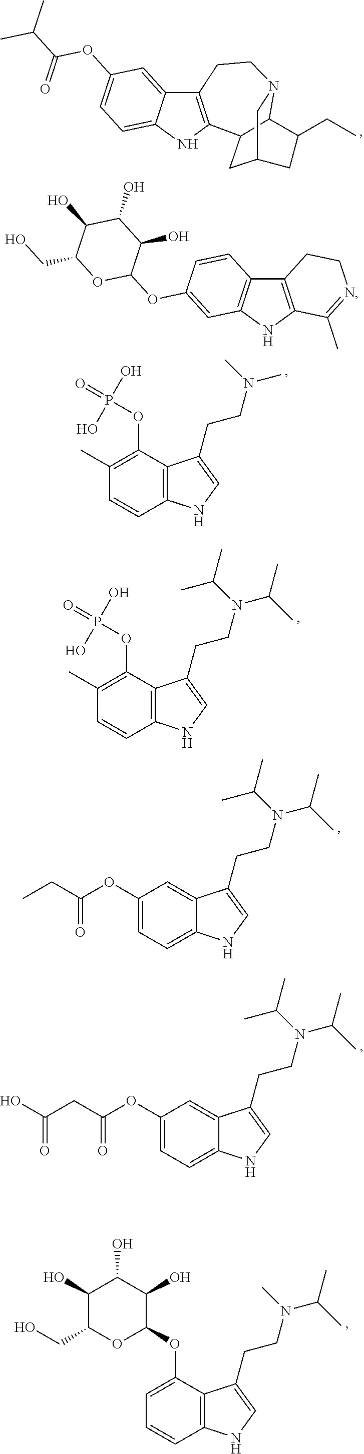

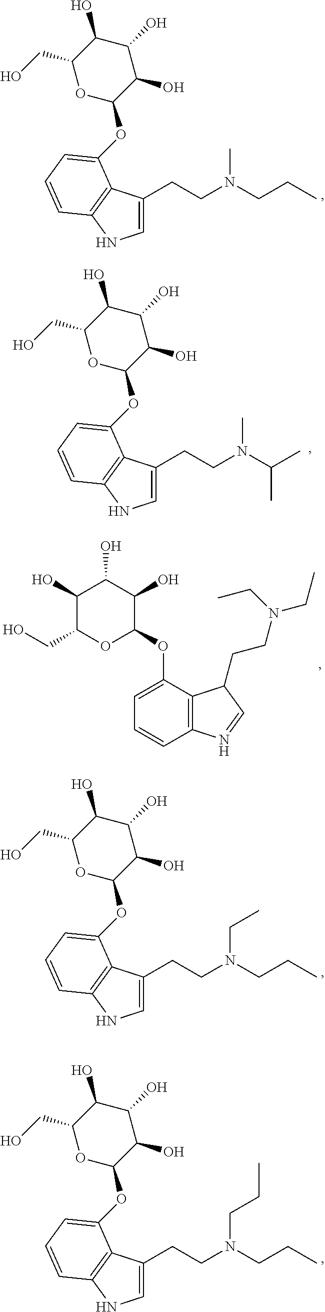

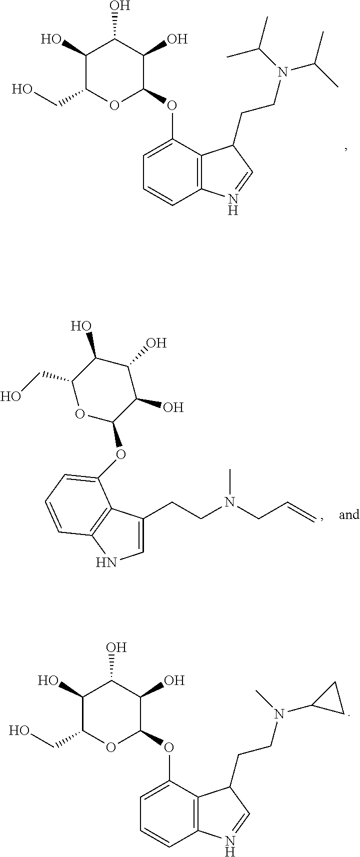 [SMILES: CC(C)N(CCC1C=Nc2cccc(O[C@H]3O[C@H](CO)[C@@H](O)[C@H](O)[C@H]3O)c21)C(C)C ; C=CCN(C)CCc1c[nH]c2cccc(O[C@H]3O[C@H](CO)[C@@H](O)[C@H](O)[C@H]3O)c12 ; CN(CCC1C=Nc2cccc(O[C@H]3O[C@H](CO)[C@@H](O)[C@H](O)[C@H]3O)c21)C1CC1]

In certain embodiments, the compound provided herein is a compound of Formula (Ia) selected from the group consisting of:

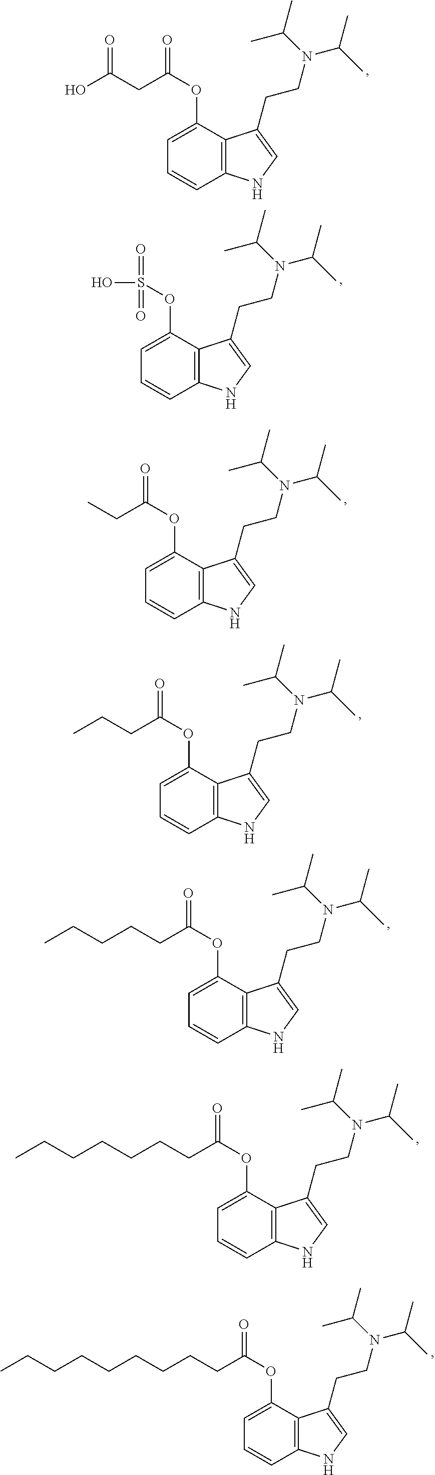

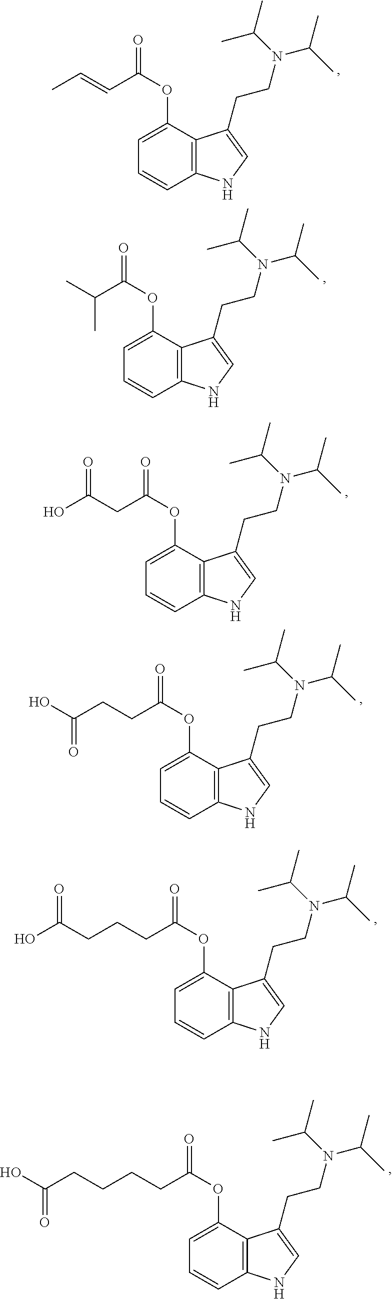

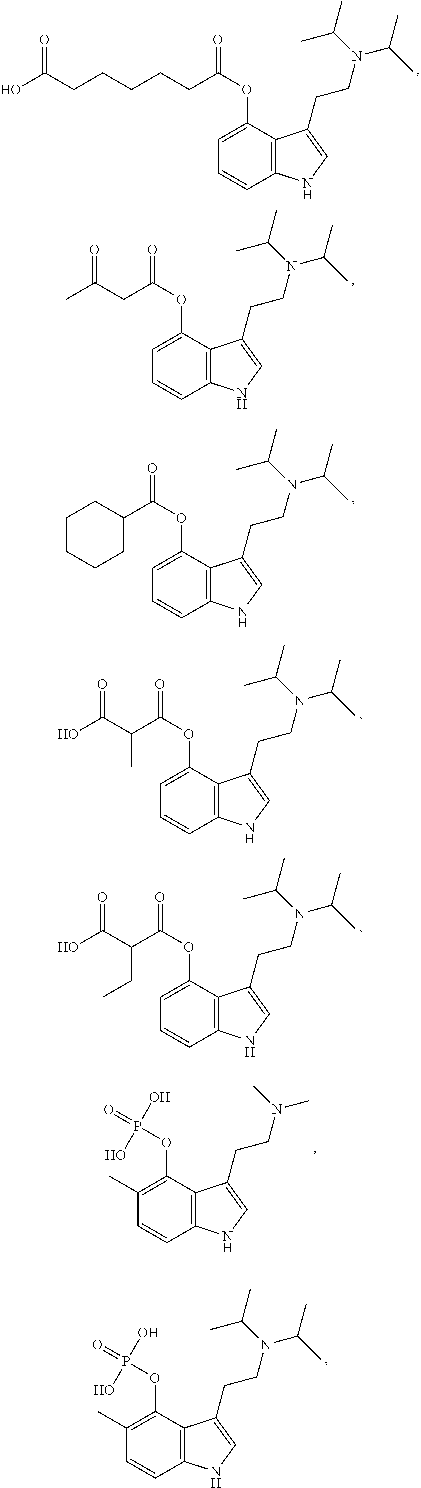

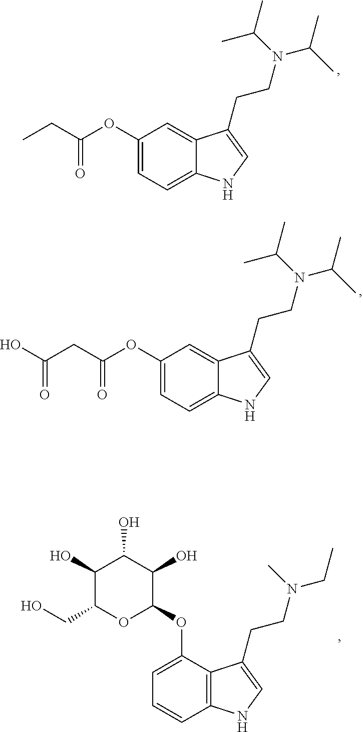 [SMILES: CCC(=O)Oc1ccc2[nH]cc(CCN(C(C)C)C(C)C)c2c1 ; CC(C)N(CCc1c[nH]c2ccc(OC(=O)CC(=O)O)cc12)C(C)C ; CCN(C)CCc1c[nH]c2cccc(O[C@H]3O[C@H](CO)[C@@H](O)[C@H](O)[C@H]3O)c12]

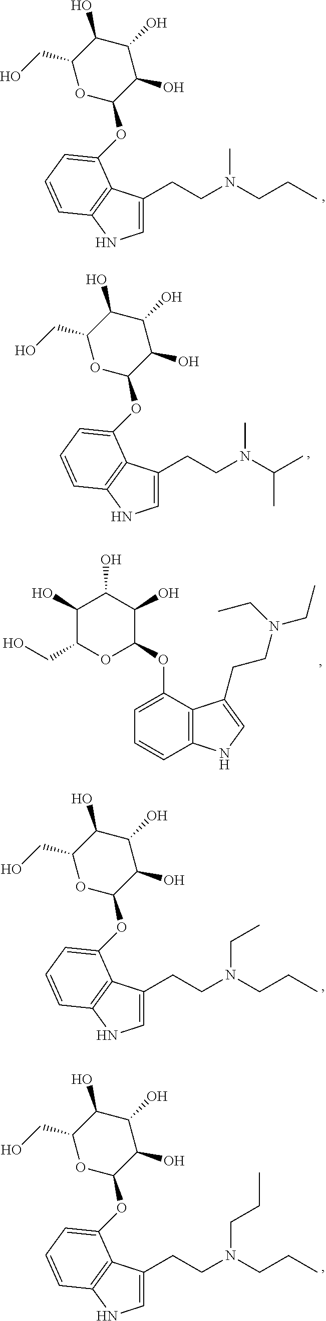

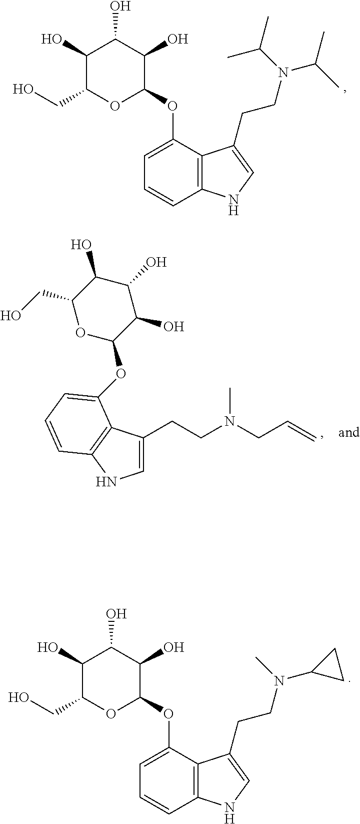 [SMILES: CC(C)N(CCc1c[nH]c2cccc(O[C@H]3O[C@H](CO)[C@@H](O)[C@H](O)[C@H]3O)c12)C(C)C ; C=CCN(C)CCc1c[nH]c2cccc(O[C@H]3O[C@H](CO)[C@@H](O)[C@H](O)[C@H]3O)c12 ; CN(CCc1c[nH]c2cccc(O[C@H]3O[C@H](CO)[C@@H](O)[C@H](O)[C@H]3O)c12)C1CC1]

In certain embodiments, the compound provided herein is a compound of Formula (Ia) selected from the group consisting of:

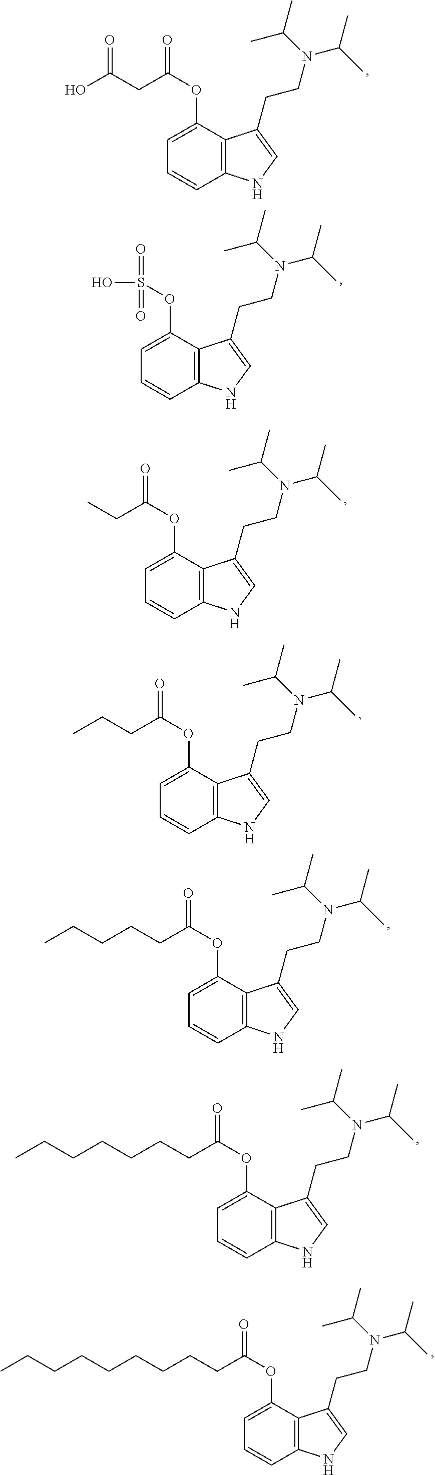

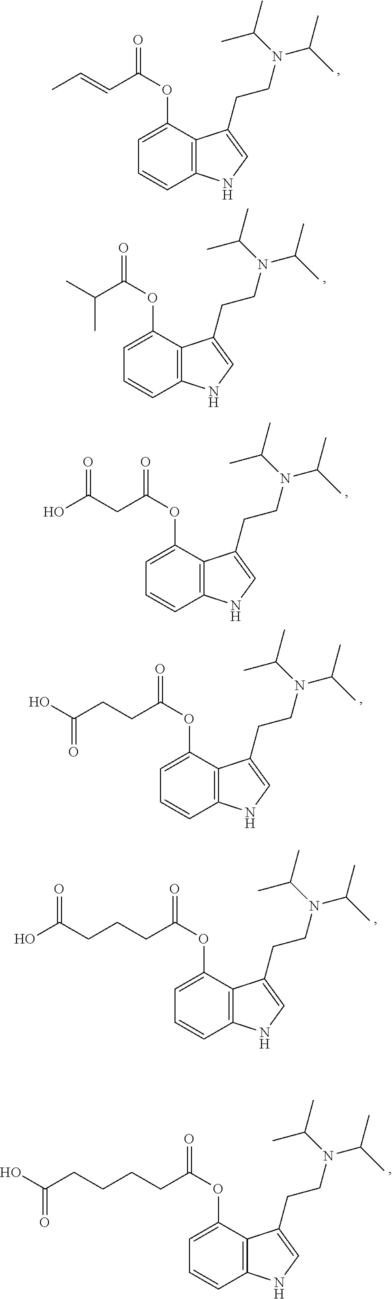

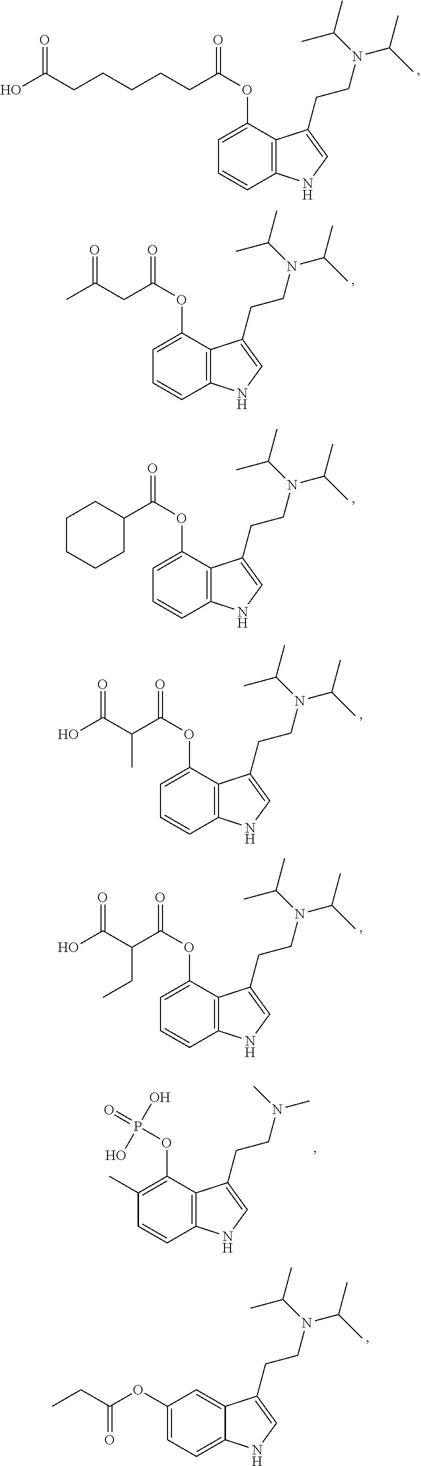

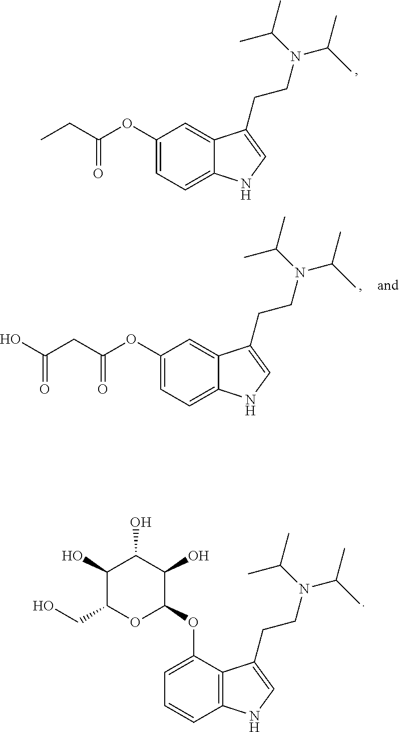 [SMILES: CCC(=O)Oc1ccc2[nH]cc(CCN(C(C)C)C(C)C)c2c1 ; CC(C)N(CCc1c[nH]c2ccc(OC(=O)CC(=O)O)cc12)C(C)C ; CC(C)N(CCc1c[nH]c2cccc(O[C@H]3O[C@H](CO)[C@@H](O)[C@H](O)[C@H]3O)c12)C(C)C]

In certain embodiments, the compound provided herein is a compound of Formula (Ia) selected from the group consisting of:

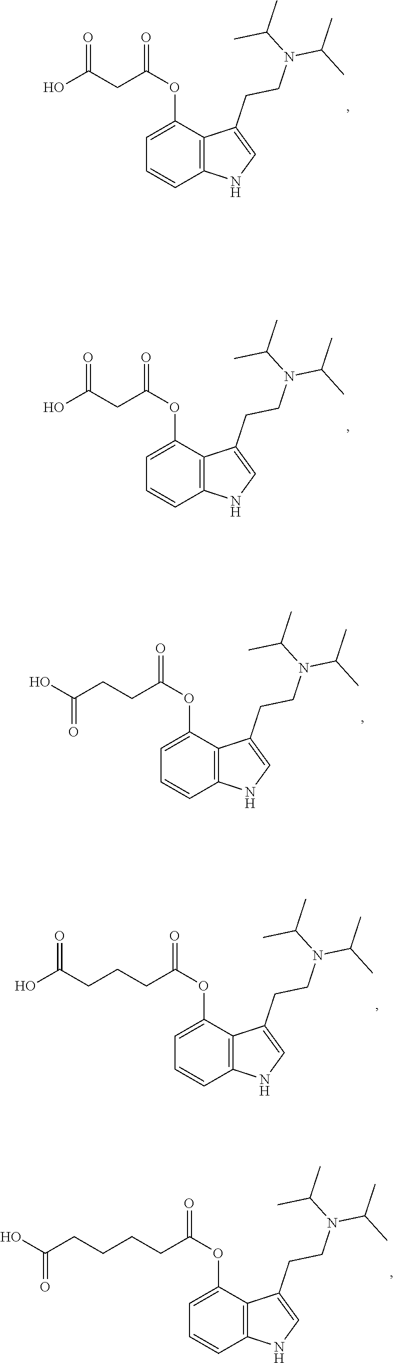 [SMILES: CC(C)N(CCc1c[nH]c2cccc(OC(=O)CC(=O)O)c12)C(C)C ; CC(C)N(CCc1c[nH]c2cccc(OC(=O)CC(=O)O)c12)C(C)C ; CC(C)N(CCc1c[nH]c2cccc(OC(=O)CCC(=O)O)c12)C(C)C ; CC(C)N(CCc1c[nH]c2cccc(OC(=O)CCCC(=O)O)c12)C(C)C ; CC(C)N(CCc1c[nH]c2cccc(OC(=O)CCCCC(=O)O)c12)C(C)C]

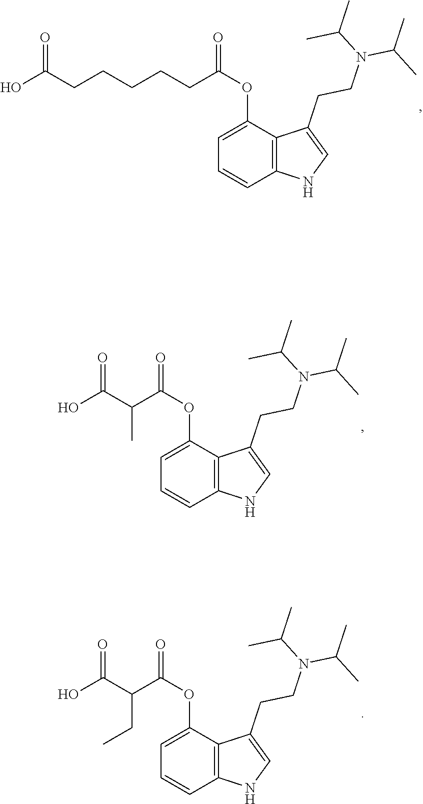 [SMILES: CC(C)N(CCc1c[nH]c2cccc(OC(=O)CCCCCC(=O)O)c12)C(C)C ; CC(C(=O)O)C(=O)Oc1cccc2[nH]cc(CCN(C(C)C)C(C)C)c12 ; CCC(C(=O)O)C(=O)Oc1cccc2[nH]cc(CCN(C(C)C)C(C)C)c12]

In certain embodiments, the compound of Formula (Ia) is,

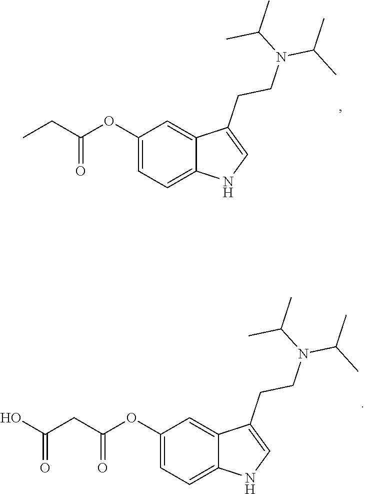 [SMILES: CCC(=O)Oc1ccc2[nH]cc(CCN(C(C)C)C(C)C)c2c1 ; CC(C)N(CCc1c[nH]c2ccc(OC(=O)CC(=O)O)cc12)C(C)C]

In certain embodiments, the compound of Formula (Ia) is selected from the group consisting of:

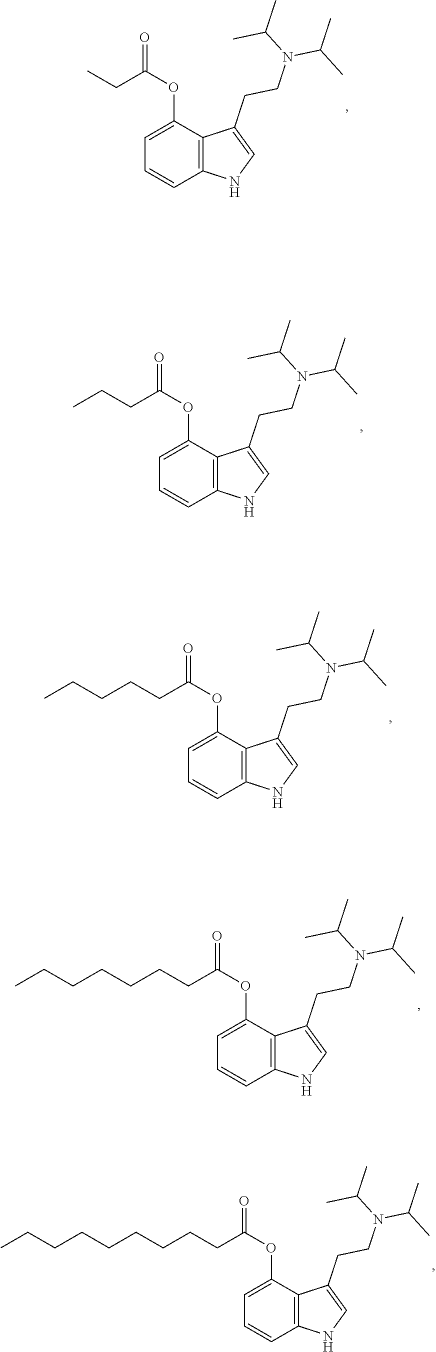

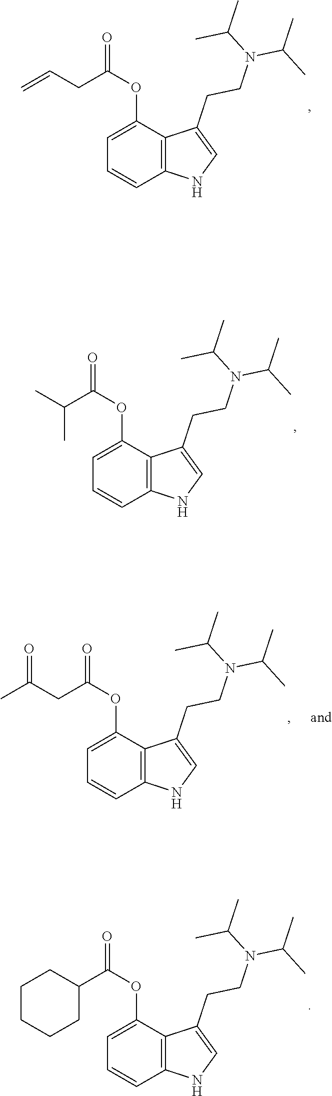

In certain embodiments, the compound of Formula (Ia) is selected from the group consisting of:

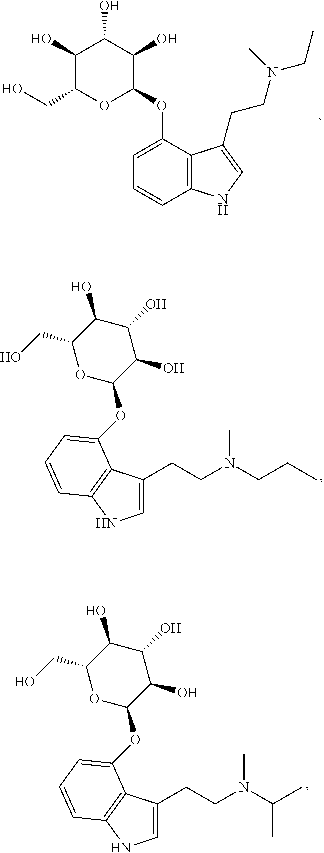 [SMILES: CCN(C)CCc1c[nH]c2cccc(O[C@H]3O[C@H](CO)[C@@H](O)[C@H](O)[C@H]3O)c12 ; CCCN(C)CCc1c[nH]c2cccc(O[C@H]3O[C@H](CO)[C@@H](O)[C@H](O)[C@H]3O)c12 ; CC(C)N(C)CCc1c[nH]c2cccc(O[C@H]3O[C@H](CO)[C@@H](O)[C@H](O)[C@H]3O)c12]

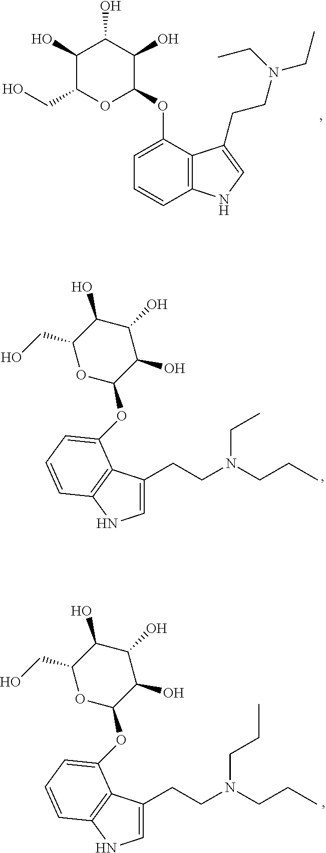 [SMILES: CCN(CC)CCc1c[nH]c2cccc(O[C@H]3O[C@H](CO)[C@@H](O)[C@H](O)[C@H]3O)c12 ; CCCN(CC)CCc1c[nH]c2cccc(O[C@H]3O[C@H](CO)[C@@H](O)[C@H](O)[C@H]3O)c12 ; CCCN(CCC)CCc1c[nH]c2cccc(O[C@H]3O[C@H](CO)[C@@H](O)[C@H](O)[C@H]3O)c12]

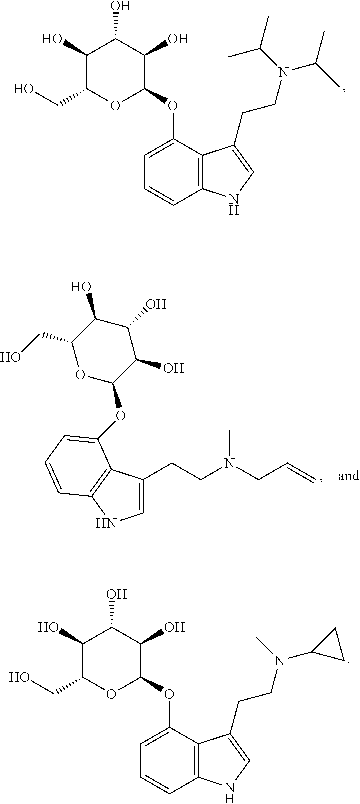 [SMILES: CC(C)N(CCc1c[nH]c2cccc(O[C@H]3O[C@H](CO)[C@@H](O)[C@H](O)[C@H]3O)c12)C(C)C ; C=CCN(C)CCc1c[nH]c2cccc(O[C@H]3O[C@H](CO)[C@@H](O)[C@H](O)[C@H]3O)c12 ; CN(CCc1c[nH]c2cccc(O[C@H]3O[C@H](CO)[C@@H](O)[C@H](O)[C@H]3O)c12)C1CC1]

In certain embodiments, the compound of Formula (Ia) is

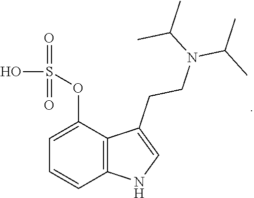 [SMILES: CC(C)N(CCc1c[nH]c2cccc(OS(=O)(=O)O)c12)C(C)C]

In certain embodiments, the compound of Formula (Ia) is H or

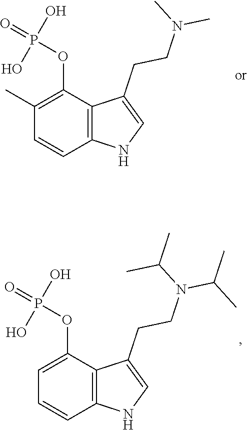 [SMILES: Cc1ccc2[nH]cc(CCN(C)C)c2c1OP(=O)(O)O ; CC(C)N(CCc1c[nH]c2cccc(OP(=O)(O)O)c12)C(C)C]

In certain embodiments, the compound of Formula (Ia) is selected from the group consisting of:

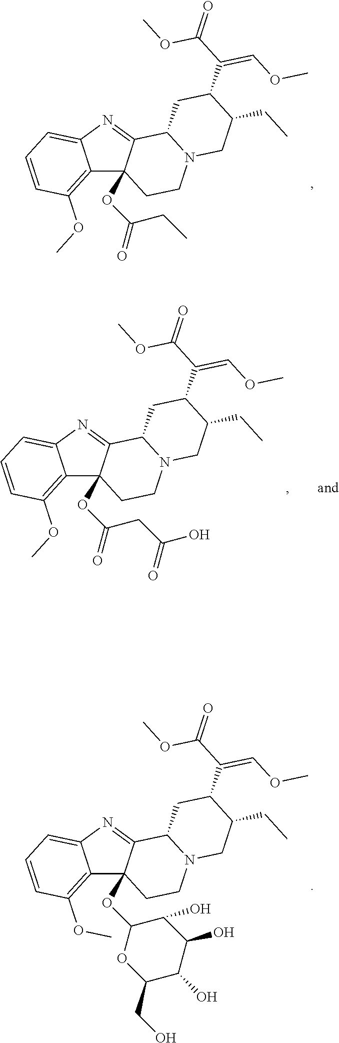 [SMILES: CCC(=O)O[C@]12CCN3C[C@@H](CC)[C@@H](/C(=C\OC)C(=O)OC)C[C@H]3C1=Nc1cccc(OC)c12 ; CC[C@@H]1CN2CC[C@@]3(OC(=O)CC(=O)O)C(=Nc4cccc(OC)c43)[C@@H]2C[C@@H]1/C(=C\OC)C(=O)OC ; CC[C@@H]1CN2CC[C@@]3(OC4O[C@H](CO)[C@@H](O)[C@H](O)[C@H]4O)C(=Nc4cccc(OC)c43)[C@@H]2C[C@@H]1/C(=C\OC)C(=O)OC]

In certain embodiments, the compound of Formula (Ia) is selected from the group consisting of:

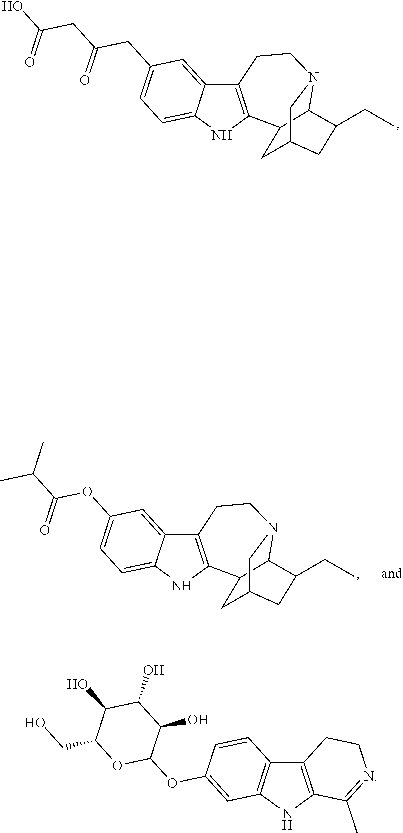

### Enzymes

In another aspect, provided herein is a novel enzyme mixture that transfers functional groups to indole alkaloids. In some cases, the enzyme mixture transfers donor functional groups to acceptor functional groups on indole alkaloids. In some cases, the enzyme mixture can be a mixture of enzymes, buffers, and reactants. In some cases, reactants can include donor functional groups and indole alkaloids. In some cases, the enzyme mixture can be a cell-free solution. In some cases, the enzyme mixture contains unmodified host cells containing transferase enzymes. In some cases, the enzyme mixture contains modified host cells containing transferase enzymes. In some cases, the enzyme mixture contains unmodified host cells producing indole alkaloids. In some cases, the enzyme mixture contains modified host cells producing indole alkaloids. Compositions of modified indoles may be used for therapeutic and consumer applications. Enzyme and whole-cell biocatalysts are increasingly attractive as a renewable method for producing specialty chemicals and pharmaceuticals. These biocatalysts are referred to as modified host cells. Using in vitro enzymatic reactions and intact microorganisms as a catalyst offers several advantages over conventional synthesis, such as high enantioselectivity and regioselectivity. An advantage of whole-cell biocatalysts is the ability to achieve multipart syntheses, whereby multiple intermediates generated in parallel in the same vessel are combined into a final product. Another feature of whole-cell biocatalysts is the ability to catalyze reactions under ambient temperatures and in aqueous solutions. This advantage can also be realized in ester synthesis. The modifications of the present disclosure are achieved by utilizing a novel enzyme mixture. The general classes of these modifications are listed below in Table 1.

TABLE 1
General Classes of Modifications for Forming Biobased Esters

| Modification | Sources | Donor | Features |
|---|---|---|---|
| Glycosylation | Plants, Animals, | UDP-Sugar | Increased water solubility |
| | Bacteria, Fungi | NDP-Sugar | Stable |
| | | | Cost to charge: 2 ATP equivalents |
| | | | Non-limiting sugar group |
| | | | examples: glucose, galactose, |
| | | | rhamnose, rutinose, glucuronate, |
| | | | xylose and others |
| Phosphorylation | Insects, | ATP | Increased water solubility |
| | Bacteria, Fungi | | Stable |
| | | | Cost to charge: 1 ATP equivalent |
| Sulfonation | Animals | PAPS | Increased water solubility |
| | | | Stable |
| | | | Cost to charge: 3 ATP equivalents |
| Acylation | Animals, | Acyl-CoA | Modified water solubility |
| | Bacteria, Fungi | | Enhanced membrane permeance |
| | | | Acid/base labile |
| | | | Cost to charge: 2 ATP equivalents |
| | | | Non-limiting acyl group examples: |
| | | | acetate, malonate, butyrate, |
| | | | coumarate, glutarate, adipate, and |
| | | | others |
| Methylation | Animals, Plants, | SAM | Stable |
| | Bacteria, Fungi | | Decreased water solubility |

Kinases are enzymes that can transfer a phosphate group from the donor, adenosine triphosphate (ATP), to an indole alkaloid to form a phosphorylated indole alkaloid. In some cases, the transferase in the enzyme mixture is a kinase. These kinase enzymes can be utilized using in vitro systems. Kinase enzymes can also be expressed in a microbial host cell. In some cases, the one or more enzymes comprises a kinase. In some cases, the kinase comprises an amino acid sequence having at least 50%, at least 600%, at least 70%, at least 80%, at least 900%, or at least 95% sequence identity to SEQ ID NOs: 1 and 2.

Methyltransferases are enzymes that can transfer a methyl group from the donor, 5-adenosyl methionine (SAM), to an indole alkaloid to form a methylated indole alkaloid. In some cases, the transferase in the enzyme mixture is a methyltransferase. These Methyltransferase enzymes can be utilized using in vitro systems. Methyltransferase enzymes can also be expressed in a microbial host cell as a component of the enzyme mixture. In some cases, the one or more enzymes comprises a methyltransferase. In some cases, the methyltransferases comprise an amino acid sequence having at least 50%, at least 600%, at least 70%, at least 80%, at least 900%, or at least 95% sequence identity to any one SEQ ID NOs: 3 and 4.

Sulfotransferase are enzymes that can transfer a sulfur group from the donor, 3′-phosphoadenosine-5′-phosphosulfate (PAPS), to an indole alkaloid to form a sulfated indole alkaloid. In some cases, the transferase in the enzyme mixture is a sulfotransferase. These sulfotransferase enzymes can be utilized using in vitro systems. Sulfotransferase enzymes can also be expressed in a microbial host cell as a component of the enzyme mixture. In some cases, the one or more enzymes comprises a sulfotransferase. In some cases, the sulfotransferase comprises an amino acid sequence having at least 50%, at least 60%, at least 70%, at least 80%, at least 90%, or at least 95% sequence identity to SEQ ID NOs: 5 and 6.

Acetyl-CoA is the most abundant acyl-CoA unit in the cell and can be added in in vitro enzymatic reactions. Acyl-CoA can readily be used to generate a wide range of acyl esters catalyzed by an acyl-transferase enzyme. Other acyl-CoA donor units for donating acyl groups to indole alkaloids include, but are not limited to, isobutyryl-CoA, butyryl-CoA, succinyl-CoA, malonyl-CoA, coumarate-CoA, glutaryl-CoA, adipoyl-CoA, and enoyl-CoA. Other donor units can be isoprenoid precursors, including, but not limited to, farnesyl pyrophosphate, geranylgeranyl pyrophosphate, and/or dimethylallyl pyrophosphate. Acyltransferases are enzymes that can transfer an acyl from molecule from the donor, acyl-CoA, to an indole alkaloid to form an acylated indole alkaloid. In some cases, the transferase in the enzyme mixture is an acyltransferase. These acyltransferase enzymes can be utilized using in vitro systems. Acyltransferase enzymes can also be expressed in a microbial host cell as a component of the enzyme mixture. In some cases, the one or more enzymes comprises an acyltransferase. In some cases, the acyltransferases comprise an amino acid sequence having at least 50%, at least 60%, at least 70%, at least 80%, at least 90%, or at least 95% sequence identity to any one SEQ ID NOs: 7 and 8.

Glycosylation can modulate the physiological properties of small molecules and peptides, with specific impacts such as improved metabolic stability, membrane permeability, biodistribution, and ligand-target interactions. Thus, numerous glycosylated natural products and synthetic glycopeptides are important biochemical probes and therapeutic agents. Although methods for the glycosylation of organic compounds are numerous, glycosylation methods typically require protection of glycosyl donors and acceptors. The most common exceptions generally involve the application of glycosyl transferases in the enzymatic context. The chemical synthesis of glycosides is, however, far from trivial and involves inefficient multistep routes. In some cases, the glycosyl donor molecule can be a nucleotide diphosphate sugar. In some cases, the nucleotide component of the nucleotide sugar can be uracil diphosphate (aka UDP). The sugar component can be, but is not limited to, glucose, glucuronic acid, galacturonic acid, xylose, galactose, rhamnose, and rutinose. The sugar component can be, but is not limited to, D-glucose, D-glucuronic acid, D-galacturonic acid, D-xylose, D-galactose, D-rhamnose, and D-rutinose. Nucleotide diphospho sugars (NDP-sugars) or sugar nucleotides are activated monosaccharide donors used by glycosyl transferases (GTs) for glycosylation of a variety of acceptors. NDP-sugars originate from primary metabolism of common precursors, such as UDP-glucose, which are transformed to a diverse range of NDP-sugars by sugar nucleotide processing enzymes. The UDP-sugars can be chosen from a group including, but not limited to, UDP-glucose, UDP-glucuronic acid, UDP-galacturonic acid, UDP-xylose, UDP-galactose, UDP-rhamnose, and UDP-rutinose. In some cases, the transferase in the enzyme mixture is a glucosyltransferase. These glucosyltransferase enzymes can be utilized using in vitro systems.

Glucosyltransferase enzymes can also be expressed in a microbial host cell as a component of the enzyme mixture. In some cases, the one or more enzymes comprises a glucosyltransferase. In some cases, the glucosyltransferase comprises an amino acid sequence having at least 50%, at least 60%, at least 70%, at least 80%, at least 90%, or at least 95% sequence identity to any one SEQ ID NOs: 9-12.

Prenyltransferases are a class of enzymes that transfer allylic prenyl groups to acceptor molecules. Prenyl transferases commonly refer to prenyl diphosphate synthases. Prenyltransferases are commonly divided into two classes, cis (or Z) and trans (or E), depending upon the stereochemistry of the resulting products. Examples of trans-prenyltransferases include dimethylallyltranstransferase, and geranylgeranyl pyrophosphate synthase. Cis-prenyltransferases include dehydrodolichol diphosphate synthase (involved in the production of a precursor to dolichol). Prenyltransferases are enzymes that can transfer a prenyl molecule from the donor, prenyl diphosphate, to an indole alkaloid to form a sulfated indole alkaloid. In some cases, the transferase in the enzyme mixture is a prenyltransferase. These prenyltransferase enzymes can be utilized using in in vitro systems. Prenyltransferase enzymes can also be expressed in a microbial host cell as a component of the enzyme mixture. In some cases, the one or more enzymes comprises a prenyltransferase. In some cases, the sulfotransferase comprises an amino acid sequence having at least 50%, at least 60%, at least 70%, at least 80%, at least 90%, or at least 95% sequence identity to SEQ ID NO: 13-14.

In some embodiments, the one or more enzymes are enzymes disclosed below in Table 2.

TABLE 2
Enzymes

| Enzyme/Source | Genbank/Uniprot | |
|---|---|---|
| Organism | Number | Sequence |
| 4-hydroxytryptamine | PPQ83229.1 | MAFDLKTPEGLLLYLTRHLSLDVDPSGVKRISG |
| kinase/P. cyanescens | | GFVNVTWRIRLNAPYQGHTSIILKHAQPHLSSD |
| | | EDFKIGVERSAYEYQALKVMSANQEVLGGDDS |
| | | RVSVPEGLHYDVENNALIMQDVGTMKTLLDY |
| | | ATAKPPLSTEIASLVGTEIGAFIARLHNLGRKRR |
| | | DQPAFKFFSGNIVGRTTADQLYQTIIPNAAKYGI |
| | | NDPLLPTVVKDLVGEVMNSEETLIMADLWSGN |
| | | ILLEFVEGNPSELKKIWLVDWELCKYGPASLDM |
| | | GYFLGDCYLIARFQDELVGTTMRKAYLKGYAR |
| | | TAKGTINYSKVTASIGAHLVMWTDEMKWGNY |
| | | EEREEFVKKGVEALHDAWEDNNDGEITSVLVN |
| | | EASST (SEQ ID NO: 1) |
| 4-hydroxytryptamine | P0DPA8 | MAFDLKTEDGLITYLTKHLSLDVDTSGVKRLSG |
| kinase/P. cubensis | | GFVNVTWRIKLNAPYQGHTSIILKHAQ |
| | | PHMSTDEDFKIGVERSVYEYQAIKLMMANREV |
| | | LGGVDGIVSVPEGLNYDLENNALIMQDV |
| | | GKMKTLLDYVTAKPPLATDIARLVGTEIGGFVA |
| | | RLHNIGRERRDDPEFKFFSGNIVGRTT |
| | | SDQLYQTIPNAAKYGVDDPLLPTVVKDLVDD |
| | | VMHSEETLVMADLWSGNILLQLEEGNPS |
| | | KLQKIYILDWELCKYGPASLDLGYFLGDCYLIS |
| | | RFQDEQVGTTMRQAYLQSYARTSKHSI |
| | | NYAKVTAGIAAHIVMWTDFMQWGSEEERINFV |
| | | KKGVAAFHDARGNNDNGEITSTLLKESS |
| | | TA (SEQ ID NO: 2) |
| Acetylserotonin O- | P46597 | MGSSEDQAYRLLNDYANGFMVSQVLFAACEL |
| methyltransferase/ | | GVFDLLAEAPGPLDVAAVAAGVRASAHGTELL |
| Homo sapiens | | LDICVSLKLLKVETRGGKAFYRNTELSSDYLTT |
| | | VSPTSQCSMLKYMGRTSYRCWGHLADAVREG |
| | | RNQYLETFGVPAFELFTAIYRSEGERLOFMQAL |
| | | QEVWSVNGRSVLTAFDLSVFPLMCDLGGGAG |
| | | ALAKECMSLYPGCKITVFDIPEVVWTAKQHFSF |
| | | QEEEQIDFQEGDFFKDPLPEADLYILARVLHDW |
| | | ADGKCSHLLERIYHTCKPGGGILVIESLLDEDRR |
| | | GPLLTQLYSLNMLVQTEGQERTPTHYHMLLSS |
| | | AGFRDFQFKKTGAIYDAILARK (SEQ ID NO: 3) |
| Tryptamine n- | PPQ80976.1 | MHNRNPYRDVIDYQALAEAYPPLKPHVTVNAD |
| methyltransferase/P. | | NTASIDLTIPEVQRQYTAALLERDEGLTITLPED |
| cyanescens | | RLCPTVPNRLNYVLWIEDIFQCTNKALGLSDDR |
| | | PVKGVDIGTGASAIYPMLACARFKQWSMIATE |
| | | VERKCIDTARENVLANNLQDRLSILEVSVDGPIL |
| | | VPIFDTFERATSDYEFEFTMCNPPFYDGAADMQ |
| | | TSDAAKGFGFGVNAPHSGTVIEMATEGGEAAF |
| | | VAQMVRESMKLQTRCRWFTSNLGKLKSLHEIV |
| | | ALLRESQITNYAINEYVOGTTRRYALAWSFTDI |
| | | KLTEELYRPSNPELGPLCSTEV (SEQ ID NO: 4) |
| Sulfotransferase 1A1/ | P50225 | MELIQDTSRPPLEYVKGVPLIKYFAEALGPLQSF |
| Homo sapiens | | QARPDDLLISTYPKSGTTWVSQILDMIYQGGDL |
| | | EKCHRAPIFMRVPFLEFKAPGIPSGMETLKDTPA |
| | | PRLLKTHLPLALLPQTLLDQKVKVVYVARNAK |
| | | DVAVSYYHFYHMAKVHPEPGTWDSFLEKFMV |
| | | GEVSYGSWYQHVQEWWELSRTHPVLYLFYED |
| | | MKENPKREIQKILEFVGRSLPEETVDFVVQHTSF |
| | | KEMKKNPMTNYTTVPQEFMDHSISPFMRKGM |
| | | AGDWKTTFTVAQNERFDADYAEKMAGCSLSF |
| | | RSEL (SEQ ID NO: 5) |
| Sulfotransferase LA3/ | P0DMM9 | MELIQDTSRPPLEYVKGVPLIKYFAEALGPLQSF |
| Homo sapiens | | QARPDDLLINTYPKSGTTWVSQILDMIYQGGDI. |
| | | EKCNRAPIYVRVPFLEVNDPGEPSGLETLKDTPP |
| | | PRLIKSHLPLALLPQTLLDQKVKVVYVARNPKD |
| | | VAVSYYHFHRMEKAHPEPGTWDSFLEKFMAG |
| | | EVSYGSWYQHVQEWWELSRTHPVLYLFYEDM |
| | | KENPKREIQKILEFVGRSLPEETMDFMVQHTSF |
| | | KEMKKNPMTNYTTVPQELMDHSISPFMRKGM |
| | | AGDWKTTFTVAQNERFDADYAEKMAGCSLSF |
| | | RSEL (SEQ ID NO: 6) |
| Alcohol O- | P40353 | MNEIDEKNQAPVQQECLKEMIQNGHARRMGS |
| acetyltransferase 1/ | | VEDLYVALNRONLYRNFCTYGELSDYCTRDQL |
| Saccharomyces | | TLALREICLKNPTLLHIVLPTRWPNHENYYRSSE |
| cerevisiae | | YYSRPHPVHDYISVLQELKLSGVVLNEQPEYSA |
| | | VMKQILEEFKNSKGSYTAKIFKLITTLTIPYFGP |
| | | TGPSWRLICLPEEHTEKWKKFIFVSNHCMSDGR |
| | | SSIHFFHDLRDELNNIKTPPKKLDYIFKYEEDYQ |
| | | LLRKLPEPIEKVIDERPPYIFIPKSLLSGFIYNHLR |
| | | FSSKGVCMRMDDVEKTDDVVTEIINISPTEFQAI |
| | | KANIKSNIQGKCTITPFLHVCWFVSLHKWGKFF |
| | | KPLNFEWLTDIFIPADCRSQLPDDDEMRQMYR |
| | | YGANVGFIDFTPWISEFDMNDNKENFWPLIEHY |
| | | HEVISEALRNKKHLHGLGFNIQGFVQKYVNIDK |
| | | VMCDRAIGKRRGGTLLSNVGLFNQLEEPDAKY |
| | | SICDLAFGOFOGSWHOAFSLGVCSTNVKGMNI |
| | | VVASTKNVVGSQESLEELCSIYKALLLGP (SEQ |
| | | ID NO: 7) |
| Chloramphenicol | P26841 | MGNYFESPFRGKLLSEQVSNPNIRVGRYSYYSG |
| acetyltransferase/ | | YYHGHSFDDCARYLMPDRDDVDKLVIGSFCSI |
| Pseudomonas | | GSGAAFIMAGNQGHRAEWASTEPFHFMHEEPV |
| aeruginosa | | FAGAVNGYQPAGDTLIGHDVWIGTEAMFMPG |
| | | VRVGHGAIIGSRALVTGDVEPYAIVGGNPARTI |
| | | RKRFSDGDIQNLLEMAWWDWPLADIEAAMPL |
| | | LCTGDIPALYRHWKQRQATA (SEQ ID NO: 8) |
| UDP- | P19224 | MACLLRSFQRISAGVFFLALWGMVVGDKLLVV |
| glucuronosyltransferase | | PQDGSHWLSMKDIVEVLSDRGHEIVVVVPEVN |
| 1-6/Homo sapiens | | LLLKESKYYTRKIYPVPYDQEELKNRYQSFGNN |
| | | HFAERSFLTAPOTEYRNNMIVIGLYFINCQSLLQ |
| | | DRDTLNFFKESKFDALFTDPALPCGVILABYLG |
| | | LPSVYLFRGFPCSLEHTFSRSPDPVSYIPRCYTKF |
| | | SDHMTFSQRVANFLVNLLEPYLFYCLFSKYEEL |
| | | ASAVLKRDVDIITLYQKVSVWLLRYDFVLEYPR |
| | | PVMPNMVFIGGINCKKRKDLSQEFEAYINASGE |
| | | HGIVVFSLGSMVSEIPEKKAMAIADALGKIPQT |
| | | VLWRYTGTRPSNLANNTILVKWLPQNDLLGHP |
| | | MTRAFITHAGSHGVYESICNGVPMVMMPLFGD |
| | | QMDNAKRMETKGAGVTLNVLEMTSEDLENAL |
| | | KAVINDKSYKENIMRLSSLHKDRPVEPLDLAVF |
| | | WVEFVMRHKGAPHLRPAAHDLTWYQYHSLDV |
| | | IGFLLAVVLTVAFITFKCCAYGYRKCLGKKGRV |
| | | KKAHKSKTH (SEQ ID NO: 9) |
| Oleandomycin | Q53685 | MTTQTTPAHIAMFSIAAHGHVNPSLEVIRELVA |
| glycosyltransferase/ | | RGHRVTYAIPPVFADKVAATGPRPVLYHSTLPG |
| Streptomyces | | PDADPEAWGSTLLDNRRTFINDAIQALPQLAD |
| antibioticus | | AYADDIPDLVLHDITSYPARVLARRWGVPAVS |
| | | LSPNLVAWKGYEEEVAEPMWREPRQTERGRA |
| | | YYARFEAWLKENGITEHPDTFASHPPRSLVLIPK |
| | | ALQPHADRVDEDVYTFVGACQGDRAEEGGWQ |
| | | RPAGAEKVVLVSLGSAFTKQPAFYRECVRAFG |
| | | NLPGWHLVLQIGRKVTPAELGELPDNVEVHDW |
| | | VPQLAILROADLFVTHAGAGGSQEGLATATPMI |
| | | AVPQAVDQFGNADMLQGLGVARKLATEEATA |
| | | DLLRETALALVDDPEVARRLRRIQAEMAQEGG |
| | | TRRAADLIEAELPARHERQEPVGDRPNVGDRPA |
| | | GVRSDRQRSAL (SEQ ID NO: 10) |
| Glycosyltransferase/ | A0A2L2R220 | MESPAAPPTTAPPPHVIIMPSAGMGHLIPLAEFA |
| Persicaria tinctoria | | KRLLPRFTFTFAVPTSGPPSSSQRDFLSSLPASID |
| | | TSFLPEVDLSDAPSDAQIETLMSLMVVRSLPSLR |
| | | DLIASYSASGRRVAALVVDLFATDAIDVALELG |
| | | IRPFIFFPSTAMTLSFFLHLEKLDETVSCEFAELS |
| | | DPVQIPGCIPVHGKDLIDPVQDRKNDAYKWLL |
| | | HHSKRYKLAEGVIVNSFEGLEGGPIRELLHPEPG |
| | | KPRVYPVGPLIQAGSCEKGAAARPECLKWLDQ |
| | | QPRGSVLFVNFGSGGVISTEQQNELAGVLAHS |
| | | QQRFLWVVRPPNDGIANATYFSVDGEIDPLKLL |
| | | PEGFLEQTAGRGLVLPMWAPQIDVLSHESTGGF |
| | | LTHCGWNSTLESVFHGVPLITWPLYAEQKMNA |
| | | VMLTEGLRVGLRPSVGKDGIIRGDEIARVIGEL |
| | | MEGEEGKRIRSKMQELKRAASAVLSKDGSSTR |
| | | ALEEVAKIWESKV (SEQ ID NO: 11) |
| UDP- | O60656 | MACTGWTSPLPLCVCLLLTCGFAEAGKLLVVP |
| glucuronosyltransferase | | MDGSHWFTMRSVVEKLILRGHEVVVVMPEVS |
| 1-9/Homo sapiens | | WQLGRSLNCTVKTYSTSYTLEDLDREFKAFAH |
| | | AQWKAQVRSIYSLLMGSYNDIFDLFFSNCRSLF |
| | | KDKKLVEYLKESSFDAVFLDPFDNCGLIVAKYF |
| | | SLPSVVFARGILCHYLEEGAQCPAPLSYVPRILL |
| | | GFSDAMTFKERVRNHIMHLEEHLLCHRFFKNA |
| | | LEIASEILQTPVTEYDLYSHTSIWLLRTDFVLDY |
| | | PKPVMPNMIFIGGINCHQGKPLPMEFEAYINAS |
| | | GEHGIVVFSLGSMVSEIPEKKAMAIADALGKIP |
| | | QTVLWRYTGTRPSNLANNTILVKWLPQNDLLG |
| | | HPMTRAFITHAGSHGVYESICNGVPMVMMPLF |
| | | GDQMDNAKRMETKGAGVTLNVLEMTSEDLEN |
| | | ALKAVINDKSYKENIMRLSSLHKDRPVEPLDLA |
| | | VFWVEFVMRHKGAPHLRPAAHDLTWYQYHSL |
| | | DVIGFLLAVVLTVAFITFKCCAYGYRKCLGKKG |
| | | RVKKAHKSKTH (SEQ ID NO: 12) |
| UDP- | Q9HAW8 | MARAGWTSPVPLCVCLLLTCGFAEAGKLLVVP |
| glucuronosyltransferase | | MDGSHWFTMQSVVEKLILRGHEVVVVMPEVS |
| 1-10/Homo sapiens | | WQLERSLNCTVKTYSTSYTLEDQNREFMVFAH |
| | | AQWKAQAQSIFSLLMSSSSGFLDLFFSHCRSLF |
| | | NDRKLVEYLKESSFDAVFLDPFDTCGLIVAKYF |
| | | SLPSVVFTRGIFCHHLEEGAQCPAPLSYVPNDLL |
| | | GFSDAMTFKERVWNHIVHLEDHLFCQYLFRNA |
| | | LEIASEILQTPVTAYDLYSHTSIWLLRTDFVLDY |
| | | PKPVMPNMIFIGGINCHQGKPLPMEFEAYINAS |
| | | GEHGIVVFSLGSMVSEIPEKKAMAIADALGKIP |
| | | QTVLWRYTGTRPSNLANNTILVKWLPQNDLLG |
| | | HPMTRAFITHAGSHGVYESICNGVPMVMMPLF |
| | | GDQMDNAKRMETKGAGVTLNVLEMTSEDLEN |
| | | ALKAVINDKSYKENIMRLSSLHKDRPVEPLDLA |
| | | VFWVEFVMRHKGAPHLRPAAHDLTWYQYHSL |
| | | DVIGFLLAVVLTVAFITEKCCAYGYRKCLGKKG |
| | | RVKKAHKSKTH (SEQ ID NO: 13) |
| 4-dimethylallyl | M1WA41 | MSTAKDPGNGVYEILSLIFDFPSNEQRLWWHST |
| tryptophan synthase/ | | APMFAAMLDNAGYNIHDQYRHLGIFKKHIIPFL |
| Claviceps purpurea | | GVYPTKDKERWLSILTRCGLPLELSLNCTDSVV |
| | | RYTYEPINEVTGTEKDPFNTLAIMASVQKLAQI |
| | | QAGIDLEWFSYFKDELTLDESESATLQSNELVK |
| | | EQIKTQNKLALDLKESQFALKVYFYPHLKSIAT |
| | | GKSTHDLIFDSVFKLSOKHDSIQPAFQVLCDYV |
| | | SRRNHSAESDQHIALHARLLSCDLIDPAKSRVKI |
| | | YLLEKTVSLSVMEDLWTLGGQRVDASTMDGL |
| | | DMLRELWSLLKVPTGHLEYPKGYLELGEIPNEQ |
| | | LPSMANYTLHHNNPMPEPQVYFTVFGMNDAEI |
| | | SNALTIFFQRHGFDDMAKKYRVFLQDSYPYHD |
| | | FESLNYLHAYISFSYRRNKPYLSVYLHTFETGR |
| | | WPVVADSPISFDAYRRCDLSTK (SEQ ID |
| | | NO: 14) |
| 7- | Q4WYG3 | MSIGAEIDSLVPAPPGLNGTAAGYPAKTQKELS |
| dimethylallyltryptophan | | NGDFDAHDGLSLAQLTPYDVLTAALPLPAPASS |
| synthase/ | | TGFWWRETGPVMSKLLAKANYPLYTHYKYLM |
| Neosartorya fumigata | | LYHTHILPLLGPRPPLENSTHPSPSNAPWRSFLT |
| | | DDFTPLEPSWNVNGNSEAQSTIRLGIEPIGFEAG |
| | | AAADPFNQAAVTQFMHSYEATEVGATLTLFEH |
| | | FRNDMFVGPETYAALRAKIPEGEHTTQSFLAFD |
| | | LDAGRVTTKAYFFPILMSLKTGQSTTKVVSDSI |
| | | LHLALKSEVWGVQTIAAMSVMEAWIGSYGGA |
| | | AKTEMISVDCVNEADSRIKIYVRMPHTSLRKVK |
| | | EAYCLGGRLTDENTKEGLKLLDELWRTVFGID |
| | | DEDAELPONSHRTAGTIENFELRPGKWFPEPKV |
| | | YLPVRHYCESDMQIASRLQTFFGRLGWHNMEK |
| | | DYCKHLEDLFPHHPLSSSTGTHTFLSFSYKKQK |
| | | GVYMTMYYNLRVYST (SEQ ID NO: 15) |

Accordingly, the objective of the present disclosure is to provide novel compositions and processes for the production of indole alkaloids. In some embodiments, the enzyme mixture capable of transferring donor functional groups to acceptor functional groups on the indole alkaloid may be biosynthetically produced by metabolic pathways in the cell.

In some case, enzymes incorporated in the enzyme mixture are engineered enzymes. In some case, enzymes incorporated in the enzyme mixture have modified sequences of amino acids.

### Enzyme Mixture Conditions and Product Production

In some cases, the enzyme mixture is reacted under aerobic conditions. In some cases, the enzyme mixture is reacted under anaerobic conditions.

In some cases, the enzyme may be buffered, for example, by phosphate salts, HEPES, or Tris. In some cases, the enzyme mixture may be a minimal media, including, but not limited to, M9, MOPS, YNB, ammonia salts, or a complex media containing, for example, yeast extract, casamino acids, peptone, or tryptone. In some cases, the enzyme mixture may contain a reducing agent, for example, L-ascorbic acid, dithiothreitol, or mercaptoethanol. In some cases, the enzyme mixture may be supplemented with additional amino acids, such as L-methionine, Histidine, Arginine, Alanine, Isoleucine, Cysteine, Aspartic acid, Leucine, Glutamine, Asparagine, Lysine, Glycine, Glutamic acid, Proline, Serine, Phenylalanine, Tyrosine, Selenocysteine, Threonine, Pyrrolysine, Tryptophan, or Valine. In some cases, additional vitamins and cofactors may be added, for example, L-ascorbic acid, thiamine, pyridoxal phosphate, niacin, pyridoxine, biotin, folic acid, tetrahydrofolic acid, riboflavin, pantothenic acid, copper salts, magnesium salts, manganese salts, molybdenum salts, iron salts, zinc salts, nickel salts, glutathione, heme, or D-aminolevulinic acid.

In some cases, the enzyme mixture may be fed a substituted anthranilate by single addition, batch feeding, or constant dilution in culture. In some cases, the enzyme mixture may be fed a substituted indole by single addition, batch feeding, or constant dilution in culture.

In some cases, a downstream product may be produced. In some cases, the downstream product may be purified. e.g., isolated and purified from the culture medium, from a cell lysate, or both. In some cases, the downstream product may be at least, or about, 25%, 30%, 40%, 50%, 60%, 70%, 75%, 80%, 90%, 95%, or 99%, by weight, pure. Purification can be carried out by any known method or combination of methods, which methods include, e.g., column chromatography, phase separation, precipitation, crystallization, decantation, gas stripping, membrane enhanced separation, fractionation, adsorption/desorption, pervaporation, thermal or vacuum desorption from a solid phase, extraction of the product that is immobilized or absorbed to a solid phase with a solvent, etc. Purity can be assessed by any appropriate method, e.g., by column chromatography, high performance liquid chromatography (HPLC) analysis, or gas chromatography-mass spectrometry (GC-MS) analysis.

In some cases, the enzyme mixture may convert greater than or about 0.0015%, 0.002%, 0.005%, 0.01%, 0.02%, 0.05%, 0.1%, 0.12%, 0.14%, 0.16%, 0.2%, 0.3%, 0.4%, 0.5%, 0.6%, 0.7%, 0.8%, 0.9%, 1.0%, 1.2%, 1.4%, 1.6%, 1.8%, 2.0%, 2.5%, 3.0%, 3.5%, 4.0%, 5.0%, 6.0%, 7.0%, or 8.0% of the fed precursor in the enzyme mixture into the desired product. In some cases, the enzyme mixture may produce at least 2 g/L, at least 3 g/L, at least 4 g/L, at least 5 g/L, at least 7 g/L, at least 10 g/L, or more than 50 g/L of the desired product in liquid culture medium.

In some cases, the enzyme mixture may convert greater than or about 0.0015%, 0.002%, 0.005%, 0.01%, 0.02%, 0.05%, 0.1%, 0.12%, 0.14%, 0.16%, 0.2%, 0.3%, 0.4%, 0.5%, 0.6%, 0.7%, 0.8%, 0.9%, 1.0%, 1.2%, 1.4%, 1.6%, 1.8%, 2.0%, 2.5%, 3.0%, 3.5%, 4.0%, 5.0%, 6.0%, 7.0%, or 8.0% of the carbon in the enzyme mixture into the desired product. In some cases, the enzyme mixture may produce at least 2 g/L, at least 3 g/L, at least 4 g/L, at least 5 g/L, at least 7 g/L, at least 10 g/L, or more than 50 g/L of the desired product in the enzyme mixture.

### Host Cells

Suitable host cells include cells that can be cultured in the enzyme mixture, e.g., unicellular organisms. Suitable host cells include yeast cells, fungal cells, insect cells, mammalian cells, algal cells, and bacterial cells. Suitable host cells may further include filamentous fungal cells; suitable filamentous fungal cells include, e.g., Aspergillus. Neurospora, and the like.

The host cell can be a prokaryotic cell. Suitable prokaryotic cells include, but are not limited to, any of a variety of laboratory strains of Escherichia coli, Corynebacterium glutamicum, Lactobacillus sp., Salmonella sp., Shigella sp., Citrobacter, Enterobacter, Clostridium, Klebsiella, Aerobacter, and the like. See, e.g., Carrier et al. (1992) J. Immunol. 148:1176-1181; U.S. Pat. No. 6,447,784; and Sizemore et al. (1995) Science 270:299-302. Examples of Salmonella strains which can be employed in the present disclosure include, but are not limited to, Salmonella typhi and S. typhimurium. Suitable Shigella strains include, but are not limited to, Shigella flexneri, Shigella sonnei, and Shigella disenteriae. Typically, the laboratory strain is one that is non-pathogenic. Non-limiting examples of other suitable bacteria include, but are not limited to, Bacillus subtilis, Pseudomonas pudita, Pseudomonas aeruginosa, Pseudomonas mevalonii, Rhodobacter sphaeroides, Rhodobacter capsulatus, Rhodospirillum rubrum, Rhodococcus sp., and the like. In some cases, the host cell is Escherichia coli.

Non-limiting examples of suitable yeast host cells are strains selected from a cell of a species of Candida, Kluyveromyces, Saccharomyces, Schizosaccharomyces, Pichia, Hansenula, and Yarrowia. In some cases, the yeast host cell may be selected from the group consisting of: Saccharomyces carlsbergensis, Saccharomyces cerevisiae, Saccharomyces diastaticus, Saccharomyces douglasii, Saccharomyces kluyveri, Saccharonyces norbensis, Saccharomyces oviformis, Schizosaccharomyces pombe, Saccharomyces uvarum, Pichia kluyveri, Yarrowia lipolytica, Candida utilis, Candida cacaoi, and Geotrichum fermentans. Other useful yeast host cells are Kluyveromyces lactis, Kluyveromwyces fragilis, Hansenula polymorpha, Pichia pastoris, Yarrowia lipolvtica, Schizosaccharomyces pombe, Ustilgo maylis, Candida maltose, Pichia guillermondii, and Pichia methanoliol. Suitable yeast host cells may include, but are not limited to, Pichia pastoris, Pichia finlandica, Pichia trehalophila, Pichia koclamae, Pichia membranaefaciens, Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, and the like. In some cases, a yeast host cell may be Saccharomyces cerevisiae; e.g., a genetically modified cell of the present disclosure may be a genetically modified Saccharomyces cerevisiae cell.

The filamentous fungi may be characterized by a mycelial wall composed of chitin, cellulose, glucan, chitosan, mannan, and other complex polysaccharides. Vegetative growth may be by hyphal elongation and carbon catabolism may be obligately aerobic. Suitable filamentous fungal strains include, but are not limited to, strains of Acremonium, Agaricus, Aspergillus, Aureobasidium, Chrysosporium, Coprinus, Cryptococcus, Filibasidium, Fusarium, Humicola, Magnaporthe, Mucor, Myceliophthora, Neocallimastix, Neurospora, Paecilomyces, Penicillium, Piromyces, Phanerochaete, Pleurotus, Schizophyllum, Talaromyces, Thermoascus, Thielavia, Tolypocladium, and Trichoderma. Non-limiting examples of suitable filamentous fungal cells include, e.g., Aspergillus niger, Aspergillus awamori, Aspergillus foetidus, Aspergillus sojae, Aspergillus fumigatus, and Aspergillus oryzae. Another example of a suitable fungal cell is a Neurospora crassa cell.

### Heterologous Protein Expression in Modified Host Cells

In some cases, a nucleotide sequence encoding a heterologous polypeptide may be operably linked to a transcriptional control element.

Suitable promoters for expression in bacteria may include, but are not limited to, pT7, ptac, pLac, pLacUV5, pret, pBAD, and the constitutive BBa series of promoters of the Anderson promoter library (Kelly et al, “Measuring the activity of BioBrick promoters using an in vivo reference standard” Journal of Biological Engineering 2009 3:4). Suitable promoters for expression in yeast may include, but are not limited to, TDH3, CCW12, CYC1, HIS3, GAL1, GAL10, ADH1, PGK, PHO5, GAPDH, ADC1, TRP1, URA3, LEU2, ENO, and TP1; and, AOX1 (e.g., for use in Pichia).

The expression vector may also contain a ribosome binding site for translation initiation and a transcription terminator. The expression vector may also include appropriate sequences for amplifying expression.

In some cases, the expression of the amino acid sequence may be codon optimized or biased to increase expression of protein in vivo. This may be achieved by several algorithms (Hanson and Coller, Nature Reviews Molecular Cell Biology volume 19, pages 20-30 (2018)), (Quax, et al Molecular Cell Review volume 59, Jul. 16, 2015). In some cases, the native amino acid sequence may be used for coding an amino acid sequence in vivo.

### Pharmaceutical Compositions

The compounds described herein may be formulated as a pharmaceutical composition. A pharmaceutical composition may comprise: (i) a modified indole alkaloid provided herein; and (ii) a pharmaceutically acceptable carrier, diluent, or excipient. A pharmaceutical composition comprising a modified indole alkaloid described herein can be formulated according to known methods to prepare pharmaceutically useful compositions, whereby the therapeutic molecule is combined in a mixture with a pharmaceutically acceptable carrier, diluent, or excipient.

Sterile phosphate-buffered saline is one example of a pharmaceutically acceptable carrier. Other suitable carriers, diluents, or excipients are well-known to those in the art. (See, e.g., Gennaro (ed.), Remington's Pharmaceutical Sciences (Mack Publishing Company, 19th ed. 1995).) Formulations can further include one or more excipients, preservatives, solubilizers, buffering agents, albumin to prevent protein loss on vial surfaces, etc.

A pharmaceutical composition comprising a modified indole alkaloid described herein may be formulated in a dosage form selected from the group consisting of: an oral unit dosage form, an intravenous unit dosage form, an intranasal unit dosage form, a suppository unit dosage form, an intradermal unit dosage form, an intramuscular unit dosage form, an intraperitoneal unit dosage form, a subcutaneous unit dosage form, an epidural unit dosage form, a sublingual unit dosage form, a liquid, a lozenge, a fast disintegrating tablet, a lyophilized preparation, a film, a spray (including a nasal spray, an oral spray, or a topical spray), or a mucoadhesive. The oral unit dosage form may be selected from the group consisting of: tablets, pills, pellets, capsules, powders, lozenges, granules, solutions, suspensions, emulsions, syrups, elixirs, sustained-release formulations, aerosols, and sprays. In some embodiments, the modified indole alkaloid is formulated as a liquid, a lozenge, a fast-disintegrating tablet, a lyophilized preparation, a film, a spray, or a mucoadhesive.

Pharmaceutical compositions comprising modified indole alkaloids as described herein may also contain one or more additional ingredients including, but not limited to, a mucoadhesive compound, a buffering agent, a plasticizing agent, a stabilizing agent, a taste-masking agent, a flavoring agent, a coloring agent, an antiseptic, an inert filler agent, a preservative, and combinations thereof.

In some embodiments, the formulations comprise one or more solubilizing agents that increase the solubility of active compounds in the formulation. Suitable solubilizing agents include, for example, complexing agents, surfactants, and the like. Suitable complexing agents include unsubstituted cyclodextrins (such as alpha-cyclodextrin, beta-cyclodextrin) and substituted cyclodextrins, (such as hydroxypropyl beta-cyclodextrin, sulfobutylether-beta-cyclodextrin). Suitable surfactants include polyoxyethylene sorbitan monolaurate (for example, Tween 20), polyoxyethylene sorbitans molooleate (for example, Tween 80), polyethylene glycol (15)-hydroxystearate (for example, Kolliphor® HS 15), PEG-35 castor oil (for example, Kolliphor® EL) and PEG-60 hydrogenated castor oil (for example, Cremophor® RH 60).

In some embodiments, the formulations comprise one or more buffer agents that maintain the pH of the IV solution within a pharmaceutically acceptable range. In certain embodiments, the buffer maintains the pH of the IV solution between about 5 and 9. In specific embodiments, the buffer maintains the pH of the IV solution at about 7.4. Suitable buffers include, for example, citrates, lactate, acetate, maleate, phosphates, and the like.

In some embodiments, the formulations comprise one or more density modifiers that is used to control the density of the IV formulation. Suitable density modifiers include, for example, dextrose.

In some embodiments, the formulations comprise one or more isotonicity modifiers that provide a formulation that is iso-osmotic with tissue to prevent pain and irritation when the formulation is administered. Suitable isotonicity modifiers include, for example, electrolytes, monosaccharides, and disaccharides. Examples of isotonicity modifiers include glycerin, dextrose, potassium chloride, and sodium chloride.

In some embodiments, the formulations comprise one or more viscosity enhancers. Suitable viscosity enhancers include, for example, povidone, hydroxyethylcellulose, polyvinyl alcohol, and carbomer (such as, acrylic acid homopolymers and acrylic acid copolymers).

In some embodiments, the formulations comprise one or more preservatives that increase the stability of active compounds in the formulation and/or provide antimicrobial activity. Suitable preservatives include, for example, antimicrobial agents and antioxidants. Examples of antimicrobial agents (with ranges of anti-microbial effective amounts shown as weight of antimicrobial agent per volume of IV formulation, i.e., % w/v) include benzyl alcohol (about 0.1-3.0% w/v”), methyl paraben (about 0.08-0.1% w/v), propyl paraben (0.001-0.023% w/v), phenol (0.2-0.5% w/v), cresol (0.2-0.5% w/v), methyl paraben (0.1% w/v), chlorbutanol (0.25-0.5% w/v), sodium metabisulphite (0.025-0.66% w/v), sodium bisulphite (0.13-0.2% w/v), benzethonium chloride (0.08-0.1% w/v), and benzalkonium chloride (0.08-0.1% w/v). Examples of antioxidants include sodium bisulphite and other sulfurous acid salts, ascorbic acid, salts of ethylenediaminetetraacetic acid (including sodium), alpha tocopherol, butylated hydroxyl hydroxytoluene, and butylated hydroxyanisole.

### Routes of Administration

According to the methods of the present disclosure, a modified indole alkaloid described herein can be administered to subjects by a variety of administration modes, including, for example, by intramuscular, subcutaneous, intravenous, intra-atrial, intra-articular, parenteral, intranasal, intrapulmonary, transdermal, intrapleural, intrathecal, and oral routes of administration. For prevention and treatment purposes, a modified indole alkaloid described herein can be administered to a subject in a single bolus delivery, via continuous delivery (e.g., continuous transdermal delivery) over an extended time period, or in a repeated administration protocol (e.g., on an hourly, dally, weekly, or monthly basis).

### Kits/Articles of Manufacture

Pharmaceutical compositions comprising a modified indole alkaloid described herein can be supplied as a kit comprising a container that comprises the pharmaceutical composition as described herein. A pharmaceutical composition can be provided, for example, in the form of an injectable solution for single or multiple doses, or as a sterile powder that will be reconstituted before injection. Alternatively, such a kit can include a dry-powder disperser, liquid aerosol generator, or nebulizer for administration of a pharmaceutical composition. Such a kit can further comprise written information on indications and usage of the pharmaceutical composition.

### Methods of Enzymatic Preparation of Modified Indole Alkaloids

In yet another aspect, provided herein are a method of enzymatically preparing an indole alkaloid, comprising:
contacting a compound of Formula (Ia′) with an enzyme and a co-substrate;
wherein the compound of Formula (Ia′) has a structure of:

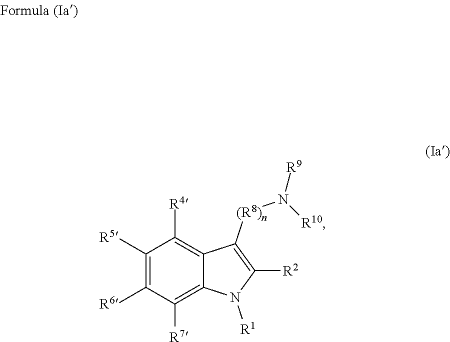

or a pharmaceutically acceptable salt thereof, wherein,
   R¹ and R¹⁰ are independently selected from hydrogen, C₁-C₆ alkyl, C₂-C₆ alkenyl, and C₂-C₆ alkynyl, wherein alkyl, alkenyl, and alkynyl are optionally substituted with one or more substituents independently selected from halo, —OMe, —CN, —NH₂, and —NO₂;
   R⁸ is —CR′₂—, wherein each R′ is independently selected from hydrogen, halo, haloalkyl, alkoxy, haloalkoxy, and amine;
   n is selected from 2, 3, and 4;
   R⁹ is selected from C₂-C₆ alkyl, C₂-C₆ alkenyl, and C₂-C₆ alkynyl, wherein alkyl, alkenyl, and alkynyl are optionally substituted with one or more substituents independently selected from halo, —OMe, —CN, —NH₂, —NO₂, and 3- to 8-membered heterocycle, and wherein 3- to 8-membered heterocycle is optionally substituted with one or more substituents independently selected from halo, —OMe, —CN, —NH₂, and —NO₂;
   R² is selected from hydrogen, halogen, C₁-C₆ alkyl, and C₁-C₆ haloalkyl;
   R⁴, R⁵, R⁶, and R⁷ are each independently selected from hydrogen, —OH, and C₁-C₆ alkyl; and wherein at least one of R⁴, R⁵, R⁶, and R⁷ is —OH.

In some embodiments, R⁴ is —OH.

In some embodiments, R⁵ is —OH.

In some embodiments, R⁶ is —OH.

In some embodiments, R⁷ is —OH.

In some embodiments, the enzyme is a 4-hydroxytryptamine kinase.

In some embodiments, the enzyme is an acetylserotonin O-methyltransferase.

In some embodiments, the enzyme is a tryptamine n-methyltransferase.

In some embodiments, the enzyme is a sulfotransferase 1A1.

In some embodiments, the enzyme is a sulfotransferase 1A3.

In some embodiments, the enzyme is an alcohol 0-acetyltransferase 1.

In some embodiments, the enzyme is a chloramphenicol acetyltransferase.

In some embodiments, the enzyme is an UDP-glucuronosyltransferase. In some embodiments, the UDP-glucuronosyltransferase is an UDP-glucuronosyltransferase 1-6. In some embodiments, the UDP-glucuronosyltransferase is an UDP-glucuronosyltransferase 1-9. In some embodiments, the UDP-glucuronosyltransferase is an UDP-glucuronosyltransferase 1-10.

In some embodiments, the enzyme is an oleandomycin glycosyltransferase.

In some embodiments, the enzyme is a glycosyltransferase.

In some embodiments, the enzyme is a 4-dimethylallyl tryptophan synthase.

In some embodiments, the enzyme is a 7-dimethylallyltryptophan synthase.

As an example, when R⁴ is —OH, various modified indole alkaloids of Formula (Ia) can be made as shown in the general synthetic Scheme 1 below:

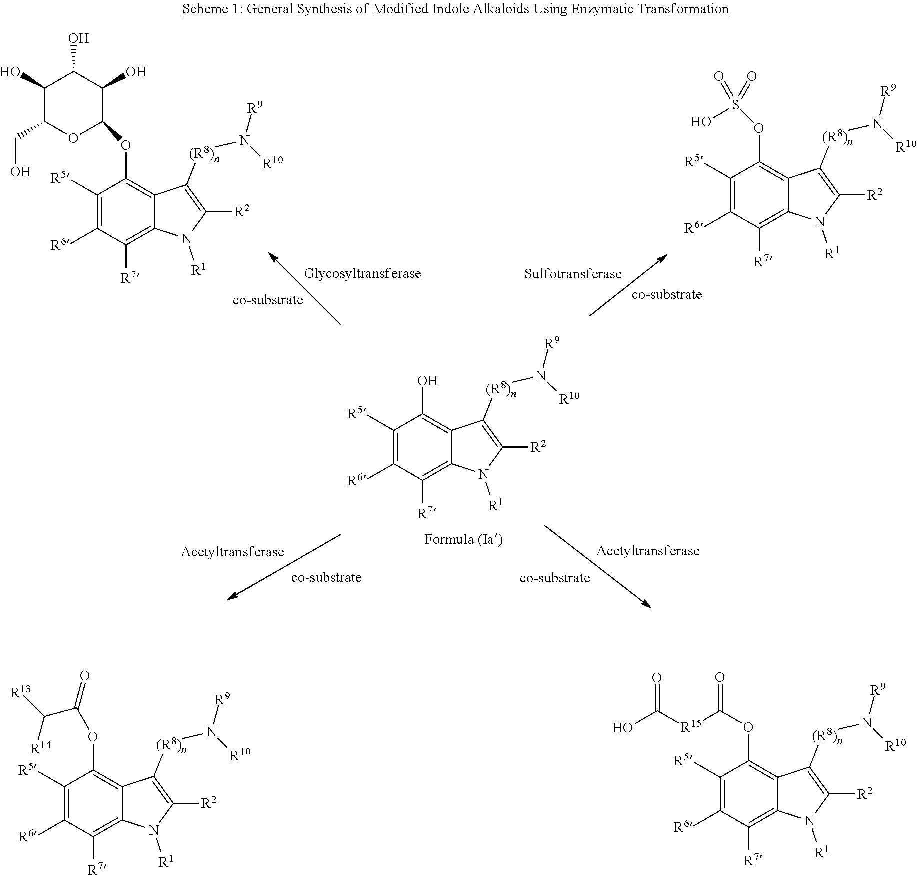

### Methods of Treatment

Compositions described within this disclosure include modified indole alkaloids which have therapeutic uses for mental disorders including, but not limited to, depressive and anxiety disorders, alcoholism, terminal illness, depression and anxiety associated with terminal illness, prolonged grief disorder, complicated grief disorder, and post-traumatic stress disorder. The modified indole alkaloids provided herein have therapeutic uses including, but not limited to, treatment of major depression, treatment resistant depression, anxiety, post-traumatic mania, psychosis, insomnia, hypersomnia, Alzheimer's disease, Parkinson's disease, burnout, cluster headaches, migraine headaches and other neurological disorders.

In some embodiments, the present disclosure provides methods of treating mental disorders by administering a modified indole alkaloid or salts thereof to the patient in need thereof. In some embodiments, the method comprises administration of the modified indole alkaloid or salts thereof to the patient in need of treatment.

In some embodiments, the methods provided herein do not result in a concurrent increase or onset of negative symptoms such as depression or anxiety. Additional negative symptoms may include feeling agitated, shaky, or anxious, indigestion, diarrhea or constipation, loss of appetite and weight loss, dizziness, blurred vision, dry mouth, excessive sweating, sleeping problems (insomnia) or drowsiness, and/or headaches.

In another aspect, provided herein is a method of treating a disease or disorder in a subject in need thereof comprising administering a compound of Formula (Ia):

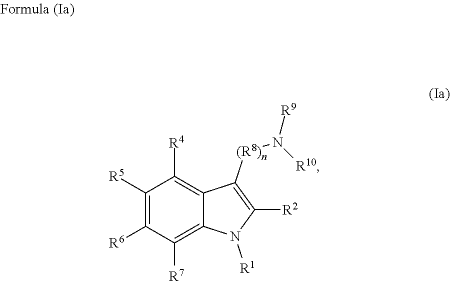

or a pharmaceutically acceptable salt thereof, wherein,
R¹ and R¹⁰ are independently selected from hydrogen, C₁-C₆ alkyl, C₂-C₆ alkenyl, and C₂-C₆ alkynyl, wherein alkyl, alkenyl, and alkynyl are optionally substituted with one or more substituents independently selected from halo, —OMe, —CN, —NH₂, and —NO₂;
R⁸ is —CR′₂—, wherein each R′ is independently selected from hydrogen, halo, haloalkyl, alkoxy, haloalkoxy, and amine;
n is selected from 2, 3, and 4;
R⁹ is selected from C₂-C₆ alkyl, C₂-C₆ alkenyl, and C₂-C₆ alkynyl, wherein alkyl, alkenyl, and alkynyl are optionally substituted with one or more substituents independently selected from halo, —OMe, —CN, —NH₂, —NO₂, and 3- to 8-membered heterocycle, and wherein 3- to 8-membered heterocycle is optionally substituted with one or more substituents independently selected from halo, —OMe, —CN, —NH₂, and —NO₂;
R² is selected from hydrogen, halogen, C₁-C₆ alkyl, and C₁-C₆ haloalkyl;
R⁴, R⁵, R⁶, and R⁷ are each independently selected from hydrogen, C₁-C₆ alkyl, A, J, Q, and X;
A is

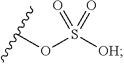 [SMILES: *OS(=O)(=O)O]

J is

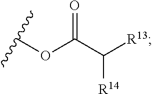

Q is

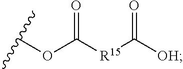

X is selected from glucose, xylose, galactose, rhamnose, rutinose, and disaccharide;
R¹³ is selected from hydrogen, and C₁-C₆ alkyl optionally substituted with one or more substituents independently selected from C₁-C₆ alkyl, oxo, halo, —OMe, —CN, —NH₂, and —NO₂;
R¹⁴ is selected from C₁-C₆ alkyl and C₂-C₆ alkenyl, wherein C₁-C₆ alkyl and C₂-C₆ alkenyl are optionally substituted with one or more substituents independently selected from halo, —OMe, —CN, —NH₂, and —NO₂;
or R¹³ and R¹⁴ taken together with the atom to which they are attached to form a substituted or unsubstituted C₃-C₈cycloalkyl or substituted or unsubstituted 3- to 8-membered heterocycloalkyl having 1 or 2 heteroatoms each independently selected from N, O, and S;
R¹⁵ is selected from C₁-C₆ alkylene and C₂-C₆ alkenylene, wherein C₁-C₆ alkylene and C₂-C₆ alkenylene are optionally substituted with one or more substituents independently selected from C₁-C₆ alkyl, halo, —OMe, —CN, —NH₂, and —NO₂; and
wherein at least one of R⁴, R⁵, R⁶, and R⁷ is A, J, Q, or X.

In some embodiments, provided herein is a method of treating a disease or disorder in a subject in need thereof comprising administering a compound of Formula (l):

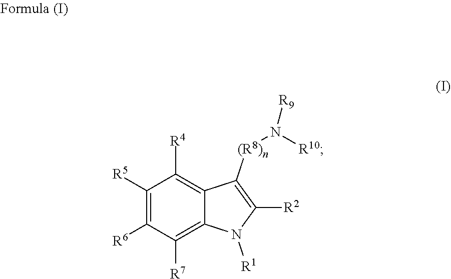

or a pharmaceutically acceptable salt thereof, wherein,
R¹ and R¹⁰ are independently selected from hydrogen, C₁-C₆ alkyl, C₂-C₆ alkenyl, and C₂-C₆ alkynyl, wherein alkyl, alkenyl, and alkynyl are optionally substituted with one or more substituents independently selected from halo, —OMe, —CN, —NH₂, and —NO₂;
R⁸ is —CR′₂—, wherein each R′ is independently selected from hydrogen, halo, haloalkyl, alkoxy, haloalkoxy, and amine;
n is selected from 2, 3, and 4;
R⁹ is selected from C₂-C₆ alkyl, C₂-C₆ alkenyl, and C₂-C₆ alkynyl, wherein alkyl, alkenyl, and alkynyl are optionally substituted with one or more substituents independently selected from halo, —OMe, —CN, —NH₂, —NO₂, and 3- to 8-membered heterocycle, and wherein 3- to 8-membered heterocycle is optionally substituted with one or more substituents independently selected from halo, —OMe, —CN, —NH₂, and —NO₂;
R² is selected from hydrogen, halogen, C₁-C₆ alkyl, and C₁-C₆ haloalkyl;
R⁴, R⁵, R⁶, and R⁷ are each independently selected from hydrogen, C₁-C₆ alkyl, A, J, Q, and X;
A is

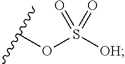 [SMILES: *OS(=O)(=O)O]

J is

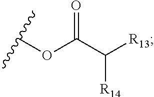

Q is

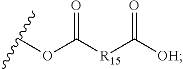

X is selected from glucose, xylose, galactose, rhamnose, and rutinose;
R¹³ is selected from hydrogen, and C₁-C₆ alkyl optionally substituted with one or more substituents independently selected from halo, —OMe, —CN, —NH₂, and —NO₂;
R¹⁴ is selected from C₁-C₆ alkyl and C₂-C₆ alkenyl, wherein C₁-C₆ alkyl and C₂-C₆ alkenyl are optionally substituted with one or more substituents independently selected from halo, —OMe, —CN, —NH₂, and —NO₂;
R¹⁵ is selected from C₁-C₆ alkylene and C₂-C₆ alkenylene, wherein C₁-C₆ alkylene and C₂-C₆ alkenylene are optionally substituted with one or more substituents independently selected from halo, —OMe, —CN, —NH₂, and —NO₂; and
   wherein at least one of R⁴, R⁵, R⁶, and R⁷ is A or Q, or at least one of R⁵, R⁶, and R⁷ is J or X.

In some embodiments, R¹ is selected from hydrogen, C₁-C₆ alkyl, C₂-C₆ alkenyl, and C₂-C₆ alkynyl, wherein alkyl, alkenyl, and alkynyl are optionally substituted with one or more substituents independently selected from halo, —OMe, —CN, —NH₂, and —NO₂. In some embodiments, R¹ is selected from hydrogen, C₁-C₆ alkyl, and C₂-C₆ alkenyl, wherein alkyl and alkenyl are optionally substituted with one or more substituents independently selected from halo, —OMe, —CN, —NH₂, and —NO₂. In some embodiments, R¹ is selected from hydrogen and C₁-C₆ alkyl, wherein alkyl is optionally substituted with one or more substituents independently selected from halo, —OMe, and —CN. In some embodiments, R¹ is selected from hydrogen and C₁-C₃ alkyl. In some embodiments, R¹ is hydrogen. In some embodiments, R¹ is C₁-C₃ alkyl.

In some embodiments, R¹⁰ is selected from hydrogen, C₁-C₆ alkyl, C₂-C₆ alkenyl, and C₂-C₆ alkynyl, wherein alkyl, alkenyl, and alkynyl are optionally substituted with one or more substituents independently selected from halo, —OMe, —CN, —NH₂, and —NO₂. In some embodiments, R¹⁰ is selected from hydrogen, C₁-C₆ alkyl, and C₂-C₆ alkenyl, wherein alkyl and alkenyl are optionally substituted with one or more substituents independently selected from halo, —OMe, —CN, —NH₂, and —NO₂. In some embodiments, R¹⁰ is independently selected from hydrogen, C₁-C₃ alkyl, and C₂-C₃ alkenyl, wherein alkyl and alkenyl are optionally substituted with one or more substituents independently selected from halo, —OMe, and —CN. In some embodiments, R¹⁰ is independently selected from hydrogen, C₁-C₃ alkyl, and C₂-C₃ alkenyl. In some embodiments, R¹⁰ is hydrogen. In some embodiments, R¹⁰ is C₁-C₃ alkyl. In some embodiments, R¹⁰ is C₂—C alkenyl.

In some embodiments, each R′ is independently selected from hydrogen, halo, haloalkyl, alkoxy, haloalkoxy, and amine. In some embodiments, each R′ is independently selected from hydrogen, halo, and haloalkyl. In some embodiments, each R′ is hydrogen. In some embodiments, each R′ is halo. In some embodiments, each R′ is haloalkyl. In some embodiments, each R′ is alkoxy. In some embodiments, each R′ is haloalkoxy. In some embodiments, each R′ is amine.

In some embodiments, n is selected from 2, 3, and 4. In some embodiments, n is selected from 2 and 3. In some embodiments, n is 2. In some embodiments, n is 3. In some embodiments, n is 4.

In some embodiments, R⁹ is selected from C₁-C₆ alkyl, C₂-C₆ alkenyl, and C₁-C₆ alkynyl, wherein alkyl, alkenyl, and alkynyl are optionally substituted with one or more substituents independently selected from halo, —OMe, —CN, —NH₂, —NO₂, and 3- to 8-membered heterocycle, and wherein 3- to 8-membered heterocycle is optionally substituted with one or more substituents independently selected from halo, —OMe, —CN, —NH₂, and —NO₂. In some embodiments, R⁹ is selected from C₂-C₆ alkyl, C₂-C₆ alkenyl, and C₂-C₆ alkynyl, wherein alkyl, alkenyl, and alkynyl are optionally substituted with one or more substituents independently selected from halo, —OMe, —CN, —NH₂, —NO₂, and 3- to 8-membered heterocycle, and wherein 3-to 8-membered heterocycle is optionally substituted with one or more substituents independently selected from halo, —OMe, and —CN. In some embodiments, R⁹ is selected from C₂—C(alkyl and C₂-C₆ alkenyl, wherein alkyl and alkenyl are optionally substituted with one or more substituents independently selected from halo, —OMe, —CN, and —NH₂. In some embodiments, R⁹ is selected from C₂-C₃ alkyl and C₂-C₃ alkenyl, wherein alkyl and alkenyl are optionally substituted with one or more substituents independently selected from halo, —OMe, and —CN. In some embodiments, R⁹ is C₂-C₃ alkyl. In some embodiments, R¹ is C₂-C₃ alkenyl.

In some embodiments, R² is selected from hydrogen, halogen, C₁-C₆ alkyl, and C₁-C₆ haloalkyl. In some embodiments, R² is selected from hydrogen, halogen, and C₁-C₆ alkyl. In some embodiments, R² is hydrogen. In some embodiments, R² is halogen. In some embodiments, R₂ is C₁-C₆ alkyl. In some embodiments, R² is C₁-C₆ haloalkyl.

In some embodiments. R⁴, R⁵, R⁶, and R⁷ are each independently selected from hydrogen, A, J, Q, and X. In some embodiments, R⁴, R⁵, R⁶, and R⁷ are each independently selected from hydrogen, J, and Q. In some embodiments, at least one of R⁴, R⁵, R⁶, and R⁷ is A, J, Q, or X. In some embodiments, at least one of R⁴, R⁵, R⁶, and R⁷ is A. In some embodiments, R⁴ is A. In some embodiments, R⁵ is A. In some embodiments, R⁶ is A. In some embodiments, R⁷ is A. In some embodiments, at least one of R⁴, R⁵, R⁶, and R⁷ is J. In some embodiments, R⁴ is J. In some embodiments, R⁵ is J. In some embodiments, R⁶ is J. In some embodiments, R⁷ is J. In some embodiments, at least one of R⁴, R⁵, R⁶, and R⁷ is Q. In some embodiments, R⁴ is Q. In some embodiments, R⁵ is Q. In some embodiments, R⁶ is Q. In some embodiments, R¹⁷ is Q. In some embodiments, at least one of R⁴, R⁵, R⁶, and R⁷ is X. In some embodiments, R⁴ is X. In some embodiments, R⁵ is X. In some embodiments, R⁶ is X. In some embodiments, R⁷ is X.

In some embodiments, R¹³ is selected from hydrogen, and C₁-C₆ alkyl optionally substituted with one or more substituents independently selected from halo, —OMe, —CN, —NH₂, and —NO₂. In some embodiments, R¹³ is selected from hydrogen, and C₁-C₆ alkyl optionally substituted with one or more substituents independently selected from halo, —OMe, —CN, and —NH₂. In some embodiments, R¹³ is selected from hydrogen and C₁-C₃ alkyl optionally substituted with one or more substituents independently selected from halo, —OMe, and —CN. In some embodiments, R¹³ is hydrogen. In some embodiments, R¹³ is C₁-C₃ alkyl.

In some embodiments, R¹⁴ is selected from C₁-C₆ alkyl and C₂-C₆ alkenyl, wherein C₁-C₆ alkyl and C₂-C₆ alkenyl are optionally substituted with one or more substituents independently selected from halo, —OMe, —CN, —NH₂, and —NO₂. In some embodiments, R¹⁴ is selected from C₁-C₆ alkyl and C₂-C₆ alkenyl, wherein C₁-C₆ alkyl and C₂-C₆ alkenyl are optionally substituted with one or more substituents independently selected from halo, —OMe, and —CN. In some embodiments, R¹⁴ is C₁-C₃ alkyl optionally substituted with one or more substituents independently selected from halo, —OMe, and —CN. In some embodiments, R¹⁴ is C₁-C₃ alkyl. In some embodiments, R¹⁴ is C₂-C₃ alkenyl.

In some embodiments, R¹⁵ is selected from C₁-C₆ alkylene and C₂-C₆ alkenylene, wherein C₁-C₆ alkylene and C₂-C₆ alkenylene are optionally substituted with one or more substituents independently selected from halo, —OMe, —CN, —NH₂, and —NO₂. In some embodiments, R¹⁵ is selected from C₁-C₆ alkylene and C₂-C₆, alkenylene, wherein C₁-C₆ alkylene and C₂-C₆ alkenylene are optionally substituted with one or more substituents independently selected from halo, —OMe, and —CN. In some embodiments, R¹⁵ is selected from C₁-C₃ alkylene and C₂-C₃ alkenylene, wherein C₁-C₃ alkylene and C₂-C₃ alkenylene are optionally substituted with one or more substituents independently selected from halo, —OMe, and —CN. In some embodiments, R¹⁵ is C₁-C₃ alkylene optionally substituted with one or more substituents independently selected from halo, —OMe, and —CN. In some embodiments, R¹⁵ is C₁-C₃ alkylene.

In some embodiments, R¹⁵ is C₂-C₃ alkenylene.

In some embodiments, X is selected from glucose, xylose, galactose, rhamnose, rutinose, and disaccharide. In some embodiments, X is selected from glucose, galactose, rhamnose, rutinose, and disaccharide. In some embodiments, X is selected from glucose, xylose, rhamnose, rutinose, and disaccharide. In some embodiments, X is selected from glucose, xylose, galactose, rutinose, and disaccharide. In some embodiments, X is selected from glucose, xylose, galactose, rhamnose, and disaccharide.

In some embodiments, X is disaccharide.

In some embodiments, X is disaccharide selected from the group consisting of Sucrose, Lactose, Maltose, Trehalose, Cellobiose, Chitobiose, Kojibiose, Nigerose, Isomaltose, β,β-Trehalose, α,β-Trehalose, Sophorose, Laminaribiose, Gentiobiose, Trehalulose, Turanose, Maltulose, Leucrose, Isomaltulose, Gentiobiulose, Mannobiose, Melibiose, Melibiulose, Rutinose, Rutinulose, and Xylobiose.

In some embodiments, X is disaccharide selected from the group consisting of Sucrose, Lactose, Maltose, Trehalose, Cellobiose, and Chitobiose.

In some embodiments, X is selected from glucose, xylose, galactose, rhamnose, and rutinose. In some embodiments, X is selected from glucose, galactose, and rhamnose. In some embodiments, X is glucose. In some embodiments, X is xylose. In some embodiments, X is galactose. In some embodiments, X is rhamnose. In some embodiments, X is rutinose.

In some embodiments, the disease or disorder is major depression, treatment resistant depression, addiction, anxiety, post-traumatic stress disorder, prolonged grief disorder, complicated grief disorder, mania, psychosis, insomnia, hypersomnia, pain, Alzheimer's disease, Parkinson's disease, cluster headaches, binge eating, migraine headaches, or irritable bowel syndrome. In some embodiments, the disease or disorder is major depression, treatment resistant depression, addiction, anxiety, post-traumatic stress disorder, prolonged grief disorder, complicated grief disorder, or binge eating. In some embodiments, the disease or disorder is major depression. In some embodiments, the disease or disorder is treatment resistant depression.

In some embodiments, the disease or disorder is addiction. In some embodiments, the disease or disorder is anxiety. In some embodiments, the disease or disorder is post-traumatic stress disorder. In some embodiments, the disease or disorder is binge eating. In some embodiments, the disease is prolonged grief disorder. In some embodiments, the disease is complicated grief disorder,

In another aspect, provided herein is a method of treating a disease or disorder in a subject in need thereof comprising administering a modified indole alkaloid.

In some embodiments, the modified indole alkaloid is a modified tryptamine alkaloid, a modified ibogamine alkaloid, a modified ergoline alkaloid, a modified beta-carboline alkaloid, or a modified mitragynine alkaloid. In some embodiments, the modified indole alkaloid is a modified tryptamine alkaloid. In some embodiments, the modified indole alkaloid is a modified ibogamine alkaloid. In some embodiments, the modified indole alkaloid is a modified ergoline alkaloid. In some embodiments, the modified indole alkaloid is a modified beta-carboline alkaloid. In some embodiments, the modified indole alkaloid is a modified mitragynine alkaloid.

In some embodiments, the modified indole alkaloid is an acetylated indole alkaloid, an acylated indole alkaloid, a methylated indole alkaloid, a phosphorylated indole alkaloid, a sulfonylated indole alkaloid, or a glycosylated indole alkaloid. In some embodiments, the modified indole alkaloid is an acetylated indole alkaloid. In some embodiments, the modified indole alkaloid is an acylated indole alkaloid. In some embodiments, the modified indole alkaloid is a methylated indole alkaloid. In some embodiments, the modified indole alkaloid is a phosphorylated indole alkaloid. In some embodiments, the modified indole alkaloid is a sulfonylated indole alkaloid. In some embodiments, the modified indole alkaloid is a glycosylated indole alkaloid.

### Dosing

Effective doses of the compositions of the present disclosure vary depending upon many different factors, including means of administration, target site, physiological state of the patient, whether the patient is human or an animal, other medications administered, whether treatment is prophylactic or therapeutic, as well as the specific activity of the composition itself and its ability to elicit the desired response in the individual. In some embodiments, the patient is a human. In some embodiments, the patient is a nonhuman mammal. Typically, dosage regimens are adjusted to provide an optimum therapeutic response, i.e., to optimize safety and efficacy.

Determination of effective dosages in this context is typically based on animal model studies followed up by human clinical trials and is guided by determining effective dosages and administration protocols that significantly reduce the occurrence or severity of the subject disorder in model subjects. For example, the therapeutically effective amount of the modified indole alkaloid will depend on the condition to be treated, the severity and course of the condition, whether the modified indole alkaloid is administered for preventive or therapeutic purposes, previous therapy, the patient's clinical history and response to the modified indole alkaloid, and the discretion of the attending physician. The modified indole alkaloid described herein is suitably administered to the patient at one time or over a series of treatments and may be administered to the patent at any time from diagnosis onwards. The modified indole alkaloid described herein may be administered as the sole treatment or in conjunction with other drugs or therapies useful in treating the condition in question.

In some embodiments, the therapeutically effective amount of the modified indole alkaloid or salts thereof is at least 0.01 mg. In some embodiments, the therapeutically effective amount of the modified indole alkaloid or salts thereof is between 0.01 mg and 500 mg. In some embodiments, the therapeutically effective amount of the modified indole alkaloid or salts thereof is between 500 mg and 1000 mg. In some embodiments, the therapeutically effective amount of the modified indole alkaloid or salts thereof is between 0.01 mg and 1 mg. Disclosures of such ranges herein are intended to be a disclosure of all intervals within this range. For example, a disclosure of between 0.01 mg and 1 mg is a disclosure of 0.01 mg, 0.02 mg, 0.03 mg, 0.04 mg, 0.05 mg, 0.06 mg, 0.07 mg, 0.08 mg, 0.09 mg, 0.1 mg, 0.2 mg, 0.3 mg, 0.4 mg, 0.5 mg, 0.6 mg, 0.7 mg, 0.8 mg, 0.9 mg, and 1 mg.

In some embodiments, the therapeutically effective amount of the modified indole alkaloid or salts thereof is between 1 mg and 10 mg. In some embodiments, the therapeutically effective amount of the modified indole alkaloid or salts thereof is between 10 mg and 20 mg. In some embodiments, the therapeutically effective amount of the modified indole alkaloid or salts thereof is between 20 mg and 30 mg. In some embodiments, the therapeutically effective amount of the modified indole alkaloid or salts thereof is between 30 mg and 40 mg. In some embodiments, the therapeutically effective amount of the modified indole alkaloid or salts thereof is between 40 mg and 50 mg.

In some embodiments, the therapeutically effective amount of the modified indole alkaloid or salts thereof is between 50 mg and 100 mg. In some embodiments, the therapeutically effective amount of the modified indole alkaloid or salts thereof is between 100 mg and 150 mg.

In some embodiments, the therapeutically effective amount of the modified indole alkaloid or salts thereof is between 150 mg and 200 mg. In some embodiments, the therapeutically effective amount of the modified indole alkaloid or salts thereof is between 200 mg and 250 mg. In some embodiments, the therapeutically effective amount of the modified indole alkaloid or salts thereof is between 250 mg and 300 mg. In some embodiments, the therapeutically effective amount of the modified indole alkaloid or salts thereof is between 300 mg and 350 mg. In some embodiments, the therapeutically effective amount of the modified indole alkaloid or salts thereof is between 350 mg and 400 mg. In some embodiments, the therapeutically effective amount of the modified indole alkaloid or salts thereof is between 450 mg and 500 mg.

In some embodiments, the therapeutically effective amount of the modified indole alkaloid or salts thereof is between 500 mg and 550 mg. In some embodiments, the therapeutically effective amount of the modified indole alkaloid or salts thereof is between 550 mg and 600 mg. In some embodiments, the therapeutically effective amount of the modified indole alkaloid or salts thereof is between 600 mg and 650 mg. In some embodiments, the therapeutically effective amount of the modified indole alkaloid or salts thereof is between 650 mg and 700 mg. In some embodiments, the therapeutically effective amount of the modified indole alkaloid or salts thereof is between 700 mg and 750 mg. In some embodiments, the therapeutically effective amount of the modified indole alkaloid or salts thereof is between 750 mg and 800 mg. In some embodiments, the therapeutically effective amount of the modified indole alkaloid or salts thereof is between 800 mg and 850 mg. In some embodiments, the therapeutically effective amount of the modified indole alkaloid or salts thereof is between 850 mg and 900 mg. In some embodiments, the therapeutically effective amount of the modified indole alkaloid or salts thereof is between 900 mg and 950 mg. In some embodiments, the therapeutically effective amount of the modified indole alkaloid or salts thereof is between 950 mg and 1000 mg.

### EXAMPLES

### Example 1: Production of Modified Indole Alkaloids

Reagents All enzymes were purchased from New England Biolabs. All synthetic oligonucleotides were ordered from Integrated DNA Technologies. All chemicals for gas chromatography (GC) standards except for ethanol (VWR) and tetradecyl acetate (Ark Pharm, Inc.) were purchased from Sigma Aldrich.

Plasmid Construction. Cloning, and Transformations The target gene(s) and vector fragments were amplified with the pairs of primers from the templates. The resulting fragments were combined by sequence and golden gate cloning, 2.5 μL of the solution was used for transformation of E. coli. Plasmids were verified by colony PCR, by digestion with restriction enzymes, and by sequencing.

Enzyme Mixture Preparation Overnight cultures were grown in 5 mL Luria Broth (LB) (Fisher BioReagents) containing appropriate antibiotics. Antibiotic concentrations were as follows: kanamycin (50 μg/ml) (IBI Scientific), chloramphenicol (40 μg/ml) (Fisher BioReagents), ampicillin 250 (μg/ml) (Fisher BioReagents), tetracycline (20 μg/ml) (Fisher BioReagents). Production was carried out with M9 medium (33.7 mM Na2HPO4, 22 mM KH2PO4, 8.55 mM NaCl, 9.35 mM NH₄Cl, 1 mM MgSO4, 0.1 mM CaCl₂)) (BD Bacto), 5 g 1-1 yeast extract (BD Bacto), 50 g 1-1 or 10 g 1-1 glucose (Fisher BioReagents), and 1,000-fold dilution of A5 trace metal mix (2.86 g H3BO3 (Fisher Chemical), 1.81 g MnCl2·4H2O (MP Biomedicals), 0.222 g ZnSO4·7H2O (Sigma-Aldrich), 0.39 g Na2MoO4·2H2O (Alfa Aesar), 0.079 g CuSO4·5H2O (Sigma-Aldrich), 49.4 mg Co(NO3)2·6H2O (Sigma-Aldrich) per liter water). This media is referred to as “M9P” herein. 50 g 1-1 glucose was used for C₂-C₁₀ acetate ester experiments and 10 g 1-1 glucose was used for tetradecyl acetate, isobutyrate, and butyrate ester experiments. Optical densities (D) were measured at 600 nm with a Synergy H1 Hybrid Plate Reader (BioTek Instruments, Inc.).

Substrate Feeding Experiments Overnight cultures were inoculated 1% in 5 mL M9P in 15 mL screw-cap culture tubes. Cells were grown to a D600 nm of ˜0.4 at 37° C. in a rotary shaker (250 r.p.m.), followed by adding 1 mM isopropyl-β-d-thio-galactoside (IPTG) (Promega). The cultures were incubated for 1 h after induction at 30° C. Then substrates of interest were added to the cultures. Production was performed at 30° C. in a rotary shaker (250 r.p.m.) for 24 h. 1.5 mL of culture was taken for analysis every 24 h. The 1.5 mL of the cultures were centrifuged at 17,000 g for 3 min, and then 1 mL of the supernatants were transferred to 2-mL GC vials for GC analysis.

Production of Modified Indole Alkaloids with Enzymatic Conversion Purified enzyme or cell lysate containing purified enzyme was mixed in respective buffer with cofactor and indole alkaloid substrate. The reaction was run at room temperature or respective temperature optimum to yield a modified indole alkaloid.

Analysis was performed by chromatography/mass spectrometry (LCMS) with a 1260 Infinity LC System connected to a 6120 Quadrupole Mass Spectrometer (Agilent Technologies). Zorbax Eclipse Plus C18 guard column (4.6 cm×12.5 cm, 5 pm packing, Agilent Technologies) was connected to a Agilent ZORBAX StableBond-C18, 1.8 um, 2.1×50 mm column at 20° C. using a 0.3 mL/min flow rate. The mobile phase consisted of a mixture of 0.1% formic acid in acetonitrile (v/v) and 0.1% formic acid in water (v/v) was eluted under the following gradient conditions (shown in relation to acetonitrile content): 0 min-10%, 6 min-100%, 7 min-10%, 14 min-10%. The mobile phase was delivered at a flow rate of 0.3 mL/min and the total analytical run time was 14 min. Absorbance was measured using a diode array detector for UV-Vis analysis. MS was conducted in atmospheric pressure ionization-positive.

### Example 2: Cloning and Purification of Transferase Proteins for Modification of Indole Alkaloids

### Materials and Cloning Conditions

Cloning enzymes and buffers were purchased from New England Biolabs (lpswich, MA). Plasmids were constructed using a MoClo Golden Gate Assembly and propagated using E. coli strain TG1 (Lucigen). Strains for plasmid construction were grown in Luria Broth (LB) selected on 34 mg/L chloramphenicol, 100 mg/L ampicillin, and/or 25 mg/L kanamycin. All synthetic oligonucleotides and double stranded DNA were ordered from Integrated DNA Technologies.

### Plasmid Construction, Cloning, and Transformations

The following circular parent vectors were used for forming N- and C-terminal His ×6 transferase fusion proteins (“His ×6” disclosed as SEQ ID NO: 28):

(1) pNAB096 (N-terminal His x6 expression vector (″His x6″ disclosed as SEQ ID NO: 28))
(SEQ ID NO: 16)
GATGCGTCCGGCGTAGAGGATCGAGatctcgatcccgcgaaattaatacgactcactataggggaattgtgagcgg
ataacaattcccctctagaaataattttgtttaactttaagaaggagatataccATGggccatcaccatcaccatcacgactacgacatcccg
actaccgaaaacctgtacttccagggttcttgagacctccctatcagtgatagagattgacatccctatcagtgatagagatactgagcacgg
atctgaaagaggagaaaggatctatggcgagtagcgaagacgttatcaaagagttcatgcgtttcaaagttcgtatggaaggttccgttaac
ggtcacgagttcgaaatcgaaggtgaaggtgaaggtcgtccgtacgaaggtacccagaccgctaaactgaaagttaccaaaggtggtccg
ctgccgttcgcttgggacatcctgtccccgcagttccagtacggttccaaagcttacgttaaacacccggctgacatcccggactacctgaaa
ctgtccttcccggaaggtttcaaatgggaacgtgttatgaacttcgaagacggtggtgttgttaccgttacccaggactcctccctgcaagac
ggtgagttcatctacaaagttaaactgcgtggtaccaacttcccgtccgacggtccggttatgcagaaaaaaaccatgggttgggaagcttc
caccgaacgtatgtacccggaagacggtgctctgaaaggtgaaatcaaaatgcgtctgaaactgaaagacggtggtcactacgacgctga
agttaaaaccacctacatggctaaaaaaccggttcagctgccgggtgcttacaaaaccgacatcaaactggacatcacctcccacaacgaa
gactacaccatcgttgaacagtacgaacgtgctgaaggtcgtcactccaccggtgcttaataaggatctccaggcatcaaataaaacgaaa
ggctcagtcgaaagactgggcctttcgttttatctgttgtttgtcggtgaacgctctctactagagtcacactggctcaccttcggggggccttt
ctgcgtttataggtctccatcctgatcaatccggctgctaacaaagcccgaaaggaagctgagttggctgctgccaccgctgagcaataact
agcataaccccttggggcctctaaacgggtcttgaggggttttttgctgaaaggaggaactatatccggatatcccgcaagaggcccggca
gtaccggcataaccaagcctatgcctacagcatccagggtgacggtgccgaggatgacgatgagcgcattgttagatttCATACAC
GGTGCCTGACTGCGTTAGCAAtttaactgtgataaactaccgcattaaagcttatcgatgataagctgtcaaacatgagaa
ttcttgaagacgaaagggcctcgtgatacgcctatttttataggttaatgtcatgataataatggtttcttagacgtcaggtggcacttttcgggg
aaatgtgcgcggaacccctatttgtttatttttctaaatacattcaaatatgtatccgctcatgagacaataaccctgataaatgcttcaataata
ttgaaaaaggaagagtatgagtattcaacatttccgtgtcgcccttattcccttttttgcggcattttgccttcctgtttttgctcacccagaaac
gctggtgaaagtaaaagatgctgaagatcagttgggtgcacgagtgggttacatcgaactggatctcaacagcggtaagatccttgagagttttcg
ccccgaagaacgttttccaatgatgagcacttttaaagttctgctatgtggcgcggtattatcccgtgttgacgccgggcaagagcaactcgg
tcgccgcatacactattctcagaatgacttggttgagtactcaccagtcacagaaaagcatcttacggatggcatgacagtaagagaattatg
cagtgctgccataaccatgagtgataacactgcggccaacttacttctgacaacgatcggaggaccgaaggagctaaccgcttttttgcaca
acatgggggatcatgtaactcgccttgatcgttgggaaccggagctgaatgaagccataccaaacgacgagcgtgacaccacgatgcctg
cagcaatggcaacaacgttgcgcaaactattaactggcgaactacttactctagcttcccggcaacaattaatagactggatggaggcggat
aaagttgcaggaccacttctgcgctcggcccttccggctggctggtttattgctgataaatctggagccggtgagcgtgggtcacgcggtat
cattgcagcactggggccagatggtaagccctcccgtatcgtagttatctacacgacggggagtcaggcaactatggatgaacgaaataga
cagatcgctgagataggtgcctcactgattaagcattggtaactgtcagaccaagtttactcatatatactttagattgatttaaaacttcatttt
taatttaaaaggatctaggtgaagatcctttttgataatctcatgaccaaaatcccttaacgtgagttttcgttccactgagcgtcagaccccgta
gaaaagatcaaaggatcttcttgagatcctttttttctgcgcgtaatctgctgcttgcaaacaaaaaaaccaccgctaccagcggtggtttgtttg
ccggatcaagagctaccaactctttttccgaaggtaactggcttcagcagagcgcagataccaaatactgtccttctagtgtagccgtagttagg
ccaccacttcaagaactctgtagcaccgcctacatacctcgctctgctaatcctgttaccagtggctgctgccagtggcgataagtcgtgtctt
accgggttggactcaagacgatagttaccggataaggcgcagcggtcgggctgaacggggggttcgtgcacacagcccagcttggagc
gaacgacctacaccgaactgagatacctacagcgtgagctatgagaaagcgccacgcttcccgaagggagaaaggcggacaggtatcc
ggtaagcggcagggtcggaacaggagagcgcacgagggagcttccagggggaaacgcctggtatctttatagtcctgtcgggtttcgcc
acctctgacttgagcgtcgatttttgtgatgctcgtcaggggggggagcctatggaaaaacgccagcaacgcggcctttttacggttcctgg
ccttttgctggccttttgctcacatgttctttcctgcgttatcccctgattctgtggataaccgtattaccgcctttgagtgagctgataccgctc
gccgcagccgaacgaccgagcgcagcgagtcagtgagcgaggaagcggaagagcgcctgatgcggtattttctccttacgcatctgtgcgg
tatttcacaccgcatatatggtgcactctcagtacaatctgctctgatgccgcatagttaagccagtatacactccgctatcgctacgtgactgg
gtcatggctgcgccccgacacccgccaacacccgctgacgcgccctgacgggcttgtctgctcccggcatccgcttacagacaagctgtg
accttctccgggagctgcatgtgtcagaggttttcaccgtcatcaccgaaacgcgcgaggcagctgcggtaaagctcatcagcgtggtcgt
gaagcgattcacagatgtctgcctgttcatccgcgtccagctcgttgagtttctccagaagcgttaatgtctggcttctgataaagcgggccat
gttaagggcggttttttcctgtttggtcactgatgcctccgtgtaagggggatttctgttcatgggggtaatgataccgatgaaacgagagagg
atgctcacgatacgggttactgatgatgaacatgcccggttactggaacgttgtgagggtaaacaactggcggtatggatgcggcgggacc
agagaaaaatcactcagggtcaatgccagcgcttcgttaatacagatgtaggtgttccacagggtagccagcagcatcctgcgatgcagat
ccggaacataatggtgcagggcgctgacttccgcgtttccagactttacgaaacacggaaaccgaagaccattcatgttgttgctcaggtcg
cagacgttttgcagcagcagtcgcttcacgttcgctcgcgtatcggtgattcattctgctaaccagtaaggcaaccccgccagcctagccgg
gtcctcaacgacaggagcacgatcatgcgcacccgtggccaggacccaacgctgcccgagatgcgccgcgtgcggctgctggagatgg
cggacgcgatggatatgttctgccaagggttggtttgcgcattcacagttctccgcaagaattgattggctccaattcttggagtggtgaatcc
gttagcgaggtgccgccggcttccattcaggtcgaggtggcccggctccatgcaccgcgacgcaacgcggggaggcagacaaggtata
gggcggcgcctacaatccatgccaacccgttccatgtgctcgccgaggcggcataaatcgccgtgacgatcagcggtccagtgatcgaa
gttaggctggtaagagccgcgagcgatccttgaagctgtccctgatggtcgtcatctacctgcctggacagcatggcctgcaacgcgggca
tcccgatgccgccggaagcgagaagaatcataatggggaaggccatccagcctcgcgtcgcgaacgccagcaagacgtagcccagcg
cgtcggccgccatgccggcgataatggcctgcttctcgccgaaacgtttggtgggggaccagtgacgaaggcttgagcgagggcgtgc
aagattccgaataccgcaagcgacaggtcgatcatcgtcgcgctccagcgaaagcggtcctcgccgaaaatgacccagagcgctgccg
gcacctgtcctacgagttgcatgataaagaagacagtcataagtgcggcgacgatagtcatgccccgcgcccaccggaaggagctgactg
ggttgaaggctctcaagggcatcggtcgagatcccggtgcctaatgagtgagCTAacttacattaattgcgttgcgctcactgcccgctttc
cagtcgggaaacctgtcgtgccagctgcattaatgaatcggccaacgcgcggggagaggcggtttgcgtattgggcgccagggtggttttt
cttttcaccagtgaaacgggcaacagctgattgcccttcaccgcctggccctgagagagttgcagcaagcggtccacgctggtttgcccca
gcaggcgaaaatcctgtttgatggtggttaacggcgggatataacatgagctgtcttcggtatcgtcgtatcccactaccgagatatccgcac
caacgcgcagcccggactcggtaatggcgcgcattgcgcccagcgccatctgatcgttggcaaccagcatcgcagtgggaacgatgccc
tcattcagcatttgcatggtttgttgaaaaccggacatggcactccagtcgccttcccgttccgctatcggctgaatttgattgcgagtgagatat
ttatgccagccagccagacgcagacgcgccgagacagaacttaatgggcccgctaacagcgcgatttgctggtgacccaatgcgaccag
atgctccacgcccagtcgcgtaccgtcttcatgggagaaaataatactgttgatgggtgtctggtcagagacatcaagaaataacgccggaa
cattagtgcaggcagcttccacagcaatggcatcctggtcatccagcggatagttaatgatcagcccactgacgcgttgcgcgagaagattg
tgcaccgccgctttacaggcttcgacgccgcttcgttctaccatcgacaccaccacgctggcacccagttgatcggcgcgagatttaatcgc
cgcgacaatttgcgacggcgcgtgcagggccagactggaggtggcaacgccaatcagcaacgactgtttgcccgccagttgttgtgccac
gcggttgggaatgtaattcagctccgccatcgccgcttccactttttcccgcgttttcgcagaaacgtggctggcctggttcaccacgcggga
aacggtctgataagagacaccggcatactctgcgacatcgtataacgttactggtttCACattcaccaccctgaattgactctcttccgggc
gctatcatgccataccgcgaaaggttttgcgccattcgatggtgtccgggatctcgacgctctcccttatgcgactcctgcattaggaagcag
cccagtagtaggttgaggccgttgagcaccgccgccgcaaggaatggtgcatgcaaggagatggcgcccaacagtcccccggccacgg
ggcctgccaccatacccacgccgaaacaagcgctcatgagcccgaagtggcgagcccgatcttccccatcggtgatgtcggcgatatag
gcgccagcaaccgcacctgtggcgccggtgatgccggccac
(2) pNAB0097 (N-terminal His x6 expression vector (″His x6″ disclosed as SEQ ID NO: 28))
(SEQ ID NO: 17)
ttcttgaagacgaaagggcctcgtgatacgcctatttttataggttaatgtcatgataataatggtttcttagacgtcaggtggcacttttcgggg
aaatgtgcgcggaacccctatttgtttatttttctaaatacattcaaatatgtatccgctcatgagacaataaccctgataaatgcttcaataata
ttgaaaaaggaagagtatgagtattcaacatttccgtgtcgcccttattcccttttttgcggcattttgccttcctgtttttgctcacccagaaac
gctggtgaaagtaaaagatgctgaagatcagttgggtgcacgagtgggttacatcgaactggatctcaacagcggtaagatccttgagagttttcg
ccccgaagaacgttttccaatgatgagcacttttaaagttctgctatgtggcgcggtattatcccgtgttgacgccgggcaagagcaactcgg
tcgccgcatacactattctcagaatgacttggttgagtactcaccagtcacagaaaagcatcttacggatggcatgacagtaagagaattatg
cagtgctgccataaccatgagtgataacactgcggccaacttacttctgacaacgatcggaggaccgaaggagctaaccgcttttttgcaca
acatgggggatcatgtaactcgccttgatcgttgggaaccggagctgaatgaagccataccaaacgacgagcgtgacaccacgatgcctg
cagcaatggcaacaacgttgcgcaaactattaactggcgaactacttactctagcttcccggcaacaattaatagactggatggaggcggat
aaagttgcaggaccacttctgcgctcggcccttccggctggctggtttattgctgataaatctggagccggtgagcgtggctctcgcggtatc
attgcagcactggggccagatggtaagccctcccgtatcgtagttatctacacgacggggagtcaggcaactatggatgaacgaaatagac
agatcgctgagataggtgcctcactgattaagcattggtaactgtcagaccaagtttactcatatatactttagattgatttaaaacttcattttt
aatttaaaaggatctaggtgaagatcctttttgataatctcatgaccaaaatcccttaacgtgagttttcgttccactgagcgtcagaccccgtag
aaaagatcaaaggatcttcttgagatcctttttttctgcgcgtaatctgctgcttgcaaacaaaaaaaccaccgctaccagcggtggtttgtttgc
cggatcaagagctaccaactctttttccgaaggtaactggcttcagcagagcgcagataccaaatactgtccttctagtgtagccgtagttaggcc
accacttcaagaactctgtagcaccgcctacatacctcgctctgctaatcctgttaccagtggctgctgccagtggcgataagtcgtgtcttac
cgggttggactcaagacgatagttaccggataaggcgcagcggtcgggctgaacggggggttcgtgcacacagcccagcttggagcga
acgacctacaccgaactgagatacctacagcgtgagctatgagaaagcgccacgcttcccgaagggagaaaggcggacaggtatccgg
taagcggcagggtcggaacaggagagcgcacgagggagcttccagggggaaacgcctggtatctttatagtcctgtcgggtttcgccacc
tctgacttgagcgtcgatttttgtgatgctcgtcaggggggcggagcctatggaaaaacgccagcaacgcggcctttttacggttcctggcct
tttgctggccttttgctcacatgttctttcctgcgttatcccctgattctgtggataaccgtattaccgcctttgagtgagctgataccgctcgcc
gcagccgaacgaccgagcgcagcgagtcagtgagcgaggaagcggaagagcgcctgatgcggtattttctccttacgcatctgtgcggtattt
cacaccgcatatatggtgcactctcagtacaatctgctctgatgccgcatagttaagccagtatacactccgctatcgctacgtgactgggtca
tggctgcgccccgacacccgccaacacccgctgacgcgccctgacgggcttgtctgctcccggcatccgcttacagacaagctgtgacca
tctccgggagctgcatgtgtcagaggttttcaccgtcatcaccgaaacgcgcgaggcagctgcggtaaagctcatcagcgtggtcgtgaag
cgattcacagatgtctgcctgttcatccgcgtccagctcgtgagtttctccagaagcgttaatgtctggcttctgataaagcgggccatgttaa
gggcggttttttcctgtttggtcactgatgcctccgtgtaagggggatttctgttcatgggggtaatgataccgatgaaacgagagaggatgct
cacgatacgggttactgatgatgaacatgcccggttactggaacgttgtgagggtaaacaactggcggtatggatgcggcgggaccagag
aaaaatcactcagggtcaatgccagcgcttcgttaatacagatgtaggtgttccacagggtagccagcagcatcctgcgatgcagatccgg
aacataatggtgcagggcgctgacttccgcgtttccagactttacgaaacacggaaaccgaagaccattcatgttgttgctcaggtcgcaga
cgttttgcagcagcagtcgcttcacgttcgctcgcgtatcggtgattcattctgctaaccagtaaggcaaccccgccagcctagccgggtcct
caacgacaggagcacgatcatgcgcacccgtggccaggacccaacgctgcccgagatgcgccgcgtgcggctgctggagatggcgga
cgcgatggatatgttctgccaagggttggtttgcgcattcacagttctccgcaagaattgattggctccaattcttggagtggtgaatccgttag
cgaggtgccgccggcttccattcaggtcgaggtggcccggctccatgcaccgcgacgcaacgcggggaggcagacaaggtatagggc
ggcgcctacaatccatgccaacccgttccatgtgctcgccgaggcggcataaatcgccgtgacgatcagcggtccagtgatcgaagttag
gctggtaagagccgcgagcgatccttgaagctgtccctgatggtcgtcatctacctgcctggacagcatggcctgcaacgcgggcatccc
gatgccgccggaagcgagaagaatcataatggggaaggccatccagcctcgcgtcgcgaacgccagcaagacgtagcccagcgcgtc
ggccgccatgccggcgataatggcctgcttctcgccgaaacgtttggtggcgggaccagtgacgaaggcttgagcgagggcgtgcaaga
ttccgaataccgcaagcgacaggtcgatcatcgtcgcgctccagcgaaagcggtcctcgccgaaaatgacccagagcgctgccggcac
ctgtcctacgagttgcatgataaagaagacagtcataagtgcggcgacgatagtcatgccccgcgcccaccggaaggagctgactgggtt
gaaggctctcaagggcatcggtcgagatcccggtgcctaatgagtgagctaacttacattaattgcgttgcgctcactgcccgctttccagtc
gggaaacctgtgtgccagctgcattaatgaatcggccaacgcgcggggagaggcggtttgcgtattgggcgccagggtggtttttcttttc
accagtgaaacgggcaacagctgattgcccttcaccgcctggccctgagagagttgcagcaagcggtccacgctggtttgccccagcag
gcgaaaatcctgtttgatggtggttaacggcgggatataacatgagctgtcttcggtatcgtcgtatcccactaccgagatatccgcaccaac
gcgcagcccggactcggtaatggcgcgcattgcgcccagcgccatctgatcgttggcaaccagcatcgcagtgggaacgatgccctcatt
cagcatttgcatggtttgttgaaaaccggacatggcactccagtcgccttcccgttccgctatcggctgaatttgattgcgagtgagatatttat
gccagccagccagacgcagacgcgccgagacagaacttaatgggcccgctaacagcgcgatttgctggtgacccaatgcgaccagatg
ctccacgcccagtcgcgtaccgtcttcatgggagaaaataatactgttgatgggtgtctggtcagagacatcaagaaataacgccggaacat
tagtgcaggcagcttccacagcaatggcatcctggtcatccagcggatagttaatgatcagcccactgacgcgttgcgcgagaagattgtg
caccgccgctttacaggcttcgacgccgcttcgttctaccatcgacaccaccacgctggcacccagttgatcggcgcgagatttaatcgccg
cgacaatttgcgacggcgcgtgcagggccagactggaggtggcaacgccaatcagcaacgactgtttgcccgccagttgttgtgccacgc
ggttgggaatgtaattcagctccgccatcgccgcttccactttttcccgcgttttcgcagaaacgtggctggcctggttcaccacgcgggaaa
cggtctgataagagacaccggcatactctgcgacatcgtataacgttactggtttcacattcaccaccctgaattgactctcttccgggcgctat
catgccataccgcgaaaggttttgcgccattcgatggtgtccgggatctcgacgctctcccttatgcgactcctgcattaggaagcagccca
gtagtaggttgaggccgttgagcaccgccgccgcaaggaatggtgcatgtaaggagatggcgcccaacagtcccccggccacggggc
ctgccaccatacccacgccgaaacaagcgctcatgagcccgaagtggcgagcccgatcttccccatcggtgatgtcggcgatataggcg
ccagcaaccgcacctgtggcgccggtgatgccggccacgatgcgtccggcgtagaggatcgagatctcgatcccgcgaaattaatacga
ctcactataggggaattgtgagcggataacaattcccctctagaaataattttgtttaactttaagaaggagatatactatgtgagacctccctat
cagtgatagagattgacatccctatcagtgatagagatactgagcacggatctgaaagaggagaaaggatctatggcgagtagcgaagac
gttatcaaagagttcatgcgtttcaaagttcgtatggaaggttccgttaacggtcacgagttcgaaatcgaaggtgaaggtgaaggtcgtccg
tacgaaggtacccagaccgctaaactgaaagttaccaaaggtggtccgctgccgttcgcttgggacatcctgtccccgcagttccagtacg
gttccaaagcttacgttaaacacccggctgacatcccggactacctgaaactgtccttcccggaaggtttcaaatgggaacgtgttatgaactt
cgaagacggtggtgttgttaccgttacccaggactcctccctgcaagacggtgagttcatctacaaagttaaactgcgtggtaccaacttccc
gtccgacggtccggttatgcagaaaaaaaccatgggttgggaagcttccaccgaacgtatgtacccggaagacggtgctctgaaaggtga
aatcaaaatgcgtctgaaactgaaagacggtggtcactacgacgctgaagttaaaaccacctacatggctaaaaaaccggttcagctgccg
ggtgcttacaaaaccgacatcaaactggacatcacctcccacaacgaagactacaccatcgttgaacagtacgaacgtgctgaaggtcgtc
actccaccggtgcttaataaggatctccaggcatcaaataaaacgaaaggctcagtcgaaagactgggcctttcgttttatctgttgtttgtcgg
tgaacgctctctactagagtcacactggctcaccttcggggggcctttctgcgtttataggtctccatccgaaaacctgtacttccagggccat
caccatcaccatcactgatcaatccggctgctaacaaagcccgaaaggaagctgagttggctgctgccaccgctgagcaataactagcata
accccttggggcctctaaacgggtcttgaggggttttttgctgaaaggaggaactatatccggatatcccgcaagaggcccggcagtaccg
gcataaccaagcctatgcctacagcatccagggtgacggtgccgaggatgacgatgagcgcattgttagatttcatacacggtgcctgact
gcgttagcaatttaactgtgataaactaccgcattaaagcttatcgatgataagctgtcaaacatgagaa
(3) pNAB0098 (C-terminal His x6 expression vector (″His x6″ disclosed as SEQ ID NO: 28))
(SEQ ID NO: 18)
ttcttgaagacgaaagggcctcgtgatacgcctatttttataggttaatgtcatgataataatggtttcttagacgtcaggtggcacttttcgggg
aaatgtgcgcggaacccctatttgtttatttttctaaatacattcaaatatgtatccgctcatgagacaataaccctgataaatgcttcaataata
ttgaaaaaggaagagtatgagtattcaacatttccgtgtcgcccttattcccttttttgcggcattttgccttcctgtttttgctcacccagaaac
gctggtgaaagtaaaagatgctgaagatcagttgggtgcacgagtgggttacatcgaactggatctcaacagcggtaagatccttgagagttttcg
ccccgaagaacgttttccaatgatgagcacttttaaagttctgctatgtggcgcggtattatcccgtgttgacgccgggcaagagcaactcgg
tcgccgcatacactattctcagaatgacttggttgagtactcaccagtcacagaaaagcatcttacggatggcatgacagtaagagaattatg
cagtgctgccataaccatgagtgataacactgcggccaacttacttctgacaacgatcggaggaccgaaggagctaaccgcttttttgcaca
acatgggggatcatgtaactcgccttgatcgttgggaaccggagctgaatgaagccataccaaacgacgagcgtgacaccacgatgcctg
cagcaatggcaacaacgttgcgcaaactattaactggcgaactacttactctagcttcccggcaacaattaatagactggatggaggcggat
aaagttgcaggaccacttctgcgctcggcccttccggctggctggtttattgctgataaatctggagccggtgagcgtggctctcgcggtatc
attgcagcactggggccagatggtaagccctcccgtatcgtagttatctacacgacggggagtcaggcaactatggatgaacgaaatagac
agatcgctgagataggtgcctcactgattaagcattggtaactgtcagaccaagtttactcatatatactttagattgatttaaaacttcattttt
aatttaaaaggatctaggtgaagatcctttttgataatctcatgaccaaaatcccttaacgtgagttttcgttccactgagcgtcagaccccgtag
aaaagatcaaaggatcttcttgagatcctttttttctgcgcgtaatctgctgcttgcaaaaaaaaaaccaccgctaccagcggtggtttgtttgcc
ggatcaagagctaccaactctttttccgaaggtaactggcttcagcagagcgcagataccaaatactgtccttctagtgtagccgtagttaggcc
accacttcaagaactctgtagcaccgcctacatacctcgctctgctaatcctgttaccagtggctgctgccagtggcgataagtcgtgtcttac
cgggttggactcaagacgatagttaccggataaggcgcagcggtcgggctgaacggggggttcgtgcacacagcccagcttggagcga
acgacctacaccgaactgagatacctacagcgtgagctatgagaaagcgccacgcttcccgaagggagaaaggcggacaggtatccgg
taagcggcagggtcggaacaggagagcgcacgagggagcttccagggggaaacgcctggtatctttatagtcctgtcgggtttcgccacc
tctgacttgagcgtcgatttttgtgatgctcgtcaggggggggagcctatggaaaaacgccagcaacgcggcctttttacggttcctggcct
tttgctggccttttgctcacatgttctttcctgcgttatcccctgattctgtggataaccgtattaccgcctttgagtgagctgataccgctcgcc
gcagccgaacgaccgagcgcagcgagtcagtgagcgaggaagcggaagagcgcctgatgcggtattttctccttacgcatctgtgcggtattt
cacaccgcatatatggtgcactctcagtacaatctgctctgatgccgcatagttaagccagtatacactccgctatcgctacgtgactgggtca
tggctgcgcccccgacacccgccaacacccgctgacgcgccctgacgggcttgtctgctcccggcatccgcttacagacaagctgtgacca
tctccgggagctgcatgtgtcagaggttttcaccgtcatcaccgaaacgcgcgaggcagctgcggtaaagctcatcagcgtggtcgtgaag
cgattcacagatgtctgcctgttcatccgcgtccagctcgttgagtttctccagaagcgttaatgtctggcttctgataaagcgggccatgttaa
gggcggttttttcctgtttggtcactgatgcctccgtgtaagggggatttctgttcatgggggtaatgataccgatgaaacgagagaggatgct
cacgatacgggttactgatgatgaacatgcccggttactggaacgttgtgagggtaaacaactggcggtatggatgcggcgggaccagag
aaaaatcactcagggtcaatgccagcgcttcgttaatacagatgtaggtgttccacagggtagccagcagcatcctgcgatgcagatccgg
aacataatggtgcagggcgctgacttccgcgtttccagactttacgaaacacggaaaccgaagaccattcatgttgttgctcaggtcgcaga
cgttttgcagcagcagtcgcttcacgttcgctcgcgtatcggtgattcattctgctaaccagtaaggcaaccccgccagcctagccgggtcct
caacgacaggagcacgatcatgcgcacccgtggccaggacccaacgctgcccgagatgcgccgcgtgcggctgctggagatggcgga
cgcgatggatatgttctgccaagggttggtttgcgcattcacagttctccgcaagaattgattggctccaattcttggagtggtgaatccgttag
cgaggtgccgccggcttccattcaggtcgaggtggcccggctccatgcaccgcgacgcaacgcggggaggcagacaaggtatagggc
ggcgcctacaatccatgccaacccgttccatgtgctcgccgaggcggcataaatcgccgtgacgatcagcggtccagtgatcgaagttag
gctggtaagagccgcgagcgatccttgaagctgtccctgatggtcgtcatctacctgcctggacagcatggcctgcaacgcgggcatccc
gatgccgccggaagcgagaagaatcataatggggaaggccatccagcctcgcgtcgcgaacgccagcaagacgtagcccagcgcgtc
ggccgccatgccggcgataatggcctgcttctcgccgaaacgtttggtggcgggaccagtgacgaaggcttgagcgagggcgtgcaaga
ttccgaataccgcaagcgacaggtcgatcatcgtcgcgctccagcgaaagcggtcctcgccgaaaatgacccagagcgctgccggcac
ctgtcctacgagttgcatgataaagaagacagtcataagtgcggcgacgatagtcatgccccgcgcccaccggaaggagctgactgggtt
gaaggctctcaagggcatcggtcgagatcccggtgcctaatgagtgagctaacttacattaattgcgttgcgctcactgcccgctttccagtc
gggaaacctgtcgtgccagctgcattaatgaatcggccaacgcgcggggagaggcggtttgcgtattgggcgccagggtggtttttcttttc
accagtgaaacgggcaacagctgattgcccttcaccgcctggccctgagagagttgcagcaagcggtccacgctggtttgccccagcag
gcgaaaatcctgtttgatggtggttaacggcgggatataacatgagctgtcttcggtatcgtcgtatcccactaccgagatatccgcaccaac
gcgcagcccggactcggtaatggcgcgcattgcgcccagcgccatctgatcgttggcaaccagcatcgcagtgggaacgatgccctcatt
cagcatttgcatggtttgttgaaaaccggacatggcactccagtcgccttcccgttccgctatcggctgaatttgattgcgagtgagatatttat
gccagccagccagacgcagacgcgccgagacagaacttaatgggcccgctaacagcgcgatttgctggtgacccaatgcgaccagatg
ctccacgcccagtcgcgtaccgtcttcatgggagaaaataatactgttgatgggtgtctggtcagagacatcaagaaataacgccggaacat
tagtgcaggcagcttccacagcaatggcatcctggtcatccagcggatagttaatgatcagcccactgacgcgttgcgcgagaagattgtg
caccgccgctttacaggcttcgacgccgcttcgttctaccatcgacaccaccacgctggcacccagttgatcggcgcgagatttaatcgccg
cgacaatttgcgacggcgcgtgcagggccagactggaggtggcaacgccaatcagcaacgactgtttgcccgccagttgttgtgccacgc
ggttgggaatgtaattcagctccgccatcgccgcttccactttttcccgcgttttcgcagaaacgtggctggcctggttcaccacgcgggaaa
cggtctgataagagacaccggcatactctgcgacatcgtataacgttactggtttcacattcaccaccctgaattgactctcttccgggcgctat
catgccataccgcgaaaggttttgcgccattcgatggtgtccgggatctcgacgctctcccttatgcgactcctgcattaggaagcagccca
gtagtaggttgaggccgttgagcaccgccgccgcaaggaatggtgcatgaaggagatggcgcccaacagtcccccggccacggggc
ctgccaccatacccacgccgaaacaagcgctcatgagcccgaagtggcgagcccgatcttccccatcggtgatgtcggcgatataggcg
ccagcaaccgcacctgtggcgccggtgatgccggccacgatgcgtccggcgtagaggatcgagatctcgatcccgcgaaattaatacga
ctcactataggggaattgtgagcggataacaattcccctctagaaataattttgtttaactttaagaaggagatatactatgtgagacctccctat
cagtgatagagattgacatccctatcagtgatagagatactgagcacggatctgaaagaggagaaaggatctatggcgagtagcgaagac
gttatcaaagagttcatgcgtttcaaagttcgtatggaaggttccgttaacggtcacgagttcgaaatcgaaggtgaaggtgaaggtcgtccg
tacgaaggtacccagaccgctaaactgaaagttaccaaaggggtccgctgccgttcgcttgggacatcctgtccccgcagttccagtacg
gttccaaagcttagttaaacacccggctgacatcccggactacctgaaactgtccttcccggaaggtttcaaatgggaacgtgttatgaactt
cgaagacggtggtgttgttaccgttacccaggactcctccctgcaagacggtgagttcatctacaaagttaaactgcgtggtaccaacttccc
gtccgacggtccggttatgcagaaaaaaaccatgggttgggaagcttccaccgaacgtatgtacccggaagacggtgctctgaaaggtga
aatcaaaatgcgtctgaaactgaaagacggtggtcactacgacgctgaagttaaaaccacctacatggctaaaaaaccggttcagctgccg
ggtgcttacaaaaccgacatcaaactggacatcacctcccacaacgaagactacaccatcgttgaacagtacgaacgtgctgaaggtcgtc
actccaccggtgcttaataaggatctccaggcatcaaataaaacgaaaggctcagtcgaaagactgggcctttcgttttatctgttgtttgtcgg
tgaacgctctctactagagtcacactggctcaccttcggggggcctttctgcgtttataggtctccatccgaaaacctgtacttccagggccat
caccatcaccatcactgatcaatccggctgctaacaaagcccgaaaggaagctgagttggctgctgccaccgctgagcaataactagcata
accccttggggcctctaaacgggtcttgaggggttttttgctgaaaggaggaactatatccggatatcccgcaagaggcccggcagtaccg
gcataaccaagcctatgcctacagcatccagggtgacggtgccgaggatgacgatgagcgcattgttagatttcatacacggtgcctgact
gcgttagcaatttaactgtgataaactaccgcattaaagcttatcgatgataagctgtcaaacatgagaa

An N-terminal 6×His expression vector (“His ×6” disclosed as SEQ ID NO: 28) was prepared for 4-hydroxytryptamine kinase protein sequence (SEQ ID NO:2). SEQ ID NO:2 was codon optimized for E. coli using the IDTDNA codon optimization tool. BsaI type II restriction enzyme sequences were appended to the 5′ and 3′ of the sequence and synthesized as a Gblock by IDTDNA (Coralville, IA). The resulting linear dsDNA was reacted with pNAB0096 using the NEB® Golden Gate Assembly Kit (BsaI-HF®v2) according to the manufacturer's instructions. The reaction was transformed into E. coli strain TG1 (Lucigen) and plated onto LB agar containing ampicillin/chloramphenicol. A clone harboring the sequence of pNAB2002 was obtained after sequence verification by colony PCR, miniprep and plasmid DNA sequencing. The resulting circular plasmid sequence is listed below.

(4) pNAB2002
(SEQ ID NO: 19)
gcgttcgatctcaagactgaagacggcctcatcacatatctcactaaacatctttctttggacgtcgacacgagcggagtgaagcgccttagc
ggaggctttgtcaatgtaacctggcgcattaagctcaatgctccttatcaaggtcatacgagcatcatcctgaagcatgctcagccgcacatgt
ctacggatgaggattttaagataggtgtagaacgttcggtttacgaataccaggctatcaagctcatgatggccaatcgggaggttctgggag
gcgtggatggcatagtttctgtgccagaaggcctgaactacgacttagagaataatgcattgatcatgcaagatgtcgggaagatgaagacc
cttttagattatgtcaccgccaaaccgccacttgcgacggatatagcccgccttgttgggacagaaattggggggttcgttgccagactccat
aacataggccgcgagaggcgagatgatcctgagttcaaattcttctctggaaatattgtcggaaggacgacttcagaccagctgtatcaaac
catcatacccaacgcagcgaaatatggcgtcgatgaccccttgctgcctactgtggttaaggaccttgtggacgatgtcatgcacagcgaag
aaacccttgtcatggcggacctgtggagtggaaatattcttctccagttggaggagggaaacccatcgaagctgcagaagatatatatcctg
gattgcgaactttgcaagtacggcccagcgtcgttggacctgggctatttcttgggtgactgctatttgatatcccgctttcaagacgagcagg
tcggtacgacgatgcggcaagcctacttgcaaagctatgcgcgtacgagcaagcattcgatcaactacgccaaagtcactgcaggtattgc
tgctcatattgtgatgtggaccgactttatgcagtgggggagcgaggaagaaaggataaattttgtgaaaaagggggtagctgcctttcacg
acgccaggggcaacaacgacaatggggaaattacgtctaccttactgaaggaatcatccactgcgGGATCCtgatcaatccggctgc
taacaaagcccgaaaggaagctgagttggctgctgccaccgctgagcaataactagcataaccccttggggcctctaaacgggtcttgag
gggttttttgctgaaaggaggaactatatccggatatcccgcaagaggcccggcagtaccggcataaccaagcctatgcctacagcatcca
gggtgacggtgccgaggatgacgatgagcgcattgttagatttCATACACGGTGCCTGACTGCGTTAGCAAttta
actgtgataaactaccgcattaaagcttatcgatgataagctgtcaaacatgagaattcttgaagacgaaagggcctcgtgatacgcctattttt
ataggttaatgtcatgataataatggtttcttagacgtcaggtggcacttttcggggaaatgtgcgcggaacccctatttgtttatttttctaaa
tacattcaaatatgtatccgctcatgagacaataaccctgataaatgcttcaataatattgaaaaaggaagagtatgagtattcaacatttccgt
gtcgcccttattcccttttttgcggcattttgccttcctgtttttgctcacccagaaacgctggtgaaagtaaaagatgctgaagatcagttgg
gtgcacgagtgggttacatcgaactggatctcaacagcggtaagatccttgagagttttcgccccgaagaacgttttccaatgatgagcactttt
aaagttctgctatgtggcgcggtattatcccgtgttgacgcccgggcaagagcaactcggtcgccgcatacactattctcagaatgacttggttg
agtactcaccagtcacagaaaagcatcttacggatggcatgacagtaagagaattatgcagtgctgccataaccatgagtgataacactgcggcc
aacttacttctgacaacgatcggaggaccgaaggagctaaccgcttttttgcacaacatgggggatcatgtaactcgccttgatcgttgggaa
ccggagctgaatgaagccataccaaacgacgagcgtgacaccacgatgcctgcagcaatggcaacaacgttgcgcaaactattaactgg
cgaactacttactctagcttcccggcaacaattaatagactggatggaggcggataaagttgcaggaccacttctgcgctcggcccttccgg
ctggctggtttattgctgataaatctggagccggtgagcgtgggtcacgcggtatcattgcagcactggggccagatggtaagccctcccgt
atcgtagttatctacacgacggggagtcaggcaactatggatgaacgaaatagacagatcgctgagataggtgcctcactgattaagcattg
gtaactgtcagaccaagtttactcatatatactttagattgatttaaaacttcatttttaatttaaaaggatctaggtgaagatcctttttgata
atctcatgaccaaaatcccttaacgtgagttttcgttccactgagcgtcagaccccgtagaaaagatcaaaggatcttcttgagatccttttttt
ctgcgcgtaatctgctgcttgcaaacaaaaaaaccaccgctaccagcggtggtttgtttgccggatcaagagctaccaactctttttccgaaggt
aactggcttcagcagagcgcagataccaaatactgtccttctagtgtagccgtagttaggccaccacttcaagaactctgtagcaccgcctacat
acctcgctctgctaatcctgttaccagtggctgctgccagtggcgataagtcgtgtcttaccgggttggactcaagacgatagttaccggataag
gcgcagcggtcgggctgaacggggggttcgtgcacacagcccagcttggagcgaacgacctacaccgaactgagatacctacagcgtgag
ctatgagaaagcgccacgcttcccgaagggagaaaggcggacaggtatccggtaagcggcagggtcggaacaggagagcgcacgag
ggagcttccagggggaaacgcctggtatctttatagtcctgtcgggtttcgccacctctgacttgagcgtcgatttttgtgatgctcgtcaggg
gggcggagcctatggaaaaacgccagcaacgcggcctttttacggttcctggccttttgctggccttttgctcacatgttctttcctgcgttatc
ccctgattctgtggataaccgtattaccgcctttgagtgagctgataccgctcgccgcagccgaacgaccgagcgcagcgagtcagtgagc
gaggaagcggaagagcgcctgatgcggtattttctccttacgcatctgtgcggtatttcacaccgcatatatggtgcactctcagtacaatctg
ctctgatgccgcatagttaagccagtatacactccgctatcgctacgtgactgggtcatggctgcgccccgacacccgccaacacccgctg
acgcgccctgacgggcttgtctgctcccggcatccgcttacagacaagctgtgaccttctccgggagctgcatgtgtcagaggttttcaccgt
catcaccgaaacgcgcgaggcagctgcggtaaagctcatcagcgtggtcgtgaagcgattcacagatgtctgcctgttcatccgcgtccag
ctcgttgagtttctccagaagcgttaatgtctggcttctgataaagcgggccatgttaagggcggttttttcctgtttggtcactgatgcctccgt
gtaagggggatttctgttcatgggggtaatgataccgatgaaacgagagaggatgctcacgatacgggttactgatgatgaacatgcccggtt
actggaacgttgtgagggtaaacaactggcggtatggatgcggcgggaccagagaaaaatcactcagggtcaatgccagcgcttcgttaa
tacagatgtaggtgttccacagggtagccagcagcatcctgcgatgcagatccggaacataatggtgcagggcgctgacttccgcgtttcc
agactttacgaaacacggaaaccgaagaccattcatgttgttgctcaggtcgcagacgttttgcagcagcagtcgcttcacgttcgctcgcgt
atcggtgattcattctgctaaccagtaaggcaaccccgccagcctagccgggtcctcaacgacaggagcacgatcatgcgcacccgtggc
caggacccaacgctgcccgagatgcgccgcgtgcggctgctggagatggcggacgcgatggatatgttctgccaagggttggtttgcgc
attcacagttctccgcaagaattgattggctccaattcttggagtggtgaatccgttagcgaggtgccgccggcttccattcaggtcgaggtg
gcccggctccatgcaccgcgacgcaacgcggggaggcagacaaggtatagggcggcgcctacaatccatgccaacccgttccatgtgc
tcgccgaggcggcataaatcgccgtgacgatcagcggtccagtgatcgaagttaggctggtaagagccgcgagcgatccttgaagctgtc
cctgatggtcgtcatctacctgcctggacagcatggcctgcaacgcgggcatcccgatgccgccggaagcgagaagaatcataatgggg
aaggccatccagcctcgcgtcgcgaacgccagcaagacgtagcccagcgcgtcggccgccatgccggcgataatggcctgcttctcgc
cgaaacgtttggtggcgggaccagtgacgaaggcttgagcgagggcgtgcaagattccgaataccgcaagcgacaggtcgatcatcgtc
gcgctccagcgaaagcggtcctcgccgaaaatgacccagagcgctgccggcacctgtcctacgagttgcatgataaagaagacagtcat
aagtgcggcgacgatagtcatgccccgcgcccaccggaaggagctgactgggttgaaggctctcaagggcatcggtcgagatcccggt
gcctaatgagtgagCTAacttacattaattgcgttgcgctcactgcccgctttccagtcgggaaacctgtcgtgccagctgcattaatgaat
cggccaacgcgcggggagaggcggtttgcgtattgggcgccagggtggtttttcttttcaccagtgaaacgggcaacagctgattgccctt
caccgcctggccctgagagagttgcagcaagcggtccacgctggtttgccccagcaggcgaaaatcctgtttgatggtggttaacggcgg
gatataacatgagctgtcttcggtatcgtcgtatcccactaccgagatatccgcaccaacgcgcagcccggactcggtaatggcgcgcattg
cgcccagcgccatctgatcgttggcaaccagcatcgcagtgggaacgatgccctcattcagcatttgcatggtttgttgaaaaccggacatg
gcactccagtcgcccttcccgttccgctatcggctgaatttgattgcgagtgagatatttatgccagccagccagacgcagacgcgccgagac
agaacttaatgggcccgctaacagcgcgatttgctggtgacccaatgcgaccagatgctccacgcccagtcgcgtaccgtcttcatgggag
aaaataatactgttgatgggtgtctggtcagagacatcaagaaataacgccggaacattagtgcaggcagcttccacagcaatggcatcctg
gtcatccagcggatagttaatgatcagcccactgacgcgttgcgcgagaagattgtgcaccgcccgctttacaggcttcgacgccgcttcgtt
ctaccatcgacaccaccacgctggcacccagttgatcggcgcgagatttaatcgccgcgacaatttgcgacggcgcgtgcagggccaga
ctggaggtggcaacgccaatcagcaacgactgtttgcccgccagttgttgtgccacgcggttgggaatgtaattcagctccgccatcgccg
cttccactttttcccgcgttttcgcagaaacgtggctggcctggttcaccacgcgggaaacggtctgataagagacaccggcatactctgcg
acatcgtataacgttactggtttCACattcaccaccctgaattgactctcttccgggcgctatcatgccataccgcgaaaggttttgcgccatt
cgatggtgtccgggatctcgacgctctcccttatgcgactcctgcattaggaagcagcccagtagtaggttgaggccgttgagcaccgccg
ccgcaaggaatggtgcatgcaaggagatggcgcccaacagtcccccggccacggggcctgccaccatacccacgccgaaacaagcgc
tcatgagcccgaagtggcgagcccgatcttccccatcggtgatgtcggcgatataggcgccagcaaccgcacctgtggcgccggtgatgc
cggccacGATGCGTCCGGCGTAGAGGATCGAGatctcgatcccgcgaaattaatacgactcactataggggaattg
tgagcggataacaattcccctctagaaataattttgtttaactttaagaaggagatataccATGggccatcaccatcaccatcacgactacg
acatcccgactaccgaaaacctgtacttccagggttct

O-methyl transferase protein sequence (SEQ ID NO:3) was codon optimized for E. coli using the IDTDNA codon optimization tool. BsaI type II restriction enzyme sequences were appended to the 5′ and 3′ of the sequence and synthesized as a Gblock by IDTDNA (Coralville, IA). The resulting linear dsDNA was reacted with pNAB0096 using the NEB® Golden Gate Assembly Kit (BsaI-HF®v2) according to the manufacturer's instructions. The reaction was transformed into E. coli strain TG1 (Lucigen) and plated onto LB agar containing ampicillin/chloramphenicol. A clone harboring the sequence of pNAB2003 was obtained after sequence verification by colony PCR, miniprep and plasmid DNA sequencing. The resulting circular plasmid sequence is listed below.

(5) pNAB2003
(SEQ ID NO: 20)
GGGAGCTCAGAAGACCAGGCCTATCGTTTATTGAATGACTACGCTAATGGCTTCATG
GTTTCTCAAGTTCTTTTCGCCGCCTGTGAGCTGGGGGTGTTCGACTTGTTGGCGGAA
GCTCCCGGTCCCCTTGATGTTGCTGCAGTAGCGGCGGGGGTGCGCGCCTCGGCTCAC
GGAACGGAACTGTTATTGGACATCTGTGTCAGCCTGAAACTTCTTAAAGTGGAAACA
CGCGGCGGTAAAGCATTTTACCGTAACACTGAGTTGTCGTCTGATTATTTGACTACA
GTATCGCCGACATCTCAGTGCTCCATGTTAAAGTATATGGGCCGCACATCATATCGC
TGTTGGGGACATTTAGCTGACGCGGTACGCGAAGGTCGCAATCAGTATTTGGAAAC
CTTCGGGGTGCCAGCGGAAGAGTTATTCACGGCTATCTACCGCTCGGAGGGTGAAC
GCCTTCAATTTATGCAAGCATTACAAGAAGTCTGGTCCGTAAATGGTCGCTCAGTCC
TGACTGCGTTCGACCTGTCTGTGTTTCCCCTGATGTGTGACCTGGGAGGCGGCGCAG
GGGCACTTGCAAAAGAATGTATGTCACTTTATCCGGGGTGTAAAATTACTGTGTTTG
ATATCCCTGAAGTGGTCTGGACCGCGAAACAACATTTCAGCTTCCAAGAGGAAGAA
CAAATCGACTTCCAGGAAGGCGACTTTTTTAAAGACCCTTTACCCGAGGCTGATTTA
TACATCTTAGCTCGTGTGTTGCACGACTGGGCGGATGGTAAATGCTCGCACTTACTT
GAACGCATTTATCACACTTGTAAGCCTGGGGGAGGCATCTTAGTCATTGAAAGTTTG
CTGGACGAGGACCGTCGCGGGCCTCTTTTGACCCAGTTATATTCCTTAAACATGTTG
GTCCAGACAGAAGGCCAAGAGCGTACGCCAACACACTATCACATGCTGCTGTCATC
AGCCGGGTTCCGCGACTTTCAGTTCAAAAAGACAGGCGCCATCTACGATGCAATTTT
AGCACGTAAGGGATCCtgatcaatccggctgctaacaaagcccgaaaggaagctgagttggctgctgccaccgctgagca
ataactagcataaccccttggggcctctaaacgggtcttgaggggttttttgctgaaaggaggaactatatccggatatcccgcaagaggcc
cggcagtaccggcataaccaagcctatgcctacagcatccagggtgacggtgccgaggatgacgatgagcgcattgttagatttCATA
CACGGTGCCTGACTGCGTTAGCAAtttaactgtgataaactaccgcattaaagcttatcgatgataagctgtcaaacatg
agaattcttgaagacgaaagggcctcgtgatacgcctatttttataggttaatgtcatgataataatggtttcttagacgtcaggtggcacttt
tcggggaaatgtgcgcggaacccctatttgtttatttttctaaatacattcaaatatgtatccgctcatgagacaataaccctgataaatgctt
caataatattgaaaaaggaagagtatgagtattcaacatttccgtgtcgcccttattcccttttttgcggcattttgccttcctgtttttgctc
acccagaaacgctggtgaaagtaaaagatgctgaagatcagttgggtgcacgagtgggttacatcgaactggatctcaacagcggtaagatcct
tgagagttttcgccccgaagaacgttttccaatgatgagcacttttaaagttctgctatgtggcgcggtattatcccgtgttgacgccgggcaa
gagcaactcggtcgccgcatacactattctcagaatgacttggttgagtactcaccagtcacagaaaagcatcttacggatggcatgacagtaa
gagaattatgcagtgctgccataaccatgagtgataacactgcggccaacttacttctgacaacgatcggaggaccgaaggagctaaccgcttt
tttgcacaacatgggggatcatgtaactcgccttgatcgttgggaaccggagctgaatgaagccataccaaacgacgagcgtgacaccacgatgc
ctgcagcaatggcaacaacgttgcgcaaactattaactggcgaactacttactctagcttcccggcaacaattaatagactggatggaggcg
gataaagttgcaggaccacttctgcgctcggcccttccggctggctggtttattgctgataaatctggagccggtgagcgtgggtcacgcgg
tatcattgcagcactggggccagatggtaagccctcccgtatcgtagttatctacacgacggggagtcaggcaactatggatgaacgaaata
gacagatcgctgagataggtgcctcactgattaagcattggtaactgtcagaccaagtttactcatatatactttagattgatttaaaacttca
tttttaatttaaaaggatctaggtgaagatcctttttgataatctcatgaccaaaatcccttaacgtgagttttcgttccactgagcgtcagac
cccgtagaaaagatcaaaggatcttcttgagatccttttttttgcgcgtaatctgctgcttgcaaacaaaaaaaccaccgctaccagcggtgg
tttgtttgccggatcaagagctaccaactctttttccgaaggtaactggcttcagcagagcgcagataccaaatactgtccttctagtgtagcc
gtagttaggccaccacttcaagaactctgtagcaccgcctacatacctcgctctgctaatcctgttaccagtggctgctgccagtggcgataag
tcgtgtcttaccgggttggactcaagacgatagttaccggataaggcgcagcggtcgggctgaacggggggttcgtgcacacagcccagcttg
gagcgaacgacctacaccgaactgagatacctacagcgtgagctatgagaaagcgccacgcttcccgaagggagaaaggcggacaggtatcc
ggtaagcggcagggtcggaacaggagagcgcacgagggagcttccagggggaaacgcctggtatctttatagtcctgtcgggtttcgcc
acctctgacttgagcgtcgatttttgtgatgctcgtcaggggggcggagcctatggaaaaacgccagcaacgcggcctttttacggttcctgg
ccttttgctggccttttgctcacatgttctttcctgcgttatcccctgattctgtggataaccgtattaccgcctttgagtgagctgataccgc
tcgccgcagccgaacgaccgagcgcagcgagtcagtgagcgaggaagcggaagagcgcctgatgcggtattttctccttacgcatctgtgcgg
tatttcacaccgcatatatggtgcactctcagtacaatctgctctgatgccgcatagttaagccagtatacactccgctatcgctacgtgactgg
gtcatggctgcgccccgacacccgccaacacccgctgacgcgccctgacgggcttgtctgctcccggcatccgcttacagacaagctgtg
accttctccgggagctgcatgtgtcagaggttttcaccgtcatcaccgaaacgcgcgaggcagctgcggtaaagctcatcagcgtggtcgt
gaagcgattcacagatgtctgcctgttcatccgcgtccagctcgttgagtttctccagaagcgttaatgtctggcttctgataaagcgggccat
gttaagggcggttttttcctgtttggtcactgatgcctccgtgtaagggggatttctgttcatgggggtaatgataccgatgaaacgagagagg
atgctcacgatacgggttactgatgatgaacatgcccggttactggaacgttgtgagggtaaacaactggcggtatggatgcggcgggacc
agagaaaaatcactcagggtcaatgccagcgcttcgttaatacagatgtaggtgttccacagggtagccagcagcatcctgcgatgcagat
ccggaacataatggtgcagggcgctgacttccgcgtttccagactttacgaaacacggaaaccgaagaccattcatgttgttgctcaggtcg
cagacgttttgcagcagcagtcgcttcacgttcgctcgcgtatcggtgattcattctgctaaccagtaaggcaaccccgccagcctagccgg
gtcctcaacgacaggagcacgatcatgcgcacccgtggccaggacccaacgctgcccgagatgcgccgcgtgcggctgctggagatgg
cggacgcgatggatatgttctgccaagggttggtttgcgcattcacagttctccgcaagaattgattggctccaattcttggagtggtgaatcc
gttagcgaggtgccgccggcttccattcaggtcgaggtggcccggctccatgcaccgcgacgcaacgggggaggcagacaaggtata
gggcggcgcctacaatccatgccaacccgttccatgtgctcgccgaggcggcataaatcgccgtgacgatcagcggtccagtgatcgaa
gttaggctggtaagagccgcgagcgatccttgaagctgtccctgatggtcgtcatctacctgcctggacagcatggcctgcaacgcgggca
tcccgatgccgccggaagcgagaagaatcataatggggaaggccatccagcctcgcgtcgcgaacgccagcaagacgtagcccagcg
cgtcggccgccatgccggcgataatggcctgcttctcgccgaaacgtttggtgggggaccagtgacgaaggcttgagcgagggcgtgc
aagattccgaataccgcaagcgacaggtcgatcatcgtcgcgctccagcgaaagcggtcctcgccgaaaatgacccagagcgctgccg
gcacctgtcctacgagttgcatgataaagaagacagtcataagtgcggcgacgatagtcatgccccgcgcccaccggaaggagctgactg
ggttgaaggctctcaagggcatcggtcgagatcccggtgcctaatgagtgagCTAacttacattaattgcgttgcgctcactgcccgctttc
cagtcgggaaacctgtcgtgccagctgcattaatgaatcggccaacgcgcggggagaggcggtttgcgtattgggcgccagggtggttttt
cttttcaccagtgaaacgggcaacagctgattgcccttcaccgcctggccctgagagagttgcagcaagcggtccacgctggtttgcccca
gcaggcgaaaatcctgtttgatggtggttaacggcgggatataacatgagctgtcttcggtatcgtcgtatcccactaccgagatatccgcac
caacgcgcagcccggactcggtaatggcgcgcattgcgcccagcgccatctgatcgttggcaaccagcatcgcagtgggaacgatgccc
tcattcagcatttgcatggtttgttgaaaaccggacatggcactccagtcgccttcccgttccgctatcggctgaatttgattgcgagtgagat
atttatgccagccagccagacgcagacgcgccgagacagaacttaatgggcccgctaacagcgcgatttgctggtgacccaatgcgaccag
atgctccacgcccagtcgcgtaccgtcttcatgggagaaaataatactgttgatgggtgtctggtcagagacatcaagaaataacgccggaa
cattagtgcaggcagcttccacagcaatggcatcctggtcatccagcggatagttaatgatcagcccactgacgcgttgcgcgagaagattg
tgcaccgccgctttacaggcttcgacgccgcttcgttctaccatcgacaccaccacgctggcacccagttgatcggcgcgagatttaatcgc
cgcgacaatttgcgacggcgcgtgcagggccagactggaggtggcaacgccaatcagcaacgactgtttgcccgccagttgttgtgccac
gcggttgggaatgtaattcagctccgccatcgccgcttccactttttcccgcgttttcgcagaaacgtggctggcctggttcaccacgcggga
aacggtctgataagagacaccggcatactctgcgacatcgtataacgttactggtttCACattcaccaccctgaattgactctcttccgggc
gctatcatgccataccgcgaaaggttttgcgccattcgatggtgtccgggatctcgacgctctcccttatgcgactcctgcattaggaagcag
cccagtagtaggttgaggtcgttgagcaccgccgccgcaaggaatggtgcatgcaaggagatggcgcccaacagtcccccggccacgg
ggcctgccaccatacccacgccgaaacaagcgctcatgagcccgaagtggcgagcccgatcttccccatcggtgatgtcggcgatatag
gcgccagcaaccgcacctgtggcgccggtgatgccggccacGATGCGTCCGGCGTAGAGGATCGAGatctcgat
cccgcgaaattaatacgactcactataggggaattgtgagcggataacaattcccctctagaaataattttgtttaactttaagaaggagatata
ccATGggccatcaccatcaccatcacgactacgacatcccgactaccgaaaacctgtacttccagggttct

Sulfotransferase protein sequences (SEQ ID NOs: 5 and 6) were codon optimized for E. coli using the IDTDNA codon optimization tool. BsaI type II restriction enzyme sequences were appended to the 5′ and 3′ of each sequence and synthesized as a Oblock by IDTDNA (Coralville, IA). The resulting linear dsDNA was reacted with pNAB0098 using the NEB Golden Gate Assembly Kit (BsaI-HF®v2) according to the manufacturer's instructions. The reaction was transformed into E. coli strain TG1 (Lucigen) and plated onto LB agar containing ampicillin/chloramphenicol. Clones harboring the sequence of pNAB2005 and pNAB2006 were obtained after sequence verification by colony PCR, miniprep and plasmid DNA sequencing.

(6) pNAB2005
(SEQ ID NO: 21)
gaaaacctgtacttccagggccatcaccatcaccatcactgatcaatccggctgctaacaaagcccgaaaggaagctgagttggctgctgc
caccgctgagcaataactagcataaccccttggggcctctaaacgggtcttgaggggttttttgctgaaaggaggaactatatccggatatcc
cgcaagaggcccggcagtaccggcataaccaagcctatgcctacagcatccagggtgacggtgccgaggatgacgatgagcgcattgtt
agatttcatacacggtgcctgactgcgttagcaatttaactgtgataaactaccgcattaaagcttatcgatgataagctgtcaaacatgagaat
tcttgaagacgaaagggcctcgtgatacgcctatttttataggttaatgtcatgataataatggtttcttagacgtcaggtggcacttttcgggga
aatgtgcgcggaacccctatttgtttatttttctaaatacattcaaatatgtatccgctcatgagacaataaccctgataaatgcttcaataata
ttgaaaaaggaagagtatgagtattcaacatttccgtgtcgcccttattcccttttttgcggcattttgccttcctgtttttgctcacccagaaac
gctggtgaaagtaaaagatgctgaagatcagttgggtgcacgagtgggttacatcgaactggatctcaacagcggtaagatccttgagagttttcg
ccccgaagaacgttttccaatgatgagcacttttaaagttctgctatgtggcgcggtattatcccgtgttgacgccgggcaagagcaactcggt
cgccgcatacactattctcagaatgacttggttgagtactcaccagtcacagaaaagcatcttacggatggcatgacagtaagagaattatgc
agtgctgccataaccatgagtgataacactgcggccaacttacttctgacaacgatcggaggaccgaaggagctaaccgcttttttgcacaa
catgggggatcatgtaactcgccttgatcgttgggaaccggagctgaatgaagccataccaaacgacgagcgtgacaccacgatgcctgc
agcaatggcaacaacgttgcgcaaactattaactggcgaactacttactctagcttcccggcaacaattaatagactggatggaggcggata
aagttgcaggaccacttctgcgctcggcccttccggctggctggtttattgctgataaatctggagccggtgagcgtggctctcgcggtatca
ttgcagcactggggccagatggtaagccccccgtatcgtagttatctacacgacggggagtcaggcaactatggatgaacgaaatagaca
gatcgctgagataggtgcctcactgattaagcattggtaactgtcagaccaagtttactcatatatactttagattgatttaaaacttcattttt
aatttaaaaggatctaggtgaagatcctttttgataatctcatgaccaaaatcccttaacgtgagttttcgttccactgagcgtcagaccccgta
gaaaagatcaaaggatcttcttgagatcctttttttctgcgcgtaatctgctgcttgcaaacaaaaaaaccaccgctaccagcggtggtttgttt
gccggatcaagagctaccaactctttttccgaaggtaactggcttcagcagagcgcagataccaaatactgtccttctagtgtagccgtagttag
gccaccacttcaagaactctgtagcaccgcctacatacctcgctctgctaatcctgttaccagtggctgctgccagtggcgataagtcgtgtctt
accgggttggactcaagacgatagttaccggataaggcgcagcggtcgggctgaacggggggttcgtgcacacagcccagcttggagcga
acgacctacaccgaactgagatacctacagcgtgagctatgagaaagcgccacgcttcccgaagggagaaaggcggacaggtatccgg
taagcggcagggtcggaacaggagagcgcacgagggagcttccagggggaaacgcctggtatctttatagtcctgtcgggtttcgccacc
tctgacttgagcgtcgatttttgtgatgctcgtcaggggggggagcctatggaaaaacgccagcaacgcggcctttttacggttcctggcct
tttgctggccttttgctcacatgttctttcctgcgttatcccctgattctgtggataaccgtattaccgcctttgagtgagctgataccgctcgc
cgcagccgaacgaccgagcgcagcgagtcagtgagcgaggaagcggaagagcgcctgatgcggtattttctccttacgcatctgtgcggtattt
cacaccgcatatatggtgcactctcagtacaatctgctctgatgccgcatagttaagccagtatacactccgctatcgctacgtgactgggtca
tggctgcgccccgacacccgccaacacccgctgacgcgccctgacgggcttgtctgctcccggcatccgcttacagacaagctgtgacca
tctccgggagctgcatgtgtcagaggttttcaccgtcatcaccgaaacgcgcgaggcagctgcggtaaagctcatcagcgtggtcgtgaag
cgattcacagatgtctgcctgttcatccgcgtccagctcgttgagtttctccagaagcgttaatgtctggcttctgataaagcgggccatgttaa
gggcggttttttctgtttggtcactgatgcctccgtgtaagggggatttctgttcatgggggtaatgataccgatgaaacgagagaggatgct
cacgatacgggttactgatgatgaacatgcccggttactggaacgttgtgagggtaaacaactggcggtatggatgcggcgggaccagag
aaaaatcactcagggtcaatgccagcgcttcgttaatacagatgtaggtgttccacagggtagccagcagcatcctgcgatgcagatccgg
aacataatggtgcagggcgctgacttccgcgtttccagactttacgaaacacggaaaccgaagaccattcatgttgttgctcaggtcgcaga
cgttttgcagcagcagtcgcttcacgttcgctcgcgtatcggtgattcattctgctaaccagtaaggcaaccccgccagcctagccgggtcct
caacgacaggagcacgatcatgcgcacccgtggccaggacccaacgctgcccgagatgcgccgcgtgcggctgctggagatggcgga
cgcgatggatatgttctgccaagggttggtttgcgcattcacagttctccgcaagaattgattggctccaattcttggagtggtgaatccgttag
cgaggtgccgccggcttccattcaggtcgaggtggcccggctccatgcaccgcgacgcaacgcggggaggcagacaaggtatagggc
ggcgcctacaatccatgccaacccgttccatgtgctcgccgaggcggcataaatcgccgtgacgatcagcggtccagtgatcgaagttag
gctggtaagagccgcgagcgatccttgaagctgtccctgatggtcgtcatctacctgcctggacagcatggcctgcaacgcgggcatccc
gatgccgccggaagcgagaagaatcataatggggaaggccatccagcctcgcgtcgcgaacgccagcaagacgtagcccagcgcgtc
ggccgccatgccggcgataatggcctgcttctcgccgaaacgtttggtggcgggaccagtgacgaaggcttgagcgagggcgtgcaaga
ttccgaataccgcaagcgacaggtcgatcatcgtcgcgctccagcgaaagcggtcctcgccgaaaatgacccagagcgctgccggcac
ctgtcctacgagttgcatgataaagaagacagtcataagtgcggcgacgatagtcatgccccgcgcccaccggaaggagctgactgggtt
gaaggctctcaagggcatcggtcgagatcccggtgcctaatgagtgagctaacttacattaattgcgttgcgctcactgcccgctttccagtc
gggaaacctgtcgtgccagctgcattaatgaatcggccaacgcgcggggagaggcggtttgcgtattgggcgccagggggtttttcttttc
accagtgaaacgggcaacagctgattgcccttcaccgcctggccctgagagagttgcagcaagcggtccacgctggtttgccccagcag
gcgaaaatcctgtttgatggtggttaacggcgggatataacatgagctgtcttcggtatcgtcgtatcccactaccgagatatccgcaccaac
gcgcagcccggactcggtaatggcgcgcattgcgcccagcgccatctgatcgttggcaaccagcatcgcagtgggaacgatgccctcatt
cagcatttgcatggtttgttgaaaaccggacatggcactccagtcgccttcccgttccgctatcggctgaatttgattgcgagtgagatatttat
gccagccagccagacgcagacgcgccgagacagaacttaatgggcccgctaacagcgcgatttgctggtgacccaatgcgaccagatg
ctccacgcccagtcgcgtaccgtcttcatgggagaaaataatactgttgatgggtgtctggtcagagacatcaagaaataacgccggaacat
tagtgcaggcagcttccacagcaatggcatcctggtcatccagcggatagttaatgatcagcccactgacgcgttgcgcgagaagattgtg
caccgccgctttacaggcttcgacgccgcttcgttctaccatcgacaccaccacgctggcacccagttgatcggcgcgagatttaatcgccg
cgacaatttgcgacggcgcgtgcagggccagactggaggtggcaacgccaatcagcaacgactgtttgcccgccagttgttgtgccacgc
ggttgggaatgtaattcagctccgccatcgccgcttccactttttcccgcgttttcgcagaaacgtggctggcctggttcaccacgcgggaaa
cggtctgataagagacaccggcatactctgcgacatcgtataacgttactggtttcacattcaccaccctgaattgactctcttccgggcgctat
catgccataccgcgaaaggttttgcgccattcgatggtgtccgggatctcgacgctctcccttatgcgactcctgcattaggaagcagccca
gtagtaggttgaggccgttgagcaccgccgccgcaaggaatggtgcatgcaaggagatggcgcccaacagtcccccggccacggggc
ctgccaccatacccacgccgaaacaagcgctcatgagcccgaagtggcgagcccgatcttccccatcggtgatgtcggcgatataggcg
ccagcaaccgcacctgtggcgccggtgatgccggccacgatgcgtccggcgtagaggatcgagatctcgatcccgcgaaattaatacga
ctcactataggggaattgtgagcggataacaattcccctctagaaataattttgtttaactttaagaaggagatatactatggaattgattcaaga
cacttcaagaccaccattggaatacgttaagggtgtacctttgatcaagtatttcgcagaggcattaggtccattacagtcttttcaagcaagac
cagatgatttgttaatttctacatatccaaaatctggaactacttgggtatctcagatattggatatgatctatcaaggaggagacttggaaaagt
gtcatagagcacctatcttcatgagagtcccattcttggaatttaaagctccaggtattccttctggtatggaaactttaaaagatactccagctc
caaggttcttgaaaacccatttgccattggctttattacctcaaacattattagatcagaaagttaaagtagtttatgttgctagaaatgcaaaag
atgtagcagtttcatattaccacttctatcatatggctaaagttcaccctgaacctggtacatgggattcattcttagaaaagtttatggtaggag
aggtttcatatggttcatggtatcagcacgtccaggaatggtgggaattatctaggacacatccagtcttgtacttgttttatgaagacatgaaag
aaaaccctaaaagagagatccagaagattttggaatttgtcggtaggtctttaccagaggaaactgtcgattttgtcgttcaacacacatcattt
aaggaaatgaagaagaacccaatgacaaactacacaactgttcctcaagaatttatggaccactctatatctccatttatgaggaaaggtatggc
cggtgattggaaaactacttttacagtagcacaaaacgaaagattcgatgccgattatgctgagaaaatggcaggatgctctttgtcattcagg
tctgaattaGGATCC
(7) pNAB2006
(SEQ ID NO: 22)
gaaaacctgtacttccagggccatcaccatcaccatcactgatcaatccggctgctaacaaagcccgaaaggaagctgagttggctgctgc
caccgctgagcaataactagcataaccccttggggcctctaaacgggtcttgaggggttttttgctgaaaggaggaactatatccggatatcc
cgcaagaggcccggcagtaccggcataaccaagcctatgcctacagcatccagggtgacggtgccgaggatgacgatgagcgcattgtt
agatttcatacacggtgcctgactgcgttagcaatttaactgtgataaactaccgcattaaagcttatcgatgataagctgtcaaacatgagaat
tcttgaagacgaaagggcctcgtgatacgcctatttttataggttaatgtcatgataataatggtttcttagacgtcaggtggcacttttcgggga
aatgtgcgcggaacccctatttgtttatttttctaaatacattcaaatatgtatccgctcatgagacaataaccctgataaatgcttcaataatat
tgaaaaaggaagagtatgagtattcaacatttccgtgtcgcccttattcccttttttgcggcattttgccttcctgtttttgctcacccagaaac
gctggtgaaagtaaaagatgctgaagatcagttgggtgcacgagtgggttacatcgaactggatctcaacagcggtaagatccttgagagttttcg
ccccgaagaacgttttccaatgatgagcacttttaaagttctgctatgtggcgcggtattatcccgtgttgacgccgggcaagagcaactcggt
cgccgcatacactattctcagaatgacttggttgagtactcaccagtcacagaaaagcatcttacggatggcatgacagtaagagaattatgc
agtgctgccataaccatgagtgataacactgcggccaacttacttctgacaacgatcggaggaccgaaggagctaaccgcttttttgcacaa
catgggggatcatgtaactcgccttgatcgttgggaaccggagctgaatgaagccataccaaacgacgagcgtgacaccacgatgcctgc
agcaatggcaacaacgttgcgcaaactattaactggcgaactacttactctagcttcccggcaacaattaatagactggatggaggcggata
aagttgcaggaccacttctgcgctcggcccttccggctggctggtttattgctgataaatctggagccggtgagcgtggctctcgcggtatca
ttgcagcactggggccagatggtaagccctcccgtatcgtagttatctacacgacggggagtcaggcaactatggatgaacgaaatagaca
gatcgctgagataggtgcctcactgattaagcattggtaactgtcagaccaagtttactcatatatactttagattgatttaaaacttcattttt
aatttaaaaggatctaggtgaagatcctttttgataatctcatgaccaaaatcccttaacgtgagttttcgttccactgagcgtcagaccccgta
gaaaagatcaaaggatcttcttgagatcctttttttctgcgcgtaatctgctgcttgcaaacaaaaaaaccaccgctaccagcggtggtttgttt
gccggatcaagagctaccaactctttttccgaaggtaactggcttcagcagagcgcagataccaaatactgtccttctagtgtagccgtagttag
gccaccacttcaagaactctgtagcaccgcctacatacctcgctctgctaatcctgttaccagtggctgctgccagtggcgataagtcgtgtctta
ccgggttggactcaagacgatagttaccggataaggcgcagcggtcgggctgaacggggggttcgtgcacacagcccagcttggagcga
acgacctacaccgaactgagatacctacagcgtgagctatgagaaagcgccacgcttcccgaagggagaaaggcggacaggtatcgg
taagcggcagggtcggaacaggagagcgcacgagggagcttccagggggaaacgcctggtatctttatagtcctgtcgggtttcgccacc
tctgacttgagcgtcgatttttgtgatgctcgtcaggggggcggagcctatggaaaaacgccagcaacgcggcctttttacggttcctggcct
tttgctggccttttgctcacatgttctttcctgcgttatcccctgattctgtggataaccgtattaccgcctttgagtgagctgataccgctcgc
cgcagccgaacgaccgagcgcagcgagtcagtgagcgaggaagcggaagagcgcctgatgcggtattttctccttacgcatctgtgcggtattt
cacaccgcatatatggtgcactctcagtacaatctgctctgatgccgcatagttaagccagtatacactccgctatcgctacgtgactgggtca
tggctgcgccccgacacccgccaacacccgctgacgcgccctgacgggcttgtctgctcccggcatccgcttacagacaagctgtgacca
tctccgggagctgcatgtgtcagaggttttcaccgtcatcaccgaaacgcgcgaggcagctgcggtaaagctcatcagcgtggtcgtgaag
cgattcacagatgtctgcctgttcatccgcgtccagctcgttgagtttctccagaagcgttaatgtctggcttctgataaagcgggccatgttaa
gggcggttttttcctgtttggtcactgatgcctccgtgtaagggggatttctgttcatgggggtaatgataccgatgaaacgagagaggatgct
cacgatacgggttactgatgatgaacatgcccggttactggaacgttgtgagggtaaacaactggcggtatggatgcggcgggaccagag
aaaaatcactcagggtcaatgccagcgcttcgttaatacagatgtaggtgttccacagggtagccagcagcatcctgcgatgcagatccgg
aacataatggtgcagggcgctgacttccgcgtttccagactttacgaaacacggaaaccgaagaccattcatgttgttgctcaggtcgcaga
cgttttgcagcagcagtcgcttcacgttcgctcgcgtatcggtgattcattctgctaaccagtaaggcaaccccgccagcctagccgggtcct
caacgacaggagcacgatcatgcgcacccgtggccaggacccaacgctgcccgagatgcgccgcgtgcggctgctggagatggcgga
cgcgatggatatgttctgccaagggttggtttgcgcattcacagttctccgcaagaattgattggctccaattcttggagtggtgaatccgttag
cgaggtgccgccggcttccattcaggtcgaggtggcccggctccatgcaccgcgacgcaacgcggggaggcagacaaggtatagggc
ggcgcctacaatccatgccaacccgttccatgtgctcgccgaggcggcataaatcgccgtgacgatcagcggtccagtgatcgaagttag
gctggtaagagccgcgagcgatccttgaagctgtccctgatggtcgtcatctacctgcctggacagcatggcctgcaacgcgggcatccc
gatgccgccggaagcgagaagaatcataatggggaaggccatccagcctcgcgtcgcgaacgccagcaagacgtagcccagcgcgtc
ggccgccatgccggcgataatggcctgcttctcgccgaaacgtttggtgggggaccagtgacgaaggcttgagcgagggcgtgcaaga
ttccgaataccgcaagcgacaggtcgatcatcgtcgcgctccagcgaaagcggtcctcgccgaaaatgacccagagcgctgccggcac
ctgtcctacgagttgcatgataaagaagacagtcataagtgcggcgacgatagtcatgccccgcgcccaccggaaggagctgactgggtt
gaaggctctcaagggcatcggtcgagatcccggtgcctaatgagtgagctaacttacattaattgcgttgcgctcactgcccgctttccagtc
gggaaacctgtcgtgccagctgcattaatgaatcggccaacgcgcggggagaggcggtttgcgtattgggcgccagggtggtttttcttttc
accagtgaaacgggcaacagctgattgcccttcaccgcctggccctgagagagttgcagcaagcggtccacgctggtttgccccagcag
gcgaaaatcctgtttgatggtggttaacggcgggatataacatgagctgtcttcggtatcgtcgtatcccactaccgagatatccgcaccaac
gcgcagcccggactcggtaatggcgcgcattgcgcccagcgccatctgatcgttggcaaccagcatcgcagtgggaacgatgccctcatt
cagcatttgcatggtttgttgaaaaccggacatggcactccagtcgccttcccgttccgctatcggctgaatttgattgcgagtgagatatttat
gccagccagccagacgcagacgcgccgagacagaacttaatgggcccgctaacagcgcgatttgctggtgacccaatgcgaccagatg
ctccacgcccagtcgcgtaccgtcttcatgggagaaaataatactgttgatgggtgtctggtcagagacatcaagaaataacgccggaacat
tagtgcaggcagcttccacagcaatggcatcctggtcatccagcggatagttaatgatcagcccactgacgcgttgcgcgagaagattgtg
caccgccgctttacaggcttcgacgccgcttcgttctaccatcgacaccaccacgctggcacccagttgatcggcgcgagatttaatcgccg
cgacaatttgcgacggcgcgtgcagggccagactggaggtggcaacgccaatcagcaacgactgtttgcccgccagttgttgtgccacgc
ggttgggaatgtaattcagctccgccatcgccgcttccactttttcccgcgttttcgcagaaacgtggctggcctggttcaccacgcgggaaa
cggtctgataagagacaccggcatactctgcgacatcgtataacgttactggtttcacattcaccaccctgaattgactctcttccgggcgctat
catgccataccgcgaaaggttttgcgccattcgatggtgtccgggatctcgacgctctcccttatgcgactcctgcattaggaagcagccca
gtagtaggttgaggccgttgagcaccgccgccgtaaggaatggtgcatgcaaggagatggcgcccaacagtcccccggccacggggc
ctgccaccatacccacgccgaaacaagcgctcatgagcccgaagtggcgagcccgatcttccccatcggtgatgtcggcgatataggcg
ccagcaaccgcacctgtggcgccggtgatgccggccacgatgcgtccggcgtagaggatcgagatctcgatcccgcgaaattaatacga
ctcactataggggaattgtgagcggataacaattcccctctagaaataattttgtttaactttaagaaggagatatactatgGAATTGAT
TCAAGATACTTCAAGACCACCATTGGAATATGTTAAGGGTGTTCCTTTAATTAAGTA
CTTTGCCGAAGCCTTGGGTCCATTGCAATCTTTTCAAGCTAGACCTGATGATTTGTTG
ATCAATACTTATCCTAAATCTGGTACTACTTGGGTATCACAGATTTTGGACATGATTT
ACCAAGGTGGTGATTTGGAAAAGTGCAATAGGGCACCTATATATGTTAGGGTTCCAT
TCTTAGAAGTTAACGACCCAGGAGAACCTTCTGGTTTGGAAACATTGAAAGATACCC
CACCACCAAGATTAATTAAATCTCACTTGCCATTGGCTTTATTGCCACAGACTTTATT
GGACCAAAAGGTTAAGGTTGTTTACGTCGCCAGAAATCCAAAGGACGTAGCAGTTT
CTTATTACCATTTCCATAGAATGGAAAAGGCACACCCAGAACCAGGTACATGGGAC
TCATTCTTGGAGAAATTCATGGCTGGAGAAGTTTCTTACGGTTCTTGGTATCAGCAC
GTACAAGAATGGTGGGAATTATCAAGAACTCATCCAGTCTTATATTTATTTTATGAA
GATATGAAAGAAAATCCTAAGAGAGAAATCCAGAAAATCTTAGAGTTCGTAGGTAG
GTCTTTGCCAGAAGAAACAATGGATTTCATGGTACAACACACATCTTTTAAAGAGAT
GAAGAAGAATCCAATGACTAATTACACTACTGTTCCACAAGAATTAATGGACCACT
CTATCTCTCCTTTTATGAGGAAAGGTATGGCCGGAGATTGGAAAACCACTTTCACTG
TTGCTCAGAATGAGAGGTTTGATGCCGATTATGCTGAAAAGATGGCAGGTTGTTCTT
TGTCTTTTAGATCAGAATTGGGATCC

Acyl transferase protein sequence (SEQ ID NO:8) for chloramphenicol acetyltransferase (CAT) from Pseudomonas aeruginosa enzymes was codon optimized for E. coli using the IDTDNA codon optimization tool. BsaI type II restriction enzyme sequences were appended to the 5′ and 3′ of each sequence and synthesized as a Gblock by IDTDNA (Coralville, IA). The resulting linear dsDNA was reacted with pNAB0098 using the NEB® Golden Gate Assembly Kit (BsaI-HF®v2) according to the manufacturer's instructions. The reaction was transformed into E. coli strain TG1 (Lucigen) and plated onto LB agar containing ampicillin/chloramphenicol. A clone harboring the sequence of pNAB2008 was obtained after sequence verification by colony PCR, miniprep and plasmid DNA sequencing.

(8) pNAB2008
(SEQ ID NO: 23)
gaaaacctgtacttccagggccatcaccatcaccatcactgatcaatccggctgctaacaaagcccgaaaggaagctgagttggctgctgc
caccgctgagcaataactagcataaccccttggggcctctaaacgggtcttgaggggttttttgctgaaaggaggaactatatccggatatcc
cgcaagaggcccggcagtaccggcataaccaagcctatgcctacagcatccagggtgacggtgccgaggatgacgatgagcgcattgtt
agatttcatacacggtgcctgactgcgttagcaatttaactgtgataaactaccgcattaaagcttatcgatgataagctgtcaaacatgagaat
tcttgaagacgaaagggcctcgtgatacgcctatttttataggttaatgtcatgataataatggtttcttagacgtcaggtggcacttttcgggga
aatgtgcgcggaacccctatttgtttatttttctaaatacattcaaatatgtatccgctcatgagacaataaccctgataaatgcttcaataatat
tgaaaaaggaagagtatgagtattcaacatttccgtgtcgcccttattcccttttttgcggcattttgccttcctgtttttgctcacccagaaac
gctggtgaaagtaaaagatgctgaagatcagttgggtgcacgagtgggttacatcgaactggatctcaacagcggtaagatccttgagagttttcg
ccccgaagaacgttttccaatgatgagcacttttaaagttctgctatgtggcgcggtattatcccgtgttgacgccgggcaagagcaactcggt
cgccgcatacactattctcagaatgacttggttgagtactcaccagtcacagaaaagcatcttacggatggcatgacagtaagagaattatgc
agtgctgccataaccatgagtgataacactgcggccaacttacttctgacaacgatcggaggaccgaaggagctaaccgcttttttgcacaa
catgggggatcatgtaactcgccttgatcgttgggaaccggagctgaatgaagccataccaaacgacgagcgtgacaccacgatgcctgc
agcaatggcaacaacgttgcgcaaactattaactggcgaactacttactctagcttcccggcaacaattaatagactggatggaggcggata
aagttgcaggaccacttctgcgctcggcccttccggctggctggtttattgctgataaatctggagccggtgagcgtggctctcgcggtatca
ttgcagcactggggccagatggtaagccctcccgtatcgtagttatctacacgacggggagtcaggcaactatggatgaacgaaatagaca
gatcgctgagataggtgcctcactgattaagcattggtaactgtcagaccaagtttactcatatatactttagattgatttaaaacttcattttt
aatttaaaaggatctaggtgaagatcctttttgataatctcatgaccaaaatcccttaacgtgagttttcgttccactgagcgtcagaccccgta
gaaaagatcaaaggatcttcttgagatcctttttttctgcgcgtaatctgctgcttgcaaacaaaaaaaccaccgctaccagcggtggtttgttt
gccggatcaagagctaccaactctttttccgaaggtaactggcttcagcagagcgcagataccaaatactgtccttctagtgtagccgtagttagg
ccaccacttcaagaactctgtagcaccgcctacatacctcgctctgctaatcctgttaccagtggctgctgccagtggcgataagtcgtgtcttac
cgggttggactcaagacgatagttaccggataaggcgcagcggtcgggctgaacggggggttcgtgcacacagcccagcttggagcga
acgacctacaccgaactgagatacctacagcgtgagctatgagaaagcgccacgcttcccgaagggagaaaggcggacaggtatccgg
taagcggcagggtcggaacaggagagcgcacgagggagcttccagggggaaacgcctggtatctttatagtcctgtcgggtttcgccacc
tctgacttgagcgtcgatttttgtgatgctcgtcaggggggcggagcctatggaaaaacgccagcaacgcggcctttttacggttcctggcct
tttgctggccttttgctcacatgttctttcctgcgttatcccctgattctgtggataaccgtattaccgcctttgagtgagctgataccgctcgcc
gcagccgaacgaccgagcgcagcgagtcagtgagcgaggaagcggaagagcgcctgatgcggtattttctccttacgcatctgtgcggtattt
cacaccgcatatatggtgcactctcagtacaatctgctctgatgccgcatagttaagccagtatacactccgctatcgctacgtgactgggtca
tggctgcgccccgacacccgccaacacccgctgacgcgccctgacgggcttgtctgctcccggcatccgcttacagacaagctgtgacca
tctccgggagctgcatgtgtcagaggttttcaccgtcatcaccgaaacgcgcgaggcagctgcggtaaagctcatcagcgtggtcgtgaag
cgattcacagatgtctgcctgttcatccgcgtccagctcgttgagtttctccagaagcgttaatgtctggcttctgataaagcgggccatgttaa
gggcggttttttcctgtttggtcactgatgcctccgtgtaagggggatttctgttcatgggggtaatgataccgatgaaacgagagaggatgct
cacgatacgggttactgatgatgaacatgcccggttactggaacgttgtgagggtaaacaactggcggtatggatgcggcgggaccagag
aaaaatcactcagggtcaatgccagcgcttcgttaatacagatgtaggtgttccacagggtagccagcagcatcctgcgatgcagatccgg
aacataatggtgcagggcgctgacttccgcgtttccagactttacgaaacacggaaaccgaagaccattcatgttgttgctcaggtcgcaga
cgttttgcagcagcagtcgcttcacgttcgctcgcgtatcggtgattcattctgctaaccagtaaggcaaccccgccagcctagccgggtcct
caacgacaggagcacgatcatgcgcacccgtggccaggacccaacgctgcccgagatgcgccgcgtgcggctgctggagatggcgga
cgcgatggatatgttctgccaagggttggtttgcgcattcacagttctccgcaagaattgattggctccaattcttggagtggtgaatccgttag
cgaggtgccgccggcttccattcaggtcgaggtggcccggctccatgcaccgcgacgcaacgcggggaggcagacaaggtatagggc
ggcgcctacaatccatgccaacccgttccatgtgctcgccgaggcggcataaatcgccgtgacgatcagcggtccagtgatcgaagttag
gctggtaagagccgcgagcgatccttgaagctgtccctgatggtcgtcatctacctgcctggacagcatggcctgcaacgcgggcatccc
gatgccgccggaagcgagaagaatcataatggggaaggccatccagcctcgcgtcgcgaacgccagcaagacgtagcccagcgcgtc
ggccgccatgccggcgataatggcctgcttctcgccgaaacgtttggtgggggaccagtgacgaaggcttgagcgagggcgtgcaaga
ttccgaataccgcaagcgacaggtcgatcatcgtcgcgctccagcgaaagcggtcctcgccgaaaatgacccagagcgctgccggcac
ctgtcctacgagttgcatgataaagaagacagtcataagtgcggcgacgatagtcatgccccgcgcccaccggaaggagctgactgggtt
gaaggctctcaagggcatcggtcgagatcccggtgcctaatgagtgagctaacttacattaattgcgttgcgctcactgcccgctttccagtc
gggaaacctgtcgtgccagctgcattaatgaatcggccaacgcgcggggagaggcggtttgcgtattgggcgccagggtggtttttcttttc
accagtgaaacgggcaacagctgattgcccttcaccgcctggccctgagagagttgcagcaagcggtccacgctggtttgccccagcag
gcgaaaatcctgtttgatggtggttaacggcgggatataacatgagctgtcttcggtatcgtcgtatcccactaccgagatatccgcaccaac
gcgcagcccggactcggtaatggcgcgcattgcgcccagcgccatctgatcgttggcaaccagcatcgcagtgggaacgatgccctcatt
cagcatttgcatggtttgttgaaaaccggacatggcactccagtcgccttcccgttccgctatcggctgaatttgattgcgagtgagatatttat
gccagccagccagacgcagacgcgccgagacagaacttaatgggcccgctaacagcgcgatttgctggtgacccaatgcgaccagatg
ctccacgcccagtcgcgtaccgtcttcatgggagaaaataatactgttgatgggtgtctggtcagagacatcaagaaataacgccggaacat
tagtgcaggcagcttccacagcaatggcatcctggtcatccagcggatagttaatgatcagcccactgacgcgttgcgcgagaagattgtg
caccgccgctttacaggcttcgacgccgcttcgttctaccatcgacaccaccacgctggcacccagttgatcggcgcgagatttaatcgccg
cgacaatttgcgacggcgcgtgcagggccagactggaggtggcaacgccaatcagcaacgactgtttgcccgccagttgttgtgccacgc
ggttgggaatgtaattcagctccgccatcgccgcttccactttttcccgcgttttcgcagaaacgtggctggcctggttcaccacgcgggaaa
cggtctgataagagacaccggcatactctgcgacatcgtataacgttactggtttcacattcaccaccctgaattgactctcttccgggcgctat
catgccataccgcgaaaggttttgcgccattcgatggtgtccgggatctcgacgctctcccttatgcgactcctgcattaggaagcagccca
gtagtaggttgaggccgttgagcaccgccgccgcaaggaatggtgcatgaaggagatggcgcccaacagtcccccggccacggggc
ctgccaccatacccacgccgaaacaagcgctcatgagcccgaagtggcgagcccgatcttccccatcggtgatgtcggcgatataggcg
ccagcaaccgcacctgtggcgccggtgatgccggccacgatgcgtccggcgtagaggatcgagatctcgatcccgcgaaattaatacga
ctcactataggggaattgtgagcggataacaattcccctctagaaataattttgtttaactttaagaaggagatatactatggagaaaaaaatca
ctggatataccaccgttgatatatcccaatggcatcgtaaagaacattttgaggcatttcagtcagttgctcaatgtacctataaccagaccgttc
agctggatattacggcctttttaaagaccgtaaagaaaaataagcacaagttttatccggcctttattcacattcttgcccgcctgatgaatgctc
atccggaatttcgtatggcaatgaaagacggtgagctggtgatatgggatagtgttcacccttgttacaccgttttccatgagcaaactgaaac
gttttcatcgctctggagtgaataccacgacgatttccggcagtttctacacatatattcgcaagatgtggcgtgttacggtgaaaacctggcct
atttccctaaagggtttattgagaatatgtttttcgtttcagccaatccctgggtgagtttcaccagttttgatttaaacgtggccaatatggaca
acttcttcgcccccgttttcaccatgggcaaatattatacgcaaggcgacaaggtgctgatgccgctggcgattcaggttcatcatgccgtttgtg
atggcttccatgtcggcagaatgcttaatgaattacaacagtactgcgatgagtggcagggcggggcgggatcc

Glucosyltransferase protein sequences (SEQ ID Nos: 9-12) were codon optimized for E. coli using the IDTDNA codon optimization tool. BsaI type II restriction enzyme sequences were appended to the 5′ and 3′ of the each sequence and synthesized as a Gblock by IDTDNA (Coralville, IA). The resulting linear dsDNA was reacted with pNAB0098 (C-terminal His ×6 fusion (“His ×6” disclosed as SEQ ID NO: 28)) using the NEB® Golden Gate Assembly Kit (BsaI-HF®v2) according to the manufacturer's instructions. The reaction was transformed into E. coli strain TG1 (Lucigen) and plated onto LB agar containing ampicillin/chloramphenicol. Clones harboring the sequences of pNAB2009 (SEQ ID NO:24), pNAB2010 (SEQ ID NO:25), pNAB2011 (SEQ ID NO:26), and pNAB2012 (SEQ ID NO:27) corresponding to SEQ ID NO:9, SEQ ID NO:10, SEQ ID NO:11 and SEQ ID NO:12, respectively, were obtained after sequence verification by colony PCR, miniprep and plasmid DNA sequencing.

(9) pNAB2009
(SEQ ID NO: 24)
gaaaacctgtacttccagggccatcaccatcaccatcactgatcaatccggctgctaacaaagcccgaaaggaagctgagttggctgctgc
caccgctgagcaataactagcataaccccttggggcctctaaacgggtcttgaggggttttttgctgaaaggaggaactatatccggatatcc
cgcaagaggcccggcagtaccggcataaccaagcctatgcctacagcatccagggtgacggtgccgaggatgacgatgagcgcattgtt
agatttcatacacggtgcctgactgcgttagcaatttaactgtgataaactaccgcattaaagcttatcgatgataagctgtcaaacatgagaat
tcttgaagacgaaagggcctcgtgatacgcctatttttataggttaatgtcatgataataatggtttcttagacgtcaggtggcacttttcgggga
aatgtgcgcggaacccctatttgtttatttttctaaatacattcaaatatgtatccgctcatgagacaataaccctgataaatgcttcaataata
ttgaaaaaggaagagtatgagtattcaacatttccgtgtcgcccttattcccttttttgcggcattttgccttcctgtttttgctcacccagaaa
cgctggtgaaagtaaaagatgctgaagatcagttgggtgcacgagtgggttacatcgaactggatctcaacagcggtaagatccttgagagtttt
cgccccgaagaacgttttccaatgatgagcacttttaaagttctgctatgtggcgcggtattatcccgtgttgacgccgggcaagagcaactcggt
cgcgcatacactattctcagaatgacttggttgagtactcaccagtcacagaaaagcatcttacggatggcatgacagtaagagaattatgc
agtgctgccataaccatgagtgataacactgcggccaacttacttctgacaacgatcggaggaccgaaggagctaaccgcttttttgcacaa
catgggggatcatgtaactcgccttgatcgttgggaaccggagctgaatgaagccataccaaacgacgagcgtgacaccacgatgcctgc
agcaatggcaacaacgttgcgcaaactattaactggcgaactacttactctagcttcccggcaacaattaatagactggatggaggcggata
aagttgcaggaccacttctgcgctcggcccttccggctggctggtttattgctgataaatctggagccggtgagcgtggctctcgcggtatca
ttgcagcactggggccagatggtaagccctcccgtatcgtagttatctacacgacggggagtcaggcaactatggatgaacgaaatagaca
gatcgctgagataggtgcctcactgattaagcattggtaactgtcagaccaagtttactcatatatactttagattgatttaaaacttcattttt
aatttaaaaggatctaggtgaagatcctttttgataatctcatgaccaaaatcccttaacgtgagttttcgttccactgagcgtcagaccccgta
gaaaagatcaaaggatcttcttgagatcctttttttctgcgcgtaatctgctgcttgcaaacaaaaaaaccaccgctaccagcggtggtttgttt
gccggatcaagagctaccaactctttttccgaaggtaactggcttcagcagagcgcagataccaaatactgtccttctagtgtagccgtagttag
gccaccacttcaagaactctgtagcaccgcctacatacctcgctctgctaatcctgttaccagtggctgctgcaagtggcgataagtcgtgtctt
accgggttggactcaagacgatagttaccggataaggcgcagcggtcgggctgaacggggggttcgtgcacacagcccagcttggagcga
acgacctacaccgaactgagatacctacagcgtgagctatgagaaagcgccacgcttcccgaagggagaaaggcggacaggtatccgg
taagcggcagggtcggaacaggagagcgcacgagggagcttccagggggaaacgcctggtatctttatagtcctgtcgggtttcgccacc
tctgacttgagcgtcgatttttgtgatgctcgtcaggggggcggagcctatggaaaaacgccagcaacgcggcctttttacggttcctggcct
tttgctggccttttgctcacatgttctttcctgcgttatcccctgattctgtggataaccgtattaccgcctttgagtgagctgataccgctcgc
cgcagccgaacgaccgagcgcagcgagtcagtgagcgaggaagcggaagagcgcctgatgcggtattttctccttacgcatctgtgcggtattt
cacaccgcatatatggtgcactctcagtacaatctgctctgatgccgcatagttaagccagtatacactccgctatcgctacgtgactgggtca
tggctgcgccccgacacccgccaacacccgctgacgcgccctgacgggcttgtctgctcccggcatccgcttacagacaagctgtgacca
tctccgggagctgcatgtgtcagaggttttcaccgtcatcaccgaaacgcgcgaggcagctgcggtaaagctcatcagcgtggtcgtgaag
cgattcacagatgtctgcctgttcatccgcgtccagctcgttgagtttctccagaagcgttaatgtctggcttctgataaagcgggccatgttaa
gggcggttttttcctgtttggtcactgatgcctccgtgtaagggggatttctgttcatgggggtaatgataccgatgaaacgagagaggatgct
cacgatacgggttactgatgatgaacatgcccggttactggaacgttgtgagggtaaacaactggcggtatggatgcggcgggaccagag
aaaaatcactcagggtcaatgccagcgcttcgttaatacagatgtaggtgttccacagggtagccagcagcatcctgcgatgcagatccgg
aacataatggtgcagggcgctgacttccgcgtttccagactttacgaaacacggaaaccgaagaccattcatgttgttgctcaggtcgaga
cgttttgcagcagcagtcgcttcacgttcgctcgcgtatcggtgattcattctgctaaccagtaaggcaaccccgccagcctagccgggtcct
caacgacaggagcacgatcatgcgcacccgtggccaggacccaacgctgcccgagatgcgccgcgtgcggctgctggagatggcgga
cgcgatggatatgttctgccaagggttggtttgcgcattcacagttctccgcaagaattgattggctccaattcttggagtggtgaatccgttag
cgaggtgccgccggcttccattcaggtcgaggtggcccggctccatgcaccgcgacgcaacgcggggaggcagacaaggtatagggc
ggcgcctacaatccatgccaacccgttccatgtgctcgccgaggcggcataaatcgccgtgacgatcagcggtccagtgatcgaagttag
gctggtaagagccgcgagcgatccttgaagctgtccctgatggtcgtcatctacctgcctggacagcatggcctgcaacgcgggcatccc
gatgccgccggaagcgagaagaatcataatggggaaggccatccagcctcgcgtcgcgaacgccagcaagacgtagcccagcgcgtc
ggccgccatgccggcgataatggcctgcttctcgccgaaacgtttggtggcgggaccagtgacgaaggcttgagcgagggcgtgcaaga
ttccgaataccgcaagcgacaggtcgatcatcgtcgcgctccagcgaaagcggtcctcgccgaaaatgacccagagcgctgccggcac
ctgtcctacgagttgcatgataaagaagacagtcataagtgcggcgacgatagtcatgccccgcgcccaccggaaggagctgactgggtt
gaaggctctcaagggcatcggtcgagatcccggtgcctaatgagtgagctaacttacattaattgcgttgcgctcactgcccgctttccagtc
gggaaacctgtcgtgccagctgcattaatgaatcggccaacgcgcggggagaggcggtttgcgtattgggcgccagggtggtttttcttttc
accagtgaaacgggcaacagctgattgcccttcaccgcctggccctgagagagttgcagcaagcggtccacgctggtttgccccagcag
gcgaaaatcctgtttgatggtggttaacgggggatataacatgagctgtcttcggtatcgtcgtatcccactaccgagatatccgcaccaac
gcgcagcccggactcggtaatggcgcgcattgcgcccagcgccatctgatcgttggcaaccagcatcgcagtgggaacgatgccctcatt
cagcatttgcatggtttgttgaaaaccggacatggcactccagtcgccttcccgttccgctatcggctgaatttgattgcgagtgagatatttat
gccagccagccagacgcagacgcgccgagacagaacttaatgggcccgctaacagcgcgatttgctggtgacccaatgcgaccagatg
ctccacgcccagtcgcgtaccgtcttcatgggagaaaataatactgttgatgggtgtctggtcagagacatcaagaaataacgccggaacat
tagtgcaggcagcttccacagcaatggcatcctggtcatccagcggatagttaatgatcagcccactgacgcgttgcgcgagaagattgtg
caccgccgctttacaggcttcgacgccgcttcgttctaccatcgacaccaccacgctggcacccagttgatcggcgcgagatttaatcgccg
cgacaatttgcgacggcgcgtgcagggccagactggaggtggcaacgccaatcagcaacgactgtttgcccgccagttgttgtgccacgc
ggttgggaatgtaattcagctccgccatcgccgcttccactttttcccgcgttttcgcagaaacgtggctggcctggttcaccacgcgggaaa
cggtctgataagagacaccggcatactctgcgacatcgtataacgttactggtttcacattcaccaccctgaattgactctcttccgggcgctat
catgccataccgcgaaaggttttgcgccattcgatggtgtccgggatctcgacgctctcccttatgcgactcctgcattaggaagcagccca
gtagtaggttgaggccgttgagcaccgccgccgcaaggaatggtgcatgcaaggagatggcgcccaacagtcccccggccacggggc
ctgccaccatacccacgccgaaacaagcgctcatgagcccgaagtggcgagcccgatcttccccatcggtgatgtcggcgatataggcg
ccagcaaccgcacctgtggcgccggtgatgccggccacgatgcgtccggcgtagaggatcgagatctcgatcccgcgaaattaatacga
ctcactatagggg
gggaattgtgagcggataacaattcccctctagaaataattttgtttaactttaagaaggagatatactatgGCCTGTTTG
TTGAGAGCCTTTCAAAGAATATCTGCCGGTGTCTTCTTCTTGGCTTTGTGGGGTATGG
TOGTAGGTGATAAATTATTGGTAGTTCCACAAGATGGTTCACACTGGTTATCTATGA
AGGATATCGTTGAAGTCTTGTCAGATAGAGGTCACGAAATTGTTGTTGTAGTTCCTG
AAGTCAATTTATTGTTGAAGGAATCTAAGTATTATACAAGGAAAATTTATCCTGTCC
CTTACGACCAAGAAGAGTTGAAGAATAGATATCAGTCTTTTGGTAATAATCACTTCG
CTGAGAGGTCTTTCTTGACCGCTCCTCAAACTGAATACAGAAACAACATGATTGTAA
TAGGTTTGTATTTTATAAATTGTCAGTCTTTGTTACAAGATAGGGATACTTTGAACTT
CTTTAAAGAATCAAAATTTGATGCATTGTTTACTGATCCAGCTTTACCTTGCGGTGTC
ATCTTAGCCGAATACTTAGGTTTACCATCAGTTTATTTATTCAGAGGTTTCCCATGTT
CTTTAGAGCACGCATTTTCATCATCTCCTGACCCTGTATCATATATACCTAGATGCTA
CACAAAATTTTCAGATCACATGACATTTTCTCAGAGGGTCGCCAATTTCTTGGTCAA
CTTGTTAGAACCTTACTTGTTTTACTGTTTGTTCTCTAAATATGAAGAATTAGCTTCA
GCAGTCTTGAAGAGAGATGTAGATATAATAACATTGTACCAGAAAGTTTCAGTCTG
GTTATTGAGGTACGATTTTGTATTGGAGTACCCAAGACCTGTTATGCCTAACATGGT
TTTCATAGGAGGTATAAATTGTAAGAAAAGAAAAGATTTGTCTCAAGAGTTTGAGG
CATACATTAACGCATCTGGAGAACACGGTATAGTAGTTTTCTCTTtGGGTTCAATGG
TTTCAGAGATACCAGAAAAGAAAGCAATGGCAATCGCAGATGCTTTAGGTAAAATA
CCACAAACCGTCTTGTGGAGATATACAGGTACTAGGCCTTCTAACTTGGCCAATAAC
ACTATTTTAGTCAAATGGTTGCCACAAAACGATTTGTTGGGTCATCCTATGACCAGA
GCCTTTATTACACACGCCGGTTCACACGGAGTATATGAATCAATCTGTAACGGTGTT
CCTATGGTTATGATGCCATTGTTTGGTGATCAAATGGATAACGCTAAGAGGATGGAA
ACAAAAGGTGCTGGTGTAACTTTAAATGTTTTAGAAATGACTTCAGAAGATTTAGAA
AACGCTTTGAAGGCCGTTATTAATGACAAGTCATATAAAGAAAATATAATGAGATT
GTCATCTTTACATAAAGATAGACCTGTTGAACCTTTAGACTTGGCTGTTTTCTGGGTC
GAGTTTGTCATGAGACATAAAGGTGCTCCTCATTTAAGGCCAGCTGCCCATGATTTG
ACCTGGTATCAATACCATTCTTTGGACGTAATTGGTTTCTTATTAGCCGTOGTTTTGA
CAGTTGCATTCATCACTTTTAAGTGTTGCGCATACGGTTATAGAAAGTGCTTAGGTA
AGAAAGGAAGGGTTAAGAAAGCTCACAAATCTAAGACTCACGGATCC
(10) pNAB2010
(SEQ ID NO: 25)
gaaaacctgtacttccagggccatcaccatcaccatcactgatcaatccggctgctaacaaagcccgaaaggaagctgagttggctgctgc
caccgctgagcaataactagcataaccccttggggcctctaaacgggtcttgaggggttttttgctgaaaggaggaactatatccggatatcc
cgcaagaggcccggcagtaccggcataaccaagcctatgcctacagcatccagggtgacggtgccgaggatgacgatgagcgcattgtt
agatttcatacacgggcctgactgcgttagcaatttaactgtgataaactaccgcattaaagcttatcgatgataagctgtcaaacatgagaat
tcttgaagacgaaagggcctcgtgatacgcctatttttataggttaatgtcatgataataatggtttcttagacgtcaggtggcacttttcgggga
aatgtgcgcggaacccctatttgtttatttttctaaatacattcaaatatgtatccgctcatgagacaataaccctgataaatgcttcaataata
ttgaaaaaggaagagtatgagtattcaacattccgtgtcgcccttattcccttttttgcggcattttgccttcctgtttttgctcacccagaaac
gctggtgaaagtaaaagatgctgaagatcagttgggtgcacgagtgggttacatcgaactggatctcaacagcggtaagatccttgagagttttc
gccccgaagaacgttttccaatgatgagcacttttaaagttctgctatgtggcgcggtattatcccgtgttgacgccgggcaagagcaactcggt
cgccgcatacactattctcagaatgacttggttgagtactcaccagtcacagaaaagcatcttacggatggcatgacagtaagagaattatgc
agtgctgccataaccatgagtgataacactgcggccaacttacttctgacaacgatcggaggaccgaaggagctaaccgcttttttgcacaa
gggggatcatgtaactcgccttgatcgttgggaaccggagctgaatgaagccataccaaacgacgagcgtgacaccacgatgcctgc
agcaatggcaacaacgttgcgcaaactattaactggcgaactacttactctagcttcccggcaacaattaatagactggatggaggcggata
aagttgcaggaccacttctgcgctcggcccttccggctggctggtttattgctgataaatctggagccggtgagcgtggctctcgcggtatca
ttgcagcactggggccagatggtaagccctcccgtatcgtagttatctacacgacggggagtcaggcaactatggatgaacgaaatagaca
gatcgctgagataggtgcctcactgattaagcattggtaactgtcagaccaagtttactcatatatactttagattgatttaaaacttcattttt
aatttaaaaggatctaggtgaagatcctttttgataatctcatgaccaaaatcccttaacgtgagttttcgttccactgagcgtcagaccccgtag
aaaagatcaaaggatcttcttgagatcctttttttctgcgcgtaatctgctgcttgcaaacaaaaaaaccaccgctaccagcggtggtttgtttg
ccggatcaagagctaccaactctttttccgaaggtaactggcttcagcagagcgcagataccaaatactgtccttctagtgtagccgtagttagg
ccaccacttcaagaactctgtagcaccgcctacatacctcgctctgctaatcctgttaccagtggctgctgccagtggcgataagtcgtgtcttac
cgggttggactcaagacgatagttaccggataaggcgcagcggtcgggctgaacggggggttcgtgcacacagcccagcttggagcga
acgacctacaccgaactgagatacctacagcgtgagctatgagaaagcgccacgcttcccgaagggagaaaggcggacaggtatccgg
taagcggcagggtcggaacaggagagcgcacgagggagcttccagggggaaacgcctggtatctttatagtcctgtcgggtttcgccacc
tctgacttgagcgtcgatttttgtgatgctcgtcaggggggcggagcctatggaaaaacgccagcaacgcggcctttttacggttcctggcct
tttgctggccttttgctcacatgttctttcctgcgttatcccctgattctgtggataaccgtattaccgcctttgagtgagctgataccgctcgc
cgcagccgaacgaccgagcgcagcgagtcagtgagcgaggaagcggaagagcgcctgatgcggtattttctccttacgcatctgtgcggtattt
cacaccgcatatatggtgcactctcagtacaatctgctctgatgccgcatagttaagccagtatacactccgctatcgctacgtgactgggtca
tggctgcgccccgacacccgccaacacccgctgacgcgccctgacgggcttgtctgctcccggcatccgcttacagacaagctgtgacca
tctccgggagctgcatgtgtcagaggttttcaccgtcatcaccgaaacgcgcgaggcagctgcggtaaagctcatcagcgtggtcgtgaag
cgattcacagatgtctgcctgttcatccgcgtccagctcgttgagtttctccagaagcgttaatgtctggcttctgataaagcgggccatgttaa
gggcggttttttcctgtttggtcactgatgcctccgtgtaagggggatttctgttcatgggggtaatgataccgatgaaacgagagaggatgct
cacgatacgggttactgatgatgaacatgcccggttactggaacgttgtgagggtaaacaactggcggtatggatgcggcgggaccagag
aaaaatcactcagggtcaatgccagcgcttcgttaatacagatgtaggtgttccacagggtagccagcagcatcctgcgatgcagatccgg
aacataatggtgcagggcgctgacttccgcgtttccagactttacgaaacacggaaaccgaagaccattcatgttgttgctcaggtcgcaga
cgttttgcagcagcagtcgcttcaccttcgctcgcgtatcggtgattcattctgctaaccagtaaggcaaccccgccagcctagccgggtcct
caacgacaggagcacgatcatgcgcacccgtggccaggacccaacgctgcccgagatgcgccgcgtgcggctgctggagatggcgga
cgcgatggatatgttctgccaagggttggtttgcgcattcacagttctccgcaagaattgattggctccaattcttggagtggtgaatccgttag
cgaggtgccgccggcttccattcaggtcgaggtggcccggctccatgcaccgcgacgcaacgcggggaggcagacaaggtatagggc
ggcgcctacaatccatgccaacccgttccatgtgctcgccgaggcggcataaatcgccgtgacgatcagcggtccagtgatcgaagttag
gctggtaagagccgcgagcgatccttgaagctgtccctgatggtcgtcatctacctgcctggacagcatggcctgcaacgcgggcatccc
gatgccgccggaagcgagaagaatcataatggggaaggccatccagcctcgcgtcgcgaacgccagcaagacgtagcccagcgcgtc
ggccgccatgccggcgataatggcctgcttctcgccgaaacgtttggtggcgggaccagtgacgaaggcttgagcgagggcgtgcaaga
ttccgaataccgcaagcgacaggtcgatcatcgtcgcgctccagcgaaagcggtcctcgccgaaaatgacccagagcgctgccggcac
ctgtcctacgagttgcatgataaagaagacagtcataagtgcggcgacgatagtcatgccccgcgcccaccggaaggagctgactgggtt
gaaggctctcaagggcatcggtcgagatcccggtgcctaatgagtgagctaacttacattaattgcgttgcgctcactgcccgctttccagtc
gggaaacctgtcgtgccagctgcattaatgaatcggccaacgcgcggggagaggcggtttgcgtattgggcgccagggtggtttttcttttc
accagtgaaacgggcaacagctgattgcccttcaccgcctggccctgagagagttgcagcaagcggtccacgctggtttgccccagcag
gcgaaaatcctgtttgatggtggttaacggcgggatataacatgagctgtcttcggtatcgtcgtatcccactaccgagatatccgcaccaac
gcgcagcccggactcggtaatggcgcgcattgcgcccagcgccatctgatcgttggcaaccagcatcgcagtgggaacgatgccctcatt
cagcatttgcatggtttgttgaaaaccggacatggcactccagtcgccttcccgttccgctatcggctgaatttgattgcgagtgagatatttat
gccagccagccagacgcagacgcgccgagacagaacttaatgggcccgctaacagcgcgatttgctggtgacccaatgcgaccagatg
ctccacgcccagtcgcgtaccgtcttcatgggagaaaataatactgttgatgggtgtctggtcagagacatcaagaaataacgccggaacat
tagtgcaggcagcttccacagcaatggcatcctggtcatccagcggatagttaatgatcagcccactgacgcgttgcgcgagaagattgtg
caccgccgctttacaggcttcgacgccgcttcgttctaccatcgacaccaccacgctggcacccagttgatcggcgcgagatttaatcgccg
cgacaatttgcgacggcgcgtgcagggccagactggaggtggcaacgccaatcagcaacgactgtttgcccgccagttgttgtgccacgc
ggttgggaatgtaattcagctccgccatcgccgcttccactttttcccgcgttttcgcagaaacgtggctggcctggttcaccacgcgggaaa
cggtctgataagagacaccggcatactctgcgacatcgtataacgttactggtttcacattcaccaccctgaattgactctcttccgggcgctat
catgccataccgcgaaaggttttgcgccattcgatggtgtccgggatctcgacgctctcccttatgcgactcctgcattaggaagcagccca
gtagtaggttgaggccgttgagcaccgccgccgcaaggaatggtgcatgcaaggagatggcgcccaacagtcccccggccacggggc
ctgccaccatacccacgccgaaacaagcgctcatgagcccgaagtggcgagcccgatcttccccatcggtgatgtcggcgatataggcg
ccagcaaccgcacctgtggcgccggtgatgccggccacgatgcgtccggcgtagaggatcgagatctcgatcccgcgaaattaatacga
ctcactataggggaattgtgagcggataacaattcccctctagaaataattttgtttaactttaagaaggagatatactatgGCTACCAC
CCAGACCACTCCCGCCCACATCGCCATGTTCTCCATCGCCGCCCACGGCCATGTGAA
CCCCAGCCTGGAGGTGATCCGTGAACTCGTCGCCCGCGGCCACCGGGTCACGTACG
CCATTCCGCCCGTCTTCGCCGACAAGGTGGCCGCCACCGGCGCCCGGCCCGTCCTCT
ACCACTCCACCCTGCCCGGCACCGACGCCGACCCGGAGGCATGGGGAAGCACCCTG
CTGGACAACGTCGAACCGTTCCTGAACGACGCGATCCAGGCGCTCCCGCAGCTCGC
CGATGCCTACGCCGACGACATCCCCGATCTCGTCCTGCACGACATCACCTCCTACCC
GGCCCGCGTCCTGGCCCGCCGCTGGGGCGTCCCGGCGGTtTCCCTCTTCCCGAACCTC
GTCGCCTGGAAGGGTTACGAGGAGGAGGTCGCCGAGCCGATGTGGCGCGAACCCCG
GCAGACCGAGCGCGGACGGGCCTACTACGCCCGGTTCGAGGCATOGCTGAAGGAGA
ACGGGATCACCGAGCACCCGGACACGTTCGCCAGTCATCCGCCGCGCTCCCTGGTGC
TCATCCCGAAGGCGCTCCAGCCGCACGCCGACCGGGTGGACGAAGACGTGTACACC
TTCGTCGGCGCCTGCCAGGGAGAtCGCGCCGAGGAAGGCGGCTGGCAGCGGCCCGC
CGGCGCGGAGAAGGTCGTCCTGGTGTCGCTCGGCTCGGTGTTCACCAAGCAGCCCG
CCTTCTACCGGGAGTGCGTGCGCGCCTTCGGGAACCTGCCCGGCTGGCACCTCGTCC
TCCAGATCGGCCGGAAGGTGACCCCCGCCGAACTGGGGGAGCTGCCGGACAACGTG
GAGGTGCACGACTGGGTGCCGCAGCTCGCGATCCTGCGCCAGGCCGATCTGTTCGTC
ACCCACGCGGGCGCCGGCGGCAGCCAGGAGGGGCTGGCCACCGCGACGCCCATGAT
CGCCGTACCGCAGGCCGTCGACCAGTTCGGCAACGCCGACATGCTCCAAGGGCTCG
GCGTCGCCCGGAAGCTGGCGACCGAGGAGGCCACCGCCGACCTGCTCCGCGAaaccgc
cctcgctctggtggacgacccggaggtcgcgcgccggctccggcgaatccaggcggagatggcccaggagggcggcacccggggg
cggccgacctcatcgaggccgaactgcccgcgcgccacgagcggcaggagccggtgggcgaccgacccaacGGATCC
(11) pNAB2011
(SEQ ID NO: 26)
gaatcccccgccGCTCCACCAACCACCGCTCCACCACCGCACGTCATAATCGTGCCCTCCG
CCGGCATGGGCCACCTCATCCCCCTCGCCGAGTTCGCCAAGCGCCTCCTTCCGCGCT
TCACCTTCACCTTCGCCGTACCCACCAGCGGCCCGCCCTCATCCTCCCAGCGCGACT
TCCTCTCCTCCCTCCCTGCCTCCATCGACACCTCCTTCCTCCCCGAGGTCGACCTCTC
CGACGCCCCCTCCGACGCCCAAATCGAGACTCTCATGTCCCTCATGGTTGTCCGCTC
CCTCCCCTCGCTCCGCGACCTCATTGCCTCCTACTCCGCCTOCGGCCGCCGCGTCGCC
GCCCTCGTCGTCGACCTTTTCOCCACTGATGCAATCGACGTCGCCCTTGAGCTCGGC
ATCCGCCCTTTCATCTTCTTCCCCTCCACCGCCATGACCCTCTCCTTCTTCCTCCACCT
CGAGAAGCTTGATGAAACGGTGTCATGTGAGTTTGCCGAGCTGTCCGACCCGGTTCA
GATCCCCGGGTGTATTCCGGTCCACGGCAAGGATTTGATCGACCCGGTTCAGGATAG
GAAGAACGACGCCTACAAGTGGCTCCTCCACCACTCCAAGAGGTATAAATTGGCCG
AGGGTGTTATCGTAAATAGCTTCGAGGGTTTGGAGGGGGGACCGATCAGGGAGCTT
TTGCACCCCGAGCCGGGAAAGCCGCGGGTTTACCCGGTCGGACCGCTGATTCAGGC
CGGTTCGTGCGAGAAGGGGGCAGCTGCCCGGCCTGAGTGCTTGAAGTGGTTGGACC
AGCAGCCACGTGGATCCGTCCTATTCGTGAATTTCGGGAGTGGTGGGGTCCTCAGTA
CGGAGCAGCAGAACGAGCTTGCAGGTGTGCTGGCCCACAGCCAGCAGAGGTTCCTA
TGGGTGGTTAGGCCTCCAAACGACGGCATTGCCAACGCCACGTATTTCAGCGTCGAC
GGGGAGATCGACCCGTTGAAACTCCTGCCCGAGGGGTTCTTGGAGCAGACCGCGGG
CAGGGGTTTGGTCTTGCCAATGTGGGCCCCGCAGATCGATGTCTTAAGCCATGAGTC
GACGGGCGGGTTCTTGACGCATTGTGGGTGGAATTCAACACTGGAGAGCGTGTTCC
ATGGGGTACCACTAATTACATGGCCCCTCTATGCAGAGCAAAAGATGAACGCTGTT
ATGCTAACCGAGGGCCTGAGGGTGGGACTCAGACCCTCAGTGGGTAAGGATGGAAT
CATCCGAGGTGCTGAGATCGCACGAGTTATAGGGGAGTTGATGGAAGGTGAGGAAG
GGAAACGAATACGGAGTAAGATGCAGGAGTTGAAGCGTGCGGCTTCTGCTGTATTG
AGCAAAGATGGATCATCTACTCGAGCTCTTGAAGAGGTTGCAAAAATTTGGGAAAG
CAAGGTTGGATCCgaaaacctgtacttccagggccatcaccatcaccatcactgatcaatccggctgctaacaaagcccgaaa
ggaagctgagttggctgctgccaccgctgagcaataactagcataaccccttggggcctctaaacgggtcttgaggggttttttgctgaaag
gaggaactatatccggatatcccgcaagaggcccggcagtaccggcataaccaagcctatgcctacagcatccagggtgacggtgccga
ggatgacgatgagcgcattgttagatttcatacacggtgcctgactgcgttagcaatttaactgtgataaactaccgcattaaagcttatcgatg
ataagctgtcaaacatgagaattcttgaagacgaaagggcctcgtgatacgcctatttttataggttaatgtcatgataataatggtttcttagac
gtcaggtggcacttttcggggaaatgtgcgcggaacccctatttgtttatttttctaaatacattcaaatatgtatccgctcatgagacaataacc
ctgataaatgcttcaataatattgaaaaaggaagagtatgagtattcaacatttccgtgtcgcccttattcccttttttgcggcattttgccttc
ctgtttttgctcacccagaaacgctggtgaaagtaaaagatgctgaagatcagttgggtgcacgagtgggttacatcgaactggatctcaacagcg
gtaagatccttgagagttttcgccccgaagaacgttttccaatgatgagcacttttaaagttctgctatgtggcgcggtattatcccgtgttgacg
ccgggcaagagcaactcggtcgccgcatacactattctcagaatgacttggttgagtactcaccagtcacagaaaagcatcttacggatggc
atgacagtaagagaattatgcagtgctgccataaccatgagtgataacactgcggccaacttacttctgacaacgatcggaggaccgaagg
agctaaccgcttttttgcacaacatgggggatcatgtaactcgccttgatcgttgggaaccggagctgaatgaagccataccaaacgacgag
cgtgacaccacgatgcctgcagcaatggcaacaacgttgcgcaaactattaactggcgaactacttactctagcttcccggcaacaattaata
gactggatggaggcggataaagttgcaggaccacttctgcgctcggcccttcggctggctggtttattgctgataaatctggagccggtga
gcgtggctctcgcggtatcattgcagcactggggccagatggtaagccctcccgtatcgtagttatctacacgacggggagtcaggcaact
atggatgaacgaaatagacagatcgctgagataggtgcctcactgattaagcattggtaactgtcagaccaagtttactcatatatactttagat
tgatttaaaacttcatttttaatttaaaaggatctaggtgaagatcctttttgataatctcatgaccaaaatcccttaacgtgagttttcgttcc
actgagcgtcagaccccgtagaaaagatcaaaggatcttcttgagatcctttttttctgcgcgtaatctgctgcttgcaaacaaaaaaaccaccg
ctaccagcggtggtttgtttgccggatcaagagctaccaactctttttccgaaggtaactggcttcagcagagcgcagataccaaatactgtcct
tctagtgtagccgtagttaggccaccacttcaagaactctgtagcaccgcctacatacctcgctctgctaatcctgttaccagtggctgctgcca
gtggcgataagtcgtgtcttaccgggttggactcaagacgatagttaccggataaggcgcagcggtcgggctgaacggggggttcgtgcaca
cagcccagcttggagcgaacgacctacaccgaactgagatacctacagcgtgagctatgagaaagcgccacgcttcccgaagggagaa
aggcggacaggtatccggtaagcggcagggtcggaacaggagagcgcacgagggagcttccagggggaaacgcctggtatctttatag
tcctgtcgggtttcgccacctctgacttgagcgtcgatttttgtgatgctcgtcaggggggcggagcctatggaaaaacgccagcaacgcgg
cctttttacggttcctggccttttgctggccttttgctcacatgttctttcctgcgttatcccctgattctgtggataaccgtattaccgccttt
gagtgagctgataccgctcgccgcagccgaacgaccgagcgcagcgagtcagtgagcgaggaagcggaagagcgcctgatgcggtattttctc
cttacgcatctgtgcggtatttcacaccgcatatatggtgcactctcagtacaatctgctctgatgccgcatagttaagccagtatacactccgc
tatcgctacgtgactgggtcatggctgcgccccgacacccgccaacacccgctgacgcgccctgacgggcttgtctgctcccggcatccg
cttacagacaagctgtgaccatctccgggagctgcatgtgtcagaggttttcaccgtcatcaccgaaacgcgcgaggcagctgcggtaaag
ctcatcagcgtggtcgtgaagcgattcacagatgtctgcctgttcatccgcgtccagctcgttgagtttctccagaagcgttaatgtctggcttc
tgataaagcgggccatgttaagggcggttttttcctgtttggtcactgatgcctccgtgtaagggggatttctgttcatgggggtaatgataccg
atgaaacgagagaggatgctcacgatacgggttactgatgatgaacatgcccggttactggaacgttgtgagggtaaacaactggcggtat
ggatgcggcgggaccagagaaaaatcactcagggtcaatgccagcgcttcgttaatacagatgtaggtgttccacagggtagccagcagc
atcctgcgatgcagatccggaacataatggtgcagggcgctgacttccgcgtttccagactttacgaaacacggaaaccgaagaccattcat
gttgttgctcaggtcgcagacgttttgcagcagcagtcgcttcacgttcgctcgcgtatcggtgattcattctgctaaccagtaaggcaacccc
gccagcctagccgggtcctcaacgacaggagcacgatcatgcgcacccgtggccaggacccaacgctgcccgagatgcgccgcgtgc
ggctgctggagatggcggacgcgatggatatgttctgccaagggttggtttgcgcattcacagttctccgcaagaattgattggctccaattct
tggagtggtgaatccgttagcgaggtgccgccggcttccattcaggtcgaggtggcccggctccatgcaccgcgacgcaacgcggggag
gcagacaaggtatagggcggcgcctacaatccatgccaacccgttccatgtgctgccgaggcggcataaatcgccgtgacgatcagcg
gtccagtgatcgaagttaggctggtaagagccgcgagcgatccttgaagctgtccctgatggtcgtcatctacctgcctggacagcatggcc
tgcaacgcgggcatcccgatgccgccggaagcgagaagaatcataatggggaaggccatccagcctcgcgtcgcgaacgccagcaag
acgtagcccagcgcgtcggccgccatgccggcgataatggcctgcttctcgccgaaacgtttggtgggggaccagtgacgaaggcttg
agcgagggcgtgcaagattccgaataccgcaagcgacaggtcgatcatcgtcgcgctccagcgaaagcggtcctcgccgaaaatgacc
cagagcgctgccggcacctgtcctacgagttgcatgataaagaagacagtcataagtgcggcgacgatagtcatgccccgcgcccaccg
gaaggagctgactgggttgaaggctctcaagggcatcggtcgagatcccggtgcctaatgagtgagctaacttacattaattgcgttgcgct
cactgcccgctttccagtcgggaaacctgtcgtgccagctgcattaatgaatcggccaacgcgcggggagaggcggtttgcgtattgggc
gccagggtggtttttcttttcaccagtgaaacgggcaacagctgattgcccttcaccgcctggccctgagagagttgcagcaagcggtccac
gctggtttgccccagcaggcgaaaatcctgtttgatggtggttaacggcgggatataacatgagctgtcttcggtatcgtcgtatcccactacc
gagatatccgcaccaacgcgcagcccggactcggtaatggcgcgcattgcgcccagcgccatctgatcgttggcaaccagcatcgcagt
gggaacgatgccctcattcagcatttgcatggtttgttgaaaaccggacatggcactccagtcgccttcccgttccgctatcggctgaatttga
ttgcgagtgagatatttatgccagccagccagacgcagacgcgccgagacagaacttaatgggcccgctaacagcgcgatttgctggtga
cccaatgcgaccagatgctccacgcccagtcgcgtaccgtcttcatgggagaaaataatactgttgatgggtgtctggtcagagacatcaag
aaataacgccggaacattagtgcaggcagcttccacagcaatggcatcctggtcatccagcggatagttaatgatcagcccactgacgcgtt
gcgcgagaagattgtgcaccgccgctttacaggcttcgacgccgcttcgttctaccatcgacaccaccacgctggcacccagttgatcggc
gcgagatttaatcgccgcgacaatttgcgacggcgcgtgcagggccagactggaggtggcaacgccaatcagcaacgactgtttgcccg
ccagttgttgtgccacgcggttgggaatgtaattcagctccgccatcgccgcttccactttttcccgcgttttcgcagaaacgtggctggcctg
gttcaccacgcgggaaacggtctgataagagacaccggcatactctgcgacatcgtataacgttactggtttcacattcaccaccctgaattg
actctcttccgggcgctatcatgccataccgcgaaaggttttgcgccattcgatggtgtccgggatctcgacgctctcccttatgcgactcctg
cattaggaagcagcccagtagtaggttgaggccgttgagcaccgccgccgcaaggaatggtgcatgcaaggagatggcgcccaacagt
cccccggccacggggcctgccaccatacccacgccgaaacaagcgctcatgagcccgaagtggcgagcccgatcttccccatcggtgat
gtcggcgatataggcgccagcaaccgcacctgtggcgccggtgatgccggccacgatgcgtccggcgtagaggatcgagatctcgatcc
cgcgaaattaatacgactcactataggggaattgtgagcggataacaattcccctctagaaataattttgtttaactttaagaaggagatatact
atg
(12) pNAB2012
(SEQ ID NO: 27)
GCGTGTACTGGCTGGACTTCGCCGTTGCCGTTGTGTGTGTGCTTATTGTTAACTTGCG
GATTCGCGGAAGCGGGCAAATTGCTGGTGGTCCCGATGGATGGCAGTCACTGGTTC
ACTATGCGTTCCGTGGTAGAAAAATTAATCTTGCGCGGCCACGAAGTGGTAGTAGTC
ATGCCCGAAGTATCGTGGCAACTTGGACGCTCCTTGAATTGTACAGTGAAGACATAT
AGCACGAGCTATACTCTGGAAGATTTGGATCGCGAGtTTAAGGCGTTTGCACACGCT
CAATGGAAAGCTCAAGTCCGCAGCATTTATTCGCTTTTGATGGGATCCTATAACGAC
ATCTTCGACCTTTTTTTCTCAAATTGTCGTTCATTGTTTAAAGACAAAAAACTTGTCG
AGTATCTGAAGGAATCCTCGTTTGATGCAGTCTTCCTGGACCCCTTTGACAATTGCG
GGTTAATTGTGGCTAAATACTTTTGGTTGCCGTCAGTCGTGTTCGCTCGCGGTATCTT
GTGTCACTATCTGGAGGAAGGTGCCCAATGCCCTGCACCGCTTTCTTATGTTCCTCG
CATCTTGTTGGGCTTCTCGGATGCGATGACGTTTAAGGAGCGTGTGCGCAATCATAT
CATGCACCTTGAAGAGCATCTGTTATGCCACCGCTTTTTTAAAAACGCGCTGGAAAT
CGCCTCCGAGATTTTGCAGACTCCAGTAACTGAATACGACCTGTACAGCCACACATC
AATCTGGTTGCTTCGCACGGATTTCGTCTTAGATTACCCGAAACCGGTCATGCCTAA
TATGATTTTCATCGGTGGAATTAACTGCCATCAAGGGAAGCCTTTGCCGATGGAGTT
TGAGGCATATATTAATGCGTCAGGGGAACATGGGATTGTCGTCTTCAGTTTAGGCTC
AATGGTTTCCGAAATCCCAGAGAAAAAGGCCATGGCTATTGCTGACGCACTGGGAA
AAATCCCTCAAACTGTGTTGTGGCGCTATACGGGAACACGCCCGAGTAACTTGGCTA
ACAATACGATTCTTGTCAAGTGGTTACCACAGAATGATCTTCTTGGCCATCCCATGA
CTCGTGCATTTATCACACATGCGGGATCCCACGGAGTCTATGAGTCCATCTGTAATG
GAGTTCCCATGGTGATGATGCCTCTGTTCGGGGATCAAATGGATAACGCTAAGCGTA
TGGAAACTAAAGGCGCTGGAGTGACATTGAACGTACTTGAGATGACGAGCGAGGAC
TTAGAAAACGCACTTAAAGCAGTCATTAATGATAAATCCTATAAAGAGAATATCAT
GCGTTTAAGTTCCTTGCACAAGGACCGTCCAGTTGAACCCTTAGATCTGGCAGTATT
CTGGGTGGAATTTGTCATGCGCCATAAAGGAGCACCCCACTTACGCCCCGCGGCTCA
TGATTTGACCTGGTACCAGTACCACAGTCTTGACGTGATCGGGTTTTTATTGGCCGTT
GTGTTGACCGTTGCCTTTATTACATTCAAGTGTTGCGCGTATGGATACCGTAAGTGTT
TGGGCAAGAAGGGGCGCGTAAAAAAGGCGCACAAAAGTAAGACACACGGATCCgaa
aacctgtacttccagggccatcaccatcaccatcactgatcaatccggctgctaacaaagcccgaaaggaagctgagttggctgctgccac
cgctgagcaataactagcataaccccttggggcctctaaacgggtcttgaggggttttttgctgaaaggaggaactatatccggatatcccgc
aagaggcccggcagtaccggcataaccaagcctatgcctacagcatccagggtgacggtgccgaggatgacgatgagcgcattgttaga
tttcatacacggtgcctgactgcgttagcaatttaactgtgataaactaccgcattaaagcttatcgatgataagctgtcaaacatgagaattctt
gaagacgaaagggcctcgtgatacgcctatttttataggttaatgtcatgataataatggtttcttagacgtcaggtggcacttttcggggaaat
gtgcgcggaacccctatttgtttatttttctaaatacattcaaatatgtatccgctcatgagacaataaccctgataaatgcttcaataatattg
aaaaaggaagagtatgagtattcaacatttccgtgtcgcccttattcccttttttgcggcattttgccttcctgtttttgctcacccagaaacg
ctggtgaaagtaaaagatgctgaagatcagttgggtgcacgagtgggttacatcgaactggatctcaacagcggtaagatccttgagagttttcg
ccccgaagaacgttttccaatgatgagcacttttaaagttctgctatgtggcgcggtattatcccgtgttgacgccgggcaagagcaactcggtc
gccgcatacactattctcagaatgacttggttgagtactcaccagtcacagaaaagcatcttacggatggcatgacagtaagagaattatgcagt
gctgccataaccatgagtgataacactgcggccaacttacttctgacaacgatcggaggaccgaaggagctaaccgcttttttgcacaacat
gggggatcatgtaactcgccttgatcgttgggaaccggagctgaatgaagccataccaaacgacgagcgtgacaccacgatgcctgcag
caatggcaacaacgttgcgcaaactattaactggcgaactacttactctagcttcccggcaacaattaatagactggatggaggcggataaa
gttgcaggaccacttctgcgctcggcccttccggctggctggtttattgctgataaatctggagccggtgagcgtggctctcgcggtatcattg
cagcactggggccagatggtaagccctcccgtatcgtagttatctacacgacggggagtcaggcaactatggatgaacgaaatagacaga
tcgctgagataggtgcctcactgattaagcattggtaactgtcagaccaagtttactcatatatactttagattgatttaaaacttcatttttaa
tttaaaaggatctaggtgaagatcctttttgataatctcatgaccaaaatcccttaacgtgagttttcgttccactgagcgtcagaccccgtaga
aaagatcaaaggatcttcttgagatcctttttttctgcgcgtaatctgctgcttgcaaacaaaaaaaccaccgctaccagcggtggtttgtttgc
cggatcaagagctaccaactctttttccgaaggtaactggcttcagcagagcgcagataccaaatactgtccttctagtgtagccgtagttaggc
caccacttcaagaactctgtagcaccgcctacatacctcgctctgctaatcctgttaccagtggctgctgccagtggcgataagtcgtgtcttac
cgggttggactcaagacgatagttaccggataaggcgcagcggtcgggctgaacggggggttcgtgcacacagcccagcttggagcgaacg
acctacaccgaactgagatacctacagcgtgagctatgagaaagcgccacgcttcccgaagggagaaaggcggacaggtatccggtaa
gcggcagggtcggaacaggagagcgcacgagggagcttccagggggaaacgcctggtatctttatagtcctgtcgggtttcgccacctct
gacttgagcgtcgatttttgtgatgctcgtcaggggggcggagcctatggaaaaacgccagcaacgcggcctttttacggttcctggcctttt
gctggccttttgctcacatgttctttcctgcgttatcccctgattctgtggataaccgtattaccgcctttgagtgagctgataccgctcgccgca
gccgaacgaccgagcgcagcgagtcagtgagcgaggaagcggaagagcgcctgatgcggtattttctccttacgcatctgtgcggtatttc
acaccgcatatatggtgcactctcagtacaatctgctctgatgccgcatagttaagccagtatacactccgctatcgctacgtgactgggtcat
ggctgcgccccgacacccgccaacacccgctgacgcgccctgacgggcttgtctgctcccggcatccgcttacagacaagctgtgaccat
ctccgggagctgcatgtgtcagaggttttcaccgtcatcaccgaaacgcgcgaggcagctgcggtaaagctcatcagcgtggtcgtgaag
cgattcacagatgtctgcctgttcatccgcgtccagctcgttgagtttctccagaagcgttaatgtctggcttctgataaagcgggccatgttaa
gggcggttttttcctgtttggtcactgatgcctccgtgtaagggggatttctgttcatgggggtaatgataccgatgaaacgagagaggatgct
cacgatacgggttactgatgatgaacatgcccggttactggaacgttgtgagggtaaacaactggcggtatggatgcggcgggaccagag
aaaaatcactcagggtcaatgccagcgcttcgttaatacagatgtaggtgttccacagggtagccagcagcatcctgcgatgcagatccgg
aacataatggtgcagggcgctgacttccgcgtttccagactttacgaaacacggaaaccgaagaccattcatgttgttgctcaggtcgcaga
cgttttgcagcagcagtcgcttcacgttcgctcgcgtatcggtgattcattctgctaaccagtaaggcaaccccgccagcctagccgggtcct
caacgacaggagcacgatcatgcgcacccgtggccaggacccaacgctgcccgagatgcgccgcgtgcggctgctggagatggcgga
cgcgatggatatgttctgccaagggttggtttgcgcattcacagttctccgcaagaattgattggctccaattcttggagtggtgaatccgttag
cgaggtgccgccggcttccattcaggtcgaggtggcccggctccatgcaccgcgacgcaacgcggggaggcagacaaggtatagggc
ggcgcctacaatccatgccaacccgttccatgtgctcgccgaggcggcataaatcgccgtgacgatcagcggtccagtgatcgaagttag
gctggtaagagccgcgagcgatccttgaagctgtccctgatggtcgtcatctacctgcctggacagcatggcctgcaacgcgggcatccc
gatgccgccggaagcgagaagaatcataatggggaaggccatccagcctcgcgtcgcgaacgccagcaagacgtagcccagcgcgtc
ggccgccatgccggcgataatggcctgcttctcgccgaaacgtttggtggcgggaccagtgacgaaggcttgagcgagggcgtgcaaga
ttccgaataccgcaagcgacaggtcgatcatcgtcgcgctccagcgaaagcggtcctcgcccgaaaatgacccagagcgctgccggcac
ctgtcctacgagttgcatgataaagaagacagtcataagtgcggcgacgatagtcatgccccgcgcccaccggaaggagctgactgggtt
gaaggctctcaagggcatcggtcgagatcccggtgcctaatgagtgagctaacttacattaattgcgttgcgctcactgcccgctttccagtc
gggaaacctgtcgtgccagctgcattaatgaatcggccaacgcgcggggagaggcggtttgcgtattgggcgccagggtggtttttcttttc
accagtgaaacgggcaacagctgattgcccttcaccgcctggccctgagagagttgcagcaagcggtccacgctggtttgccccagcag
gcgaaaatcctgtttgatggtggttaacggcgggatataacatgagctgtcttcggtatcgtcgtatcccactaccgagatatccgcaccaac
gcgcagcccggactcggtaatggcgcgcattgcgcccagcgccatctgatcgttggcaaccagcatcgcagtgggaacgatgccctcatt
cagcatttgcatggtttgttgaaaaccggacatggcactccagtcgccttcccgttccgctatcggctgaatttgattgcgagtgagatatttat
gccagccagccagacgcagacgcgccgagacagaacttaatgggcccgctaacagcgcgatttgctggtgacccaatgcgaccagatg
ctccacgcccagtcgcgtaccgtcttcatgggagaaaataatactgttgatgggtgtctggtcagagacatcaagaaataacgcggaacat
tagtgcaggcagcttccacagcaatggcatcctggtcatccagcggatagttaatgatcagcccactgacgcgttgcgcgagaagattgtg
caccgccgctttacaggcttcgacgccgcttcgttctaccatcgacaccaccacgctggcacccagttgatcggcgcgagatttaatcgccg
cgacaatttgcgacggcgcgtgcagggccagactggaggtggcaacgccaatcagcaacgactgtttgcccgccagttgttgtgccacgc
ggttgggaatgtaattcagctccgccatcgccgcttccactttttcccgcgttttcgcagaaacgtggctggcctggttcaccacgcgggaaa
cggtctgataagagacaccggcatactctgcgacatcgtataacgttactggtttcacattcaccaccctgaattgactctcttccgggcgctat
catgccataccgcgaaaggttttgcgccattcgatggtgtccgggatctcgacgctctcccttatgcgactcctgcattaggaagcagccca
gtagtaggttgaggccgttgagcaccgccgccgcaaggaatggtgcatgcaaggagatggcgcccaacagtcccccggccacggggc
ctgccaccatacccacgccgaaacaagcgctcatgagcccgaagtggcgagcccgatcttccccatcggtgatgtcggcgatataggcg
ccagcaaccgcacctgtggcgccggtgatgccggccacgatgcgtccggcgtagaggatcgagatctcgatcccgcgaaattaatacga
ctcactataggggaattgtgagcggataacaattcccctctagaaataattttgtttaactttaagaaggagatatactatg

Those skilled in the art will appreciate that DNA sequences can be obtained through various cloning techniques and DNA synthesis methods. Those skilled in the art will appreciate that several DNA sequences can yield identical protein products.

### Expression and Purification of Transferase Proteins

Rosetta(DE3) cells (Novagen) were independently transformed with plasmids pNAB2002, pNAB2003, pNAB2005, pNAB2006, pNAB2008, pNAB2009, pNAB2010, pNAB2011, and pNAB2012, and selected on LB agar plates containing chloramphenicol and ampicillin. Overnight cultures were diluted into 4 L Terrific Broth (Fisher) with ampicillin selection, grown at 37° C. in an Innova 44 shaker (New Brunswick Scientific) at 200 rpm, and induced with 1 mM isopropyl β-D-1-thiogalactopyranoside (IPTG) at OD600 ˜3. Cultures were then grown at 18° C. for 21 h for protein expression, and the cells were harvested by centrifugation.

The cell pellets were resuspended in 50 mM HEPES pH 7.0, 300 mM NaCl, 25 mM imidazole pH 8.0, and 1 mM DTT. The cell suspension was lysed by freeze/thaw and sonication. Lysates were purified using Ni-NTA agarose beads (Qiagen), and the proteins dialyzed against 25 mM HEPES pH 7.0, 50 mM NaCl, and 1 mM DTT.

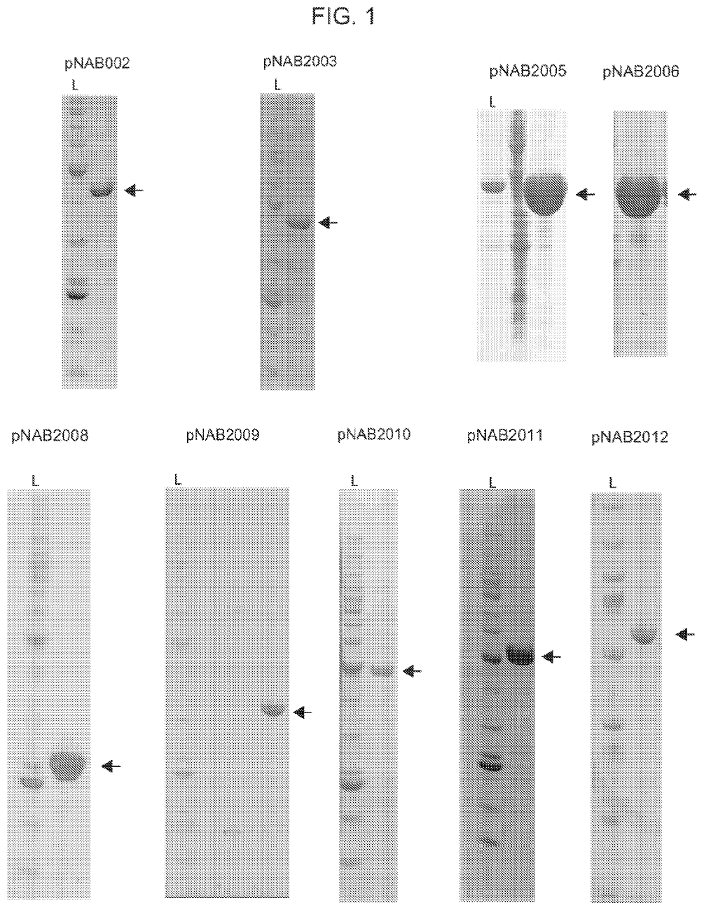

The N-terminal and C-terminal 6×His tags (“His ×6” disclosed as SEQ ID NO: 28) were TEV-cleaved, and the final purified protein was concentrated to 15 mg/mL using a 10,000 MWCO Amicon Ultra-15 Centrifugal Filter Unit (EMD Millipore). Protein molecular weights and purities of the respective expressed enzyme protein were confirmed by SDS-PAGE as shown in FIG. 1, as compared to Invitrogen™ BenchMark™ Protein Ladder (Catalog number: 10747012) as a standard. Proteins were used immediately for reactions or frozen at −80° C. for long term storage and thawed on ice before use in reactions.

### Example 3: Enzymatic Malonylation of 4-Hydroxy-N,N-Disopropyltryptamine

4-Hydroxy-N,N-diisopropyltryptamine (Cayman Chemicals MI, USA) was prepared by at 0.5 mg/mL concentration in a 1:1 mixture of DMSO:PBS at pH 7.5. Malonyl coenzyme A tetralithium salt and all buffers and reagents were purchased from Sigma-Aldrich, Inc., unless otherwise noted.

100-μL reaction mixture was prepared for each of Reactions 1-3 using the buffer, indole alkaloid substrate, co-substrate, enzyme, and reagents described in Table 3 below.

TABLE 3
Reaction Conditions of Reactions 1-3

| Reaction | | | Indole alkaloid | | |
|---|---|---|---|---|---|
| # | Buffer and Reagents | | Substrate | Co-Substrate | Enzyme |
| 1 | 50 | mM HEPES pH 7.5 | 1 mM 4-hydroxy-N,N- | 5 mM coenzyme A | 20 nM Purified |
| | 50 | mM NaCl | diisopropyltryptamine | sodium salt hydrate | enzyme of SEQ |
| | 5 | mM MgCl₂ | hydrochloride | | ID NO: 8 |
| | 1 | mM DTT | | | |
| 2 | 50 | mM HEPES pH 7.5 | 1 mM 4-hydroxy-N,N- | 5 mM malonyl | 20 nM purified |
| | 50 | mM NaCl | diisopropyltryptamine | coenzyme A | enzyme of SEQ |
| | 5 | mM MgCl₂ | hydrochloride | tetralithium salt | ID NO: 8 (pre- |
| | 1 | mM DTT | | | heated to 80 C. |
| | | | | | for 10 minutes to |
| | | | | | inactivate) |
| 3 | 50 | mM HEPES pH 7.5 | 1 mM 4-hydroxy-N,N- | 5 mM malonyl | 20 nM Purified |
| | 50 | mM NaCl | diisopropyltryptamine | coenzyme A | enzyme of SEQ |
| | 5 | mM MgCl₂ | hydrochloride | tetralithium salt | ID NO: 8 |
| | 1 | mM DTT | | | |

The reactions were carried out for 6 hours at 37° C., and then were quenched with 100 μL 100 mM NaOH. The reactions were centrifuged at 30,000 ref and the supernatant transferred to a fresh sample tube.

Five microliters of sample were injected onto a ZORBAX StableBond-C18, 1.8 um, 2.1×50 mm column at 20° C. using a 0.3 mL/min flow rate and major ion species were detected with a 6520 Accurate-Mass Q-TOF LC-MS (Agilent) in positive mode. The mobile phase consisted of a mixture of 0.1% formic acid in acetonitrile (viv) and 0.1% formic acid in water (viv) was eluted under the following gradient conditions (shown in relation to acetonitrile content): 0 min-10%, 6 min-100%, 7 min-10%, 14 min-10%. The mobile phase was delivered at a flow rate of 0.3 mL/min and the total analytical run time was 14 min.

A m/z value of 347.18 for the [M+H]+ adduct with the predicted parent compound was used to detect the malonylation products based on the predicted parent compound mass (346.18 g/mol) using Agilent MassHunter software.

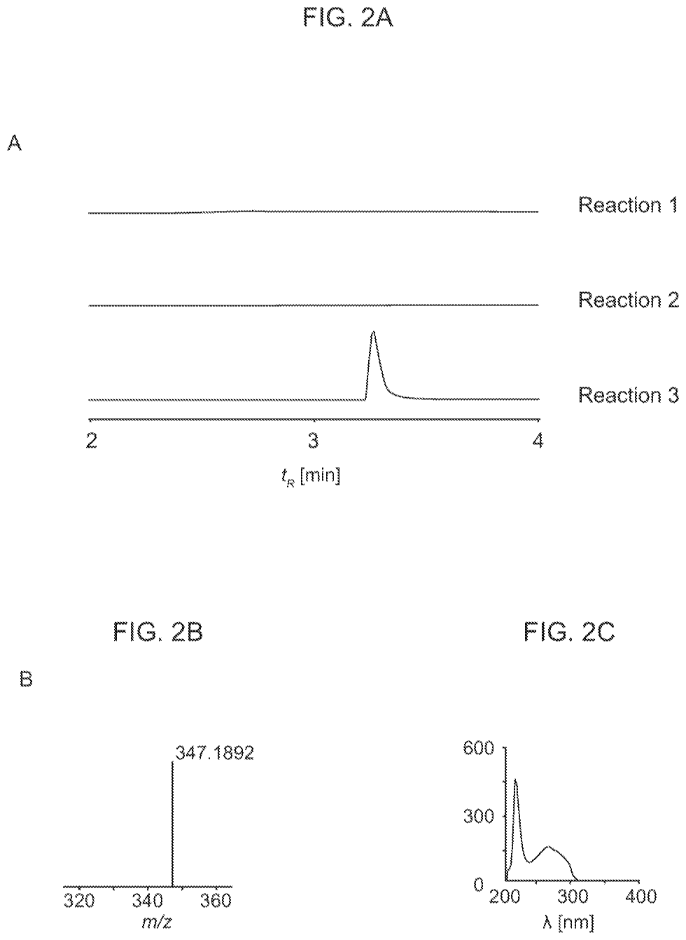

As shown in FIG. 2A, the LC-MS traces of the products of Reactions 1-3 showed that the desired product of 3-(3-(2-(diisopropylamino)ethyl)-1H-indol-4-yl)oxy)-3-oxopropanoic acid was not detected in Reaction 1 and Reaction 2, but was detected in Reaction 3. The desired acylation of the 4-hydroxy-N,N-diisopropyltryptamine substrate is dependent on the presence of both the malonyl coenzyme A and active enzyme (SEQ ID NO:8). FIGS. 2B and 2C show the mass peak of 347.1892 and the UV-vis absorption spectrum of Reaction 3 product, respectively. The product of Reaction 3 and mass analysis thereof are summarized in Table 4 below.

TABLE 4
Product of Enzymatic Malonylation of 4-Hydroxy-N,N-Diisopropyltryptamine

| | | Chemical Name | Product | Product | Indole alkaloid | Co- | |
|---|---|---|---|---|---|---|---|
| Reaction | Product Structure | of the Product | MW | (M + H)+ | substrate | substrate | Enzyme |
| 3 | 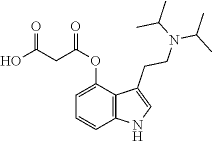 [SMILES: CC(C)N(CCc1c[nH]c2cccc(OC(=O)CC(=O)O)c12)C(C)C] | 3-((3-(2-(diiso- propylamino)- ethyl)-1H-indol- 4-yl)oxy)-3- oxopropanoic acid | 346.19 | 347.19 | 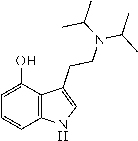 [SMILES: CC(C)N(CCc1c[nH]c2cccc(O)c12)C(C)C] | malonyl co- enzyme A | Purified enzyme of SEQ ID NO: 8 |

### Example 4: Enzymatic Glycosylation of 4-hydroxy-N,N-diisopropyltryptamine

4-Hydroxy-N,N-diisopropyltryptamine (Cayman Chemicals MI, USA) was prepared by at 0.5 mg/mL concentration in a 1:1 mixture of DMSO:PBS at pH 7.5. Uridine 5′-diphosphoglucose disodium salt hydrate (UDP-glucose) was purchased from Sigma-Aldrich, Inc MO, USA. All buffers and reagents were purchased from Sigma-Aldrich, Inc, unless otherwise noted.

100-μL reaction mixture was prepared for each of Reactions 4-8 using the buffer, indole alkaloid substrate, co-substrate, enzyme, and reagents described in Table 5 below.

TABLE 5
Reaction Conditions of Reactions 4-8

| Reaction | | | Indole alkaloid | | |
|---|---|---|---|---|---|
| # | Buffer and Reagents | | Substrate | Co-Substrate | Enzyme |
| 4 | 50 | mM HEPES pH 7.5 | 1 mM 4-hydroxy-N,N- | 5 mM UDP- | 20 nM Purified |
| | 50 | mM NaCl | diisopropyltryptamine | glucose | enzyme of SEQ |
| | 5 | mM MgCl₂ | hydrochloride | | ID NO: 9 |
| | 1 | mM DTT | | | |
| 5 | 50 | mM HEPES pH 7.5 | 1 mM 4-hydroxy-N,N- | 5 mM UDP- | 20 nM Purified |
| | 50 | mM NaCl | diisopropyltryptamine | glucose | enzyme of SEQ |
| | 5 | mM MgCl₂ | hydrochloride | | ID NO: 10 |
| | 1 | mM DTT | | | |
| 6 | 50 | mM HEPES pH 7.5 | 1 mM 4-hydroxy-N,N- | 5 mM UDP- | 20 nM Purified |
| | 50 | mM NaCl | diisopropyltryptamine | glucose | enzyme of SEQ |
| | 5 | mM MgCl₂ | hydrochloride | | ID NO: 11 |
| | 1 | mM DTT | | | |
| 7 | 50 | mM HEPES pH 7.5 | 1 mM 4-hydroxy-N,N- | 5 mM UDP- | 20 nM Purified |
| | 50 | mM NaCl | diisopropyltryptamine | glucose | enzyme of SEQ |
| | 5 | mM MgCl₂ | hydrochloride | | ID NO: 12 |
| | 1 | mM DTT | | | |
| 8 | 50 | mM HEPES pH 7.5 | 1 mM 4-hydroxy-N,N- | 5 mM UDP- | No enzyme |
| | 50 | mM NaCl | diisopropyltryptamine | glucose | (control) |
| | 5 | mM MgCl₂ | hydrochloride | | |
| | 1 | mM DTT | | | |

The reactions were carried out for 3 hours at 37° C., and were then quenched with 100 μL 100 mM NaOH. The reactions were centrifuged at 30,000 ref and the supernatant transferred to a fresh sample tube.

Five microliters of sample were injected onto a ZORBAX StableBond-C18, 1.8 um, 2.1×50 mm column at 20° C. using a 0.3 mL/min flow rate and major ion species were detected with a 6520 Accurate-Mass Q-TOF LC-MS (Agilent) in positive mode. The mobile phase consisted of a mixture of 0.1% formic acid in acetonitrile (v/v) and 0.1% formic acid in water (v/v) was eluted under the following gradient conditions (shown in relation to acetonitrile content): 0 min-10%, 6 min-100%, 7 min-10%, 14 min-10%. The mobile phase was delivered at a flow rate of 0.3 mL/min and the total analytical run time was 14 min.

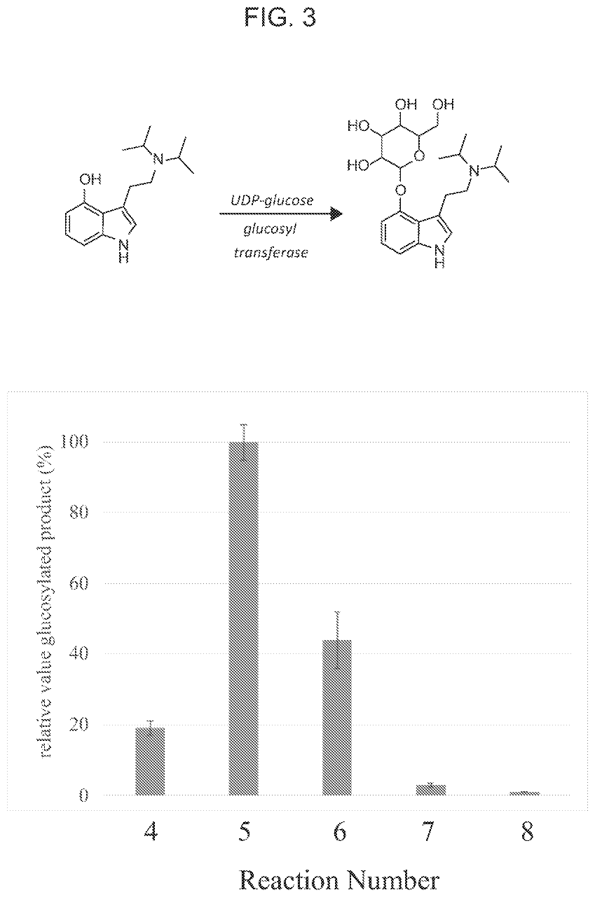

A m/z value of 423.24 for the [M+H]+ adduct with the predicted parent compound was used to detect glucosylation products based on the predicted parent compound mass (422.24 g/mol) using Agilent MassHunter software. No glycosylated product mass was observed in heat-inactivated enzyme samples or in Reaction 8. The relative ion count abundance of the glucosylated product (2-((3-(2-(diisopropylamino)ethyl)-1H-indol-4-yl)oxy)-6-(hydroxymethyl)tetrahydro-2H-pyran-3,4,5-triol) by glucosyltransferase enzymes in Reactions 4-8 are shown in FIG. 3. The reaction product of Reaction 3 and mass analysis thereof are summarized in Table 6 below.

TABLE 6
Product of Enzymatic Glucosylation of 4-Hydroxy-N,N-Diisopropyltryptamine

| Reaction | | Chemical Name | Product | Product | Indole alkaloid | Co- | |
|---|---|---|---|---|---|---|---|
| Number | Product Structure | of the Product | MW | (M + H)+ | substrate | substrate | Enzyme |
| 4 | 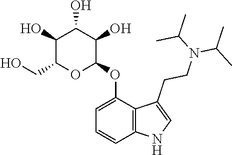 [SMILES: CC(C)N(CCc1c[nH]c2cccc(O[C@H]3O[C@H](CO)[C@@H](O)[C@H](O)[C@H]3O)c12)C(C)C] | 2-((3-(2-(diiso- propylamino)- ethyl)-1H-indol- 4-yl)oxy)-6- (hydroxymeth- yl)tetrahydro- 2H-pyran- 3,4,5-triol | 422.24 | 423.24 | 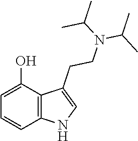 [SMILES: CC(C)N(CCc1c[nH]c2cccc(O)c12)C(C)C] | UDP- glucose | Purified enzyme of SEQ ID NO: 9 |
| 5 | 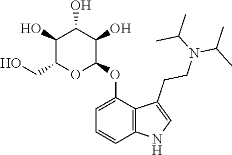 [SMILES: CC(C)N(CCc1c[nH]c2cccc(O[C@H]3O[C@H](CO)[C@@H](O)[C@H](O)[C@H]3O)c12)C(C)C] | 2-((3-(2-(diiso- propylamino)- ethyl)-1H-indol- 4-yl)oxy)-6- (hydroxymeth- yl)tetrahydro- 2H-pyran- 3,4,5-triol | 422.24 | 423.24 | 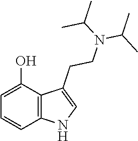 [SMILES: CC(C)N(CCc1c[nH]c2cccc(O)c12)C(C)C] | UDP- glucose | Purified enzyme of SEQ ID NO: 10 |
| 6 | 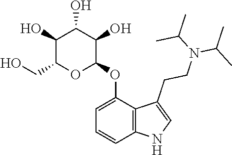 [SMILES: CC(C)N(CCc1c[nH]c2cccc(O[C@H]3O[C@H](CO)[C@@H](O)[C@H](O)[C@H]3O)c12)C(C)C] | 2-((3-(2-(diiso- propylamino)- ethyl)-1H-indol- 4-yl)oxy)-6- (hydroxymeth- yl)tetrahydro- 2H-pyran- 3,4,5-triol | 422.24 | 423.24 | 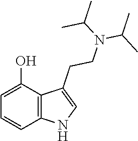 [SMILES: CC(C)N(CCc1c[nH]c2cccc(O)c12)C(C)C] | UDP- glucose | Purified enzyme of SEQ ID NO: 11 |
| 7 | 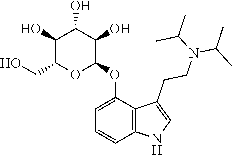 [SMILES: CC(C)N(CCc1c[nH]c2cccc(O[C@H]3O[C@H](CO)[C@@H](O)[C@H](O)[C@H]3O)c12)C(C)C] | 2-((3-(2-(diiso- propylamino)- ethyl)-1H-indol- 4-yl)oxy)-6- (hydroxymeth- yl)tetrahydro- 2H-pyran- 3,4,5-triol | 422.24 | 423.24 | 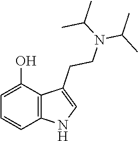 [SMILES: CC(C)N(CCc1c[nH]c2cccc(O)c12)C(C)C] | UDP- glucose | Purified enzyme of SEQ ID NO: 11 |

### Example 5: Enzymatic Sulfonation of 4-Hydroxy-N,N-diisopropyltryptamine

4-Hydroxy-N,N-diisopropyltryptamine (Cayman Chemicals MI, USA) was prepared by at 0.5 mg/mL concentration in a 1:1 mixture of DMSO:PBS at pH 7.5. Adenosine Y-phosphate 5′-phosphosulfate lithium salt hydrate (PAPS) was purchased from Sigma-Aldrich. Inc MO, USA. All buffers and reagents were purchased from Sigma-Aldrich, Inc, unless otherwise noted.

0.100-μL reaction mixture was prepared for each of Reactions 9-11 using the buffer, indole alkaloid substrate, co-substrate, enzyme, and reagents described in Table 7 below.

TABLE 7
Reaction Conditions of Reactions 9-11

| Reaction | | | Indole alkaloid | | |
|---|---|---|---|---|---|
| # | Buffer and Reagents | | Substrate | Co-Substrate | Enzyme |
| 9 | 50 | mM HEPES pH 7.5 | 1 mM 4-hydroxy-N,N- | 5 mM PAPS | 20 nM Purified |
| | 50 | mM NaCl | diisopropyltryptamine | | enzyme of SEQ |
| | 5 | mM MgCl₂ | hydrochloride | | ID NO: 5 |
| | 1 | mM DTT | | | |
| 10 | 50 | mM HEPES pH 7.5 | 1 mM 4-hydroxy-N,N- | 5 mM PAPS | 20 nM Purified |
| | 50 | mM NaCl | diisopropyltryptamine | | enzyme of SEQ |
| | 5 | mM MgCl₂ | hydrochloride | | ID NO: 6 |
| | 1 | mM DTT | | | |
| 11 | 50 | mM HEPES pH 7.5 | 1 mM 4-hydroxy-N,N- | 5 mM PAPS | No enzyme |
| | 50 | mM NaCl | diisopropyltryptamine | | (control) |
| | 5 | mM MgCl₂ | hydrochloride | | |
| | 1 | mM DTT | | | |

The reactions were carried out for 5 hours at 37° C., and then were quenched with 100 μL 100 mM NaOH. The reactions were centrifuged at 30,000 ref and the supernatant transferred to a fresh sample tube.

Five microliters of sample were injected onto a ZORBAX StableBond-C18, 1.8 um, 2.1×50 mm column at 20° C. using a 0.3 mL/min flow rate and major ion species were detected with a 6520 Accurate-Mass Q-TOF LC-MS (Agilent) in positive mode. The mobile phase consisted of a mixture of 0.1% formic acid in acetonitrile (v/v) and 0.1% formic acid in water (v/v) was eluted under the following gradient conditions (shown in relation to acetonitrile content): 0 min-10%, 6 min-100%, 7 min-10%, 14 min-10%. The mobile phase was delivered at a flow rate of 0.3 mL/min and the total analytical run time was 14 min.

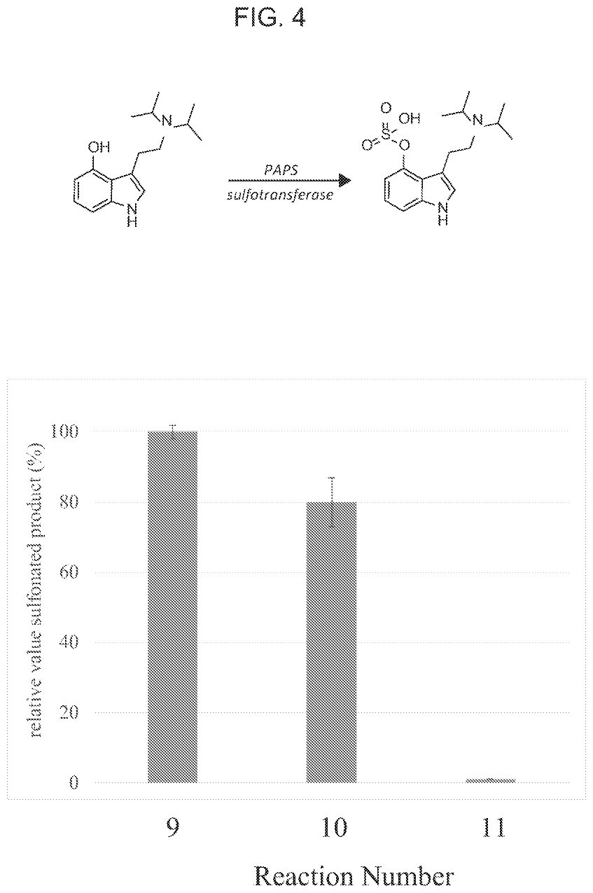

A m/z value of 341.15 for a [M+H]+ adduct with the parent compound was used to detect sulfonation products based on the predicted parent compound mass (340.15 g/mol) using Agilent MassHunter software. No sulfonated product mass was observed in heat-inactivated enzyme sample from Reaction 11. The relative ion count abundance of sulfonated product 3-(2-(diisopropylamino)ethyl)-1H-indol-4-yl hydrogen sulfate by sulfotransferase enzymes in Reactions 9-11 are shown in FIG. 4. The reaction product of Reactions 9 and 10 are summarized in Table 8 below.

TABLE 8
Product of Enzymatic Sulfonation of 4-Hydroxy-N,N-Diisopropyltryptamine

| Reaction | | Chemical Name | Product | Product | Indole alkaloid | Co- | |
|---|---|---|---|---|---|---|---|
| Number | Product Structure | of the Product | MW | (M + H)+ | substrate | substrate | Enzyme |
| 9 | 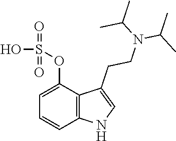 [SMILES: CC(C)N(CCc1c[nH]c2cccc(OS(=O)(=O)O)c12)C(C)C] | 3-(2-(diisopropyl- amino)ethyl)-1H- indol-4-yl hydro- gen sulfate | 340.15 | 341.15 | 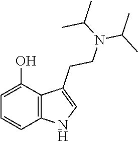 [SMILES: CC(C)N(CCc1c[nH]c2cccc(O)c12)C(C)C] | PAPS | Purified enzyme of SEQ ID NO: 5 |
| 10 | 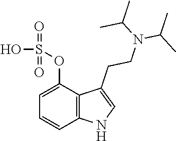 [SMILES: CC(C)N(CCc1c[nH]c2cccc(OS(=O)(=O)O)c12)C(C)C] | 3-(2-(diisopropyl- amino)ethyl)-1H- indol-4-yl hydro- gen sulfate | 340.15 | 341.15 | 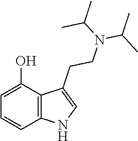 [SMILES: CC(C)N(CCc1c[nH]c2cccc(O)c12)C(C)C] | PAPS | Purified enzyme of SEQ ID NO: 6 |

### Example 6: Enzymatic Modification of Indole Alkaloids

Indole alkaloids were prepared by at 0.5 mg/ml concentration in a 1:1 mixture of DMSO:PBS at pH 7.5. All buffers and reagents were purchased from Sigma-Aldrich, Inc, unless otherwise noted. Noribogaine Hydrochloride was purchased from Toronto Research Chemicals, Toronto, ON, Canada.

### Indole Alkaloid Substrates

The following indole alkaloid substrates were used in this Example:
4-hydroxy-N,N-diisopropyltryptamine hydrochloride (Cayman Chemicals MI, USA)
4-hydroxy-N,N-dipropyltryptamine (Cayman Chemicals MI, USA)
7-hydroxymitragynine (Cayman Chemicals MI, USA)
Noribogaine Hydrochloride (Toronto Research Chemicals, Toronto, ON, Canada)
4-hydroxy-5-methyl-N,N-dimethyltryptamine (Chemspace)
5-hydroxy-N,N-diisopropyltryptamine (Chemspace)

### Co-Substrates

The following co-substrates were used in this Example:
Propanoyl Coenzyme A (sodium salt) (Avanti Polar Lipids, Inc AL, USA)
butanoyl Coenzyme A (sodium salt)(Avanti Polar Lipids, Inc AL, USA)
hexanoyl Coenzyme A (ammonium salt) (Avanti Polar Lipids, Inc AL, USA)
octanoyl Coenzyme A (ammonium salt) (Avanti Polar Lipids, Inc AL, USA)
decanoyl Coenzyme A (ammonium salt) (Avanti Polar Lipids, Inc AL, USA)
(E)-but-2-enoyl Coenzyme A (sodium salt) (Avanti Polar Lipids, Inc AL, USA)
Acetyl Coenzyme A (sodium salt) (Cayman Chemicals MI, USA)
Isobutyryl-Coenzyme A (sodium salt) (Cayman Chemicals MI, USA)
Acetoacetyl-Coenzyme A (sodium salt hydrate)(Cayman Chemicals MI, USA)
Adipoyl-Coenzyme A (sodium salt) (Cayman Chemicals MI, USA)
Cyclohexanoyl-Coenzyme A (Cayman Chemicals MI, USA)
Ethylmalonyl Coenzyme A (sodium salt) (Cayman Chemicals MI, USA)
Methylmalonyl-Coenzyme A (sodium salt)(Cayman Chemicals MI, USA)
S-(5′-Adenosyl)-L-methionine chloride (hydrochloride) (Cayman Chemicals MI, USA)
Coenzyme A sodium salt hydrate (Sigma-Aldrich, Inc MO, USA)
Malonyl coenzyme A tetralithium salt (Sigma-Aldrich, Inc MO, USA)
Succinyl coenzyme A sodium salt (Sigma-Aldrich, Inc MO, USA)
Glutaryl-Coenzyme A (Sigma-Aldrich, Inc MO, USA)
n-Heptanoyl coenzyme A (lithium salt) (Sigma-Aldrich, Inc MO, USA)
Adenosine 5′-triphosphate disodium salt hydrate (ATP) (Sigma-Aldrich, Inc MO, USA)
Uridine 5′-diphosphoglucose disodium salt hydrate (UDP-glucose) (Sigma-Aldrich, Inc MO, USA)
Adenosine 3′-phosphate 5′-phosphosulfate lithium salt hydrate (PAPS) (Sigma-Aldrich, Inc MO, USA)

### Enzymatic Modification of Indole Alkaloids

The following enzymatic modifications were performed using the buffer, indole alkaloid substrate, co-substrate, enzyme, and reagents as described below.

Acylation (Reactions 12a-29, 31-33, 38 and 39)
100-μL reaction mixtures were prepared, consisting of
50 mM HEPES pH 7.5
50 mM NaCl
5 mM MgCl₂
1 mM DTT
5 mM acyl coenzyme A
1 mM indole alkaloid substrate
50 μM purified enzyme of SEQ ID NO:8

### Phosphorylation (Reaction 35, 36 and 37)

100-μL reaction mixtures were prepared, consisting of
50 mM HEPES pH 7.5
50 mM NaCl
5 mM MgCl₂
1 mM DTT
10 mM ATP
1 mM indole alkaloid
10 μM purified enzyme of SEQ ID NO:2

### Glucosylation (Reactions 30 and 34)

100-μL reaction mixtures were prepared, consisting of
50 mM HEPES pH 7.5
50 mM NaCl
1 mM DTT
5 mM MgCl₂
10 mM UDP-glucose
1 mM indole alkaloid
25 μM purified enzyme of SEQ ID NO:10

### Methylation (Reaction 40)

100-μL reaction mixtures were prepared, consisting of
50 mM HEPES pH 7.5
50 mM NaCl
1 mM DTT
5 mM MgCl₂
10 mM S-(5-Adenosyl)-L-methionine chloride (hydrochloride) (SAM)
1 mM indole alkaloid
25 μM purified enzyme of SEQ ID NO:3

The reactions were carried out for 6 hours at 37° C., and then were quenched with 100 μL 100 mM NaOH. The reactions were centrifuged at 30,000 ref and the supernatant transferred to a fresh sample tube.

Five microliters of sample were injected onto a ZORBAX StableBond-C18, 1.8 um, 2.1×50 mm column at 20° C. using a 0.3 mL/min flow rate and major ion species were detected with a 6520 Accurate-Mass Q-TOF LC-MS (Agilent) in positive mode. The mobile phase consisted of a mixture of 0.1% formic acid in acetonitrile (v/v) and 0.1% formic acid in water (v/v) was eluted under the following gradient conditions (shown in relation to acetonitrile content): 0 min-10%, 6 min-100%, 7 min-10%, 14 min-10%. The mobile phase was delivered at a flow rate of 0.3 mL/min and the total analytical run time was 14 min. Masses for modified indole alkaloid products were not detected in heat inactivated enzyme samples, indole alkaloid samples or cofactor samples alone.

The reaction products of Reactions 12a to 40 are summarized in Table 9 below.

TABLE 9
Product of Enzymatic Modifications of Indole Alkaloid Substrates

| Reaction | | Chemical Name | Product | Product | | | |
|---|---|---|---|---|---|---|---|
| Number | Product Structure | of the Product | MW | (M + H)+ | Indole alkaloid substrate | Co-substrate | Enzyme |
| 12a | 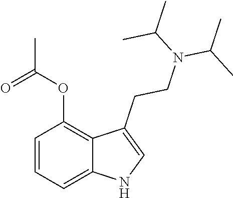 [SMILES: CC(=O)Oc1cccc2[nH]cc(CCN(C(C)C)C(C)C)c12] | 3-(2-(diisopropyl- amino)ethyl)-1H- indol-4-yl acetate | 302.2 | 303.2 | 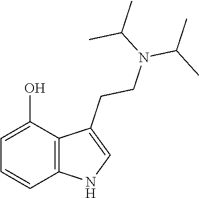 [SMILES: CC(C)N(CCc1c[nH]c2cccc(O)c12)C(C)C] | Acetyl Coenzyme A | Purified enzyme of SEQ ID NO: 8 |
| 12 | 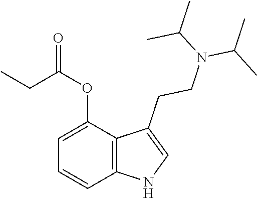 [SMILES: CCC(=O)Oc1cccc2[nH]cc(CCN(C(C)C)C(C)C)c12] | 3-(2-(diisopropyl- amino)ethyl)- 1H-indol-4-yl propionate | 316.22 | 317.22 | 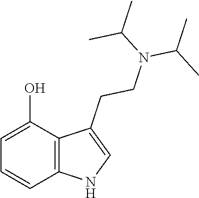 [SMILES: CC(C)N(CCc1c[nH]c2cccc(O)c12)C(C)C] | Propanoyl Coenzyme A | Purified enzyme of SEQ ID NO: 8 |
| 13 | 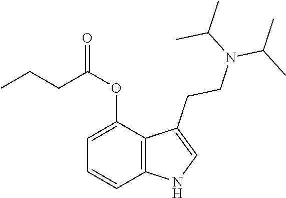 [SMILES: CCCC(=O)Oc1cccc2[nH]cc(CCN(C(C)C)C(C)C)c12] | 3-(2-(diisopropyl- amino)ethyl)-1H- indol-4-yl butyrate | 330.23 | 331.23 | 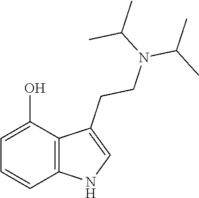 [SMILES: CC(C)N(CCc1c[nH]c2cccc(O)c12)C(C)C] | Butanoyl Coenzyme A | Purified enzyme of SEQ ID NO: 8 |
| 14 | 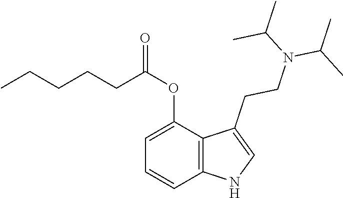 [SMILES: CCCCCC(=O)Oc1cccc2[nH]cc(CCN(C(C)C)C(C)C)c12] | 3-(2-(diisopropyl- amino)ethyl)- 1H-indol-4-yl hexanoate | 358.26 | 359.26 | 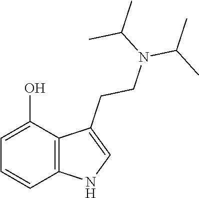 [SMILES: CC(C)N(CCc1c[nH]c2cccc(O)c12)C(C)C] | Hexanoyl Coenzyme A | Purified enzyme of SEQ ID NO: 8 |
| 15 | 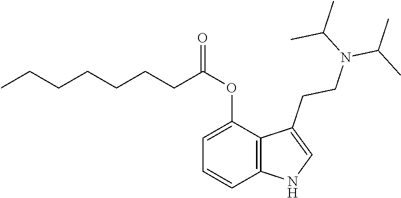 [SMILES: CCCCCCCC(=O)Oc1cccc2[nH]cc(CCN(C(C)C)C(C)C)c12] | 3-(2-(diisopropyl- amino)ethyl)- 1H-indol-4-yl octanoate | 386.29 | 387.29 | 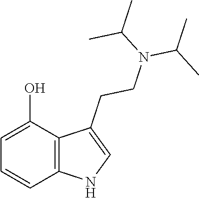 [SMILES: CC(C)N(CCc1c[nH]c2cccc(O)c12)C(C)C] | Octanoyl Coenzyme A | Purified enzyme of SEQ ID NO: 8 |
| 16 | 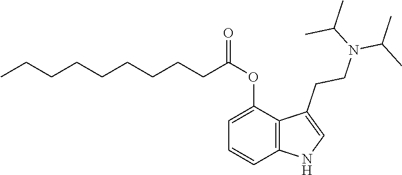 [SMILES: CCCCCCCCCC(=O)Oc1cccc2[nH]cc(CCN(C(C)C)C(C)C)c12] | 3-(2-(diisopropyl- amino)ethyl)- 1H-indol-4-yl decanoate | 414.32 | 415.32 | 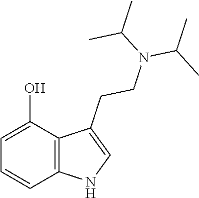 [SMILES: CC(C)N(CCc1c[nH]c2cccc(O)c12)C(C)C] | Decanoyl Coenzyme A | Purified enzyme of SEQ ID NO: 8 |
| 17 | 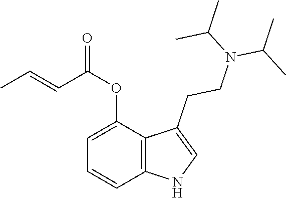 [SMILES: C/C=C/C(=O)Oc1cccc2[nH]cc(CCN(C(C)C)C(C)C)c12] | 3-(2-(diisopropyl- amino)ethyl)- 1H-indol-4-yl (E)-but-2-enoate | 328.22 | 329.22 | 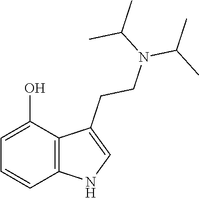 [SMILES: CC(C)N(CCc1c[nH]c2cccc(O)c12)C(C)C] | (E)-but- 2-enoyl Coenzyme A | Purified enzyme of SEQ ID NO: 8 |
| 18 | 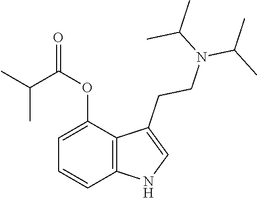 [SMILES: CC(C)C(=O)Oc1cccc2[nH]cc(CCN(C(C)C)C(C)C)c12] | 3-(2-(diisopropyl- amino)ethyl)- 1H-indol-4-yl isobutyrate | 330.23 | 331.23 | 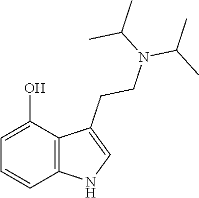 [SMILES: CC(C)N(CCc1c[nH]c2cccc(O)c12)C(C)C] | Isobutyryl- Coenzyme A | Purified enzyme of SEQ ID NO: 8 |
| 19 | 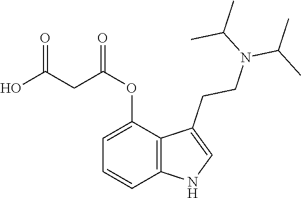 [SMILES: CC(C)N(CCc1c[nH]c2cccc(OC(=O)CC(=O)O)c12)C(C)C] | 3-((3-(2-(diiso- propylamino)- ethyl)-1H-indol-4- yl)oxy)-3-oxo- propanoic acid | 346.19 | 347.19 | 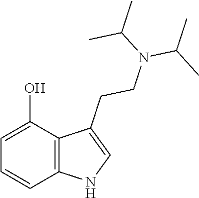 [SMILES: CC(C)N(CCc1c[nH]c2cccc(O)c12)C(C)C] | Malonyl Coenzyme A | Purified enzyme of SEQ ID NO: 8 |
| 20 | 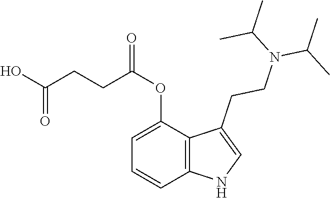 [SMILES: CC(C)N(CCc1c[nH]c2cccc(OC(=O)CCC(=O)O)c12)C(C)C] | 4-((3-(2-(diso- propylamino)- ethyl)-1H-indol- 4-yl)oxy)-4- oxobutanoic acid | 360.2 | 361.2 | 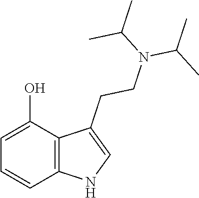 [SMILES: CC(C)N(CCc1c[nH]c2cccc(O)c12)C(C)C] | Succinyl Coenzyme A | Purified enzyme of SEQ ID NO: 8 |
| 21 | 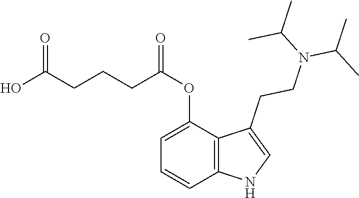 [SMILES: CC(C)N(CCc1c[nH]c2cccc(OC(=O)CCCC(=O)O)c12)C(C)C] | 5-((3-(2-(diso- propylamino)- ethyl)-1H-indol- 4-yl)oxy)-5- oxopentanoic acid | 374.22 | 375.22 | 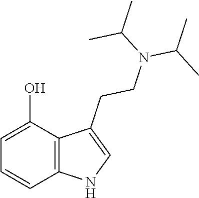 [SMILES: CC(C)N(CCc1c[nH]c2cccc(O)c12)C(C)C] | Glutaryl Coenzyme A | Purified enzyme of SEQ ID NO: 8 |
| 22 | 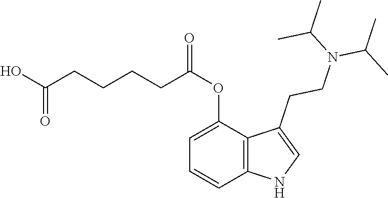 [SMILES: CC(C)N(CCc1c[nH]c2cccc(OC(=O)CCCCC(=O)O)c12)C(C)C] | 6-((3-(2-(diiso- propylamino)- ethyl)-1H-indol- 4-yl)oxy)-6- oxohexanoic acid | 388.24 | 389.24 | 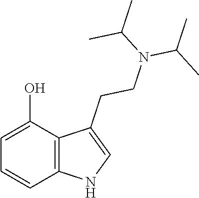 [SMILES: CC(C)N(CCc1c[nH]c2cccc(O)c12)C(C)C] | Adipoyl Coenzyme A | Purified enzyme of SEQ ID NO: 8 |
| 23 | 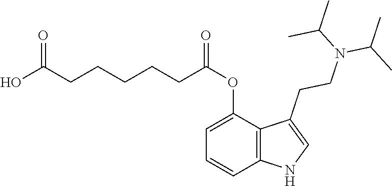 [SMILES: CC(C)N(CCc1c[nH]c2cccc(OC(=O)CCCCCC(=O)O)c12)C(C)C] | 7-((3-(2-(diiso- propylamino)- ethyl)-1H-indol- 4-yl)oxy)-7- oxoheptanoic acid | 402.25 | 403.25 | 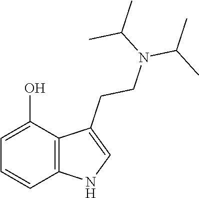 [SMILES: CC(C)N(CCc1c[nH]c2cccc(O)c12)C(C)C] | Heptanoyl Coenzyme A | Purified enzyme of SEQ ID NO: 8 |
| 24 | 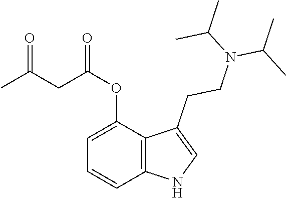 [SMILES: CC(=O)CC(=O)Oc1cccc2[nH]cc(CCN(C(C)C)C(C)C)c12] | 3-(2-(diisopropyl- amino)ethyl)-1H- indol-4-yl 3- oxobutanoate | 344.21 | 345.21 | 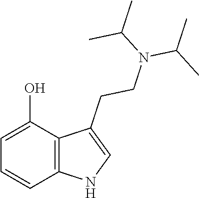 [SMILES: CC(C)N(CCc1c[nH]c2cccc(O)c12)C(C)C] | Aceto- acetyl- Coenzyme A | Purified enzyme of SEQ ID NO: 8 |
| 25 | 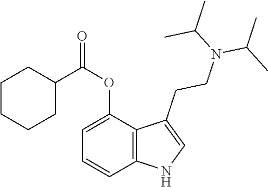 [SMILES: CC(C)N(CCc1c[nH]c2cccc(OC(=O)C3CCCCC3)c12)C(C)C] | 3-(2-(diisopropyl- amino)ethyl)-1H- indol-4-yl cyclo- hexanecarboxylate | 370.26 | 371.26 | 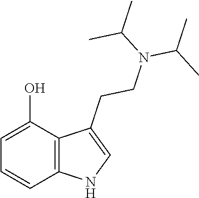 [SMILES: CC(C)N(CCc1c[nH]c2cccc(O)c12)C(C)C] | Cyclo- hexanoyl- Coenzyme A | Purified enzyme of SEQ ID NO: 8 |
| 26 | 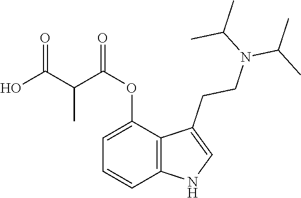 [SMILES: CC(C(=O)O)C(=O)Oc1cccc2[nH]cc(CCN(C(C)C)C(C)C)c12] | 3-((3-(2-(diiso- propylamino)- ethyl)-1H-indol- 4-yl)oxy)-2- methyl-3-oxo- propanoic acid | 360.2 | 361.2 | 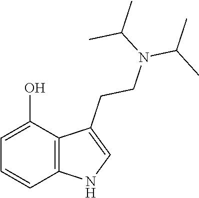 [SMILES: CC(C)N(CCc1c[nH]c2cccc(O)c12)C(C)C] | Methyl- malonyl Coenzyme A | Purified enzyme of SEQ ID NO: 8 |
| 27 | 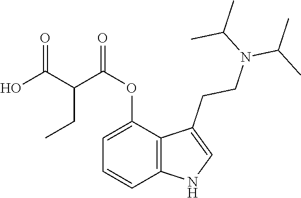 [SMILES: CCC(C(=O)O)C(=O)Oc1cccc2[nH]cc(CCN(C(C)C)C(C)C)c12] | 2-(((3-(2-(diiso- propylamino)- ethyl)-1H-indol- 4-yl)oxy)carbon- yl)butanoic acid | 374.22 | 375.22 | 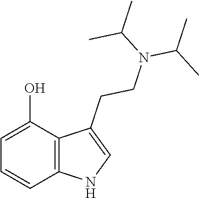 [SMILES: CC(C)N(CCc1c[nH]c2cccc(O)c12)C(C)C] | Ethyl- malonyl Coenzyme A | Purified enzyme of SEQ ID NO: 8 |
| 28 | 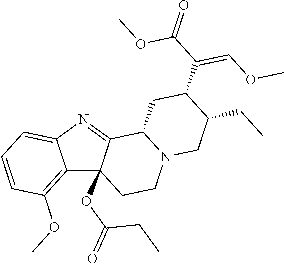 [SMILES: CCC(=O)O[C@]12CCN3C[C@@H](CC)[C@@H](/C(=C\OC)C(=O)OC)C[C@H]3C1=Nc1cccc(OC)c12] | methyl (5)-2- ((2,3,7aS,12bS)- 3-ethyl-8-meth- oxy-7a-(propionyl- oxy)-1,2,3,4,6,7, 7a,12b-octahydro- indolo[2,3-a]- quinolizin-2-yl)-3- methoxyacrylate | 470.24 | 471.24 | 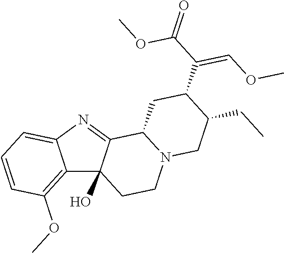 [SMILES: CC[C@@H]1CN2CC[C@@]3(O)C(=Nc4cccc(OC)c43)[C@@H]2C[C@@H]1/C(=C\OC)C(=O)OC] | Propanoyl Coenzyme A | Purified enzyme of SEQ ID NO: 8 |
| 29 | 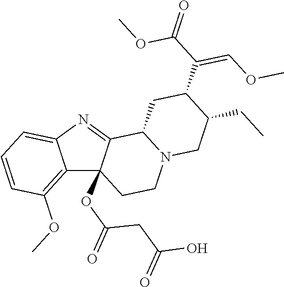 [SMILES: CC[C@@H]1CN2CC[C@@]3(OC(=O)CC(=O)O)C(=Nc4cccc(OC)c43)[C@@H]2C[C@@H]1/C(=C\OC)C(=O)OC] | 3-(((2S,3S,7aS, 12bS)-2-((E)-1,3- dimethoxy-3- oxoprop-1-en-2- yl)-3-ethyl-8-meth- oxy-1,3,4,6,7,12b- hexahydroindolo- [2,3-a]quinolizin- 7a(2H)-yl)oxy)-3- oxopropanoic acid | 500.22 | 501.22 | 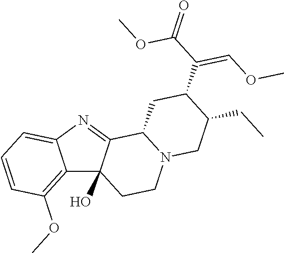 [SMILES: CC[C@@H]1CN2CC[C@@]3(O)C(=Nc4cccc(OC)c43)[C@@H]2C[C@@H]1/C(=C\OC)C(=O)OC] | Malonyl Coenzyme A | Purified enzyme of SEQ ID NO: 8 |
| 30 | 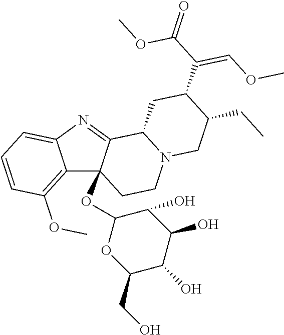 [SMILES: CC[C@@H]1CN2CC[C@@]3(OC4O[C@H](CO)[C@@H](O)[C@H](O)[C@H]4O)C(=Nc4cccc(OC)c43)[C@@H]2C[C@@H]1/C(=C\OC)C(=O)OC] | methyl (E)-2- ((2S,3S,7a.S, 12bS)-3-ethyl- 8-methoxy-7a- (((3R,4S,5S,6R)- 3,4,5-trihydroxy- 6-(hydroxymeth- yl)tetrahydro-2H- pyran-2-yl)oxy)- 1,2,3,4.6,7,7a, 12b-octahydro- indolo[2,3-a]- quinolizin-2- yl)-3-methoxy- acrylate | 576.27 | 577.27 | 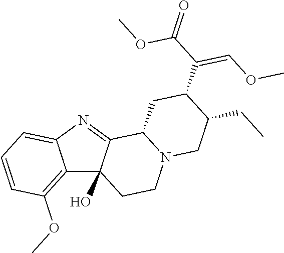 [SMILES: CC[C@@H]1CN2CC[C@@]3(O)C(=Nc4cccc(OC)c43)[C@@H]2C[C@@H]1/C(=C\OC)C(=O)OC] | UDP- glucose | Purified enzyme of SEQ ID NO: 10 |
| 31 | 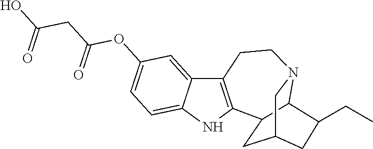 [SMILES: CCC1CC2CC3c4[nH]c5ccc(OC(=O)CC(=O)O)cc5c4CCN(C2)C13] | 3-((7-ethyl- 6,6a,7,8,9,10, 12,13-octahydro- 5H-6,9-methano- pyrido[1′,2′:1,2]- azepino[4,5-b]- indol-2-yl)oxy)- 3-oxo-propanic acid | 382.19 | 383.19 | 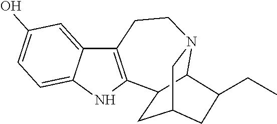 [SMILES: CCC1CC2CC3c4[nH]c5ccc(O)cc5c4CCN(C2)C13] | Malonyl Coenzyme A | Purified enzyme of SEQ ID NO: 8 |
| 32 | 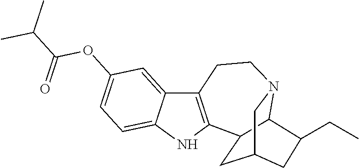 [SMILES: CCC1CC2CC3c4[nH]c5ccc(OC(=O)C(C)C)cc5c4CCN(C2)C13] | 7-ethyl-6,6a,7, 8,9,10,12,13- octahydro-5H- 6,9-methano- pyrido- [1′,2′:1,2]- azepino[4,5-b]- indol-2-yl isobutyrate | 366.23 | 367.23 | 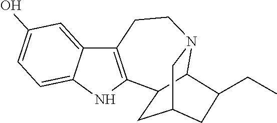 [SMILES: CCC1CC2CC3c4[nH]c5ccc(O)cc5c4CCN(C2)C13] | Isobutyryl Coenzyme A | Purified enzyme of SEQ ID NO: 8 |
| 33 | 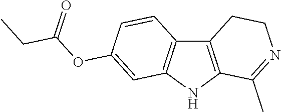 [SMILES: CCC(=O)Oc1ccc2c3c([nH]c2c1)C(C)=NCC3] | 1-methyl-4,9- dihydro-3H- pyrido[3,4-b]- indol-7-yl propionate | 256.12 | 257.12 | 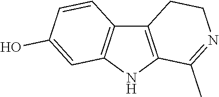 | Propanoyl Coenzyme A | Purified enzyme of SEQ ID NO: 8 |
| 34 | 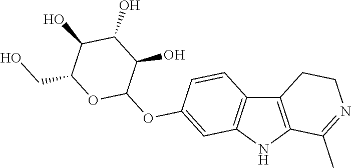 [SMILES: CC1=NCCc2c1[nH]c1cc(OC3O[C@H](CO)[C@@H](O)[C@H](O)[C@H]3O)ccc21] | 2-(hydroxy- methyl)-6-((1- methyl-4,9- dihydro-3H- pyrido[3,4-b]- indol-7-yl)- oxy)tetrahydro- 2H-pyran-3,4, 5-triol | 362.15 | 363.15 | 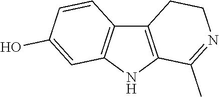 | UDP- glucose | Purified enzyme of SEQ ID NO: 10 |
| 35 | 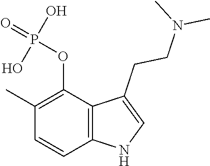 | 3-(2-(dimeth- ylamino)ethyl)-5- methyl-1H-indol- 4-yl dihydrogen phosphate | 298.15 | 299.15 | 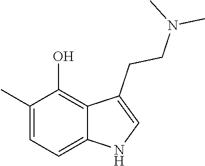 | ATP | Purified enzyme of SEQ ID NO: 2 |
| 36 | 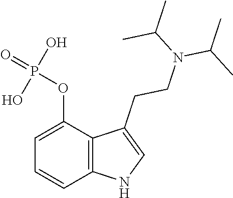 [SMILES: CC(C)N(CCc1c[nH]c2cccc(OP(=O)(O)O)c12)C(C)C] | 3-(2-(diiso- propylamino)- ethyl)-1H-indol- 4-yl dihydrogen phosphate | 340.16 | 341.16 | 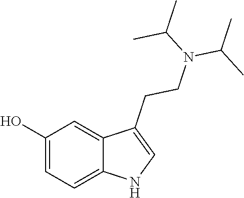 [SMILES: CC(C)N(CCc1c[nH]c2ccc(O)cc12)C(C)C] | ATP | Purified enzyme of SEQ ID NO: 2 |
| 37 | 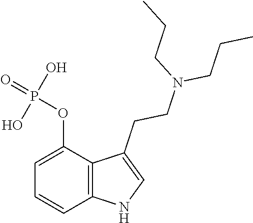 [SMILES: CCCN(CCC)CCc1c[nH]c2cccc(OP(=O)(O)O)c12] | 3-(2-(dipropyl- amino)ethyl)- 1H-indol-4-yl dihydrogen phosphate | 340.16 | 341.16 | 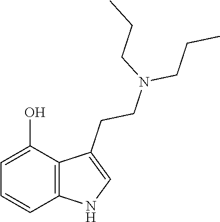 [SMILES: CCCN(CCC)CCc1c[nH]c2cccc(O)c12] | ATP | Purified enzyme of SEQ ID NO: 2 |
| 38 | 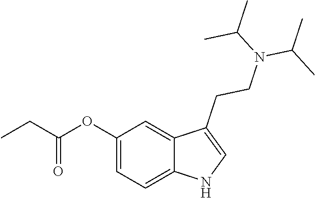 [SMILES: CCC(=O)Oc1ccc2[nH]cc(CCN(C(C)C)C(C)C)c2c1] | 3-(2-(diiso- propylamino)- ethyl)-1H-indol- 5-yl propionate | 316.23 | 317.22 | 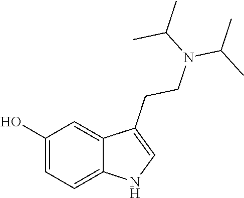 [SMILES: CC(C)N(CCc1c[nH]c2ccc(O)cc12)C(C)C] | Propanoyl Coenzyme A | Purified enzyme of SEQ ID NO: 8 |
| 39 | 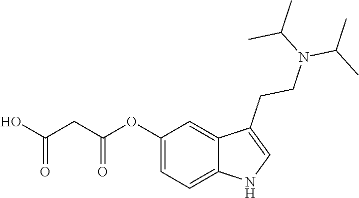 [SMILES: CC(C)N(CCc1c[nH]c2ccc(OC(=O)CC(=O)O)cc12)C(C)C] | 3-((3-(2-(diiso- propylamino)- ethyl)-1H-indol- 5-yl)oxy)-3- oxopropanoic acid | 346.19 | 347.19 | 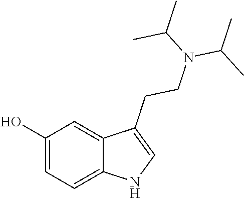 [SMILES: CC(C)N(CCc1c[nH]c2ccc(O)cc12)C(C)C] | Malonyl Coenzyme A | Purified enzyme of SEQ ID NO: 2 |
| 40 | 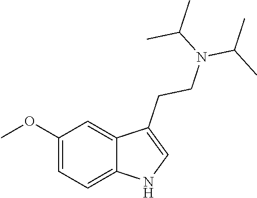 [SMILES: COc1ccc2[nH]cc(CCN(C(C)C)C(C)C)c2c1] | N-isopropyl-N-(2- (5-methoxy-1H- indol-3-yl)ethyl)- propan-2-amine | 274.2 | 275.2 | 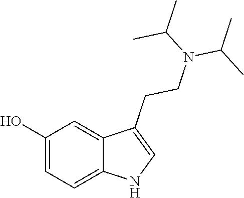 [SMILES: CC(C)N(CCc1c[nH]c2ccc(O)cc12)C(C)C] | SAM | Purified enzyme of SEQ ID NO: 3 |

### Example 7. 5HT2A Agonist Activity by Enzymatically Digested Modified Indole Alkaloids

1 mM of 4-3-(2-(dipropylamino)ethyl)-1H-indol-4-yl dihydrogen phosphate (Compound A) was incubated with 100 units of calf intestinal alkaline phosphatase for 2 hours in buffer containing 50 mM Potassium acetate, 20 mM Tris-acetate, 10 mM Magnesium acetate, and 100 μg/ml BSA at pH 7.9 and 25° C. 1 mM of 4-3-(2-(diisopropylamino)ethyl)-1H-indol-4-yl dihydrogen phosphate (Compound B) was incubated with 100 units of calf intestinal alkaline phosphatase for 2 hours in buffer containing 50 mM Potassium acetate, 20 mM Tris-acetate, 10 mM Magnesium acetate, and 100 μg/ml BSA at pH 7.9 and 25° C. The resulting solutions containing dephosphorylated products were used in a 5-HT2A receptor functional assay.

5-HT2 functional experiments (measuring Gq-mediated calcium flux) were performed with Flp-In T-REx 293 cells (Invitrogen, Carlsbad, CA) expressing human 5-HT2A (h5-HT2A) receptor cDNA under the tetracycline repressor protein. Cells were plated into black 384-well clear bottomed tissue culture plates in 40 μL of DMEM containing 1% dialyzed fetal bovine serum (FBS) at a density of approximately 10,000 cells per well, and receptor expression was induced with 2 μg/mL tetracycline.

After approximately 20-24 hrs, the medium was decanted and replaced with 20 μL per well of drug buffer (HBSS, 20 mM HEPES, pH 7.4) containing Fluo-4 Direct dye (Invitrogen) and incubated for between 1 and 2 h at 37° C. Test substances (e.g., Compound A and Compound B) were diluted in drug buffer (HBSS, 20 mM HEPES, 0.1% bovine serum albumin, 0.01% ascorbic acid, pH 7.4).

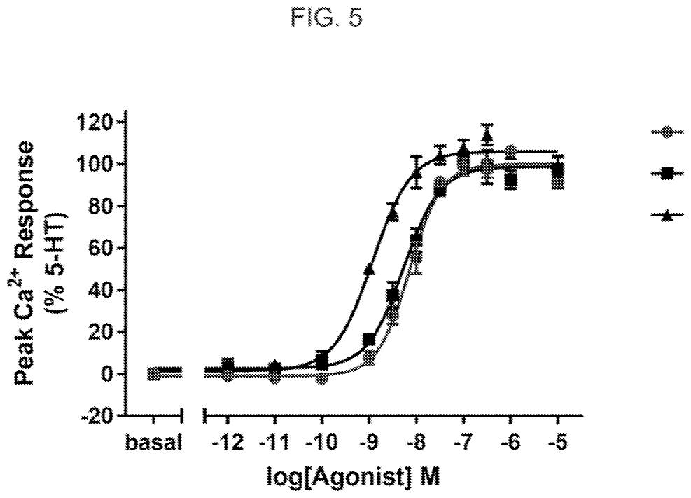

Before the experiment, plates were allowed to equilibrate to room temperature, and calcium flux was measured using a FLIPRTETRA cellular screening system (Molecular Devices, Sunnyvale, CA). Plates were read for fluorescence initially for 10 s (1 read per second) to establish a baseline and then stimulated with drug dilutions or buffer and read for an additional 120 s. Peak fluorescence in each well was normalized to the maximum fold increase over baseline. Data were normalized to the maximum peak fold over basal fluorescence produced by 5-hydroxytryptamine (5-HT) (100%) and baseline fluorescence (0%). Data were analyzed using the sigmoidal dose-response function of Prism 5.0 or 8.0 (GraphPad Software, San Diego, CA). Relative activity (RA) was expressed as the logarithm of the ratio of Eₘₐₓ over EC₅₀ parameter estimates. The obtained data showing 5HT2A agonism of Compound A and Compound B as compared to 5-HT is illustrated in FIG. 5.

### Example 8: Treatment of Modified Indole Alkaloids with Human Salivary Preparations

In this example, additional modified indole alkaloids were synthesized using enzymatic glycosylation conditions and then digested with human salivary samples.

### Modified Indole Alkaloid Synthesis

Indole alkaloid substrates were prepared by at 0.5 mg/mL concentration in a 1:1 mixture of DMSO:PBS at pH 7.5. All buffers and reagents were purchased from Sigma-Aldrich, Inc, unless otherwise noted.

The following indole alkaloid substrates were used in this example:
4-hydroxy-N-methyl-N-ethyltryptamine (Cayman Chemicals MI, USA)
4-hydroxy-N-methyl-N-propyltryptamine (Cayman Chemicals MI, USA)
4-hydroxy-N-methyl-N-isopropyltryptamine (Cayman Chemicals MI, USA)
4-hydroxy-N-methyl-N-allyltryptamine (Cayman Chemicals MI, USA)
4-hydroxy-N,N-diethyltryptamine (Cayman Chemicals MI, USA)
4-hydroxy-N-ethyl-N-propyltryptamine hemifumarate (Cayman Chemicals MI, USA)
4-hydroxy-N,N-dipropyltryptamine (Cayman Chemicals MI, USA)
4-hydroxy-N,N-diisopropyltryptamine hydrochloride (Cayman Chemicals MI, USA)
4-hydroxy-N-methyl-N-cyclopropyltryptamine hydrochloride (Cayman Chemicals MI, USA)

Uridine 5′-diphosphoglucose disodium salt hydrate Item No. U4625 (UDP-glucose) (Sigma-Aldrich, Inc MO, USA) was used as the co-substrate in this example.

100-μL reaction mixtures were prepared for each of Reactions 41-49 using the buffer, indole alkaloid substrate, co-substrate, enzyme, and reagents described below:
50 mM HEPES pH 7.5
50 mM NaCl
5 mM MgCl₂
1 mM DTT
5 mM UDP-glucose (co-substrate)
100 μM indole alkaloid substrate
50 μM purified enzyme of SEQ ID NO:10

The reactions were carried out for 3 hours at 37° C., and then were quenched with 100 μL 100 mM NaOH. The reactions were centrifuged at 30,000 ref and the supernatant transferred to a fresh sample tube.

Five microliters of sample were injected onto a ZORBAX StableBond-C18, 1.8 um, 2.1×50 mm column at 20° C. using a 0.3 mL/min flow rate and major ion species were detected with a 6520 Accurate-Mass Q-TOF LC-MS (Agilent) in positive mode. The mobile phase consisted of a mixture of 0.1% formic acid in acetonitrile (v/v) and 0.1% formic acid in water (v/v) was eluted under the following gradient conditions (shown in relation to acetonitrile content): 0 min-10%, 6 min-100%, 7 min-10%, 14 min-10%. The mobile phase was delivered at a flow rate of 0.3 mL/min and the total analytical run time was 14 min. Masses for modified indole alkaloid products were not detected in heat inactivated enzyme samples, indole alkaloid samples or cofactor samples alone.

The reaction products of Reactions 41 to 49 are summarized in Table 10 below.

TABLE 10
Product of Enzymatic Glucosylation of Indole Alkaloid Substrates

| Reaction | | Chemical Name | Product | Product | | | |
|---|---|---|---|---|---|---|---|
| Number | Product Structure | of the Product | MW | (M + H)+ | Indole alkaloid substrate | Co-substrate | Enzyme |
| 41 | 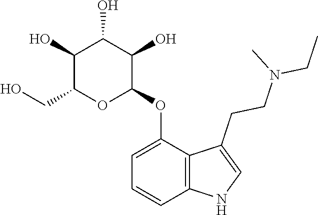 [SMILES: CCN(C)CCc1c[nH]c2cccc(O[C@H]3O[C@H](CO)[C@@H](O)[C@H](O)[C@H]3O)c12] | 2-((3-(2-ethyl(methyl)- amino)ethyl)-1H-indol-4- yl)oxy)-6-(hydroxy- methyl)tetrahydro-2H- pyran-3,4,5-triol | 380.19 | 381.19 | 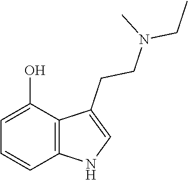 [SMILES: CCN(C)CCc1c[nH]c2cccc(O)c12] | UDP- glucose | Purified enzyme of SEQ ID NO: 10 |
| 42 | 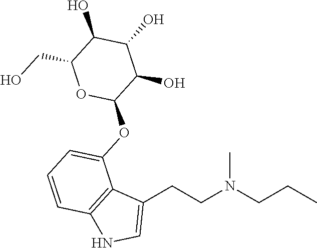 [SMILES: CCCN(C)CCc1c[nH]c2cccc(O[C@H]3O[C@H](CO)[C@@H](O)[C@H](O)[C@H]3O)c12] | 2-(hydroxymethyl)-6- ((3-(2-(methyl(propyl)- amino)ethyl)-1H-indol-4- yl)oxy)tetrahydro-2H- pyran-3,4,5-triol | 394.21 | 395.21 | 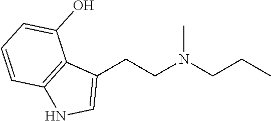 [SMILES: CCCN(C)CCc1c[nH]c2cccc(O)c12] | UDP- glucose | Purified enzyme of SEQ ID NO: 10 |
| 43 | 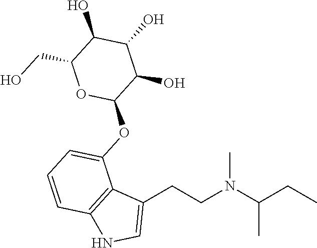 [SMILES: CCC(C)N(C)CCc1c[nH]c2cccc(O[C@H]3O[C@H](CO)[C@@H](O)[C@H](O)[C@H]3O)c12] | 2-(hydroxymethyl)-6-((3- (2-(isopropyl(methyl)- amino)ethyl)-1H-indol-4- yl)oxy)tetrahydro-2H- pyran-3,4,5-triol | 394.21 | 395.21 | 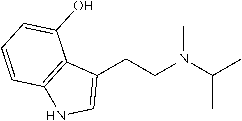 [SMILES: CC(C)N(C)CCc1c[nH]c2cccc(O)c12] | UDP- glucose | Purified enzyme of SEQ ID NO: 10 |
| 44 | 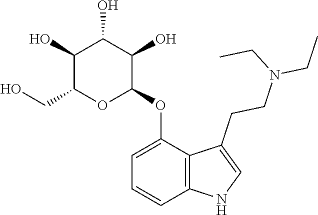 [SMILES: CCN(CC)CCc1c[nH]c2cccc(O[C@H]3O[C@H](CO)[C@@H](O)[C@H](O)[C@H]3O)c12] | 2-((3-(2-(diethylamino)- ethyl)-1H-indol-4-yl)oxy)- 6-(hydroxymethyl)tetra- hydro-2H-pyran-3,4,5-triol | 394.21 | 395.21 | 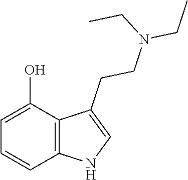 [SMILES: CCN(CC)CCc1c[nH]c2cccc(O)c12] | UDP- glucose | Purified enzyme of SEQ ID NO: 10 |
| 45 | 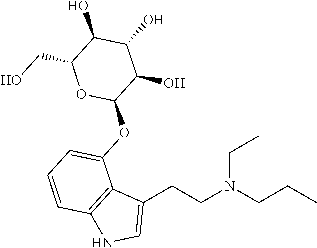 [SMILES: CCCN(CC)CCc1c[nH]c2cccc(O[C@H]3O[C@H](CO)[C@@H](O)[C@H](O)[C@H]3O)c12] | 2-((3-(2-(ethyl(propyl)- amino)ethyl)-1H-indol-4- yloxy)-6-(hydroxymethyl)- tetrahydro-2H-pyran- 3,4,5-triol | 408.23 | 409.23 | 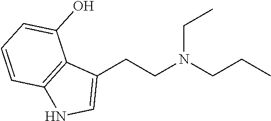 [SMILES: CCCN(CC)CCc1c[nH]c2cccc(O)c12] | UDP- glucose | Purified enzyme of SEQ ID NO: 10 |
| 46 | 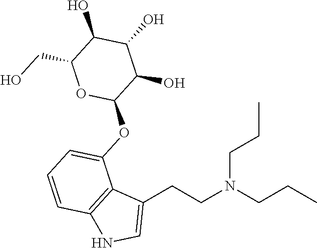 [SMILES: CCCN(CCC)CCc1c[nH]c2cccc(O[C@H]3O[C@H](CO)[C@@H](O)[C@H](O)[C@H]3O)c12] | 2-((3-(2-(dipropylamino)- ethyl)-1H-indol-4-yl)oxy)- 6-(hydroxymethyl)- tetrahydro-2H-pyran- 3,4,5-triol | 422.24 | 423.24 | 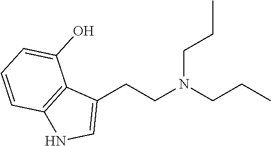 [SMILES: CCCN(CCC)CCc1c[nH]c2cccc(O)c12] | UDP- glucose | Purified enzyme of SEQ ID NO: 10 |
| 47 | 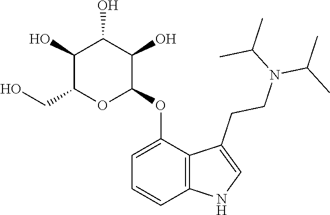 [SMILES: CC(C)N(CCc1c[nH]c2cccc(O[C@H]3O[C@H](CO)[C@@H](O)[C@H](O)[C@H]3O)c12)C(C)C] | 2-((3-(2-(diisopropyl- amino)ethyl)-1H-indol-4- yl)oxy)-6-(hydroxymeth- yl)tetrahydro-2H-pyran- 3,4,5-triol | 422.24 | 423.24 | 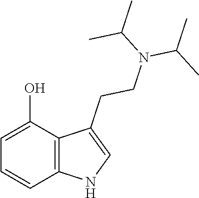 [SMILES: CC(C)N(CCc1c[nH]c2cccc(O)c12)C(C)C] | UDP- glucose | Purified enzyme of SEQ ID NO: 10 |
| 48 | 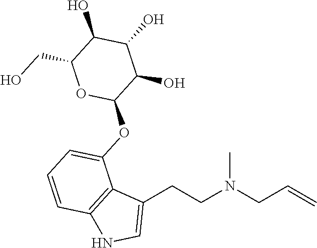 [SMILES: C=CCN(C)CCc1c[nH]c2cccc(O[C@H]3O[C@H](CO)[C@@H](O)[C@H](O)[C@H]3O)c12] | 2-((3-(2-(allyl(methyl)- amino)ethyl)-1H-indol-4- yl)oxy)-6-(hydroxymeth- yl)tetrahydro-2H-pyran- 3,4,5-triol | 392.19 | 393.19 | 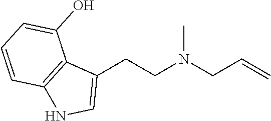 [SMILES: C=CCN(C)CCc1c[nH]c2cccc(O)c12] | UDP- glucose | Purified enzyme of SEQ ID NO: 10 |
| 49 | 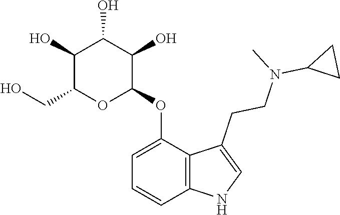 [SMILES: CN(CCc1c[nH]c2cccc(O[C@H]3O[C@H](CO)[C@@H](O)[C@H](O)[C@H]3O)c12)C1CC1] | 2-((3-(2-(cyclopropyl- (methyl)amino)ethyl)-1H- indol-4-yl)oxy)-6-(hydroxy- methyl)tetrahydro-2H- pyran-3,4,5-triol | 392.19 | 393.19 | 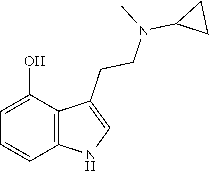 [SMILES: CN(CCc1c[nH]c2cccc(O)c12)C1CC1] | UDP- glucose | Purified enzyme of SEQ ID NO: 10 |

Digestion of Modified Indole Alkaloids with Human Salivary Samples

Human saliva samples were collected 25 minutes before food intake after 8 hours fasting from an adult subject with no clinical symptoms. For saliva collection, saliva samples were obtained by passive drool into sterile 10 mL centrifuge tubes over a 5-min period. Immediately after collection, saliva samples were centrifuged (500 g for 10 min at 4° C.) and the supernatant was recovered and used immediately or stored at −80° C. until further use.

Eleven 100-μL reaction mixtures were prepared, consisting of:
50 mM HEPES pH 7.5
50 mM NaCl
100 μM modified indole alkaloid product (from Reactions 3, 24, 39-47, respectively)
50 μL, of human saliva sample
2 mM calcium chloride
5 mM magnesium chloride

The reaction mixtures were digested for 2 hours at 37° C., and heated to 80° C. for 5 minutes to terminate the reactions. The reaction mixtures were then centrifuged at 30,000 ref and the supernatant transferred to a fresh sample tube.

One microliter of the sample was injected onto a ZORBAX StableBond-C18, 1.8 um, 2.1×50 mm column at 20° C. using a 0.3 mL/min flow rate and major ion species were detected with a 6520 Accurate-Mass Q-TOF LC-MS (Agilent) in positive mode. The mobile phase consisted of a mixture of 0.1% formic acid in acetonitrile (v/v) and 0.1% formic acid in water (v/v) was eluted under the following gradient conditions (shown in relation to acetonitrile content): 0 min-10%, 6 min-100%, 7 min-10%, 14 min-10%. The mobile phase was delivered at a flow rate of 0.3 mL/min and the total analytical run time was 14 min.

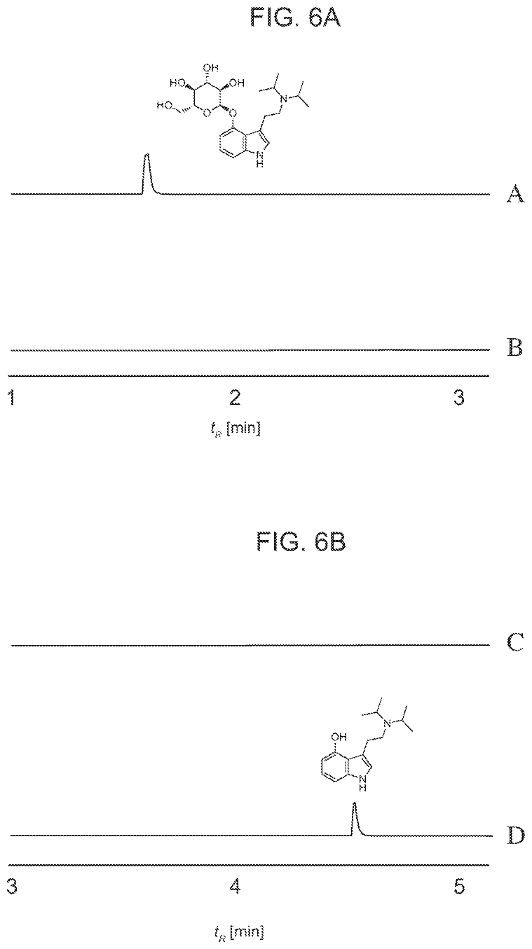

As shown in FIGS. 6A-6B, treatment with human saliva de-glycosylated the modified indole alkaloid 2-((3-(2-(diisopropylamino)ethyl)-1H-indol-4-yl)oxy)-6-(hydroxymethyl)tetrahydro-2H-pyran-3,4,5-triol (Compound C, product from enzymatic glycosylation Reaction 47). LC-MS traces of Compound C alone (traces A and C) are shown in FIG. 6A and LC-MS traces of Compound C samples treated with human saliva (traces B and D) are shown in FIG. 6B. In traces A and B, a m/z of 423.24, which corresponds to Compound C, the glycosylated product, was extracted. In traces C and D, a m/z of 261.19, which corresponds to the deglycosylated product of Compound C, which is 4-hydroxy-N,N-diisopropyltryptamine, was extracted. These results indicate that human saliva completely deglycosylated the glucosylated indole alkaloid.

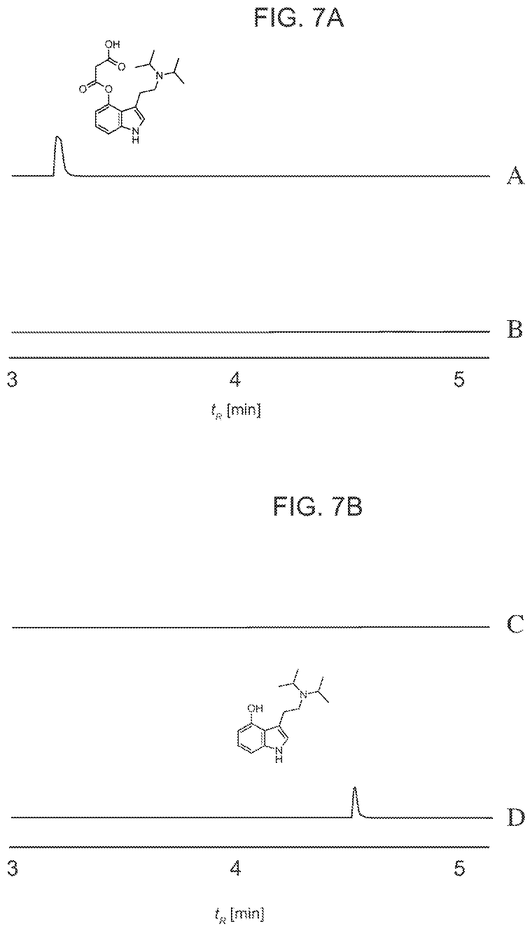

As shown in FIGS. 7A-7B, treatment with human saliva hydrolyzed the modified indole alkaloid 3-((3-(2-(diisopropylamino)ethyl)-1H-indol-4-yl)oxy)-3-oxopropanoic acid (Compound D, product from enzymatic malonylation Reaction 3). LC-MS traces of Compound D alone (traces A and C) are shown in FIG. 7A and LC-MS traces of Compound D samples treated with human saliva (traces B and D) are shown in FIG. 7B. In traces A and B, a m/z of 347.19, which corresponds to Compound D, was extracted. In traces C and D, a m/z of 261.19, which corresponds to the hydrolyzed product of Compound D, which is 4-hydroxy-N,N-diisopropyltryptamine, was extracted. These results indicate that human saliva completely hydrolyzed the malonylation product Compound D.

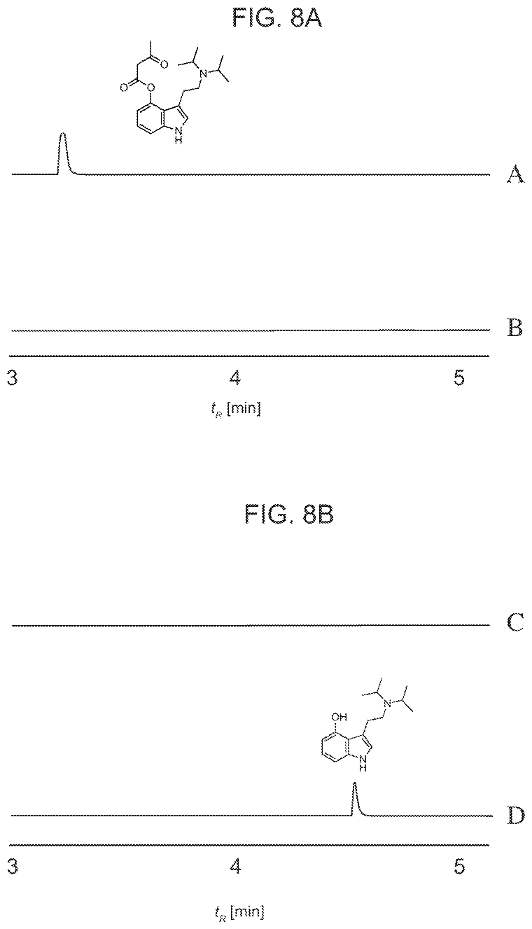

As shown in FIGS. 8A-8B, treatment with human saliva hydrolyzed the modified indole alkaloid 3-(2-(diisopropylamino)ethyl)-1H-indol-4-yl 3-oxobutanoate (Compound E; product from enzymatic acetylation Reaction 24). LC-MS traces of Compound E alone (traces A and C) are shown in FIG. 8A and LC-MS traces of Compound E samples treated with human saliva (traces B and D) are shown in FIG. 8B. In traces A and B, a m/z of 345.21, which corresponds to Compound E, was extracted. In traces C and D, a m/z of 261.19, which corresponds to the hydrolyzed product of Compound E, which is 4-hydroxy-N,N-diisopropyltryptamine, was extracted. These results indicate that human saliva completely hydrolyzed the acetylation product Compound E.

The retention times of Compound C (from Reaction 47), Compound D (from Reaction 3), Compound E (from Reaction 24), and 4-hydroxy-N,N-diisopropyltryptamine are summarized in Table 11 below.

TABLE 11
Retention Times of Compound C, Compound D, Compound E, and 4-hydroxy-N,N-
diisopropyltryptamine

| | | Product from | Retention time |
|---|---|---|---|
| Structure | Compound | Reaction # | (min) |
| 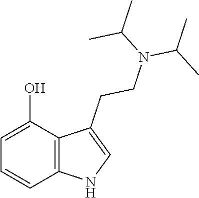 [SMILES: CC(C)N(CCc1c[nH]c2cccc(O)c12)C(C)C] | 4-hydroxy-N,N-diiso- propyltryptamine | N/A | 4.59 |
| 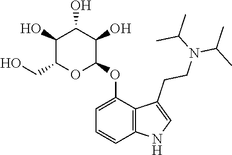 [SMILES: CC(C)N(CCc1c[nH]c2cccc(O[C@H]3O[C@H](CO)[C@@H](O)[C@H](O)[C@H]3O)c12)C(C)C] | 2-((3-(2-(diisopropyl- amino)ethyl)-1H-indol- 4-yl)oxy)-6-(hydroxy- methyl)tetrahydro-2H- pyran-3,4,5-triol (Compound C) | 47 | 1.67 |
| 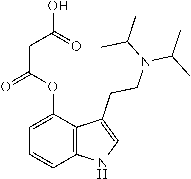 [SMILES: CC(C)N(CCc1c[nH]c2cccc(OC(=O)CC(=O)O)c12)C(C)C] | 3-((3-(2-(diisopropyl- amino)ethyl)-1H- indol-4-yl)oxy)-3- oxopropanoic acid (Compound D) | 3 | 3.23 |
| 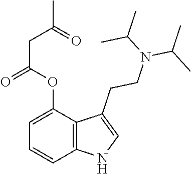 [SMILES: CC(=O)CC(=O)Oc1cccc2[nH]cc(CCN(C(C)C)C(C)C)c12] | 3-(2-(diisopropyl- amino)ethyl)-1H-indol- 4-yl 3-oxobutanoate (Compound E) | 24 | 3.32 |

### Example 9: 5HT2 Subtype Agonist Activities of Modified Indole Alkaloids

Modified indole alkaloids that were prepared in Reactions 41-49 and treated with human saliva in Example 8 were used in 5-HT2A and 5-HT2C receptor functional assays at 10 μM concentration.

5-HT2 functional experiments (measuring Gq-mediated calcium flux) were performed with Flp-In T-REx 293 cells (Invitrogen, Carlsbad, CA) independently expressing human 5-HT2A (h5-HT2A) receptor and human 5-HT2C (h5-HT2C) receptor eDNA under the tetracycline repressor protein. Cells were plated into black 384-well clear bottomed tissue culture plates in 40 μL of DMEM containing 1% dialyzed fetal bovine serum (FBS) at a density of approximately 10,000 cells per well, and receptor expression was induced with 2 μg/mL tetracycline.

After approximately 20-24 h, the medium was decanted and replaced with 20 μL per well of drug buffer (HBSS, 20 mM HEPES, pH 7.4) containing Fluo-4 Direct dye (Invitrogen) and incubated for between 1 and 2 h at 37° C. Test substances (modified indole alkaloid products from Reactions 41-49 treated with human saliva) were diluted in drug buffer (HBSS, 20 mM HEPES, 0.1% bovine serum albumin, 0.01% ascorbic acid, pH 7.4).

Before the experiment, plates were allowed to equilibrate to room temperature, and calcium flux was measured using a FLIPRTETRA cellular screening system (Molecular Devices, Sunnyvale, CA). Plates were read for fluorescence initially for 10 s (read per second) to establish a baseline and then stimulated with drug dilutions or buffer and read for an additional 120 s. Peak fluorescence in each well was normalized to the maximum fold increase over baseline. Data were normalized to the maximum peak fold over basal fluorescence produced by 5-hydroxytryptamine (5-HT) (100%) and baseline fluorescence (0%). Data were analyzed using the sigmoidal dose-response function of Prism 5.0 or 8.0 (GraphPad Software, San Diego. CA). Relative activity (RA) was expressed as the logarithm of the ratio of Eₘₐₓ over EC₅₀ parameter estimates.

EC₅₀ and Eₘₐₓ% values of 5H′T2A agonist activities of hydrolyzed (treatment with human saliva) indole alkaloids 2-((3-(2-(ethyl(methyl)amino)ethyl)-1H-indol-4-yl)oxy)-6-(hydroxymethyl)tetrahydro-2H-pyran-3,4,5-triol (Compound 1), 2-(hydroxymethyl)-6-((3-(2-(methyl(propyl)amino)ethyl)-1H-indol-4-yl)oxy)tetrahydro-2H-pyran-3,4,5-triol (Compound 2), 2-(hydroxymethyl)-6-((3-(2-(isopropyl(methyl)amino)ethyl)-1H-indol-4-yl)oxy)tetrahydro-2H-pyran-3,4,5-triol (Compound 3), 2-((3-(2-(diethylamino)ethyl)-1H-indol-4-yl)oxy)-6-(hydroxymethyl)tetrahydro-2H-pyran-3,4,5-triol (Compound 4), 2-((3-(2-(ethyl(propyl)amino)ethyl)-1H1-indol-4-yl)oxy)-6-(hydroxymethyl)tetrahydro-2H-pyran-3,4,5-triol (Compound 5), 2-((3-(2-(dipropylamino)ethyl)-1H-indol-4-yl)oxy)-6-(hydroxymethyl)tetrahydro-2H-pyran-3,4,5-triol (Compound 6), 2-((3-(2-(diisopropylamino)ethyl)-1H-indol-4-yl)oxy)-6-(hydroxymethyl)tetrahydro-2H-pyran-3,4,5-triol (Compound 7), 2-((3-(2-(allyl(methyl)amino)ethyl)-1H-indol-4-yl)oxy)-6-(hydroxymethyl)tetrahydro-2H-pyran-3,4,5-triol (Compound 8) and 2-((3-(2-(cyclopropyl(methyl)amino)ethyl)-1H-indol-4-yl)oxy)-6-(hydroxymethyl)tetrahydro-2H-pyran-3,4,5-triol (Compound 9), as compared endogenous ligand 5-hydroxytryptamine (5HT), were obtained and summarized in Table 12 below.

TABLE 12
5HT2A Agonist Activities of Modified Indole Alkaloids

| Sample Test | EC₅₀ nM | Eₘₐₓ % 5-HT (± SEM) |
|---|---|---|
| 5-hydroxytryptamine (5-HT) | 0.25 | 100 |
| Hydrolyzed Compound A-1 | 4.01 | 96.7 ± 1.1 |
| Hydrolyzed Compound A-2 | 3.83 | 98.0 ± 1.1 |
| Hydrolyzed Compound A-3 | 5.20 | 99.5 ± 1.0 |
| Hydrolyzed Compound A-4 | 6.48 | 100.1 ± 1.1 |
| Hydrolyzed Compound A-5 | 3.16 | 99.4 ± 1.2 |
| Hydrolyzed Compound A-6 | 1.65 | 103.2 ± 1.0 |
| Hydrolyzed Compound A-7 | 6.81 | 103 ± 1.2 |
| Hydrolyzed Compound A-8 | 3.66 | 102 ± 1.0 |
| Hydrolyzed Compound A-9 | 5.10 | 99.4 ± 2.0 |

EC₅₀ and Eₘₐₓ% values of 5HT2C agonist activities for hydrolyzed (treatment with human saliva) indole alkaloids Compounds 1-9, as compared endogenous ligand 5-hydroxytryptamine (5HT), were obtained and summarized in Table 13 below.

TABLE 13
5HT2A Agonist Activities of Modified Indole Alkaloids

| Sample | EC₅₀ nM | Eₘₐₓ % 5-HT (± SEM) |
|---|---|---|
| 5-hydroxytryptamine (5-HT) | 0.25 | 100 |
| Hydrolyzed Compound A-1 | 30.1 | 89.9 ± 2.1 |
| Hydrolyzed Compound A-2 | 47.8 | 90.7 ± 1.5 |
| Hydrolyzed Compound A-3 | 182 | 79.2 ± 1.0 |
| Hydrolyzed Compound A-4 | 258 | 80.1 ± 1.9 |
| Hydrolyzed Compound A-5 | 117 | 89.4 ± 2.6 |
| Hydrolyzed Compound A-6 | 214 | 84.2 ± 1.0 |
| Hydrolyzed Compound A-7 | 1551 | 94.4 ± 1.2 |
| Hydrolyzed Compound A-8 | 49.2 | 81.5 ± 2.4 |
| Hydrolyzed Compound A-9 | 148.6 | 79.1 ± 6.1 |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A compound of Formula (Ia): 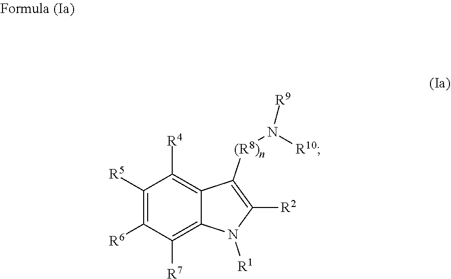
or a pharmaceutically acceptable salt thereof, wherein,
R¹ and R¹⁰ are independently selected from hydrogen, C₁-C₆ alkyl, C₂-C₆ alkenyl, and C₂-C₆ alkynyl, wherein alkyl, alkenyl, and alkynyl are optionally substituted with one or more substituents independently selected from halo, —OMe, —CN, —NH₂, and —NO₂;
R⁸ is —CR′₂—, wherein each R′ is independently selected from hydrogen, halo, haloalkyl, alkoxy, haloalkoxy, and amine;
n is selected from 2, 3, and 4;
R⁹ is selected from C₂-C₆ alkyl, C₂-C₆ alkenyl, and C₂-C₆ alkynyl, wherein alkyl, alkenyl, and alkynyl are optionally substituted with one or more substituents independently selected from halo, —OMe, —CN, —NH₂, —NO₂, and 3- to 8-membered heterocycle, and wherein 3- to 8-membered heterocycle is optionally substituted with one or more substituents independently selected from halo, —OMe, —CN, —NH₂, and —NO₂;
R² is selected from hydrogen, halogen, C₁-C₆ alkyl, and C₁-C₆ haloalkyl;
R⁴, R⁵, R⁶, and R⁷ are each independently selected from hydrogen, C₁-C₆ alkyl, A, J, Q, and X;
A is 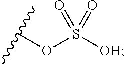 [SMILES: *OS(=O)(=O)O]
J is 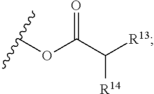
Q is 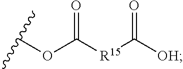
X is selected from glucose, xylose, galactose, rhamnose, rutinose, and disaccharide;
R¹³ is selected from hydrogen, and C₁-C₆ alkyl optionally substituted with one or more substituents independently selected from C₁-C₆ alkyl, oxo, halo, —OMe, —CN, —NH₂, and —NO₂;
R¹⁴ is selected from C₁-C₆ alkyl and C₂-C₆ alkenyl, wherein C₁-C₆ alkyl and C₂-C₆ alkenyl are optionally substituted with one or more substituents independently selected from halo, —OMe, —CN, —NH₂, and —NO₂;
or R¹³ and R¹⁴ taken together with the atom to which they are attached to form a substituted or unsubstituted C₃-C₈cycloalkyl or substituted or unsubstituted 3- to 8-membered heterocycloalkyl having 1 or 2 heteroatoms each independently selected from N, O, and S;
R¹⁵ is selected from C₁-C₆ alkylene and C₂-C₆ alkenylene, wherein C₁-C₆ alkylene and C₂-C₆ alkenylene are optionally substituted with one or more substituents independently selected from C₁-C₆ alkyl, halo, —OMe, —CN, —NH₂, and —NO₂; and
wherein at least one of R⁴, R⁵, R⁶, and R⁷ is A, J, Q, or X.

2. The compound or salt of claim 1, wherein R¹ is selected from hydrogen and C₁-C₃ alkyl.

3. The compound or salt of claim 1 or 2, wherein R¹ is hydrogen.

4. The compound or salt of any of claims 1 to 3, wherein R¹⁰ is independently selected from hydrogen, C₁-C₃ alkyl, and C₂-C₃ alkenyl, wherein alkyl and alkenyl are optionally substituted with one or more substituents independently selected from halo, —OMe, and —CN.

5. The compound or salt of any of claims 1 to 4, wherein each R′ is independently selected from hydrogen, halo, and haloalkyl.

6. The compound or salt of any of claims 1 to 5, wherein each R′ is hydrogen.

7. The compound or salt of any of claims 1 to 6, wherein n is selected from 2 and 3.

8. The compound or salt of any of claims 1 to 7, wherein n is 2.

9. The compound or salt of any of claims 1 to 8, wherein R⁹ is selected from C₂-C₃ alkyl and C₂-C₃ alkenyl, wherein alkyl and alkenyl are optionally substituted with one or more substituents independently selected from halo, —OMe, and —CN.

10. The compound or salt of any of claims 1 to 9, wherein R⁹ is C₂-C₃ alkyl.

11. The compound or salt of any of claims 1 to 10, wherein R² is selected from hydrogen, halogen, and C₁-C₆ alkyl.

12. The compound or salt of any of claims 1 to 11, wherein R² is hydrogen.

13. The compound or salt of any of claims 1 to 12, wherein R⁴, R⁵, R⁶, and R⁷ are each independently selected from hydrogen, A, J, Q, and X.

14. The compound or salt of any of claims 1 to 13, wherein R⁴, R⁵, R⁶, and R⁷ are each independently selected from hydrogen, J, and Q.

15. The compound or salt of any of claims 1 to 14, wherein at least one of R⁴, R⁵, R⁶, and R⁷ is A or Q.

16. The compound or salt of any of claims 1 to 14, wherein at least one of R⁵, R⁶, and R⁷ is J or X.

17. The compound or salt of any of claims 1 to 16, wherein R¹³ is selected from hydrogen and C₁-C₃ alkyl optionally substituted with one or more substituents independently selected from C₁-C₆ alkyl, oxo, halo, —OMe, and —CN.

18. The compound or salt of any of claims 1 to 17, wherein R¹³ is hydrogen.

19. The compound or salt of any of claims 1 to 17, wherein R¹³ is C₁-C₃ alkyl.

20. The compound or salt of any of claims 1 to 19, wherein R¹⁴ is C₁-C₃ alkyl optionally substituted with one or more substituents independently selected from halo, —OMe, and —CN.

21. The compound or salt of any of claims 1 to 20, wherein R¹⁵ is C₁-C₃ alkylene optionally substituted with one or more substituents independently selected from C₁-C₆ alkyl, oxo, halo, —OMe, and —CN.

22. The compound or salt of any of claims 1 to 21, wherein X is selected from glucose, galactose, and rhamnose.

23. A method of treating a disease or disorder in a subject in need thereof comprising administering a compound of Formula (Ia): 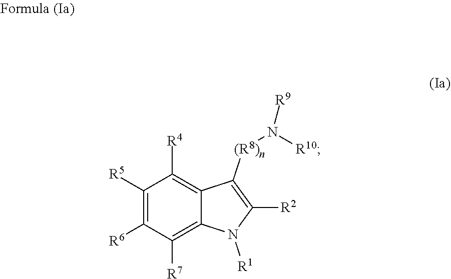
or a pharmaceutically acceptable salt thereof, wherein,
R¹ and R¹⁰ are independently selected from hydrogen, C₁-C₆ alkyl, C₂-C₆ alkenyl, and C₂-C₆ alkynyl, wherein alkyl, alkenyl, and alkynyl are optionally substituted with one or more substituents independently selected from halo, —OMe, —CN, —NH₂, and —NO₂;
R⁸ is —CR′₂—, wherein each R′ is independently selected from hydrogen, halo, haloalkyl, alkoxy, haloalkoxy, and amine;
n is selected from 2, 3, and 4;
R⁹ is selected from C₂-C₆ alkyl, C₂-C₆ alkenyl, and C₂-C₆ alkynyl, wherein alkyl, alkenyl, and alkynyl are optionally substituted with one or more substituents independently selected from halo, —OMe, —CN, —NH₂, —NO₂, and 3- to 8-membered heterocycle, and wherein 3- to 8-membered heterocycle is optionally substituted with one or more substituents independently selected from halo, —OMe, —CN, —NH₂, and —NO₂;
R² is selected from hydrogen, halogen, C₁-C₆ alkyl, and C₁-C₆ haloalkyl;
R⁴, R⁵, R⁶, and R⁷ are each independently selected from hydrogen, C₁-C₆ alkyl, A, J, Q, and X;
A is 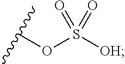 [SMILES: *OS(=O)(=O)O]
J is 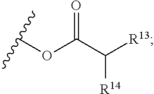
Q is 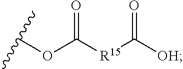
X is selected from glucose, xylose, galactose, rhamnose, rutinose, and disaccharide;
R¹³ is selected from hydrogen, and C₁-C₆ alkyl optionally substituted with one or more substituents independently selected from C₁-C₆ alkyl, oxo, halo, —OMe, —CN, —NH₂, and —NO₂;
R¹⁴ is selected from C₁-C₆ alkyl and C₂-C₆ alkenyl, wherein C₁-C₆ alkyl and C₂-C₆ alkenyl are optionally substituted with one or more substituents independently selected from halo, —OMe, —CN, —NH₂, and —NO₂;
or R¹³ and R¹⁴ taken together with the atom to which they are attached to form a substituted or unsubstituted C₃-C₈cycloalkyl or substituted or unsubstituted 3- to 8-membered heterocycloalkyl having 1 or 2 heteroatoms each independently selected from N, O, and S;
R¹⁵ is selected from C₁-C₆ alkylene and C₂-C₆ alkenylene, wherein C₁-C₆ alkylene and C₂-C₆ alkenylene are optionally substituted with one or more substituents independently selected from C₁-C₆ alkyl, halo, —OMe, —CN, —NH₂, and —NO₂; and
wherein at least one of R⁴, R⁵, R⁶, and R₇ is A, J, Q, or X.

24. The method of claim 23, wherein R¹ is selected from hydrogen and C₁-C₃ alkyl.

25. The method of claim 23 or 24, wherein R¹ is hydrogen.

26. The method of any of claims 23 to 25, wherein R¹⁰ is independently selected from hydrogen, C₁-C₃ alkyl, and C₂-C₃ alkenyl, wherein alkyl and alkenyl are optionally substituted with one or more substituents independently selected from halo, —OMe, and —CN.

27. The method of any of claims 23 to 26, wherein each R′ is independently selected from hydrogen, halo, and haloalkyl.

28. The method of any of claims 23 to 27, wherein each R′ is hydrogen.

29. The method of any of claims 23 to 28, wherein n is selected from 2 and 3.

30. The method of any of claims 23 to 29, wherein n is 2.

31. The method of any of claims 23 to 30, wherein R⁹ is selected from C₂-C₃ alkyl and C₂-C₃ alkenyl, wherein alkyl and alkenyl are optionally substituted with one or more substituents independently selected from halo, —OMe, and —CN.

32. The method of any of claims 23 to 31, wherein R⁹ is C₂-C₃ alkyl.

33. The method of any of claims 23 to 32, wherein R² is selected from hydrogen, halogen, and C₁-C₆ alkyl.

34. The method of any of claims 23 to 33, wherein R² is hydrogen.

35. The method of any of claims 23 to 34, wherein R⁴, R⁵, R⁶, and R⁷ are independently selected from hydrogen, A, J, Q, and X.

36. The method of any of claims 23 to 35, wherein R⁴, R⁵, R⁶, and R⁷ are independently selected from hydrogen, J, and Q.

37. The method of any of claims 23 to 36, wherein at least one of R⁴, R⁵, R⁶, and R⁷ is A or Q.

38. The method of any of claims 23 to 36, wherein at least one of R⁵, R⁶, and R⁷ is J or X.

39. The method of any of claims 23 to 38, wherein R¹³ is selected from hydrogen and C₁-C₃ alkyl optionally substituted with one or more substituents independently selected from C₁-C₆ alkyl, oxo, halo, —OMe, and —CN.

40. The method of any of claims 23 to 39, wherein R¹³ is hydrogen.

41. The method of any of claims 23 to 39, wherein R¹³ is C₁-C₃ alkyl.

42. The method of any of claims 23 to 41, wherein R¹⁴ is C₁-C₃ alkyl optionally substituted with one or more substituents independently selected from halo, —OMe, and —CN.

43. The method of any of claims 23 to 42, wherein R¹⁵ is C₁-C₃ alkylene optionally substituted with one or more substituents independently selected from C₁-C₆ alkyl, oxo, halo, —OMe, and —CN.

44. The method of any of claims 23 to 43, wherein X is selected from glucose, galactose, and rhamnose.

45. The method of any of claims 23 to 44, wherein the disease or disorder is major depression, treatment resistant depression, addiction, anxiety, post-traumatic stress disorder, prolonged grief disorder, complicated grief disorder, mania, psychosis, insomnia, hypersomnia, pain, Alzheimers disease, Parkinson's disease, burnout, cluster headaches, binge eating, migraine headaches, or irritable bowel syndrome.

46. The method of any of claims 23 to 45, wherein the disease or disorder is major depression, treatment resistant depression, addiction, anxiety, post-traumatic stress disorder, prolonged grief disorder, complicated grief disorder, or binge eating.

47. A method of treating a disease or disorder in a subject in need thereof comprising administering a modified indole alkaloid.

48. The method of claim 47, wherein the modified indole alkaloid is a modified tryptamine alkaloid, a modified ibogamine alkaloid, a modified ergoline alkaloid, a modified beta-carboline alkaloid, or a modified mitragynine alkaloid.

49. The method of claim 47 or 48, wherein the modified indole alkaloid is an acetylated indole alkaloid, an acylated indole alkaloid, a methylated indole alkaloid, a phosphorylated indole alkaloid, a sulfonylated indole alkaloid, or a glycosylated indole alkaloid.

50. A method of enzymatically preparing an indole alkaloid, comprising:
contacting a compound of Formula (Ia′) with an enzyme and a co-substrate;
wherein the compound of Formula (Ia′) has a structure of: 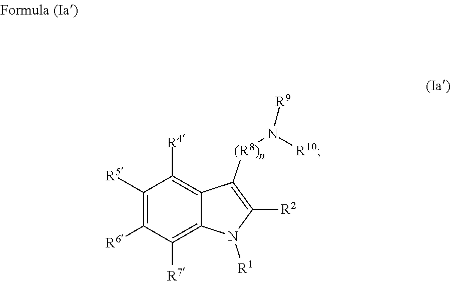
or a pharmaceutically acceptable salt thereof, wherein,
R¹ and R¹⁰ are independently selected from hydrogen, C₁-C₆ alkyl, C₂-C₆ alkenyl, and C₂-C₆ alkynyl, wherein alkyl, alkenyl, and alkynyl are optionally substituted with one or more substituents independently selected from halo, —OMe, —CN, —NH₂, and —NO₂;
R⁸ is —CR′₂—, wherein each R′ is independently selected from hydrogen, halo, haloalkyl, alkoxy, haloalkoxy, and amine;
n is selected from 2, 3, and 4;
R⁹ is selected from C₂-C₆ alkyl, C₂-C₆ alkenyl, and C₂-C₆ alkynyl, wherein alkyl, alkenyl, and alkynyl are optionally substituted with one or more substituents independently selected from halo, —OMe, —CN, —NH₂, —NO₂, and 3- to 8-membered heterocycle, and wherein 3- to 8-membered heterocycle is optionally substituted with one or more substituents independently selected from halo, —OMe, —CN, —NH₂, and —NO₂;
R² is selected from hydrogen, halogen, C₁-C₆ alkyl, and C₁-C₆ haloalkyl;
R⁴, R⁵, R⁶, and R⁷ are each independently selected from hydrogen, —OH, and C₁-C₆ alkyl; and
wherein at least one of R⁴, R⁵, R⁶, and R⁷ is —OH.

51. The method of claim 50, wherein R⁴ is —OH.

52. The method of claim 50, wherein R⁵ is —OH.

53. The method of claim 50, wherein R⁶ is —OH.

54. The method of claim 50, wherein R⁷ is —OH.

55. The method of any one of claims 50-54, wherein the enzyme is a 4-hydroxytryptamine kinase.

56. The method of any one of claims 50-54, wherein the enzyme is an acetylserotonin O-methyltransferase.

57. The method of any one of claims 50-54, wherein the enzyme is a tryptamine n-methyltransferase.

58. The method of any one of claims 50-54, wherein the enzyme is a sulfotransferase 1A1.

59. The method of any one of claims 50-54, wherein the enzyme is a sulfotransferase 1A3.

60. The method of any one of claims 50-54, wherein the enzyme is an alcohol O-acetyltransferase 1.

61. The method of any one of claims 50-54, wherein the enzyme is a chloramphenicol acetyltransferase.

62. The method of any one of claims 50-54, wherein the enzyme is an UDP-glucuronosyltransferase.

63. The method of any one of claims 50-54, wherein the enzyme is an oleandomycin glycosyltransferase.

64. The method of any one of claims 50-54, wherein the enzyme is a glycosyltransferase.

65. The method of any one of claims 50-54, wherein the enzyme is a 4-dimethylallyl tryptophan synthase.

66. The method of any one of claims 50-54, wherein the enzyme is a 7-dimethylallyltryptophan synthase.


SEQUENCE LISTING (28 sequences)

SEQ ID NO: 1 - protein, 362 aa, Psilocybe cyanescens
MAFDLKTPEGLLLYLTRHLSLDVDPSGVKRLSGGFVNVTWRIRLNAPYQGHTSIILKHAQPHLSSDEDFKIGVERSAYEYQALKVMSANQEVLGGDDSRVSVPEGLHYDVENNALIMQDVGTMKTLLDYATAKPPLSTEIASLVGTEIGAFIARLHNLGRKRRDQPAFKFFSGNIVGRTTADQLYQTIIPNAAKYGINDPLLPTVVKDLVGEVMNSEETLIMADLWSGNILLEFVEGNPSELKKIWLVDWELCKYGPASLDMGYFLGDCYLIARFQDELVGTTMRKAYLKGYARTAKGTINYSKVTASIGAHLVMWTDFMKWGNYEEREEFVKKGVEALHDAWEDNNDGEITSVLVNEASST

SEQ ID NO: 2 - protein, 362 aa, Psilocybe cubensis
MAFDLKTEDGLITYLTKHLSLDVDTSGVKRLSGGFVNVTWRIKLNAPYQGHTSIILKHAQPHMSTDEDFKIGVERSVYEYQAIKLMMANREVLGGVDGIVSVPEGLNYDLENNALIMQDVGKMKTLLDYVTAKPPLATDIARLVGTEIGGFVARLHNIGRERRDDPEFKFFSGNIVGRTTSDQLYQTIIPNAAKYGVDDPLLPTVVKDLVDDVMHSEETLVMADLWSGNILLQLEEGNPSKLQKIYILDWELCKYGPASLDLGYFLGDCYLISRFQDEQVGTTMRQAYLQSYARTSKHSINYAKVTAGIAAHIVMWTDFMQWGSEEERINFVKKGVAAFHDARGNNDNGEITSTLLKESSTA

SEQ ID NO: 3 - protein, 345 aa, Homo sapiens
MGSSEDQAYRLLNDYANGFMVSQVLFAACELGVFDLLAEAPGPLDVAAVAAGVRASAHGTELLLDICVSLKLLKVETRGGKAFYRNTELSSDYLTTVSPTSQCSMLKYMGRTSYRCWGHLADAVREGRNQYLETFGVPAEELFTAIYRSEGERLQFMQALQEVWSVNGRSVLTAFDLSVFPLMCDLGGGAGALAKECMSLYPGCKITVFDIPEVVWTAKQHFSFQEEEQIDFQEGDFFKDPLPEADLYILARVLHDWADGKCSHLLERIYHTCKPGGGILVIESLLDEDRRGPLLTQLYSLNMLVQTEGQERTPTHYHMLLSSAGFRDFQFKKTGAIYDAILARK

SEQ ID NO: 4 - protein, 317 aa, Panaeolus cyanescens
MHNRNPYRDVIDYQALAEAYPPLKPHVTVNADNTASIDLTIPEVQRQYTAALLHRDFGLTITLPEDRLCPTVPNRLNYVLWIEDIFQCTNKALGLSDDRPVKGVDIGTGASAIYPMLACARFKQWSMIATEVERKCIDTARLNVLANNLQDRLSILEVSVDGPILVPIFDTFERATSDYEFEFTMCNPPFYDGAADMQTSDAAKGFGFGVNAPHSGTVIEMATEGGEAAFVAQMVRESMKLQTRCRWFTSNLGKLKSLHEIVALLRESQITNYAINEYVQGTTRRYALAWSFTDIKLTEELYRPSNPELGPLCSTFV

SEQ ID NO: 5 - protein, 295 aa, Homo sapiens
MELIQDTSRPPLEYVKGVPLIKYFAEALGPLQSFQARPDDLLISTYPKSGTTWVSQILDMIYQGGDLEKCHRAPIFMRVPFLEFKAPGIPSGMETLKDTPAPRLLKTHLPLALLPQTLLDQKVKVVYVARNAKDVAVSYYHFYHMAKVHPEPGTWDSFLEKFMVGEVSYGSWYQHVQEWWELSRTHPVLYLFYEDMKENPKREIQKILEFVGRSLPEETVDFVVQHTSFKEMKKNPMTNYTTVPQEFMDHSISPFMRKGMAGDWKTTFTVAQNERFDADYAEKMAGCSLSFRSEL

SEQ ID NO: 6 - protein, 295 aa, Homo sapiens
MELIQDTSRPPLEYVKGVPLIKYFAEALGPLQSFQARPDDLLINTYPKSGTTWVSQILDMIYQGGDLEKCNRAPIYVRVPFLEVNDPGEPSGLETLKDTPPPRLIKSHLPLALLPQTLLDQKVKVVYVARNPKDVAVSYYHFHRMEKAHPEPGTWDSFLEKFMAGEVSYGSWYQHVQEWWELSRTHPVLYLFYEDMKENPKREIQKILEFVGRSLPEETMDFMVQHTSFKEMKKNPMTNYTTVPQELMDHSISPFMRKGMAGDWKTTFTVAQNERFDADYAEKMAGCSLSFRSEL

SEQ ID NO: 7 - protein, 525 aa, Saccharomyces cerevisiae
MNEIDEKNQAPVQQECLKEMIQNGHARRMGSVEDLYVALNRQNLYRNFCTYGELSDYCTRDQLTLALREICLKNPTLLHIVLPTRWPNHENYYRSSEYYSRPHPVHDYISVLQELKLSGVVLNEQPEYSAVMKQILEEFKNSKGSYTAKIFKLTTTLTIPYFGPTGPSWRLICLPEEHTEKWKKFIFVSNHCMSDGRSSIHFFHDLRDELNNIKTPPKKLDYIFKYEEDYQLLRKLPEPIEKVIDFRPPYLFIPKSLLSGFIYNHLRFSSKGVCMRMDDVEKTDDVVTEIINISPTEFQAIKANIKSNIQGKCTITPFLHVCWFVSLHKWGKFFKPLNFEWLTDIFIPADCRSQLPDDDEMRQMYRYGANVGFIDFTPWISEFDMNDNKENFWPLIEHYHEVISEALRNKKHLHGLGFNIQGFVQKYVNIDKVMCDRAIGKRRGGTLLSNVGLFNQLEEPDAKYSICDLAFGQFQGSWHQAFSLGVCSTNVKGMNIVVASTKNVVGSQESLEELCSIYKALLLGP

SEQ ID NO: 8 - protein, 212 aa, Pseudomonas aeruginosa
MGNYFESPFRGKLLSEQVSNPNIRVGRYSYYSGYYHGHSFDDCARYLMPDRDDVDKLVIGSFCSIGSGAAFIMAGNQGHRAEWASTFPFHFMHEEPVFAGAVNGYQPAGDTLIGHDVWIGTEAMFMPGVRVGHGAIIGSRALVTGDVEPYAIVGGNPARTIRKRFSDGDIQNLLEMAWWDWPLADIEAAMPLLCTGDIPALYRHWKQRQATA

SEQ ID NO: 9 - protein, 532 aa, Homo sapiens
MACLLRSFQRISAGVFFLALWGMVVGDKLLVVPQDGSHWLSMKDIVEVLSDRGHEIVVVVPEVNLLLKESKYYTRKIYPVPYDQEELKNRYQSFGNNHFAERSFLTAPQTEYRNNMIVIGLYFINCQSLLQDRDTLNFFKESKFDALFTDPALPCGVILAEYLGLPSVYLFRGFPCSLEHTFSRSPDPVSYIPRCYTKFSDHMTFSQRVANFLVNLLEPYLFYCLFSKYEELASAVLKRDVDIITLYQKVSVWLLRYDFVLEYPRPVMPNMVFIGGINCKKRKDLSQEFEAYINASGEHGIVVFSLGSMVSEIPEKKAMAIADALGKIPQTVLWRYTGTRPSNLANNTILVKWLPQNDLLGHPMTRAFITHAGSHGVYESICNGVPMVMMPLFGDQMDNAKRMETKGAGVTLNVLEMTSEDLENALKAVINDKSYKENIMRLSSLHKDRPVEPLDLAVFWVEFVMRHKGAPHLRPAAHDLTWYQYHSLDVIGFLLAVVLTVAFITFKCCAYGYRKCLGKKGRVKKAHKSKTH

SEQ ID NO: 10 - protein, 430 aa, Streptomyces antibioticus
MTTQTTPAHIAMFSIAAHGHVNPSLEVIRELVARGHRVTYAIPPVFADKVAATGPRPVLYHSTLPGPDADPEAWGSTLLDNRRTFLNDAIQALPQLADAYADDIPDLVLHDITSYPARVLARRWGVPAVSLSPNLVAWKGYEEEVAEPMWREPRQTERGRAYYARFEAWLKENGITEHPDTFASHPPRSLVLIPKALQPHADRVDEDVYTFVGACQGDRAEEGGWQRPAGAEKVVLVSLGSAFTKQPAFYRECVRAFGNLPGWHLVLQIGRKVTPAELGELPDNVEVHDWVPQLAILRQADLFVTHAGAGGSQEGLATATPMIAVPQAVDQFGNADMLQGLGVARKLATEEATADLLRETALALVDDPEVARRLRRIQAEMAQEGGTRRAADLIEAELPARHERQEPVGDRPNVGDRPAGVRSDRQRSAL

SEQ ID NO: 11 - protein, 477 aa, Persicaria tinctoria
MESPAAPPTTAPPPHVIIMPSAGMGHLIPLAEFAKRLLPRFTFTFAVPTSGPPSSSQRDFLSSLPASIDTSFLPEVDLSDAPSDAQIETLMSLMVVRSLPSLRDLIASYSASGRRVAALVVDLFATDAIDVALELGIRPFIFFPSTAMTLSFFLHLEKLDETVSCEFAELSDPVQIPGCIPVHGKDLIDPVQDRKNDAYKWLLHHSKRYKLAEGVIVNSFEGLEGGPIRELLHPEPGKPRVYPVGPLIQAGSCEKGAAARPECLKWLDQQPRGSVLFVNFGSGGVLSTEQQNELAGVLAHSQQRFLWVVRPPNDGIANATYFSVDGEIDPLKLLPEGFLEQTAGRGLVLPMWAPQIDVLSHESTGGFLTHCGWNSTLESVFHGVPLITWPLYAEQKMNAVMLTEGLRVGLRPSVGKDGIIRGDEIARVIGELMEGEEGKRIRSKMQELKRAASAVLSKDGSSTRALEEVAKIWESKV

SEQ ID NO: 12 - protein, 530 aa, Homo sapiens
MACTGWTSPLPLCVCLLLTCGFAEAGKLLVVPMDGSHWFTMRSVVEKLILRGHEVVVVMPEVSWQLGRSLNCTVKTYSTSYTLEDLDREFKAFAHAQWKAQVRSIYSLLMGSYNDIFDLFFSNCRSLFKDKKLVEYLKESSFDAVFLDPFDNCGLIVAKYFSLPSVVFARGILCHYLEEGAQCPAPLSYVPRILLGFSDAMTFKERVRNHIMHLEEHLLCHRFFKNALEIASEILQTPVTEYDLYSHTSIWLLRTDFVLDYPKPVMPNMIFIGGINCHQGKPLPMEFEAYINASGEHGIVVFSLGSMVSEIPEKKAMAIADALGKIPQTVLWRYTGTRPSNLANNTILVKWLPQNDLLGHPMTRAFITHAGSHGVYESICNGVPMVMMPLFGDQMDNAKRMETKGAGVTLNVLEMTSEDLENALKAVINDKSYKENIMRLSSLHKDRPVEPLDLAVFWVEFVMRHKGAPHLRPAAHDLTWYQYHSLDVIGFLLAVVLTVAFITFKCCAYGYRKCLGKKGRVKKAHKSKTH

SEQ ID NO: 13 - protein, 530 aa, Homo sapiens
MARAGWTSPVPLCVCLLLTCGFAEAGKLLVVPMDGSHWFTMQSVVEKLILRGHEVVVVMPEVSWQLERSLNCTVKTYSTSYTLEDQNREFMVFAHAQWKAQAQSIFSLLMSSSSGFLDLFFSHCRSLFNDRKLVEYLKESSFDAVFLDPFDTCGLIVAKYFSLPSVVFTRGIFCHHLEEGAQCPAPLSYVPNDLLGFSDAMTFKERVWNHIVHLEDHLFCQYLFRNALEIASEILQTPVTAYDLYSHTSIWLLRTDFVLDYPKPVMPNMIFIGGINCHQGKPLPMEFEAYINASGEHGIVVFSLGSMVSEIPEKKAMAIADALGKIPQTVLWRYTGTRPSNLANNTILVKWLPQNDLLGHPMTRAFITHAGSHGVYESICNGVPMVMMPLFGDQMDNAKRMETKGAGVTLNVLEMTSEDLENALKAVINDKSYKENIMRLSSLHKDRPVEPLDLAVFWVEFVMRHKGAPHLRPAAHDLTWYQYHSLDVIGFLLAVVLTVAFITFKCCAYGYRKCLGKKGRVKKAHKSKTH

SEQ ID NO: 14 - protein, 448 aa, Claviceps purpurea
MSTAKDPGNGVYEILSLIFDFPSNEQRLWWHSTAPMFAAMLDNAGYNIHDQYRHLGIFKKHIIPFLGVYPTKDKERWLSILTRCGLPLELSLNCTDSVVRYTYEPINEVTGTEKDPFNTLAIMASVQKLAQIQAGIDLEWFSYFKDELTLDESESATLQSNELVKEQIKTQNKLALDLKESQFALKVYFYPHLKSIATGKSTHDLIFDSVFKLSQKHDSIQPAFQVLCDYVSRRNHSAESDQHIALHARLLSCDLIDPAKSRVKIYLLEKTVSLSVMEDLWTLGGQRVDASTMDGLDMLRELWSLLKVPTGHLEYPKGYLELGEIPNEQLPSMANYTLHHNNPMPEPQVYFTVFGMNDAEISNALTIFFQRHGFDDMAKKYRVFLQDSYPYHDFESLNYLHAYISFSYRRNKPYLSVYLHTFETGRWPVVADSPISFDAYRRCDLSTK

SEQ ID NO: 15 - protein, 472 aa, Neosartorya fumigata
MSIGAEIDSLVPAPPGLNGTAAGYPAKTQKELSNGDFDAHDGLSLAQLTPYDVLTAALPLPAPASSTGFWWRETGPVMSKLLAKANYPLYTHYKYLMLYHTHILPLLGPRPPLENSTHPSPSNAPWRSFLTDDFTPLEPSWNVNGNSEAQSTIRLGIEPIGFEAGAAADPFNQAAVTQFMHSYEATEVGATLTLFEHFRNDMFVGPETYAALRAKIPEGEHTTQSFLAFDLDAGRVTTKAYFFPILMSLKTGQSTTKVVSDSILHLALKSEVWGVQTIAAMSVMEAWIGSYGGAAKTEMISVDCVNEADSRIKIYVRMPHTSLRKVKEAYCLGGRLTDENTKEGLKLLDELWRTVFGIDDEDAELPQNSHRTAGTIFNFELRPGKWFPEPKVYLPVRHYCESDMQIASRLQTFFGRLGWHNMEKDYCKHLEDLFPHHPLSSSTGTHTFLSFSYKKQKGVYMTMYYNLRVYST

SEQ ID NO: 16 - DNA, 6626 bp, Artificial Sequence
  Description of Artificial Sequence Synthetic polynucleotide
gatgcgtccggcgtagaggatcgagatctcgatcccgcgaaattaatacgactcactataggggaattgtgagcggataacaattcccctctagaaataattttgtttaactttaagaaggagatataccatgggccatcaccatcaccatcacgactacgacatcccgactaccgaaaacctgtacttccagggttcttgagacctccctatcagtgatagagattgacatccctatcagtgatagagatactgagcacggatctgaaagaggagaaaggatctatggcgagtagcgaagacgttatcaaagagttcatgcgtttcaaagttcgtatggaaggttccgttaacggtcacgagttcgaaatcgaaggtgaaggtgaaggtcgtccgtacgaaggtacccagaccgctaaactgaaagttaccaaaggtggtccgctgccgttcgcttgggacatcctgtccccgcagttccagtacggttccaaagcttacgttaaacacccggctgacatcccggactacctgaaactgtccttcccggaaggtttcaaatgggaacgtgttatgaacttcgaagacggtggtgttgttaccgttacccaggactcctccctgcaagacggtgagttcatctacaaagttaaactgcgtggtaccaacttcccgtccgacggtccggttatgcagaaaaaaaccatgggttgggaagcttccaccgaacgtatgtacccggaagacggtgctctgaaaggtgaaatcaaaatgcgtctgaaactgaaagacggtggtcactacgacgctgaagttaaaaccacctacatggctaaaaaaccggttcagctgccgggtgcttacaaaaccgacatcaaactggacatcacctcccacaacgaagactacaccatcgttgaacagtacgaacgtgctgaaggtcgtcactccaccggtgcttaataaggatctccaggcatcaaataaaacgaaaggctcagtcgaaagactgggcctttcgttttatctgttgtttgtcggtgaacgctctctactagagtcacactggctcaccttcgggtgggcctttctgcgtttataggtctccatcctgatcaatccggctgctaacaaagcccgaaaggaagctgagttggctgctgccaccgctgagcaataactagcataaccccttggggcctctaaacgggtcttgaggggttttttgctgaaaggaggaactatatccggatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtcacgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccttctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgaaacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccac

SEQ ID NO: 17 - DNA, 6599 bp, Artificial Sequence
  Description of Artificial Sequence Synthetic polynucleotide
ttcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggctctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccatctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgaaacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatactatgtgagacctccctatcagtgatagagattgacatccctatcagtgatagagatactgagcacggatctgaaagaggagaaaggatctatggcgagtagcgaagacgttatcaaagagttcatgcgtttcaaagttcgtatggaaggttccgttaacggtcacgagttcgaaatcgaaggtgaaggtgaaggtcgtccgtacgaaggtacccagaccgctaaactgaaagttaccaaaggtggtccgctgccgttcgcttgggacatcctgtccccgcagttccagtacggttccaaagcttacgttaaacacccggctgacatcccggactacctgaaactgtccttcccggaaggtttcaaatgggaacgtgttatgaacttcgaagacggtggtgttgttaccgttacccaggactcctccctgcaagacggtgagttcatctacaaagttaaactgcgtggtaccaacttcccgtccgacggtccggttatgcagaaaaaaaccatgggttgggaagcttccaccgaacgtatgtacccggaagacggtgctctgaaaggtgaaatcaaaatgcgtctgaaactgaaagacggtggtcactacgacgctgaagttaaaaccacctacatggctaaaaaaccggttcagctgccgggtgcttacaaaaccgacatcaaactggacatcacctcccacaacgaagactacaccatcgttgaacagtacgaacgtgctgaaggtcgtcactccaccggtgcttaataaggatctccaggcatcaaataaaacgaaaggctcagtcgaaagactgggcctttcgttttatctgttgtttgtcggtgaacgctctctactagagtcacactggctcaccttcgggtgggcctttctgcgtttataggtctccatccgaaaacctgtacttccagggccatcaccatcaccatcactgatcaatccggctgctaacaaagcccgaaaggaagctgagttggctgctgccaccgctgagcaataactagcataaccccttggggcctctaaacgggtcttgaggggttttttgctgaaaggaggaactatatccggatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaa

SEQ ID NO: 18 - DNA, 6599 bp, Artificial Sequence; identical to SEQ ID NO: 17
  Description of Artificial Sequence Synthetic polynucleotide

SEQ ID NO: 19 - DNA, 6802 bp, Artificial Sequence
  Description of Artificial Sequence Synthetic polynucleotide
gcgttcgatctcaagactgaagacggcctcatcacatatctcactaaacatctttctttggacgtcgacacgagcggagtgaagcgccttagcggaggctttgtcaatgtaacctggcgcattaagctcaatgctccttatcaaggtcatacgagcatcatcctgaagcatgctcagccgcacatgtctacggatgaggattttaagataggtgtagaacgttcggtttacgaataccaggctatcaagctcatgatggccaatcgggaggttctgggaggcgtggatggcatagtttctgtgccagaaggcctgaactacgacttagagaataatgcattgatcatgcaagatgtcgggaagatgaagacccttttagattatgtcaccgccaaaccgccacttgcgacggatatagcccgccttgttgggacagaaattggggggttcgttgccagactccataacataggccgcgagaggcgagatgatcctgagttcaaattcttctctggaaatattgtcggaaggacgacttcagaccagctgtatcaaaccatcatacccaacgcagcgaaatatggcgtcgatgaccccttgctgcctactgtggttaaggaccttgtggacgatgtcatgcacagcgaagaaacccttgtcatggcggacctgtggagtggaaatattcttctccagttggaggagggaaacccatcgaagctgcagaagatatatatcctggattgggaactttgcaagtacggcccagcgtcgttggacctgggctatttcttgggtgactgctatttgatatcccgctttcaagacgagcaggtcggtacgacgatgcggcaagcctacttgcaaagctatgcgcgtacgagcaagcattcgatcaactacgccaaagtcactgcaggtattgctgctcatattgtgatgtggaccgactttatgcagtgggggagcgaggaagaaaggataaattttgtgaaaaagggggtagctgcctttcacgacgccaggggcaacaacgacaatggggaaattacgtctaccttactgaaggaatcatccactgcgggatcctgatcaatccggctgctaacaaagcccgaaaggaagctgagttggctgctgccaccgctgagcaataactagcataaccccttggggcctctaaacgggtcttgaggggttttttgctgaaaggaggaactatatccggatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtcacgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccttctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgaaacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgggccatcaccatcaccatcacgactacgacatcccgactaccgaaaacctgtacttccagggttct

SEQ ID NO: 20 - DNA, 6751 bp, Artificial Sequence
  Description of Artificial Sequence Synthetic polynucleotide
gggagctcagaagaccaggcctatcgtttattgaatgactacgctaatggcttcatggtttctcaagttcttttcgccgcctgtgagctgggggtgttcgacttgttggcggaagctcccggtccccttgatgttgctgcagtagcggcgggggtgcgcgcctcggctcacggaacggaactgttattggacatctgtgtcagcctgaaacttcttaaagtggaaacacgcggcggtaaagcattttaccgtaacactgagttgtcgtctgattatttgactacagtatcgccgacatctcagtgctccatgttaaagtatatgggccgcacatcatatcgctgttggggacatttagctgacgcggtacgcgaaggtcgcaatcagtatttggaaaccttcggggtgccagcggaagagttattcacggctatctaccgctcggagggtgaacgccttcaatttatgcaagcattacaagaagtctggtccgtaaatggtcgctcagtcctgactgcgttcgacctgtctgtgtttcccctgatgtgtgacctgggaggcggcgcaggggcacttgcaaaagaatgtatgtcactttatccggggtgtaaaattactgtgtttgatatccctgaagtggtctggaccgcgaaacaacatttcagcttccaagaggaagaacaaatcgacttccaggaaggcgacttttttaaagaccctttacccgaggctgatttatacatcttagctcgtgtgttgcacgactgggcggatggtaaatgctcgcacttacttgaacgcatttatcacacttgtaagcctgggggaggcatcttagtcattgaaagtttgctggacgaggaccgtcgcgggcctcttttgacccagttatattccttaaacatgttggtccagacagaaggccaagagcgtacgccaacacactatcacatgctgctgtcatcagccgggttccgcgactttcagttcaaaaagacaggcgccatctacgatgcaattttagcacgtaagggatcctgatcaatccggctgctaacaaagcccgaaaggaagctgagttggctgctgccaccgctgagcaataactagcataaccccttggggcctctaaacgggtcttgaggggttttttgctgaaaggaggaactatatccggatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtcacgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccttctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgaaacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgggccatcaccatcaccatcacgactacgacatcccgactaccgaaaacctgtacttccagggttct

SEQ ID NO: 21 - DNA, 6574 bp, Artificial Sequence
  Description of Artificial Sequence Synthetic polynucleotide
gaaaacctgtacttccagggccatcaccatcaccatcactgatcaatccggctgctaacaaagcccgaaaggaagctgagttggctgctgccaccgctgagcaataactagcataaccccttggggcctctaaacgggtcttgaggggttttttgctgaaaggaggaactatatccggatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggctctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccatctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgaaacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatactatggaattgattcaagacacttcaagaccaccattggaatacgttaagggtgtacctttgatcaagtatttcgcagaggcattaggtccattacagtcttttcaagcaagaccagatgatttgttaatttctacatatccaaaatctggaactacttgggtatctcagatattggatatgatctatcaaggaggagacttggaaaagtgtcatagagcacctatcttcatgagagtcccattcttggaatttaaagctccaggtattccttctggtatggaaactttaaaagatactccagctccaaggttgttgaaaacccatttgccattggctttattacctcaaacattattagatcagaaagttaaagtagtttatgttgctagaaatgcaaaagatgtagcagtttcatattaccacttctatcatatggctaaagttcaccctgaacctggtacatgggattcattcttagaaaagtttatggtaggagaggtttcatatggttcatggtatcagcacgtccaggaatggtgggaattatctaggacacatccagtcttgtacttgttttatgaagacatgaaagaaaaccctaaaagagagatccagaagattttggaatttgtcggtaggtctttaccagaggaaactgtcgattttgtcgttcaacacacatcatttaaggaaatgaagaagaacccaatgacaaactacacaactgttcctcaagaatttatggaccactctatatctccatttatgaggaaaggtatggccggtgattggaaaactacttttacagtagcacaaaacgaaagattcgatgccgattatgctgagaaaatggcaggatgctctttgtcattcaggtctgaattaggatcc

SEQ ID NO: 22 - DNA, 6574 bp, Artificial Sequence
  Description of Artificial Sequence Synthetic polynucleotide
gaaaacctgtacttccagggccatcaccatcaccatcactgatcaatccggctgctaacaaagcccgaaaggaagctgagttggctgctgccaccgctgagcaataactagcataaccccttggggcctctaaacgggtcttgaggggttttttgctgaaaggaggaactatatccggatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggctctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccatctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgaaacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatactatggaattgattcaagatacttcaagaccaccattggaatatgttaagggtgttcctttaattaagtactttgccgaagccttgggtccattgcaatcttttcaagctagacctgatgatttgttgatcaatacttatcctaaatctggtactacttgggtatcacagattttggacatgatttaccaaggtggtgatttggaaaagtgcaatagggcacctatatatgttagggttccattcttagaagttaacgacccaggagaaccttctggtttggaaacattgaaagataccccaccaccaagattaattaaatctcacttgccattggctttattgccacagactttattggaccaaaaggttaaggttgtttacgtcgccagaaatccaaaggacgtagcagtttcttattaccatttccatagaatggaaaaggcacacccagaaccaggtacatgggactcattcttggagaaattcatggctggagaagtttcttacggttcttggtatcagcacgtacaagaatggtgggaattatcaagaactcatccagtcttatatttattttatgaagatatgaaagaaaatcctaagagagaaatccagaaaatcttagagttcgtaggtaggtctttgccagaagaaacaatggatttcatggtacaacacacatcttttaaagagatgaagaagaatccaatgactaattacactactgttccacaagaattaatggaccactctatctctccttttatgaggaaaggtatggccggagattggaaaaccactttcactgttgctcagaatgagaggtttgatgccgattatgctgaaaagatggcaggttgttctttgtcttttagatcagaattgggatcc

SEQ ID NO: 23 - DNA, 6346 bp, Artificial Sequence
  Description of Artificial Sequence Synthetic polynucleotide
gaaaacctgtacttccagggccatcaccatcaccatcactgatcaatccggctgctaacaaagcccgaaaggaagctgagttggctgctgccaccgctgagcaataactagcataaccccttggggcctctaaacgggtcttgaggggttttttgctgaaaggaggaactatatccggatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggctctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccatctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgaaacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatactatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtttcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgggatcc

SEQ ID NO: 24 - DNA, 7285 bp, Artificial Sequence
  Description of Artificial Sequence Synthetic polynucleotide
gaaaacctgtacttccagggccatcaccatcaccatcactgatcaatccggctgctaacaaagcccgaaaggaagctgagttggctgctgccaccgctgagcaataactagcataaccccttggggcctctaaacgggtcttgaggggttttttgctgaaaggaggaactatatccggatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggctctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccatctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgaaacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatactatggcctgtttgttgagagcctttcaaagaatatctgccggtgtcttcttcttggctttgtggggtatggtcgtaggtgataaattattggtagttccacaagatggttcacactggttatctatgaaggatatcgttgaagtcttgtcagatagaggtcacgaaattgttgttgtagttcctgaagtcaatttattgttgaaggaatctaagtattatacaaggaaaatttatcctgtcccttacgaccaagaagagttgaagaatagatatcagtcttttggtaataatcacttcgctgagaggtctttcttgaccgctcctcaaactgaatacagaaacaacatgattgtaataggtttgtattttataaattgtcagtctttgttacaagatagggatactttgaacttctttaaagaatcaaaatttgatgcattgtttactgatccagctttaccttgcggtgtcatcttagccgaatacttaggtttaccatcagtttatttattcagaggtttcccatgttctttagagcacgcattttcatcatctcctgaccctgtatcatatatacctagatgctacacaaaattttcagatcacatgacattttctcagagggtcgccaatttcttggtcaacttgttagaaccttacttgttttactgtttgttctctaaatatgaagaattagcttcagcagtcttgaagagagatgtagatataataacattgtaccagaaagtttcagtctggttattgaggtacgattttgtattggagtacccaagacctgttatgcctaacatggttttcataggaggtataaattgtaagaaaagaaaagatttgtctcaagagtttgaggcatacattaacgcatctggagaacacggtatagtagttttctctttgggttcaatggtttcagagataccagaaaagaaagcaatggcaatcgcagatgctttaggtaaaataccacaaaccgtcttgtggagatatacaggtactaggccttctaacttggccaataacactattttagtcaaatggttgccacaaaacgatttgttgggtcatcctatgaccagagcctttattacacacgccggttcacacggagtatatgaatcaatctgtaacggtgttcctatggttatgatgccattgtttggtgatcaaatggataacgctaagaggatggaaacaaaaggtgctggtgtaactttaaatgttttagaaatgacttcagaagatttagaaaacgctttgaaggccgttattaatgacaagtcatataaagaaaatataatgagattgtcatctttacataaagatagacctgttgaacctttagacttggctgttttctgggtcgagtttgtcatgagacataaaggtgctcctcatttaaggccagctgcccatgatttgacctggtatcaataccattctttggacgtaattggtttcttattagccgtcgttttgacagttgcattcatcacttttaagtgttgcgcatacggttatagaaagtgcttaggtaagaaaggaagggttaagaaagctcacaaatctaagactcacggatcc

SEQ ID NO: 25 - DNA, 6931 bp, Artificial Sequence
  Description of Artificial Sequence Synthetic polynucleotide
gaaaacctgtacttccagggccatcaccatcaccatcactgatcaatccggctgctaacaaagcccgaaaggaagctgagttggctgctgccaccgctgagcaataactagcataaccccttggggcctctaaacgggtcttgaggggttttttgctgaaaggaggaactatatccggatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggctctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccatctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgaaacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatactatggctaccacccagaccactcccgcccacatcgccatgttctccatcgccgcccacggccatgtgaaccccagcctggaggtgatccgtgaactcgtcgcccgcggccaccgggtcacgtacgccattccgcccgtcttcgccgacaaggtggccgccaccggcgcccggcccgtcctctaccactccaccctgcccggcaccgacgccgacccggaggcatggggaagcaccctgctggacaacgtcgaaccgttcctgaacgacgcgatccaggcgctcccgcagctcgccgatgcctacgccgacgacatccccgatctcgtcctgcacgacatcacctcctacccggcccgcgtcctggcccgccgctggggcgtcccggcggtttccctcttcccgaacctcgtcgcctggaagggttacgaggaggaggtcgccgagccgatgtggcgcgaaccccggcagaccgagcgcggacgggcctactacgcccggttcgaggcatggctgaaggagaacgggatcaccgagcacccggacacgttcgccagtcatccgccgcgctccctggtgctcatcccgaaggcgctccagccgcacgccgaccgggtggacgaagacgtgtacaccttcgtcggcgcctgccagggagatcgcgccgaggaaggcggctggcagcggcccgccggcgcggagaaggtcgtcctggtgtcgctcggctcggtgttcaccaagcagcccgccttctaccgggagtgcgtgcgcgccttcgggaacctgcccggctggcacctcgtcctccagatcggccggaaggtgacccccgccgaactgggggagctgccggacaacgtggaggtgcacgactgggtgccgcagctcgcgatcctgcgccaggccgatctgttcgtcacccacgcgggcgccggcggcagccaggaggggctggccaccgcgacgcccatgatcgccgtaccgcaggccgtcgaccagttcggcaacgccgacatgctccaagggctcggcgtcgcccggaagctggcgaccgaggaggccaccgccgacctgctccgcgaaaccgccctcgctctggtggacgacccggaggtcgcgcgccggctccggcgaatccaggcggagatggcccaggagggcggcacccggcgggcggccgacctcatcgaggccgaactgcccgcgcgccacgagcggcaggagccggtgggcgaccgacccaacggatcc

SEQ ID NO: 26 - DNA, 7120 bp, Artificial Sequence
  Description of Artificial Sequence Synthetic polynucleotide
gaatcccccgccgctccaccaaccaccgctccaccaccgcacgtcataatcgtgccctccgccggcatgggccacctcatccccctcgccgagttcgccaagcgcctccttccgcgcttcaccttcaccttcgccgtacccaccagcggcccgccctcatcctcccagcgcgacttcctctcctccctccctgcctccatcgacacctccttcctccccgaggtcgacctctccgacgccccctccgacgcccaaatcgagactctcatgtccctcatggttgtccgctccctcccctcgctccgcgacctcattgcctcctactccgcctccggccgccgcgtcgccgccctcgtcgtcgaccttttcgccactgatgcaatcgacgtcgcccttgagctcggcatccgccctttcatcttcttcccctccaccgccatgaccctctccttcttcctccacctcgagaagcttgatgaaacggtgtcatgtgagtttgccgagctgtccgacccggttcagatccccgggtgtattccggtccacggcaaggatttgatcgacccggttcaggataggaagaacgacgcctacaagtggctcctccaccactccaagaggtataaattggccgagggtgttatcgtaaatagcttcgagggtttggaggggggaccgatcagggagcttttgcaccccgagccgggaaagccgcgggtttacccggtcggaccgctgattcaggccggttcgtgcgagaagggggcagctgcccggcctgagtgcttgaagtggttggaccagcagccacgtggatccgtcctattcgtgaatttcgggagtggtggggtcctcagtacggagcagcagaacgagcttgcaggtgtgctggcccacagccagcagaggttcctatgggtggttaggcctccaaacgacggcattgccaacgccacgtatttcagcgtcgacggggagatcgacccgttgaaactcctgcccgaggggttcttggagcagaccgcgggcaggggtttggtcttgccaatgtgggccccgcagatcgatgtcttaagccatgagtcgacgggcgggttcttgacgcattgtgggtggaattcaacactggagagcgtgttccatggggtaccactaattacatggcccctctatgcagagcaaaagatgaacgctgttatgctaaccgagggcctgagggtgggactcagaccctcagtgggtaaggatggaatcatccgaggtgctgagatcgcacgagttataggggagttgatggaaggtgaggaagggaaacgaatacggagtaagatgcaggagttgaagcgtgcggcttctgctgtattgagcaaagatggatcatctactcgagctcttgaagaggttgcaaaaatttgggaaagcaaggttggatccgaaaacctgtacttccagggccatcaccatcaccatcactgatcaatccggctgctaacaaagcccgaaaggaagctgagttggctgctgccaccgctgagcaataactagcataaccccttggggcctctaaacgggtcttgaggggttttttgctgaaaggaggaactatatccggatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggctctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccatctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgaaacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatactatg

SEQ ID NO: 27 - DNA, 7279 bp, Artificial Sequence
  Description of Artificial Sequence Synthetic polynucleotide
gcgtgtactggctggacttcgccgttgccgttgtgtgtgtgcttattgttaacttgcggattcgcggaagcgggcaaattgctggtggtcccgatggatggcagtcactggttcactatgcgttccgtggtagaaaaattaatcttgcgcggccacgaagtggtagtagtcatgcccgaagtatcgtggcaacttggacgctccttgaattgtacagtgaagacatatagcacgagctatactctggaagatttggatcgcgagtttaaggcgtttgcacacgctcaatggaaagctcaagtccgcagcatttattcgcttttgatgggatcctataacgacatcttcgacctttttttctcaaattgtcgttcattgtttaaagacaaaaaacttgtcgagtatctgaaggaatcctcgtttgatgcagtcttcctggacccctttgacaattgcgggttaattgtggctaaatacttttcgttgccgtcagtcgtgttcgctcgcggtatcttgtgtcactatctggaggaaggtgcccaatgccctgcaccgctttcttatgttcctcgcatcttgttgggcttctcggatgcgatgacgtttaaggagcgtgtgcgcaatcatatcatgcaccttgaagagcatctgttatgccaccgcttttttaaaaacgcgctggaaatcgcctccgagattttgcagactccagtaactgaatacgacctgtacagccacacatcaatctggttgcttcgcacggatttcgtcttagattacccgaaaccggtcatgcctaatatgattttcatcggtggaattaactgccatcaagggaagcctttgccgatggagtttgaggcatatattaatgcgtcaggggaacatgggattgtcgtcttcagtttaggctcaatggtttccgaaatcccagagaaaaaggccatggctattgctgacgcactgggaaaaatccctcaaactgtgttgtggcgctatacgggaacacgcccgagtaacttggctaacaatacgattcttgtcaagtggttaccacagaatgatcttcttggccatcccatgactcgtgcatttatcacacatgcgggatcccacggagtctatgagtccatctgtaatggagttcccatggtgatgatgcctctgttcggggatcaaatggataacgctaagcgtatggaaactaaaggcgctggagtgacattgaacgtacttgagatgacgagcgaggacttagaaaacgcacttaaagcagtcattaatgataaatcctataaagagaatatcatgcgtttaagttccttgcacaaggaccgtccagttgaacccttagatctggcagtattctgggtggaatttgtcatgcgccataaaggagcaccccacttacgccccgcggctcatgatttgacctggtaccagtaccacagtcttgacgtgatcgggtttttattggccgttgtgttgaccgttgcctttattacattcaagtgttgcgcgtatggataccgtaagtgtttgggcaagaaggggcgcgtaaaaaaggcgcacaaaagtaagacacacggatccgaaaacctgtacttccagggccatcaccatcaccatcactgatcaatccggctgctaacaaagcccgaaaggaagctgagttggctgctgccaccgctgagcaataactagcataaccccttggggcctctaaacgggtcttgaggggttttttgctgaaaggaggaactatatccggatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggctctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccatctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgaaacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatactatg

SEQ ID NO: 28 - protein, 6 aa, Artificial Sequence
  Description of Artificial Sequence Synthetic 6xHis tag
HHHHHH